SEQUENCE LISTING (32 sequences)

SEQ ID NO: 1 - DNA, 22 bp, synthetic construct
  misc_feature 1..22: note = Description of Artificial Sequence: Synthetic oligonucleotide
  source 1..22: mol_type = other DNA
gtgatgaagtcagacaaatcgc

SEQ ID NO: 2 - DNA, 21 bp, synthetic construct
  misc_feature 1..21: note = Description of Artificial Sequence: Synthetic oligonucleotide
  source 1..21: mol_type = other DNA
acagttgctggtgcatgtaga

SEQ ID NO: 3 - DNA, 21 bp, synthetic construct
  misc_feature 1..21: note = Description of Artificial Sequence: Synthetic oligonucleotide
  source 1..21: mol_type = other DNA
ccggtagcacaccttgtaatg

SEQ ID NO: 4 - DNA, 21 bp, synthetic construct
  misc_feature 1..21: note = Description of Artificial Sequence: Synthetic oligonucleotide
  source 1..21: mol_type = other DNA
ccttgtaatggtgttgaaggt

SEQ ID NO: 5 - DNA, 21 bp, synthetic construct
  misc_feature 1..21: note = Description of Artificial Sequence: Synthetic oligonucleotide
  source 1..21: mol_type = other DNA
aagctataacgcagcctgtaa

SEQ ID NO: 6 - DNA, 20 bp, synthetic construct
  misc_feature 1..20: note = Description of Artificial Sequence: Synthetic oligonucleotide
  source 1..20: mol_type = other DNA
tgacactactgatgctgtcc

SEQ ID NO: 7 - DNA, 21 bp, synthetic construct
  misc_feature 1..21: note = Description of Artificial Sequence: Synthetic oligonucleotide
  source 1..21: mol_type = other DNA
ctgtagaataaacacgccaag

SEQ ID NO: 8 - DNA, 21 bp, synthetic construct
  misc_feature 1..21: note = Description of Artificial Sequence: Synthetic oligonucleotide
  source 1..21: mol_type = other DNA
gctgcattcagttgaatcacc

SEQ ID NO: 9 - DNA, 20 bp, synthetic construct
  misc_feature 1..20: note = Description of Artificial Sequence: Synthetic oligonucleotide
  source 1..20: mol_type = other DNA
gacaaggcgttccaattaac

SEQ ID NO: 10 - DNA, 20 bp, synthetic construct
  misc_feature 1..20: note = Description of Artificial Sequence: Synthetic oligonucleotide
  source 1..20: mol_type = other DNA
gttagcaccatagggaagtc

SEQ ID NO: 11 - DNA, 20 bp, synthetic construct
  misc_feature 1..20: note = Description of Artificial Sequence: Synthetic oligonucleotide
  source 1..20: mol_type = other DNA
ccgcattacgtttggtggac

SEQ ID NO: 12 - DNA, 20 bp, synthetic construct
  misc_feature 1..20: note = Description of Artificial Sequence: Synthetic oligonucleotide
  source 1..20: mol_type = other DNA
tgcaacccatatgatgccgt

SEQ ID NO: 13 - DNA, 20 bp, synthetic construct
  misc_feature 1..20: note = Description of Artificial Sequence: Synthetic oligonucleotide
  source 1..20: mol_type = other DNA
tgtccttccctcagtcagca

SEQ ID NO: 14 - DNA, 20 bp, synthetic construct
  misc_feature 1..20: note = Description of Artificial Sequence: Synthetic oligonucleotide
  source 1..20: mol_type = other DNA
aacgccaccagatttgcatc

SEQ ID NO: 15 - DNA, 20 bp, synthetic construct
  misc_feature 1..20: note = Description of Artificial Sequence: Synthetic oligonucleotide
  source 1..20: mol_type = other DNA
tgttgacatgttcagcccct

SEQ ID NO: 16 - DNA, 20 bp, synthetic construct
  misc_feature 1..20: note = Description of Artificial Sequence: Synthetic oligonucleotide
  source 1..20: mol_type = other DNA
gtacccgctaacagtgcaga

SEQ ID NO: 17 - DNA, 20 bp, synthetic construct
  misc_feature 1..20: note = Description of Artificial Sequence: Synthetic oligonucleotide
  source 1..20: mol_type = other DNA
ttggtgcaggtatatgcgct

SEQ ID NO: 18 - DNA, 20 bp, synthetic construct
  misc_feature 1..20: note = Description of Artificial Sequence: Synthetic oligonucleotide
  source 1..20: mol_type = other DNA
acggccttactgttttgcca

SEQ ID NO: 19 - DNA, 20 bp, synthetic construct
  misc_feature 1..20: note = Description of Artificial Sequence: Synthetic oligonucleotide
  source 1..20: mol_type = other DNA
tgtggatcacggacagcatc

SEQ ID NO: 20 - DNA, 20 bp, synthetic construct
  misc_feature 1..20: note = Description of Artificial Sequence: Synthetic oligonucleotide
  source 1..20: mol_type = other DNA
gtccgtgatccacagacact

SEQ ID NO: 21 - DNA, 20 bp, synthetic construct
  misc_feature 1..20: note = Description of Artificial Sequence: Synthetic oligonucleotide
  source 1..20: mol_type = other DNA
acagggacttctgtgcagtt

SEQ ID NO: 22 - DNA, 20 bp, synthetic construct
  misc_feature 1..20: note = Description of Artificial Sequence: Synthetic oligonucleotide
  source 1..20: mol_type = other DNA
gcctcggcaaaaacgtactg

SEQ ID NO: 23 - DNA, 20 bp, synthetic construct
  misc_feature 1..20: note = Description of Artificial Sequence: Synthetic oligonucleotide
  source 1..20: mol_type = other DNA
ctctgcggtaaggcttgagt

SEQ ID NO: 24 - DNA, 20 bp, synthetic construct
  misc_feature 1..20: note = Description of Artificial Sequence: Synthetic oligonucleotide
  source 1..20: mol_type = other DNA
tggttgacctacacaggtgc

SEQ ID NO: 25 - DNA, 20 bp, synthetic construct
  misc_feature 1..20: note = Description of Artificial Sequence: Synthetic oligonucleotide
  source 1..20: mol_type = other DNA
tgagtcagcactgctcatgg

SEQ ID NO: 26 - DNA, 21 bp, synthetic construct
  misc_feature 1..21: note = Description of Artificial Sequence: Synthetic oligonucleotide
  source 1..21: mol_type = other DNA
acagcaactggtcatacagca

SEQ ID NO: 27 - DNA, 21 bp, synthetic construct
  misc_feature 1..21: note = Description of Artificial Sequence: Synthetic oligonucleotide
  source 1..21: mol_type = other DNA
gattcgaagacccagtcccta

SEQ ID NO: 28 - DNA, 20 bp, synthetic construct
  misc_feature 1..20: note = Description of Artificial Sequence: Synthetic oligonucleotide
  source 1..20: mol_type = other DNA
ctaaagccgaaaaaccctga

SEQ ID NO: 29 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 30 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 31 - DNA, 3822 bp, Severe acute respiratory syndrome coronavirus 2
  source 1..3822: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 32 - DNA, 1260 bp, Severe acute respiratory syndrome coronavirus 2
  source 1..1260: mol_type = genomic DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

(19) United States
(12) Patent Application Publication
(10) Pub. No.: US 2024/0150855 A1
(43) Pub. Date: May 9, 2024

(54) METHODS AND COMPOSITIONS FOR IDENTIFYING VIRAL SEQUENCES

(71) Applicant: DECODE CURE LIMITED, Sha Tin (CN)

(72) Inventors: Bin YE, South Weymouth, MA (US); Ka Ming WONG, Tseung Kwan O (CN)

(21) Appl. No.: 18/506,464

(22) Filed: Nov. 10, 2023

Related U.S. Application Data
(63) Continuation of application No. PCT/CN2022/098195, filed on Jun. 10, 2022.
(60) Provisional application No. 63/209,175, filed on Jun. 10, 2021.

(51) Int. Cl. C12Q 1/70; C12Q 1/686

(52) U.S. Cl. CPC C12Q 1/701; C12Q 1/686; G01N 2333/165

(57) ABSTRACT

Provided herein are methods for identifying sequences of viruses. Also provided are compositions for carrying out the same. Compositions provided herein may comprise, consist essentially of, or consist of a plurality of primers. Furthermore, the disclosure provides kits comprising the compositions. Kits provided herein may also comprise oligonucleotides, probes, and solid supports.

### CROSS REFERENCE

This application is a continuation of International Patent Application PCT/CN2022/098195, filed on Jun. 10, 2022, which claims the benefit of U.S. Provisional Patent Application No. 63/209,175, filed on Jun. 10, 2021, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Sep. 20, 2022, is named 60617-701.301_SL.xml and is 46,600 bytes in size.

### BACKGROUND

Severe acute respiratory syndrome coronavirus 2 (SARS-CoV-2), or COVID-19 virus, has caused massive spread and catastrophic consequence globally. The virus has a markedly delayed timeframe (3-21 days) for impact and can infect people within 15 seconds. Emergence of COVID-19 variants imposes a huge health risk to people. Recently reported variants can cause more severe disease conditions and be much deadlier than previously circulating strains. For example, a variant with a 7-fold higher infection rate was first identified in the United Kingdom and has rapidly spread worldwide. Other evolving variants, such as those recently found in South Africa, Brazil and United States, contain different genetic mutations and are also highly contagious. Double and triple genetic variants have also been found, which have caused deadly outbreak in India. Some COVID-19 variants can exhibit longer incubation periods, can infect younger populations, and may not covered by mRNA-based COVID-19 vaccines.

Rapid and accurate detection of the variants can curb the pandemic and spread of the disease. Currently available methodologies, however, are limited due to the lack of speed, effectiveness, reliability, or scalability.

### BRIEF SUMMARY

Disclosed herein, are methods for identifying sequences of a virus of family Coronaviridae in a sample. In an aspect, a method for identifying sequences of a virus of family Coronaviridae in a sample comprises: a) providing substrate nucleic acids, wherein the substrate nucleic acids are amplification products of a plurality of template nucleotide sequences in the sample from the subject, or amplification products of complement sequences of the plurality of template nucleotide sequences; and b) determining sequences of the substrate nucleic acids, wherein each of the plurality of template nucleotide sequences has a sequence aligning to a separate portion in a reference genome of the virus.

Disclosed herein, are methods for identifying sequences of a virus of family Coronaviridae in a sample. In an aspect, a method for identifying sequences of a virus of family Coronaviridae in a sample comprises: a) providing substrate nucleic acids, wherein the substrate nucleic acids are amplification products of a plurality of template nucleotide sequences in the sample from the subject, or amplification products of complement sequences of the plurality of template nucleotide sequences, wherein the plurality of template nucleotide sequences have less than about 1500 nucleotides in length in total; and b) determining sequences of the substrate nucleic acids, wherein the plurality of template nucleotide sequences have sequences aligning to a Spike gene or a Nucleocapsid gene in a reference genome of the virus.

In some embodiments, the plurality of template nucleotide sequences have at least about 500 nucleotides in length in total. In some embodiments, the plurality of template nucleotide sequences has at least a sequence aligning to a Spike gene and at least a sequence aligning to a Nucleocapsid gene, respectively, in a reference genome of the virus. In some embodiments, the providing comprises amplification reactions involving a plurality of primers. In some embodiments, the plurality of primers comprises fewer than 50 primer pairs. In some embodiments, the plurality of primers comprises fewer than 9 primer pairs. In some embodiments, the plurality of primers comprises fewer than 5 primer pairs. In some embodiments, the plurality of primers comprises 3 primer pairs. In some embodiments, the plurality of template nucleotide sequences comprises at most 50 template nucleotide sequences. In some embodiments, the plurality of template nucleotide sequences comprises fewer than 9 template nucleotide sequences. In some embodiments, the plurality of template nucleotide sequences comprises fewer than 5 template nucleotide sequences. In some embodiments, the plurality of template nucleotide sequences comprises 3 template nucleotide sequences.

Disclosed herein, are methods for analyzing a sample. In an aspect, a method for analyzing a sample comprises: a) providing substrate nucleic acids by performing amplification reactions involving primers that comprise at least a nucleotide sequence having at least about 85% identity to a sequence selected from the group consisting of SEQ ID NOs: 1-26.

Disclosed herein, are methods for analyzing a sample. In an aspect, a method for analyzing a sample comprises: a) providing substrate nucleic acids by performing amplification reactions involving primers that comprise at least a nucleotide sequence having at least about 85% identity to a sequence selected from the group consisting of SEQ ID NOs: 1-28.

In some embodiments, the primers comprise at least a nucleotide sequence comprising at least about 86% sequence identity to a sequence selected from the group consisting of SEQ ID NOs: 1-26. In some embodiments, the primers comprise at least a nucleotide sequence comprising at least about 90% sequence identity to a sequence selected from the group consisting of SEQ ID NOs: 1-26. In some embodiments, the primers comprise at least a nucleotide sequence comprising at least about 94% sequence identity to a sequence selected from the group consisting of SEQ ID NOs: 1-26. In some embodiments, the primer pairs comprise at least a nucleotide sequence comprising a sequence selected from the group consisting of SEQ ID NOs: 1-26. In some embodiments, the primers comprise at least a nucleotide sequence comprising at least about 86% sequence identity to a sequence selected from the group consisting of SEQ ID NOs: 1-28. In some embodiments, the primers comprise at least a nucleotide sequence comprising at least about 90% sequence identity to a sequence selected from the group consisting of SEQ ID NOs: 1-28. In some embodiments, the primers comprise at least a nucleotide sequence comprising at least about 94% sequence identity to a sequence selected from the group consisting of SEQ ID NOs: 1-28. In some embodiments, the primer pairs comprise at least a nucleotide sequence comprising a sequence selected from the group consisting of SEQ ID NOs: 1-28.

In some embodiments, the method further comprises b) determining sequences of the substrate nucleic acids. In some embodiments, the method further comprises c) providing a report, wherein the report comprises a determination of a presence or an absence of sequences of the virus or a variant of the virus in the sample.

In some embodiments, the providing comprises an amplification reaction. In some embodiments, the amplification reaction comprises a reverse transcription, an asymmetric amplification, a helicase-dependent amplification (HDA), a ligase chain reaction (LCR), a loop mediated isothermal amplification (LAMP), a multiple displacement amplification (MDA), a nucleic acid sequence based amplification (NASBA), a polymerase chain reaction (PCR), a primer extension, a recombinase polymerase amplification (RPA), a rolling circle amplification (RCA), a self-sustained sequence replication (3SR), or a strand displacement amplification (SDA). In some embodiments, the amplification reaction comprises the PCR. In some embodiments, the PCR comprises a multiplex PCR. In some embodiments, the providing further comprises a reverse transcription.

In some embodiments, the providing comprises a barcoding reaction. In some embodiments, the determining comprises sequencing the substrate nucleic acids, or amplification products thereof. In some embodiments, the sequencing comprises a chain termination sequencing, a high-throughput sequencing, a mass spectrophotometry sequencing, a massively parallel signature sequencing, a Maxam-Gilbert sequencing, a nanopore sequencing, a primer walking, a pyrosequencing, a Sanger sequencing, a semiconductor sequencing, a sequencing-by-hybridization, a sequencing-by-ligation, a sequencing-by-synthesis, a single-molecule sequencing, or a shotgun sequencing. In some embodiments, the sequencing comprises the nanopore sequencing.

In some embodiments, the virus from family Coronaviridae comprises a virus of subfamily Orthocoronavirinae. In some embodiments, the virus from family Coronaviridae comprises a virus of species coronavirus. In some embodiments, the virus from family Coronaviridae comprises a Severe acute respiratory syndrome-related coronavirus. In some embodiments, the virus from family Coronaviridae comprises Severe acute respiratory syndrome coronavirus 2.

In some embodiments, the sample is isolated from an animal, water, a surface, droplets or a food. In some embodiments, the water comprises sewage water or drinking water. In some embodiments, the water comprises the sewage water. In some embodiments, the surface comprises a surface of an indoor compartment, a surface of a food packaging material, a surface of a mask, a surface of a medical equipment, or a surface of a furniture. In some embodiments, the surface comprises a surface of the indoor compartment. In some embodiments, the surface of indoor compartment comprises a surface of an elevator. In some embodiments, the surface of the elevator comprises a button of the elevator. In some embodiments, the surface comprises a surface of a metal, a surface of a wood, a surface of a plastic, a surface of a paper, a surface of a glass, a surface of a ceramic, a surface of a fabric, or a surface of a shoe. In some embodiments, the surface comprises a surface of the metal. In some embodiments, the surface of the metal comprises a surface of a door handle or a doorknob.

In some embodiments, the sample comprises a biological sample. In some embodiments, the biological sample is from the subject. In some embodiments, the subject is a human. In some embodiments, the biological sample comprises a blood sample, a tissue sample, a nasal swab sample, or an anal swab sample. In some embodiments, the tissue sample may comprise a body fluid sample, a muscle sample, a skin sample, a lung sample, a brain sample, a nervous system sample, a gastrointestinal sample, a hair sample, or a skin sample. In some embodiments, the biological sample may be obtained by a biopsy. In some embodiments, the biopsy may comprise a blood biopsy, a body fluid biopsy, a muscle biopsy, a skin biopsy, a lung biopsy, a brain biopsy, a nervous system biopsy, a gastrointestinal biopsy, or a skin biopsy. In some embodiments, the biopsy may comprise a fine needle aspiration biopsy, a core needle biopsy, a vacuum-assisted biopsy, an excisional biopsy, a shave biopsy, a punch biopsy, an endoscopic biopsy, a laparoscopic biopsy, a bone marrow aspiration biopsy, a liquid biopsy. In some embodiments, the biopsy may comprise an incisional biopsy or an excisional biopsy.

In some embodiments, the plurality of template nucleotide sequences comprises at least a sequence corresponding to a region spanning across nucleotide positions 331 to 670, 1027 to 1306, 1211 to 1571, 1424 to 1739, 1435 to 1972, 1710 to 1915, 1726 to 1865, 1997 to 2250, 2567 to 2642, or 3149 to 3723 of SEQ ID NO. 31. In some embodiments, the plurality of template nucleotide sequences comprises at least a sequence that encodes a peptide having at least about 70% identity to a sequence of SEQ ID NO. 29. In some embodiments, the method further comprises determining nucleotides at a region of at least one sequence of the sequences of the substrate nucleic acids that corresponds to a region of SEQ ID NO: 31 that encodes an amino acid at any one of positions 142, 143, 144, 145, 157, 158, 211, 212, 417, 440, 446, 452, 477, 478, 484, 493, 496, 498, 501, 505, 614, or any combination thereof, of SEQ ID NO. 29. In some embodiments, the method further comprises: when the at least one sequence comprises a variation relative to SEQ ID NO. 31, (a) determining the virus of family Coronaviridae in the sample is a G-clade variant of Severe acute respiratory syndrome coronavirus 2 (SARS-CoV-2), when the variation comprises nucleotides that encode a Glycine (G) at a position corresponding to position 614 of the sequence of SEQ ID NO. 29; (b) determining the virus of family Coronaviridae in the sample is an Alpha variant of SARS-CoV-2, when the variation consists of a deletion of nucleotides that encode an amino acid at a position corresponding to position 144 of the sequence of SEQ ID NO: 29, nucleotides that encode a Tyrosine (Y) at a position corresponding to position 501 of the sequence of SEQ ID NO: 29, and the nucleotides that encode a G at a position corresponding to position 614 of the sequence of SEQ ID NO: 29; (c) determining the virus of family Coronaviridae in the sample is a Delta variant of SARS-CoV-2, when the variation consists of nucleotides that encode a Aspartic acid (D) at a position corresponding to position 142 of the sequence of SEQ ID NO: 29, a deletion of nucleotides that encode an amino acid at a position corresponding to position 157 of the sequence of SEQ ID NO: 29, a deletion of nucleotides that encode an amino acid at a position corresponding to position 158 of the sequence of SEQ ID NO: 29, nucleotides that encode an Arginine (R) at a position corresponding to position 452 of SEQ ID NO: 29, nucleotides that encode an Lysine (K) at a position corresponding to position 478 of the sequence of SEQ ID NO: 29, and nucleotides that encode a G at a position corresponding to position 614 of the sequence of SEQ ID NO: 29; or (d) determining the virus of family Coronaviridae in the sample is an Omicron variant of SARS-CoV-2, when the variation consists of a deletion of nucleotides that encode an amino acid at a position corresponding to position 42 of the sequence of SEQ ID NO: 29, a deletion of nucleotides that encode an amino acid at a position corresponding to position 43 of the sequence of SEQ ID NO: 29, deletion of nucleotides that encode the amino acid at a position corresponding to position 44 of the sequence of SEQ ID NO: 29, nucleotides that encode a D at a position corresponding to position 145 of the sequence of SEQ ID NO: 29, a deletion of nucleotides that encode an amino acid at a position corresponding to position 211 of the sequence of SEQ ID NO: 29, nucleotides that encode an Isoleucine (I) at a position corresponding to position 212 of the sequence of SEQ ID NO: 29, nucleotides that encode an Asparagine (N) at a position corresponding to position 417 of the sequence of SEQ ID NO: 29, nucleotides that encode a K at a position corresponding to position 440 of the sequence of SEQ ID NO: 29, nucleotides that encode a Serine (S) at a position corresponding to position 446 of the sequence of SEQ ID NO: 29, nucleotides that encode an N at a position corresponding to position 477 of the sequence of SEQ ID NO: 29, nucleotides that encode the K at the a position corresponding to position 478 of the sequence of SEQ ID NO: 29, nucleotides that encode an Alanine (A) at a position corresponding to position 484 of the sequence of SEQ ID NO: 29, nucleotides that encode an R at position 493 of the sequence of SEQ ID NO: 29, nucleotides that encode a S at a position corresponding to position 496 of the sequence of SEQ ID NO: 29, nucleotides that encode an R at a position corresponding to position 498 of the sequence of SEQ ID NO: 29, nucleotides that encode a Y at a position corresponding to position 501 of the sequence of SEQ ID NO: 29, nucleotides that encode a Histidine (H) at a position corresponding to position 505 of the sequence of SEQ ID NO: 29, and nucleotides that encode a G at a position corresponding to position 614 of the sequence of SEQ ID NO: 29.

Disclosed herein, are composition. In an aspect, a composition comprises a mixture of a plurality of primers, wherein the plurality of primers is configured to generate substrate nucleic acids, wherein the substrate nucleic acids are amplification products of a plurality of template nucleotide sequences in a biological sample from a subject, or amplification products of complement sequences of the plurality of template nucleotide sequences, and wherein each of the plurality of template nucleotide sequences has a sequence aligning to a separate portion in a reference genome of a virus of family Coronaviridae.

Disclosed herein are compositions. In an aspect, a composition comprising a mixture of a plurality of primers, wherein the plurality of primers is configured to generate substrate nucleic acids, wherein the substrate nucleic acids are amplification products of a plurality of template nucleotide sequences, or amplification products of complement sequences of the plurality of template nucleotide sequences, wherein the plurality of template nucleotide sequences have less than about 1500 nucleotides in length in total, and wherein each of the plurality of template nucleotide sequences has a sequence aligning to a separate portion in a reference genome of a virus of family Coronaviridae.

In some embodiments, the plurality of primers comprises fewer than 50 primer pairs. In some embodiments, the plurality of primers comprises fewer than 9 primer pairs. In some embodiments, the plurality of primers comprises fewer than 5 primer pairs. In some embodiments, the plurality of primers comprises 3 primer pairs.

In some embodiments, the plurality of primers comprises a primer comprising at least a nucleotide sequence comprising at least about 85% sequence identity to a sequence of a virus of subfamily Orthocoronavirinae. In some embodiments, the plurality of primers comprises a primer comprising at least a nucleotide sequence comprising at least about 85% sequence identity to a sequence of a virus of species coronavirus. In some embodiments, the plurality of primers comprises a primer comprising at least a nucleotide sequence comprising at least about 85% sequence identity to a sequence of Severe acute respiratory syndrome-related coronavirus. In some embodiments, the plurality of primers comprises a primer comprising at least a nucleotide sequence comprising at least about 85% sequence identity to a sequence of Severe acute respiratory syndrome coronavirus 2.

Disclosed herein, are compositions. In an aspect, a composition comprises: a mixture of a plurality of primers, wherein the plurality of primers comprises at least two nucleotide sequences comprising at least about 85% sequence identity to two sequences selected from the group consisting of SEQ ID NOs: 1-26.

In some embodiments, the plurality of primers comprises at least two nucleotide sequences comprising at least about 86% sequence identity to two sequences selected from the group consisting of SEQ ID NOs: 1-26. In some embodiments, the plurality of primers comprises at least two nucleotide sequences comprising at least about 90% sequence identity to two sequences selected from the group consisting of SEQ ID NOs: 1-26. In some embodiments, the plurality of primers comprises at least two nucleotide sequences comprising at least about 94% sequence identity to two sequences selected from the group consisting of SEQ ID NOs: 1-26. In some embodiments, the plurality of primers comprises at least two nucleotide sequences selected from the group consisting of SEQ ID NOs: 1-26. In some embodiments, the plurality of primers comprises at least four nucleotide sequences comprising at least about 85% sequence identity to four sequences selected from the group consisting of SEQ ID NOs: 1-26. In some embodiments, the plurality of primers comprises at least four nucleotide sequences comprising at least about 86% sequence identity to four sequences selected from the group consisting of SEQ ID NOs: 1-26. In some embodiments, the plurality of primers comprises at least four nucleotide sequences comprising at least about 90% sequence identity to four sequences selected from the group consisting of SEQ ID NOs: 1-26. In some embodiments, the plurality of primers comprises at least four nucleotide sequences comprising at least about 94% sequence identity to four sequences selected from the group consisting of SEQ ID NOs: 1-26. In some embodiments, the plurality of primers comprises at least four nucleotide sequences selected from the group consisting of SEQ ID NOs: 1-26. In some embodiments, the plurality of primers comprises at least six nucleotide sequences comprising at least about 85% sequence identity to the group consisting of SEQ ID NOs: 1-26. In some embodiments, the plurality of primers comprises at least six nucleotide sequences comprising at least about 86% sequence identity to the group consisting of SEQ ID NOs: 1-26. In some embodiments, the plurality of primers comprises at least six nucleotide sequences comprising at least about 90% sequence identity to the group consisting of SEQ ID NOs: 1-26. In some embodiments, the plurality of primers comprises at least six nucleotide sequences comprising at least about 94% sequence identity to the group consisting of SEQ ID NOs: 1-26. In some embodiments, the plurality of primers comprises at least six nucleotide sequences selected from the group consisting of SEQ ID NOs: 1-26.

Disclosed herein, are compositions. In an aspect, a composition comprises: a mixture of a plurality of primers, wherein the plurality of primers comprises at least two nucleotide sequences comprising at least about 85% sequence identity to two sequences selected from the group consisting of SEQ ID NOs: 1-28.

In some embodiments, the plurality of primers comprises at least two nucleotide sequences comprising at least about 86% sequence identity to two sequences selected from the group consisting of SEQ ID NOs: 1-28. In some embodiments, the plurality of primers comprises at least two nucleotide sequences comprising at least about 90% sequence identity to two sequences selected from the group consisting of SEQ ID NOs: 1-28. In some embodiments, the plurality of primers comprises at least two nucleotide sequences comprising at least about 94% sequence identity to two sequences selected from the group consisting of SEQ ID NOs: 1-28. In some embodiments, the plurality of primers comprises at least two nucleotide sequences selected from the group consisting of SEQ ID NOs: 1-28. In some embodiments, the plurality of primers comprises at least four nucleotide sequences comprising at least about 85% sequence identity to four sequences selected from the group consisting of SEQ ID NOs: 1-28. In some embodiments, the plurality of primers comprises at least four nucleotide sequences comprising at least about 86% sequence identity to four sequences selected from the group consisting of SEQ ID NOs: 1-28. In some embodiments, the plurality of primers comprises at least four nucleotide sequences comprising at least about 90% sequence identity to four sequences selected from the group consisting of SEQ ID NOs: 1-28. In some embodiments, the plurality of primers comprises at least four nucleotide sequences comprising at least about 94% sequence identity to four sequences selected from the group consisting of SEQ ID NOs: 1-28. In some embodiments, the plurality of primers comprises at least four nucleotide sequences selected from the group consisting of SEQ ID NOs: 1-28. In some embodiments, the plurality of primers comprises at least six nucleotide sequences comprising at least about 85% sequence identity to the group consisting of SEQ ID NOs: 1-28. In some embodiments, the plurality of primers comprises at least six nucleotide sequences comprising at least about 86% sequence identity to the group consisting of SEQ ID NOs: 1-28. In some embodiments, the plurality of primers comprises at least six nucleotide sequences comprising at least about 90% sequence identity to the group consisting of SEQ ID NOs: 1-28. In some embodiments, the plurality of primers comprises at least six nucleotide sequences comprising at least about 94% sequence identity to the group consisting of SEQ ID NOs: 1-28. In some embodiments, the plurality of primers comprises at least six nucleotide sequences selected from the group consisting of SEQ ID NOs: 1-28. In some embodiments, the plurality of primers comprises at least a nucleotide sequence comprising a sequence selected from the group consisting of SEQ ID NOs: 1, 3, 4, 6, 13, 14, 17, 18, and 20. In some embodiments, the plurality of primers comprises at least a nucleotide sequence comprising a sequence selected from the group consisting of SEQ ID NOs: 2, 5, 7, 8, 15, 16, 19, 21, and 26. In some embodiments, the plurality of primers comprises at least a nucleotide sequence comprising a sequence selected from the group consisting of SEQ ID NOs: 1, 3, 4, 6, 13, 14, 17, 18, and 20; and at least a nucleotide sequence comprising a sequence selected from the group consisting of SEQ ID NOs: 2, 5, 7, 8, 15, 16, 19, 21, and 26. In some embodiments, the plurality of primers comprises at least a nucleotide sequence comprising a sequence selected from the group consisting of SEQ ID NOs: 9, 11, 22, and 24. The method of claim 23, wherein the plurality of primers comprises at least a nucleotide sequence comprising a sequence selected from the group consisting of SEQ ID NOs: 10, 12, 23, and 25. In some embodiments, the plurality of primers comprises at least a nucleotide sequence comprising a sequence selected from the group consisting of SEQ ID NOs: 9, 11, 22, and 24; and at least a nucleotide sequence comprising a sequence selected from the group consisting of SEQ ID NOs: 10, 12, 23, and 25. In some embodiments, the plurality of primers comprises at least a nucleotide sequence comprising a sequence selected from the group consisting of SEQ ID NOs: 1, 3, 4, 6, 13, 14, 17, 18, and 20; at least a nucleotide sequence comprising a sequence selected from the group consisting of SEQ ID NOs: 2, 5, 7, 8, 15, 16, 19 21, and 26; at least a nucleotide sequence comprising a sequence selected from the group consisting of SEQ ID NOs: 9, 11, 22; and 24 and at least a nucleotide sequence comprising a sequence selected from the group consisting of SEQ ID NOs: 10, 12, 23, and 25. In some embodiments, the plurality of primers comprises a first primer pair selected from the group consisting of: a) SEQ ID NOs: 1 and 2, b) SEQ ID NOs: 3 and 19, c) SEQ ID NOs: 4 and 15, d) SEQ ID NOs: 5 and 14, e) SEQ ID NOs: 6 and 7, f) SEQ ID NOs: 13 and 26, g) SEQ ID NOs: 16 and 18, and h) SEQ ID NOs: 20 and 21. In some embodiments, the plurality of primers further comprises a second primer pair selected from the group consisting of: a) SEQ ID NOs: 9 and 10, b) SEQ ID NOs: 11 and 12, c) SEQ ID NOs: 22 and 23, and d) SEQ ID NOs: 24 and 25.

In some embodiments, the plurality of primers comprises at least a nucleotide sequence comprising a sequence selected from the group consisting of SEQ ID NOs: 1, 3, 4, 6, 13, 14, 17, 18, 20, and 27. In some embodiments, the plurality of primers comprises at least a nucleotide sequence comprising a sequence selected from the group consisting of SEQ ID NOs: 2, 5, 7, 8, 15, 16, 19, 21, 26, and 28. In some embodiments, the plurality of primers comprises at least a nucleotide sequence comprising a sequence selected from the group consisting of SEQ ID NOs: 1, 3, 4, 6, 13, 14, 17, 18, 20, and 27; and at least a nucleotide sequence comprising a sequence selected from the group consisting of SEQ ID NOs: 2, 5, 7, 8, 15, 16, 19, 21, 26, and 28. In some embodiments, the plurality of primers comprises at least a nucleotide sequence comprising a sequence selected from the group consisting of SEQ ID NOs: 9, 11, 22, and 24. The method of claim 23, wherein the plurality of primers comprises at least a nucleotide sequence comprising a sequence selected from the group consisting of SEQ ID NOs: 10, 12, 23, and 25. In some embodiments, the plurality of primers comprises at least a nucleotide sequence comprising a sequence selected from the group consisting of SEQ ID NOs: 9, 11, 22, and 24; and at least a nucleotide sequence comprising a sequence selected from the group consisting of SEQ ID NOs: 10, 12, 23, and 25. In some embodiments, the plurality of primers comprises at least a nucleotide sequence comprising a sequence selected from the group consisting of SEQ ID NOs: 1, 3, 4, 6, 13, 14, 17, 18, 20, and 27; at least a nucleotide sequence comprising a sequence selected from the group consisting of SEQ ID NOs: 2, 5, 7, 8, 15, 16, 19 21, and 26; at least a nucleotide sequence comprising a sequence selected from the group consisting of SEQ ID NOs: 9, 11, 22; and 24 and at least a nucleotide sequence comprising a sequence selected from the group consisting of SEQ ID NOs: 10, 12, 23, and 25. In some embodiments, the plurality of primers comprises a first primer pair selected from the group consisting of: a) SEQ ID NOs: 1 and 2, b) SEQ ID NOs: 3 and 19, c) SEQ ID NOs: 4 and 15, d) SEQ ID NOs: 5 and 14, e) SEQ ID NOs: 6 and 7, f) SEQ ID NOs: 13 and 26, g) SEQ ID NOs: 16 and 18, h) SEQ ID NOs: 20 and 21, and i) SEQ ID NOs: 26 and 27. In some embodiments, the plurality of primers further comprises a second primer pair selected from the group consisting of: a) SEQ ID NOs: 9 and 10, b) SEQ ID NOs: 11 and 12, c) SEQ ID NOs: 22 and 23, and d) SEQ ID NOs: 24 and 25.

In some embodiments, the plurality of primers is configured to generate substrate nucleic acids. In some embodiments, the plurality of primers is configured to generate the substrate nucleic acids via an amplification reaction. In some embodiments, the amplification reaction comprises an asymmetric amplification, a helicase-dependent amplification (HDA), a ligase chain reaction (LCR), a loop mediated isothermal amplification (LAMP), a multiple displacement amplification (MDA), a nucleic acid sequence based amplification (NASBA), a polymerase chain reaction (PCR), a primer extension, a recombinase polymerase amplification (RPA), a rolling circle amplification (RCA), a self-sustained sequence replication (3SR), or a strand displacement amplification (SDA). In some embodiments, the amplification reaction comprises the PCR. In some embodiments, the PCR comprises a multiplex PCR.

Disclosed herein, are kits. In an aspect, a kit comprises the composition described thereof. In some embodiments, the composition further comprises an oligonucleotide that is configured to bind RNAs. In some embodiments, the oligonucleotide is configured to bind messenger RNAs (mRNAs). In some embodiments, the oligonucleotide is attached to a solid support. In some embodiments, the solid support is a magnetic bead. In some embodiments, the composition further comprises a barcoding probe. In some embodiments, the composition further comprises a sequencing probe. In some embodiments, the sequencing probe is configured to facilitate sequencing the substrate nucleic acids in a sequencing reaction. In some embodiments, the sequencing reaction comprises a chain termination sequencing, a high-throughput sequencing, a mass spectrophotometry sequencing, a massively parallel signature sequencing, a Maxam-Gilbert sequencing, a nanopore sequencing, a primer walking, a pyrosequencing, a Sanger sequencing, a semiconductor sequencing, a sequencing-by-hybridization, a sequencing-by-ligation, a sequencing-by-synthesis, a single-molecule sequencing, or a shotgun sequencing. In some embodiments, the sequencing reaction comprises the nanopore sequencing.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The features of the disclosure are set forth with particularity in the appended claims. A better understanding of the features and the disclosure will be obtained by reference to the following detailed description that sets forth illustrative embodiments, in which the principles of the present disclosure are utilized, and the accompanying drawings (also “Figure” and “FIG.” herein), of which:

FIG. 1 shows a schematic of the workflow for detecting a sequence of a virus of the family Coronaviridae.

FIG. 2 shows the 3-dimensional structure of the Spike protein (SPIKE) of Severe acute respiratory syndrome coronavirus 2 (SARS-CoV-2) binding to angiotensin-converting enzyme 2 (ACE2). The locations of the mutations identified in the variants of SARS-CoV-2 are indicated.

FIG. 3 shows that multiplex Polymerase Chain Reaction (PCR) can generate sufficient amplification products from template nucleotide sequences for downstream analysis. An image of agarose electrophoresis gel loaded with PCR reaction products using primer mixes listed in Table 8 is shown. Template nucleotide sequences for the PCR reaction are listed on top of the gel. Water was used as a negative control. The primer mix for each PCR is listed at the bottom of each lane. Reactions with the multiplex primer mix comprising the individual primer mix S1, S2, and N generated all three amplification products. The single PCR reactions with each individual primer mix are shown for comparison. The molecular size marker is shown on the far left.

FIG. 4 shows that multiplex Polymerase Chain Reaction (PCR) can generate sufficient amplification products from template nucleotide sequences for downstream analysis using various primer mixes. An image of agarose electrophoresis gel loaded with PCR reaction products using primer mixes listed in Table 9 is shown. Template nucleotide sequences for the PCR reaction are listed on top of the gel. The primer mix for each PCR is listed at the bottom of each lane. Various reactions with the multiplex primer mixes could generate multiple individual amplification products. The single PCR reactions with each individual primer mix are shown for comparison. The molecular size marker is shown on the far left.

### DETAILED DESCRIPTION

In some aspects, the present disclosure provides methods for identifying sequences of viruses. The compositions for carrying out the methods are also provided. The compositions can comprise, consist essentially of, or consist of a plurality of primers. In further aspects, the disclosure provides kits comprising the compositions. The kit can also comprise oligonucleotides, probes, and solid supports for carrying out the same.

### Definitions

The term “alignment” or “aligning” as used herein when describing nucleotide sequences can refer to the arrangement of multiple nucleotide sequences based on regions of sequence identity. In some cases, the alignment may comprise arranging a query sequence to a reference genome or sequence. The amount or percentage of sequence identity may be determined based on methods described herein. The amount or percentage of sequence identity may comprise at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or more than at least 99%. In some cases, a DNA sequence may be compared to a DNA sequence. In some cases, an RNA sequence may be compared to an RNA sequence. In some cases, a DNA sequence may be compared to an RNA sequence. In some cases, an RNA sequence may be compared to a DNA sequence. When aligning a DNA sequence and an RNA sequence, a thymine of the DNA sequence and an uracil of the RNA sequence may be considered equivalent.

The term “amplicon” as used herein can refer to an amplification synthesis product of a nucleic acid molecule. The amplicon may be synthesized and amplified based on a template nucleotide sequence using an amplification reaction. An amplicon acid may be a distinct molecular entity or part of the same molecular entity of the template nucleotide sequence, depending on the amplification reaction.

The term “amplification reaction” can refer to a chemical reaction that can generate or synthesize a nucleotide sequence using a template nucleotide sequence. Such an amplification reaction may comprise an in vitro chemical reaction. In some cases, such an amplification reaction may also comprise an in vivo reaction.

The term “barcoding” as used herein can refer to the process of providing a nucleotide sequence to a molecule to uniquely identify the molecule. The molecule being identified may comprise any substrate nucleic acids or amplification products described elsewhere in this disclosure. In some cases, a nucleotide sequence that can uniquely identify a molecule may comprise a nucleotide barcode. A nucleotide barcode may be identified by sequencing.

The term “biological sample” as used herein can refer to a biological material originated in a subject. A biological sample may be isolated from the subject. A biological sample may also be derived from the subject. A biological material may comprise nucleic acids. A biological may comprise a cell. A biological sample may comprise a group of cells. A biological sample may comprise a tissue. A biological sample may comprise an organ. A biological sample may undergo processing depending on the application of the biological sample. Such processing may comprise culture, extraction, purification, derivation, or storage. One such derivation may comprise an amplification reaction.

The term “complementary” or “complementarity” as used herein can refer to the ability of a nucleic acid to form hydrogen bond(s) with another nucleic acid sequence by either traditional Watson-Crick or other non-traditional types. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively).

The term “genome” as used herein can refer to the complete nucleotide sequence present in an organism. When referring to a virus, a genome may comprise DNA or RNA, depending on the life cycle of the virus. A genome virus may also be single-stranded or double-stranded, depending on the life cycle of the virus. For a single-stranded virus, the genome may also be positive-stranded or negative stranded. A positive-stranded RNA virus may comprise a genome sequence that can act as the messenger RNA (mRNA) of the virus. A negative-stranded RNA virus may comprise a genome sequence that can act as complement of the messenger RNA (mRNA) of the virus.

The term “magnetic bead” as used herein can refer to a type of solid substrate that comprises particles with superparamagnetic properties. In the presence of an external magnetic field, the magnetic beads may exhibit magnetic behaviors, forming clumps or aggregates with each other, separating from other non-magnetic molecules. The magnetic beads described herein may be configured to bind any nucleic acid molecules described herein.

The term “multiplex PCR” can refer to a type of Polymerase Chain Reaction (PCR) reaction that substrate nucleic acids or amplification products of multiple template nucleotide sequences are simultaneously synthesized in one PCR reaction solution. In some cases, multiple primers or primer pairs, each configured to synthesize a specific amplicon, may be combined in one PCR reaction solution to synthesize the amplicons. In some cases, the number of primers or primer pairs may be the same as the number of template nucleotide sequences being amplified or the number of amplicons being synthesized. In some cases, the number of primers or primer pairs may be the fewer than the number of template nucleotide sequences being amplified or the number of amplicons being synthesized.

The term “nanopore sequencing” as used herein can refer to a type of sequencing that the sequence of a nucleic acid molecule is determined from the changes of ionic current across a membrane when a single-stranded nucleic acid molecule passes through a nanopore.

The term “Nucleocapsid gene” or “capsid gene” can refer the gene sequence of a virus that encodes an RNA genome-binding protein protomer subunit. For example, the Nucleocapsid gene of Severe acute respiratory syndrome coronavirus 2 may comprise the genetic sequence of NCBI Gene ID: 43740575 or the genetic sequence encoding the protein sequence of NCBI Reference Sequence: YP_009724397.2.

The term “nucleotide sequence”, or “sequence” when describing a nucleotide, as used herein, can refer to a nucleic acid molecule with a specific order or arrangement of nucleotides. A nucleic acid sequence may comprise a single stranded nucleic acid molecule. A nucleic acid sequence may comprise a double stranded nucleic acid molecule. In a double stranded nucleic acid sequence, the single stranded nucleic acid sequences may bind or anneal to each other through complementary base pairing, or Watson-Crick base pairing, of the nucleotides. A nucleic acid sequence may be a nucleic acid molecule. A deoxyribonucleic acid (DNA) sequence may be a DNA molecule or a part of a DNA molecule. A ribonucleic acid (RNA) sequence may be an RNA molecule or part of an RNA molecule.

The term “PCR” or ‘polymerase chain reaction” can refer to an amplification reaction that utilizes a DNA polymerase to synthesize new strands of DNA complementary to a template nucleotide sequence. A PCR reaction may utilize at least one primer.

The term “portion” as used herein when describing a genome can refer to a specific part of the gnome. A separate portion of a genome may be an independent partition of a genome. For example, each nucleotide of a genome may comprise a coordinate number. A separate portion of a gnome may refer to the segment of nucleotides encompassed within two such coordinate numbers. When describing two separate portions of a genome, a first separate portion may not comprise any nucleotides with the coordinate numbers of a second separate portion.

The term “primer” can refer to a nucleotide sequence that can prime or hybridize to a template nucleotide sequence through complementary base-pairing in an amplification reaction. The nucleotide sequence of a primer may determine the identity of the template nucleotide sequence or the portion of a genome being amplified. In some cases, one primer may be sufficient to amplify a template nucleotide in an amplification reaction. In other cases, a pair of primers may be sufficient to amplify a template nucleotide. The number of primer sufficient to amplify a template sequence nucleotide may depend on the type of amplification reactions employed.

The term “reference genome” as used herein can refer to a genome sequence that is a representative example of the nucleotide sequence of an organism. In some cases, a reference genome of a virus may comprise the genome of a wild-type version of a virus. In some cases, a reference genome of a virus may comprise the genome of the most common variant or strain of a virus. In some cases, a reference genome of a virus may comprise the genome of the first-identified strain of a virus. For example, the reference genome of Severe acute respiratory syndrome coronavirus 2 may comprise the sequence of NC_045512.2. In some cases, even if a genome of a virus may comprise a single-stranded nucleic acid molecule, the reference genome of the virus may comprise the sequence of the single-stranded genome and the complementary sequence of the single-stranded genome. In some cases, for a particular nucleotide of a genome with a specific coordinate number, the particular nucleotide and the complementary nucleotide may be considered equivalent. The particular nucleotide and the complementary nucleotide may also be considered to have the same coordinate number.

The term “sequence identity” or “identity” as used herein can refer to sequence similarity between two nucleic acid molecules. Sequence identity can be determined by comparing a position in each sequence which can be aligned for purposes of comparison. When a position in the compared sequence can be occupied by the same base, then the molecules can be homologous at that position. A degree of sequence identity between sequences can be a function of the number of matching or homologous positions shared by the sequences. As a practical matter, whether any particular sequence can be at least 50%, at least 60%, at least 70%, at least 80%, at least 85%, at least 90%, at least 91%, at least 92%, at least 93%, at least 94%, at least 95%, at least 96%, at least 97%, at least 98%, at least 99% or more than at least 99% identical to any sequence described herein, such particular sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed.

The term “sequencing” as used herein can refer to the determination of the nucleotide order of a nucleotide sequence. Sequencing may comprise any sequencing reaction described herein.

The term “solid support” as used herein refers to a solid substrate that can immobilize a nucleic acid molecule when the nucleic acid molecule is coupled to the solid substrate. The immobilization of the nucleic acid molecule by the solid substrate may allow separation of the nucleic acid molecule from other molecules not attached to the solid substrate.

The term “Spike gene” as used herein can refer to the gene sequence of a virus that encodes a subunit of a peplomer structure of the virus. For example, the Spike gene of Severe acute respiratory syndrome coronavirus 2 may comprise the genetic sequence of NCBI Gene ID: 43740568 or the genetic sequence encoding the protein sequence of NCBI Reference Sequence: YP_009724390.

The term “template nucleotide sequence” can refer to a nucleotide sequence that is being amplified by an amplification reaction. Such an amplification reaction may generate a product of the template nucleotide sequence. A template nucleotide sequence may be a distinct molecular entity or part of the same molecular entity of a substrate nucleic acid, depending on the amplification reaction.

The term “variant” as used herein when describing a virus can refer to a version of virus based on the nucleotide sequence of a genome, a particular set of genes, or a gene of the virus. In some case, a virus variant may have a difference in the genome, the particular set of genes, or the gene of the virus.

The term “a” or “an” as used herein can refer to one or to more than one (i.e., to at least one) of the grammatical object of the article. By way of example, “an element” means one element or more than one element.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” meaning up to an acceptable error range for the particular value should be assumed. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Methods and Compositions for Identifying Sequences of Virus

Provided herein, are methods for identifying sequences of a pathogen. The sequences identified can be used to determine a strain or variant of the pathogen. Different pathogen strains may evolve from a parental or wild type pathogen strain, with a change in pathogenicity (e.g., virulence), transmissibility, resistance to vaccines or drugs, or a combination thereof. Accurate and rapid determination of the pathogen strains can allow appropriate deployment of treatments to curb the transmission of the pathogens in a population or treat diseases or conditions caused by the pathogens in a subject.

Methods for pathogen determination can comprise conventional polymerase chain reaction (PCR) or whole-genome sequencing. Conventional PCR can rapidly determine the pathogen strain/variants based on the presence/absence and/or a change in the size of an amplification product. However, without determining the sequence of the pathogen, conventional PCR may not be able to determine the pathogen strain or variant accurately and conclusively. Furthermore, with the emergence of a yet-to-be-characterized strain or variant, appropriate reagents (e.g., primers) may not be appropriately designed without the prior knowledge of the mutation(s) that characterize a particular pathogen strain or variant.

Although whole-genome sequencing can reveal the genome sequences of a pathogen, accurate determination of the pathogen genome can require a substantial sequencing depth at the sequence-of-interest (e.g., the sequence region comprising one or more mutations that can characterize the pathogen or the pathogen strain that characterize a pathogen). Acquiring sufficient sequencing depth of various sequence regions in a whole-genome sequencing can require substantial efforts. Furthermore, whole genome sequence assembly can require a significant amount of time, hindering rapid determination of the pathogen strain or variant (e.g., real-time identification).

Provided herein are methods for rapid and accurate determination of pathogen strain or variant. In some instances, a method provided herein for identifying sequences of a virus in a sample (e.g., a biological sample or an environmental sample) comprises providing substrate nucleic acids (e.g., amplicons of the sequences of the virus) and determining the sequences of the substrate nucleic acids. In some cases, a method provided herein for identifying sequences of a virus in a sample comprises providing substrate nucleic acids (e.g., amplicons of the sequences of the virus) or determining sequences of the substrate nucleic acids. In other cases, a method for analyzing a sample may comprise a) providing substrate nucleic acids by performing, wherein the substrate nucleic acids are products of amplification reactions involving primers that comprise at least a nucleotide sequence having at least about 85% identity to a sequence selected from the group consisting of SEQ ID NOs: 1-28. In some cases, the method may further comprise analyzing the plurality of template nucleotide sequences. In some cases, the analyzing may comprise determining nucleotides at a region of the sequence of the substrate nucleic acids.

Compared to other conventional methods of detecting virus or SARS-CoV-2, such as conventional PCT and whole genome sequencing of the virus, detection of virus or SARS-CoV-2 variants using multiplex polymerase chain reaction coupled with high throughput sequencing of the amplification products offers various advantages. In some cases, detection of virus or SARS-CoV-2 variants using multiplex polymerase chain reaction coupled with high throughput sequencing of the amplification products may have a very low false positive rate in identifying a virus variant. In some cases, detection of virus or SARS-CoV-2 variants using multiplex polymerase chain reaction coupled with high throughput sequencing of the amplification products may not identify a virus as a certain virus variant when the virus is not the certain virus variant. In some cases, detection of virus or SARS-CoV-2 variants using conventional PCR may have a very high false positive rate in identifying a virus variant. In some cases, detection of virus or SARS-CoV-2 variants using conventional PCR may identify a virus as a certain virus variant when the virus is not the certain virus variant. In some cases, detection of virus or SARS-CoV-2 variants using multiplex polymerase chain reaction coupled with high throughput sequencing of the amplification products may identify a virus variant. In some cases, detection of virus or SARS-CoV-2 variants using multiplex polymerase chain reaction coupled with high throughput sequencing of the amplification products may identify a novel virus variant. In some cases, detection of virus or SARS-CoV-2 variants using multiplex polymerase chain reaction coupled with high throughput sequencing of the amplification products may identify a virus variant (novel or not) based on the actual sequencing result or sequence of the virus variant. In some cases, detection of virus or SARS-CoV-2 variants using conventional PCR may not identify a virus variant. In some cases, detection of virus or SARS-CoV-2 variants using conventional PCR may not identify a novel virus variant. In some cases, detection of virus or SARS-CoV-2 variants using conventional PCR may not identify a virus variant (novel or not) because conventional PCR may not determine the actual sequencing result or sequence of the virus variant.

In some cases, detection of virus or SARS-CoV-2 variants using multiplex polymerase chain reaction coupled with high throughput sequencing of the amplification products may take about an hour from sequencing to identifying a virus variant. In some cases, detection of virus or SARS-CoV-2 variants using multiplex polymerase chain reaction coupled with high throughput sequencing of the amplification products may take less than 50 hours from sequencing to identifying a virus variant. In some cases, detection of virus or SARS-CoV-2 variants using whole-genome sequencing may take more than an hour from sequencing to identifying a virus variant. In some cases, detection of virus or SARS-CoV-2 variants using whole-genome sequencing may take at least about 50 hours from sequencing to identifying a virus variant. For example, detection of virus or SARS-CoV-2 variants using whole-genome sequencing may take about 72 hours from sequencing to identifying a virus variant. In some cases, detection of virus or SARS-CoV-2 variants using multiplex polymerase chain reaction coupled with high throughput sequencing of the amplification products may use a sequencer that costs about US$50,000 (e.g., a GridION sequencer) to identify a virus variant when sequencing 384 virus samples. In some cases, detection of virus or SARS-CoV-2 variants using whole-genome sequencing may use a sequencer that costs about US$250,000 (e.g., a NextSeq 500 sequencer) to identify a virus variant when sequencing 384 virus samples. In some cases, detection of virus or SARS-CoV-2 variants using multiplex polymerase chain reaction coupled with high throughput sequencing of the amplification products may require about 200-300 bp sequencing coverage of the sequence of a virus variant to identify the virus variant. In some cases, detection of virus or SARS-CoV-2 variants using multiplex polymerase chain reaction coupled with high throughput sequencing of the amplification products may require sequencing only the template nucleotide sequences that contain the mutation(s) that can characterize a virus variant to identify the virus variant. In some cases, detection of virus or SARS-CoV-2 variants using whole-genome sequencing may require sequencing the whole genome or about sequencing coverage of the sequence of a virus variants to identify the virus variant. In some cases, detection of virus or SARS-CoV-2 variants using multiplex polymerase chain reaction coupled with high throughput sequencing of the amplification products may allow for real-time detection or identification of a virus variant. In some cases, detection of virus or SARS-CoV-2 variants using whole-genome sequencing may not allow for real-time detection or identification of a virus variant. In some cases, detection of virus or SARS-CoV-2 variants using multiplex polymerase chain reaction coupled with high throughput sequencing of the amplification products may allow for detection or identification of virus variants during the sequencing of the samples without stopping the sequencing reactions. In some cases, detection of virus or SARS-CoV-2 variants using whole-genome sequencing may not allow for detection or identification of virus variants during the sequencing of the samples without stopping the sequencing reactions.

### Template Nucleotide Sequences

In some instances, a method for identifying sequences of a virus in a sample may comprise providing substrate nucleic acids that comprise amplification products of a plurality of template nucleotide sequences in a sample or amplification products of complement sequences of the plurality of template nucleotide sequences. In some cases, substrate nucleic acids may comprise amplification products of a plurality of template nucleotide sequences in a sample and amplification products of complement sequences of the plurality of template nucleotide sequences. In some cases, substrate nucleic acids may comprise amplification products of a plurality of template nucleotide sequences in a sample. In some cases, substrate nucleic acids may comprise amplification products of complement sequences of a plurality of template nucleotide sequences in a sample.

In some instances, each of a plurality of template nucleotide sequences may have at least a sequence aligning to a separate portion in a reference genome of a virus. In some cases, a template nucleotide sequence may have at least a sequence aligning to a separate portion in a reference genome of a virus. In some cases, a first template nucleotide sequence may have a first sequence aligning to a first separate portion in a reference genome of a virus, and a second template nucleotide sequence may have a second sequence aligning to a second separate portion in the reference genome of the virus. In some instances, each of a plurality of complements of template nucleotide sequences may have at least a sequence aligning to a separate portion in a reference genome of a virus. In some cases, a complement of a template nucleotide sequence may have at least a sequence aligning to a separate portion in a reference genome of a virus. In some cases, a first complement of a template nucleotide sequence may have at least a first sequence aligning to a first separate portion in a reference genome of a virus, and a second complement of a template nucleotide sequence may have at least a second sequence aligning to a second separate portion in the reference genome of the virus. The first separate portion and the second separate portion may not share a same nucleotide sequence. In some cases, each nucleotide of a reference genome may comprise a coordinate number. In some cases, each coordinate number of a reference genome may be unique. In some cases, with respect to the coordinate number of a reference genome, each nucleotide of a genome may be unique. In some cases, the nucleotide or nucleotides of a first template nucleotide sequence in a first separate portion of a reference genome may not overlap with that or those of a second template nucleotide sequence. In some cases, two separate portions in a reference genome may not share a nucleotide with a unique coordinate number as described thereof.

In some instances, generating substrate nucleic acids may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 template nucleotide sequences. In some instances, generating substrate nucleic acids may comprise 1, 2, 3, 4, 5, 6, 7, 8, 9, 10, 11, 12, 13, 14, 15, 16, 17, 18, 19, 20, 21, 22, 23, 24, or 25 template nucleotide sequences. In some instances, generating substrate nucleic acids may comprise 1, 2, 3, 4, 5, 6, 7, 8, 9, 10, 11, 12, 13, 14, 15, 16, 17, 18, 19, or 20 template nucleotide sequences. In some instances, generating substrate nucleic acids may comprise 1, 2, 3, 4, 5, 6, 7, 8, 9, 10, 11, 12, 13, 14, or 15 template nucleotide sequences. In some instances, generating substrate nucleic acids may comprise 1, 2, 3, 4, 5, 6, 7, 8, 9, or 10 template nucleotide sequences. In some instances, generating substrate nucleic acids may comprise 1, 2, 3, 4, 5, 6, 7, 8, or 9 template nucleotide sequences. In some instances, generating substrate nucleic acids may comprise 1, 2, 3, 4, 5, 6, 7, or 8 template nucleotide sequences. In some instances, generating substrate nucleic acids may comprise 1, 2, 3, 4, 5, 6, or 7 template nucleotide sequences. In some instances, generating substrate nucleic acids may comprise 1, 2, 3, 4, 5, or 6 template nucleotide sequences. In some instances, generating substrate nucleic acids may comprise 1, 2, 3, 4, or 5 template nucleotide sequences. In some instances, generating substrate nucleic acids may comprise 1, 2, 3, or 4 template nucleotide sequences. In some instances, generating substrate nucleic acids may comprise 1, 2, or 3 template nucleotide sequences. In some instances, generating substrate nucleic acids may comprise 1, or 2 template nucleotide sequences.

In some cases, generating substrate nucleic acids may comprise 1 template nucleotide sequence. In some cases, generating substrate nucleic acids may comprise 2 template nucleotide sequences. In some instances, generating substrate nucleic acids may comprise 3 template nucleotide sequences. In some cases, generating substrate nucleic acids may comprise 4 template nucleotide sequences. In some cases, generating substrate nucleic acids may comprise 5 template nucleotide sequences. In some cases, generating substrate nucleic acids may comprise 6 template nucleotide sequences. In some cases, generating substrate nucleic acids may comprise 7 template nucleotide sequences. In some cases, generating substrate nucleic acids may comprise 8 template nucleotide sequences. In some cases, generating substrate nucleic acids may comprise 9 template nucleotide sequences. In some cases, generating substrate nucleic acids may comprise 10 template nucleotide sequences. In some cases, generating substrate nucleic acids may comprise 11 template nucleotide sequences. In some cases, generating substrate nucleic acids may comprise 12 template nucleotide sequences. In some cases, generating substrate nucleic acids may comprise 13 template nucleotide sequences. In some cases, generating substrate nucleic acids may comprise 14 template nucleotide sequences. In some cases, generating substrate nucleic acids may comprise 15 template nucleotide sequences. In some cases, generating substrate nucleic acids may comprise 16 template nucleotide sequences. In some cases, generating substrate nucleic acids may comprise 17 template nucleotide sequences. In some cases, generating substrate nucleic acids may comprise 18 template nucleotide sequences. In some cases, generating substrate nucleic acids may comprise 19 template nucleotide sequences. In some cases, generating substrate nucleic acids may comprise 20 template nucleotide sequences. In some cases, generating substrate nucleic acids may comprise 21 template nucleotide sequences. In some cases, generating substrate nucleic acids may comprise 22 template nucleotide sequences. In some cases, generating substrate nucleic acids may comprise 23 template nucleotide sequences. In some cases, generating substrate nucleic acids may comprise 24 template nucleotide sequences. In some cases, generating substrate nucleic acids may comprise 25 template nucleotide sequences. In some cases, generating substrate nucleic acids may comprise 26 template nucleotide sequences. In some cases, generating substrate nucleic acids may comprise 27 template nucleotide sequences. In some cases, generating substrate nucleic acids may comprise 28 template nucleotide sequences. In some cases, generating substrate nucleic acids may comprise 29 template nucleotide sequences. In some cases, generating substrate nucleic acids may comprise 30 template nucleotide sequences. In some cases, generating substrate nucleic acids may comprise 31 template nucleotide sequences. In some cases, generating substrate nucleic acids may comprise 32 template nucleotide sequences. In some cases, generating substrate nucleic acids may comprise 33 template nucleotide sequences. In some cases, generating substrate nucleic acids may comprise 34 template nucleotide sequences. In some cases, generating substrate nucleic acids may comprise 35 template nucleotide sequences. In some cases, generating substrate nucleic acids may comprise 36 template nucleotide sequences. In some cases, generating substrate nucleic acids may comprise 37 template nucleotide sequences. In some cases, generating substrate nucleic acids may comprise 38 template nucleotide sequences. In some cases, generating substrate nucleic acids may comprise 39 template nucleotide sequences. In some cases, generating substrate nucleic acids may comprise 40 template nucleotide sequences. In some cases, generating substrate nucleic acids may comprise 41 template nucleotide sequences. In some cases, generating substrate nucleic acids may comprise 42 template nucleotide sequences. In some cases, generating substrate nucleic acids may comprise 43 template nucleotide sequences. In some cases, generating substrate nucleic acids may comprise 44 template nucleotide sequences. In some cases, generating substrate nucleic acids may comprise 45 template nucleotide sequences. In some cases, generating substrate nucleic acids may comprise 46 template nucleotide sequences. In some cases, generating substrate nucleic acids may comprise 47 template nucleotide sequences. In some cases, generating substrate nucleic acids may comprise 48 template nucleotide sequences. In some cases, generating substrate nucleic acids may comprise 49 template nucleotide sequences. In some cases, generating substrate nucleic acids may comprise 50 template nucleotide sequences. In some cases, generating substrate nucleic acids may comprise about 55 template nucleotide sequences, about 60 template nucleotide sequences, about 65 template nucleotide sequences, about 70 template nucleotide sequences, about 75 template nucleotide sequences, about 80 template nucleotide sequences, about 85 template nucleotide sequences, about 90 template nucleotide sequences, about 95 template nucleotide sequences, about 100 template nucleotide sequences, about 110 template nucleotide sequences, about 120 template nucleotide sequences, about 130 template nucleotide sequences, about 140 template nucleotide sequences, about 150 template nucleotide sequences, about 160 template nucleotide sequences, about 170 template nucleotide sequences, about 180 template nucleotide sequences, about 190 template nucleotide sequences, about 200 template nucleotide sequences, about 210 template nucleotide sequences, about 220 template nucleotide sequences, about 230 template nucleotide sequences, about 240 template nucleotide sequences, about 250 template nucleotide sequences, about 260 template nucleotide sequences, about 270 template nucleotide sequences, about 280 template nucleotide sequences, about 290 template nucleotide sequences, about 300 template nucleotide sequences, about 350 template nucleotide sequences, about 400 template nucleotide sequences, about 450 template nucleotide sequences, about 500 template nucleotide sequences, about 1000 template nucleotide sequences, about 1500 template nucleotide sequences, about 2000 template nucleotide sequences, about 2500 template nucleotide sequences, about 3000 template nucleotide sequences, about 3500 template nucleotide sequences, about 4000 template nucleotide sequences, about 4500 template nucleotide sequences, or about 5000 template nucleotide sequences.

In some cases, generating substrate nucleic acids may comprise from 1 to 50 template nucleotide sequences. In some cases, generating substrate nucleic acids may comprise from 2 to 50 template nucleotide sequences. In some cases, generating substrate nucleic acids may comprise from 3 to 50 template nucleotide sequences. In some cases, generating substrate nucleic acids may comprise from 4 to 50 template nucleotide sequences. In some cases, generating substrate nucleic acids may comprise from 5 to 50 template nucleotide sequences. In some cases, generating substrate nucleic acids may comprise from 2 to 20 template nucleotide sequences. In some cases, generating substrate nucleic acids may comprise from 2 to 15 template nucleotide sequences. In some cases, generating substrate nucleic acids may comprise from 2 to 10 template nucleotide sequences. In some cases, generating substrate nucleic acids may comprise from 2 to 5 template nucleotide sequences. In some cases, generating substrate nucleic acids may comprise from 2 to 3 template nucleotide sequences. In some cases, generating substrate nucleic acids may comprise from 3 to 20 template nucleotide sequences. In some cases, generating substrate nucleic acids may comprise from 3 to 15 template nucleotide sequences. In some cases, generating substrate nucleic acids may comprise from 3 to 10 template nucleotide sequences. In some cases, generating substrate nucleic acids may comprise from 3 to 5 template nucleotide sequences. In some cases, generating substrate nucleic acids may comprise from 1 to 20 template nucleotide sequences. In some cases, generating substrate nucleic acids may comprise from 1 to 15 template nucleotide sequences. In some cases, generating substrate nucleic acids may comprise from 1 to 10 template nucleotide sequences. In some cases, generating substrate nucleic acids may comprise from 1 to 5 template nucleotide sequences. In some cases, generating substrate nucleic acids may comprise from 1 to 3 template nucleotide sequences.

In some instances, generating substrate nucleic acids may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 complement sequences of template nucleotide sequences. In some instances, generating substrate nucleic acids may comprise 1, 2, 3, 4, 5, 6, 7, 8, 9, 10, 11, 12, 13, 14, 15, 16, 17, 18, 19, 20, 21, 22, 23, 24, or 25 complement sequences of template nucleotide sequences. In some instances, generating substrate nucleic acids may comprise 1, 2, 3, 4, 5, 6, 7, 8, 9, 10, 11, 12, 13, 14, 15, 16, 17, 18, 19, or 20 complement sequences of template nucleotide sequences. In some instances, generating substrate nucleic acids may comprise 1, 2, 3, 4, 5, 6, 7, 8, 9, 10, 11, 12, 13, 14, or 15 complement sequences of template nucleotide sequences. In some instances, generating substrate nucleic acids may comprise 1, 2, 3, 4, 5, 6, 7, 8, 9, or 10 complement sequences of template nucleotide sequences. In some instances, generating substrate nucleic acids may comprise 1, 2, 3, 4, 5, 6, 7, 8, or 9 complement sequences of template nucleotide sequences. In some instances, generating substrate nucleic acids may comprise 1, 2, 3, 4, 5, 6, 7, or 8 complement sequences of template nucleotide sequences. In some instances, generating substrate nucleic acids may comprise 1, 2, 3, 4, 5, 6, or 7 complement sequences of template nucleotide sequences. In some instances, generating substrate nucleic acids may comprise 1, 2, 3, 4, 5, or 6 complement sequences of template nucleotide sequences. In some instances, generating substrate nucleic acids may comprise 1, 2, 3, 4, or 5 complement sequences of template nucleotide sequences. In some instances, generating substrate nucleic acids may comprise 1, 2, 3, or 4 complement sequences of template nucleotide sequences. In some instances, generating substrate nucleic acids may comprise 1, 2, or 3 complement sequences of template nucleotide sequences. In some instances, generating substrate nucleic acids may comprise 1, or 2 complement sequences of template nucleotide sequences.

In some cases, generating substrate nucleic acids may comprise 1 complement sequence of a template nucleotide sequence. In some cases, generating substrate nucleic acids may comprise 2 complement sequences of template nucleotide sequences. In some instances, generating substrate nucleic acids may comprise 3 complement sequences of template nucleotide sequences. In some cases, generating substrate nucleic acids may comprise 4 complement sequences of template nucleotide sequences. In some cases, generating substrate nucleic acids may comprise 5 complement sequences of template nucleotide sequences. In some cases, generating substrate nucleic acids may comprise 6 complement sequences of template nucleotide sequences. In some cases, generating substrate nucleic acids may comprise 7 complement sequences of template nucleotide sequences. In some cases, generating substrate nucleic acids may comprise 8 complement sequences of template nucleotide sequences. In some cases, generating substrate nucleic acids may comprise 9 complement sequences of template nucleotide sequences. In some cases, generating substrate nucleic acids may comprise 10 complement sequences of template nucleotide sequences. In some cases, generating substrate nucleic acids may comprise 11 complement sequences of template nucleotide sequences. In some cases, generating substrate nucleic acids may comprise 12 complement sequences of template nucleotide sequences. In some cases, generating substrate nucleic acids may comprise 13 complement sequences of template nucleotide sequences. In some cases, generating substrate nucleic acids may comprise 14 complement sequences of template nucleotide sequences. In some cases, generating substrate nucleic acids may comprise 15 complement sequences of template nucleotide sequences. In some cases, generating substrate nucleic acids may comprise 16 complement sequences of template nucleotide sequences. In some cases, generating substrate nucleic acids may comprise 17 complement sequences of template nucleotide sequences. In some cases, generating substrate nucleic acids may comprise 18 complement sequences of template nucleotide sequences. In some cases, generating substrate nucleic acids may comprise 19 complement sequences of template nucleotide sequences. In some cases, generating substrate nucleic acids may comprise 20 complement sequences of template nucleotide sequences. In some cases, generating substrate nucleic acids may comprise 21 complement sequences of template nucleotide sequences. In some cases, generating substrate nucleic acids may comprise 22 complement sequences of template nucleotide sequences. In some cases, generating substrate nucleic acids may comprise 23 complement sequences of template nucleotide sequences. In some cases, generating substrate nucleic acids may comprise 24 complement sequences of template nucleotide sequences. In some cases, generating substrate nucleic acids may comprise 25 complement sequences of template nucleotide sequences. In some cases, generating substrate nucleic acids may comprise 26 complement sequences of template nucleotide sequences. In some cases, generating substrate nucleic acids may comprise 27 complement sequences of template nucleotide sequences. In some cases, generating substrate nucleic acids may comprise 28 complement sequences of template nucleotide sequences. In some cases, generating substrate nucleic acids may comprise 29 complement sequences of template nucleotide sequences. In some cases, generating substrate nucleic acids may comprise 30 complement sequences of template nucleotide sequences. In some cases, generating substrate nucleic acids may comprise 31 complement sequences of template nucleotide sequences. In some cases, generating substrate nucleic acids may comprise 32 complement sequences of template nucleotide sequences. In some cases, generating substrate nucleic acids may comprise 33 complement sequences of template nucleotide sequences. In some cases, generating substrate nucleic acids may comprise 34 complement sequences of template nucleotide sequences. In some cases, generating substrate nucleic acids may comprise 35 complement sequences of template nucleotide sequences. In some cases, generating substrate nucleic acids may comprise 36 complement sequences of template nucleotide sequences. In some cases, generating substrate nucleic acids may comprise 37 complement sequences of template nucleotide sequences. In some cases, generating substrate nucleic acids may comprise 38 complement sequences of template nucleotide sequences. In some cases, generating substrate nucleic acids may comprise 39 complement sequences of template nucleotide sequences. In some cases, generating substrate nucleic acids may comprise 40 complement sequences of template nucleotide sequences. In some cases, generating substrate nucleic acids may comprise 41 complement sequences of template nucleotide sequences. In some cases, generating substrate nucleic acids may comprise 42 complement sequences of template nucleotide sequences. In some cases, generating substrate nucleic acids may comprise 43 complement sequences of template nucleotide sequences. In some cases, generating substrate nucleic acids may comprise 44 complement sequences of template nucleotide sequences. In some cases, generating substrate nucleic acids may comprise 45 complement sequences of template nucleotide sequences. In some cases, generating substrate nucleic acids may comprise 46 complement sequences of template nucleotide sequences. In some cases, generating substrate nucleic acids may comprise 47 complement sequences of template nucleotide sequences. In some cases, generating substrate nucleic acids may comprise 48 complement sequences of template nucleotide sequences. In some cases, generating substrate nucleic acids may comprise 49 complement sequences of template nucleotide sequences. In some cases, generating substrate nucleic acids may comprise 50 complement sequences of template nucleotide sequences. In some cases, generating substrate nucleic acids may comprise about 55 complement sequences of template nucleotide sequences, about 60 complement sequences of template nucleotide sequences, about 65 complement sequences of template nucleotide sequences, about 70 complement sequences of template nucleotide sequences, about 75 complement sequences of template nucleotide sequences, about 80 complement sequences of template nucleotide sequences, about 85 complement sequences of template nucleotide sequences, about 90 complement sequences of template nucleotide sequences, about 95 complement sequences of template nucleotide sequences, about 100 complement sequences of template nucleotide sequences, about 110 complement sequences of template nucleotide sequences, about 120 complement sequences of template nucleotide sequences, about 130 complement sequences of template nucleotide sequences, about 140 complement sequences of template nucleotide sequences, about 150 complement sequences of template nucleotide sequences, about 160 complement sequences of template nucleotide sequences, about 170 complement sequences of template nucleotide sequences, about 180 complement sequences of template nucleotide sequences, about 190 complement sequences of template nucleotide sequences, about 200 complement sequences of template nucleotide sequences, about 210 complement sequences of template nucleotide sequences, about 220 complement sequences of template nucleotide sequences, about 230 complement sequences of template nucleotide sequences, about 240 complement sequences of template nucleotide sequences, about 250 complement sequences of template nucleotide sequences, about 260 complement sequences of template nucleotide sequences, about 270 complement sequences of template nucleotide sequences, about 280 complement sequences of template nucleotide sequences, about 290 complement sequences of template nucleotide sequences, about 300 complement sequences of template nucleotide sequences, about 350 complement sequences of template nucleotide sequences, about 400 complement sequences of template nucleotide sequences, about 450 complement sequences of template nucleotide sequences, about 500 complement sequences of template nucleotide sequences, about 1000 complement sequences of template nucleotide sequences, about 1500 complement sequences of template nucleotide sequences, about 2000 complement sequences of template nucleotide sequences, about 2500 complement sequences of template nucleotide sequences, about 3000 complement sequences of template nucleotide sequences, about 3500 complement sequences of template nucleotide sequences, about 4000 complement sequences of template nucleotide sequences, about 4500 complement sequences of template nucleotide sequences, or about 5000 complement sequences of template nucleotide sequences.

In some cases, generating substrate nucleic acids may comprise from 1 to 50 complement sequences of template nucleotide sequences. In some cases, generating substrate nucleic acids may comprise from 2 to 50 complement sequences of template nucleotide sequences. In some cases, generating substrate nucleic acids may comprise from 3 to 50 complement sequences of template nucleotide sequences. In some cases, generating substrate nucleic acids may comprise from 4 to 50 complement sequences of template nucleotide sequences. In some cases, generating substrate nucleic acids may comprise from 5 to 50 complement sequences of template nucleotide sequences. In some cases, generating substrate nucleic acids may comprise from 2 to 20 complement sequences of template nucleotide sequences. In some cases, generating substrate nucleic acids may comprise from 2 to 15 complement sequences of template nucleotide sequences. In some cases, generating substrate nucleic acids may comprise from 2 to 10 complement sequences of template nucleotide sequences. In some cases, generating substrate nucleic acids may comprise from 2 to 5 complement sequences of template nucleotide sequences. In some cases, generating substrate nucleic acids may comprise from 2 to 3 complement sequences of template nucleotide sequences. In some cases, generating substrate nucleic acids may comprise from 3 to 20 complement sequences of template nucleotide sequences. In some cases, generating substrate nucleic acids may comprise from 3 to 15 complement sequences of template nucleotide sequences. In some cases, generating substrate nucleic acids may comprise from 3 to 10 complement sequences of template nucleotide sequences. In some cases, generating substrate nucleic acids may comprise from 3 to 5 complement sequences of template nucleotide sequences. In some cases, generating substrate nucleic acids may comprise from 1 to 20 complement sequences of template nucleotide sequences. In some cases, generating substrate nucleic acids may comprise from 1 to 15 complement sequences of template nucleotide sequences. In some cases, generating substrate nucleic acids may comprise from 1 to 10 complement sequences of template nucleotide sequences. In some cases, generating substrate nucleic acids may comprise from 1 to 5 complement sequences of template nucleotide sequences. In some cases, generating substrate nucleic acids may comprise from 1 to 3 complement sequences of template nucleotide sequences.

In some instances, each of a plurality of template nucleotide sequences or each of complements of a plurality of template nucleotide sequences may have at least a sequence aligning to a non-separate portion in a reference genome of a virus. Two non-separate portions in a reference genome may share at least a nucleotide with the same coordinate number. In other cases, non-two separate portions in a reference genome may share at least 2 nucleotides with the same coordinate number, at least 3 nucleotides with the same coordinate number, at least 4 nucleotides with the same coordinate number, at least 5 nucleotides with the same coordinate number, at least 6 nucleotides with the same coordinate number, at least 7 nucleotides with the same coordinate number, at least 8 nucleotides with the same coordinate number, at least 9 nucleotides with the same coordinate number, at least 10 nucleotides with the same coordinate number, at least 11 nucleotides with the same coordinate number, at least 12 nucleotides with the same coordinate number, at least 13 nucleotides with the same coordinate number, at least 14 nucleotides with the same coordinate number, at least 15 nucleotides with the same coordinate number, at least about 20 nucleotides with the same coordinate number, at least about 25 nucleotides with the same coordinate number, at least about 30 nucleotides with the same coordinate number, at least about 35 nucleotides with the same coordinate number, at least about 40 nucleotides with the same coordinate number, at least about 45 nucleotides with the same coordinate number, at least about 50 nucleotides with the same coordinate number, at least about 100 nucleotides with the same coordinate number, at least about 200 nucleotides with the same coordinate number, at least about 300 nucleotides with the same coordinate number, at least about 400 nucleotides with the same coordinate number, at least about 500 nucleotides with the same coordinate number, at least about 600 nucleotides with the same coordinate number, at least about 700 nucleotides with the same coordinate number, at least about 800 nucleotides with the same coordinate number, at least about 900 nucleotides with the same coordinate number, at least about 1000 nucleotides with the same coordinate number, at least about 1500 nucleotides with the same coordinate number, at least about 2000 nucleotides with the same coordinate number, at least about 2500 nucleotides with the same coordinate number, at least about 3000 nucleotides with the same coordinate number, at least about 3500 nucleotides with the same coordinate number, at least about 4000 nucleotides with the same coordinate number, at least about 4500 nucleotides with the same coordinate number, at least about 5000 nucleotides with the same coordinate number, at least about 10000 nucleotides with the same coordinate number, at least about 15000 nucleotides with the same coordinate number, at least about 20000 nucleotides with the same coordinate number, at least about 25000 nucleotides with the same coordinate number, at least about 30000 nucleotides with the same coordinate number, at least about 35000 nucleotides with the same coordinate number, at least about 40000 nucleotides with the same coordinate number, at least about 45000 nucleotides with the same coordinate number, at least about 50000 nucleotides with the same coordinate number.

In some instances, a template nucleotide sequence may comprise a DNA sequence or molecule. In other cases, a template nucleotide sequence may comprise an RNA sequence or molecule. In some cases, a template nucleotide sequence may be isolated from a biological sample described herein and elsewhere in this disclosure. In some cases, a template nucleotide sequence may be derived from a chemical reaction described herein and elsewhere in this disclosure. For example, such a chemical reaction may comprise any chemical reactions for substrate nucleic acid generations. In one case, such a chemical reaction may comprise an amplification reaction.

In some instances, a template nucleotide sequence may comprise at least a sequence aligning to a Spike (S) gene or at least a sequence aligning a Nucleocapsid (N) gene in a reference genome of a virus. In some cases, a template nucleotide sequence may comprise at least a sequence aligning to a Spike gene and at least a sequence aligning a Nucleocapsid gene in a reference genome of a virus. In some cases, a template nucleotide sequence may comprise at least a sequence aligning to an ORFlab gene in a reference genome of a virus. In some cases, a template nucleotide sequence may comprise at least a sequence aligning to a Spike (S) gene in a reference genome of a virus. In some cases, a template nucleotide sequence may comprise at least a sequence aligning to an Envelope (E) gene in a reference genome of a virus. In some cases, a template nucleotide sequence may comprise at least a sequence aligning to a Nucleocapsid (N) gene in a reference genome of a virus. In some cases, a template nucleotide sequence may comprise at least a sequence aligning to a Membrane (M) gene in a reference genome of a virus. In some cases, a template nucleotide sequence may comprise at least a sequence aligning to an ORF3a gene in a reference genome of a virus. In some cases, a template nucleotide sequence may comprise at least a sequence aligning to an ORF3b gene in a reference genome of a virus. In some cases, a template nucleotide sequence may comprise at least a sequence aligning to an ORF6 gene in a reference genome of a virus. In some cases, a template nucleotide sequence may comprise at least a sequence aligning to an ORF7a gene in a reference genome of a virus. In some cases, a template nucleotide sequence may comprise at least a sequence aligning to an ORF7b gene in a reference genome of a virus. In some cases, a template nucleotide sequence may comprise at least a sequence aligning to an ORF8a gene in a reference genome of a virus. In some cases, a template nucleotide sequence may comprise at least a sequence aligning to an ORF8b gene in a reference genome of a virus. In some cases, a template nucleotide sequence may comprise at least a sequence aligning to an ORF9b gene in a reference genome of a virus. In one case, a template nucleotide sequence may comprise at least a sequence aligning to a hemagglutinin-esterase (HE) gene in a reference genome of a virus.

In some cases, a protein encoded by a Spike gene may comprise the sequence of NCBI Accession: YP_009724390. In some cases, a protein encoded by an Envelope gene may comprise the sequence of NCBI Accession: YP_009724392). In some cases, a protein encoded by a Nucleocapsid gene may comprise the sequence of NCBI Accession: YP_009724397). In some cases, a protein encoded by a Membrane glycoprotein M gene may comprise the sequence of NCBI Accession: YP_009724393). In some cases, a protein encoded by an ORF lab gene may comprise the sequence of NCBI Accession: YP_009724389). In some cases, a protein encoded by an ORF3a gene may comprise the sequence of NCBI Accession: YP_009724391). In some cases, a protein encoded by an ORF6 gene may comprise the sequence of NCBI Accession: YP_009724394). In some cases, a protein encoded by an ORF7a gene may comprise the sequence of NCBI Accession: YP_009724395). In some cases, a protein encoded by an ORF7b gene may comprise the sequence of NCBI Accession: YP_009725318). In some cases, a protein encoded by an ORF8 gene may comprise the sequence of NCBI Accession: YP_009724396). In some cases, a protein encoded by an ORF10 gene may comprise the sequence of NCBI Accession: YP_009725255). Table 14 shows sequences of exemplary protein sequences encoded by template nucleotide sequences.

TABLE 14
Exemplary Protein Sequences Encoded by Template Nucleotide Sequence Sequences

| SEQ ID NO. | Sequence |
|---|---|
| SEQ ID NO. 29 | MFVFLVLLPLVSSQCVNLTTRTQLPPAYTNSFTRGVYYPDKVFRSSVL |
| | HSTQDLFLPFFSNVTWFHAIHVSGTNGTKRFDNPVLPFNDGVYFASTE |
| | KSNIIRGWIFGTTLDSKTQSLLIVNNATNVVIKVCEFQFCNDPFLGVYY |
| | HKNNKSWMESEFRVYSSANNCTFEYVSQPFLMDLEGKQGNFKNLREF |
| | VFKNIDGYFKIYSKHTPINLVRDLPQGFSALEPLVDLPIGINITRFQTLLA |
| | LHRSYLTPGDSSSGWTAGAAAYYVGYLQPRTFLLKYNENGTITDAVD |
| | CALDPLSETKCTLKSFTVEKGIYQTSNFRVQPTESIVRFPNITNLCPFGE |
| | VFNATRFASVYAWNRKRISNCVADYSVLYNSASFSTFKCYGVSPTKLN |
| | DLCFTNVYADSFVIRGDEVRQIAPGQTGKIADYNYKLPDDFTGCVIAW |
| | NSNNLDSKVGGNYNYLYRLFRKSNLKPFERDISTEIYQAGSTPCNGVE |
| | GFNCYFPLQSYGFQPTNGVGYQPYRVVVLSFELLHAPATVCGPKKSTN |
| | LVKNKCVNFNFNGLTGTGVLTESNKKFLPFQQFGRDIADTTDAVRDPQ |
| | TLEILDITPCSFGGVSVITPGTNTSNQVAVLYQDVNCTEVPVAIHADQL |
| | TPTWRVYSTGSNVFQTRAGCLIGAEHVNNSYECDIPIGAGICASYQTQT |
| | NSPRRARSVASQSIIAYTMSLGAENSVAYSNNSIAIPTNFTISVTTEILPV |
| | SMTKTSVDCTMYICGDSTECSNLLLQYGSFCTQLNRALTGIAVEQDKN |
| | TQEVFAQVKQIYKTPPIKDFGGFNFSQILPDPSKPSKRSFIEDLLENKVT |
| | LADAGFIKQYGDCLGDIAARDLICAQKFNGLTVLPPLLTDEMIAQYTS |
| | ALLAGTITSGWTFGAGAALQIPFAMQMAYRENGIGVTQNVLYENQKLI |
| | ANQFNSAIGKIQDSLSSTASALGKLQDVVNQNAQALNTLVKQLSSNFG |
| | AISSVLNDILSRLDKVEAEVQIDRLITGRLQSLQTYVTQQLIRAAEIRAS |
| | ANLAATKMSECVLGQSKRVDFCGKGYHLMSFPQSAPHGVVFLHVTY |
| | VPAQEKNFTTAPAICHDGKAHFPREGVFVSNGTHWFVTQRNFYEPQIIT |
| | TDNTFVSGNCDVVIGIVNNTVYDPLQPELDSFKEELDKYFKNHTSPDV |
| | DLGDISGINASVVNIQKEIDRLNEVAKNLNESLIDLQELGKYEQYIKWP |
| | WYIWLGFIAGLIAIVMVTIMLCCMTSCCSCLKGCCSCGSCCKFDEDDS |
| | EPVLKGVKLHYT |
| SEQ ID NO. 30 | MSDNGPQNQRNAPRITFGGPSDSTGSNQNGERSGARSKQRRPQGLPNN |
| | TASWFTALTQHGKEDLKFPRGQGVPINTNSSPDDQIGYYRRATRRIRG |
| | GDGKMKDLSPRWYFYYLGTGPEAGLPYGANKDGIIWVATEGALNTPK |
| | DHIGTRNPANNAAIVLQLPQGTTLPKGFYAEGSRGGSQASSRSSSRSRN |
| | SSRNSTPGSSRGTSPARMAGNGGDAALALLLLDRLNQLESKMSGKGQ |
| | QQQGQTVTKKSAAEASKKPRQKRTATKAYNVTQAFGRRGPEQTQGN |
| | FGDQELIRQGTDYKHWPQIAQFAPSASAFFGMSRIGMEVTPSGTWLTY |
| | TGAIKLDDKDPNFKDQVILLNKHIDAYKTFPPTEPKKDKKKKADETQA |
| | LPQRQKKQQTVTLLPAADLDDFSKQLQQSMSSADSTQA |

In some cases, a plurality of template nucleotide sequences may comprise at least a sequence that encodes a peptide having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence of SEQ ID NO. 29. In some cases, a plurality of template nucleotide sequences may comprise at least a sequence that encodes a peptide having at least about 70%, 71%, 72%, 73%, 74%, 75%, 76%, 77%, 78%, 79%, 800%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence of SEQ ID NO. 30.

In some cases, a Spike gene may comprise the sequence of NCBI Accession NC_045512.2, 21563 . . . 25384). In some cases, an Envelope gene may comprise the sequence of NCBI Accession NC_045512.2, 26245 . . . 26472). In some cases, a Nucleocapsid gene may comprise the sequence of NCBI Accession NC_045512.2, 28274 . . . 29533). In some cases, a Membrane glycoprotein M gene may comprise the sequence of NCBI Accession NC_045512.2, 26523 to 27191). In some cases, an ORF lab gene may comprise the sequence of NCBI Accession NC_045512.2, 266 . . . 21555). In some cases, an ORF3a gene may comprise the sequence of NCBI Accession NC_045512.2, 25393 . . . 26220). In some cases, an ORF6 gene may comprise the sequence of NCBI AccessionNC_045512.2, 27202 . . . 27387). In some cases, an ORF7a gene may comprise the sequence of NCBI Accession NC_045512.2, 27394 . . . 27759). In some cases, an ORF7b gene may comprise the sequence of NCBI Accession NC_045512.2, 27756 . . . 27887). In some cases, an ORF8 gene may comprise the sequence of NCBI Accession NC_045512.2, 27894 . . . 28259). In some cases, an ORF10 gene may comprise the sequence of NCBI Accession NC_045512.2, 29558 . . . 29674). Table 15 shows sequences of exemplary template nucleotide sequences.

TABLE 15
Exemplary Template Nucleotide Sequence Sequences

| SEQ ID NO. | Sequence |
|---|---|
| SEQ ID | atgtttgtttttcttgttttattgccactagtctctagtcagtgtgttaatcttacaaccagaactcaattaccccctgcat |
| NO. 31 | acactaattctttcacacgtggtgtttattaccctgacaaagttttcagatcctcagttttacattcaactcaggacttgt |
| | tcttacctttcttttccaatgttacttggttccatgctatacatgtctctgggaccaatggtactaagaggtttgataacc |
| | ctgtcctaccatttaatgatggtgtttattttgcttccactgagaagtctaacataataagaggctggatttttggtacta |
| | ctttagattcgaagacccagtccctacttattgttaataacgctactaatgttgttattaaagtctgtgaatttcaattttg |
| | taatgatccatttttgggtgtttattaccacaaaaacaacaaaagttggatggaaagtgagttcagagtttattctagt |
| | gcgaataattgcacttttgaatatgtctctcagccttttcttatggaccttgaaggaaaacagggtaatttcaaaaatc |
| | ttagggaatttgtgtttaagaatattgatggttattttaaaatatattctaagcacacgcctattaatttagtgcgtgatct |
| | ccctcagggtttttcggctttagaaccattggtagatttgccaataggtattaacatcactaggtttcaaactttacttg |
| | ctttacatagaagttatttgactcctggtgattcttcttcaggttggacagctggtgctgcagcttattatgtgggttat |
| | cttcaacctaggacttttctattaaaatataatgaaaatggaaccattacagatgctgtagactgtgcacttgaccct |
| | ctctcagaaacaaagtgtacgttgaaatccttcactgtagaaaaaggaatctatcaaacttctaactttagagtcca |
| | accaacagaatctattgttagatttcctaatattacaaacttgtgcccttttggtgaagtttttaacgccaccagatttg |
| | catctgtttatgcttggaacaggaagagaatcagcaactgtgttgctgattattctgtcctatataattccgcatcattt |
| | tccacttttaagtgttatggagtgtctcctactaaattaaatgatctctgctttactaatgtctatgcagattcatttgtaat |
| | tagaggtgatgaagtcagacaaatcgctccagggcaaactggaaagattgctgattataattataaattaccagat |
| | gattttacaggctgcgttatagcttggaattctaacaatcttgattctaaggttggtggtaattataattacctgtatag |
| | attgtttaggaagtctaatctcaaaccttttgagagagatatttcaactgaaatctatcaggccggtagcacaccttg |
| | taatggtgttgaaggttttaattgttactttcctttacaatcatatggtttccaacccactaatggtgttggttaccaacc |
| | atacagagtagtagtactttcttttgaacttctacatgcaccagcaactgtttgtggacctaaaaagtctactaatttg |
| | gttaaaaacaaatgtgtcaatttcaacttcaatggtttaacaggcacaggtgttcttactgagtctaacaaaaagtttc |
| | tgcctttccaacaatttggcagagacattgctgacactactgatgctgtccgtgatccacagacacttgagattcttg |
| | acattacaccatgttcttttggtggtgtcagtgttataacaccaggaacaaatacttctaaccaggttgctgttctttat |
| | caggatgttaactgcacagaagtccctgttgctattcatgcagatcaacttactcctacttggcgtgtttattctacag |
| | gttctaatgtttttcaaacacgtgcaggctgtttaataggggctgaacatgtcaacaactcatatgagtgtgacatac |
| | ccattggtgcaggtatatgcgctagttatcagactcagactaattctcctcgggggcacgtagtgtagctagtca |
| | atccatcattgcctacactatgtcacttggtgcagaaaattcagttgcttactctaataactctattgccatacccaca |
| | aattttactattagtgttaccacagaaattctaccagtgtctatgaccaagacatcagtagattgtacaatgtacatttg |
| | tggtgattcaactgaatgcagcaatcttttgttgcaatatggcagtttttgtacacaattaaaccgtgctttaactgga |
| | atagctgttgaacaagacaaaaacacccaagaagtttttgcacaagtcaaacaaatttacaaaacaccaccaatta |
| | aagattttggtggttttaatttttcacaaatattaccagatccatcaaaaccaagcaagaggtcatttattgaagatcta |
| | cttttcaacaaagtgacacttgcagatgctggcttcatcaaacaatatggtgattgccttggtgatattgctgctaga |
| | gacctcatttgtgcacaaaagtttaacggccttactgttttgccacctttgctcacagatgaaatgattgctcaataca |
| | cttctgcactgttagcgggtacaatcacttctggttggacctttggtgcaggtgctgcattacaaataccatttgctat |
| | gcaaatggcttataggtttaatggtattggagttacacagaatgttctctatgagaaccaaaaattgattgccaacca |
| | atttaatagtgctattggcaaaattcaagactcactttcttccacagcaagtgcacttggaaaacttcaagatgtggt |
| | caaccaaaatgcacaagctttaaacacgcttgttaaacaacttagctccaattttggtgcaatttcaagtgttttaaat |
| | gatatcctttcacgtcttgacaaagttgaggctgaagtgcaaattgataggttgatcacaggcagacttcaaagttt |
| | gcagacatatgtgactcaacaattaattagagctgcagaaatcagagcttctgctaatcttgctgctactaaaatgt |
| | cagagtgtgtacttggacaatcaaaaagagttgatttttgtggaaagggctatcatcttatgtccttccctcagtcag |
| | cacctcatggtgtagtcttcttgcatgtgacttatgtccctgcacaagaaaagaacttcacaactgctcctgccattt |
| | gtcatgatggaaaagcacactttcctcgtgaaggtgtctttgtttcaaatggcacacactggtttgtaacacaaagg |
| | aatttttatgaaccacaaatcattactacagacaacacatttgtgtctggtaactgtgatgttgtaataggaattgtca |
| | acaacacagtttatgatcctttgcaacctgaattagactcattcaaggaggagttagataaatattttaagaatcata |
| | catcaccagatgttgatttaggtgacatctctggcattaatgcttcagttgtaaacattcaaaaagaaattgaccgcc |
| | tcaatgaggttgccaagaatttaaatgaatctctcatcgatctccaagaacttggaaagtatgagcagtatataaaa |
| | tggccatggtacatttggctaggttttatagctggcttgattgccatagtaatggtgacaattatgctttgctgtatgac |
| | cagttgctgtagttgtctcaagggctgttgttcttgtggatcctgctgcaaatttgatgaagacgactctgagccagt |
| | gctcaaaggagtcaaattacattacacataa |
| SEQ ID | atgtctgataatggaccccaaaatcagcgaaatgcaccccgcattacgtttggtggaccctcagattcaactggc |
| NO. 32 | agtaaccagaatggagaacgcagtggggcgcgatcaaaacaacgtcggccccaaggtttacccaataatactg |
| | cgtcttggttcaccgctctcactcaacatggcaaggaagaccttaaattccctcgaggacaaggcgttccaattaa |
| | caccaatagcagtccagatgaccaaattggctactaccgaagagctaccagacgaattcgtggtggtgacggta |
| | aaatgaaagatctcagtccaagatggtatttctactacctaggaactgggccagaagctggacttccctatggtgc |
| | taacaaagacggcatcatatgggttgcaactgagggagccttgaatacaccaaaagatcacattggcacccgca |
| | atcctgctaacaatgctgcaatcgtgctacaacttcctcaaggaacaacattgccaaaaggcttctacgcagaag |
| | ggagcagaggcggcagtcaagcctcttctcgttcctcatcacgtagtcgcaacagttcaagaaattcaactccag |
| | gcagcagtaggggaacttctcctgctagaatggctggcaatggcggtgatgctgctcttgctttgctgctgcttga |
| | cagattgaaccagcttgagagcaaaatgtctggtaaaggccaacaacaacaaggccaaactgtcactaagaaat |
| | ctgctgctgaggcttctaagaagcctcggcaaaaacgtactgccactaaagcatacaatgtaacacaagctttcg |
| | gcagacgtggtccagaacaaacccaaggaaattttggggaccaggaactaatcagacaaggaactgattacaa |
| | acattggccgcaaattgcacaatttgcccccagcgcttcagcgttcttcggaatgtcgcgcattggcatggaagtc |
| | acaccttcgggaacgtggttgacctacacaggtgccatcaaattggatgacaaagatccaaatttcaaagatcaa |
| | gtcattttgctgaataagcatattgacgcatacaaaacattcccaccaacagagcctaaaaaggacaaaaagaag |
| | aaggctgatgaaactcaagccttaccgcagagacagaagaaacagcaaactgtgactcttcttcctgctgcagat |
| | ttggatgatttctccaaacaattgcaacaatccatgagcagtgctgactcaactcaggcctaa |

In some cases, template nucleotide sequences may comprise a sequence aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise two sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise three sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise four sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise five sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise six sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise seven sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise eight sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise nine sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise ten sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise eleven sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise twelve sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise thirteen sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise fourteen sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene.

In some cases, template nucleotide sequences may comprise at least one sequence aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least two sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least three sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least four sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least five sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least six sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least seven sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least eight sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least nine sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least ten sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least eleven sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least twelve sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus. In some cases, template nucleotide sequences may comprise at least thirteen sequences aligning to a Spike gene, an Envelope gene, a Nucleocapsid gene, a Membrane glycoprotein M gene, an ORF3a gene, an ORF3b gene, an ORF6 gene, an ORF7a gene, an ORF7b gene, an ORF8a gene, an ORF8b gene, or an ORF9b gene in a reference genome of a virus.

In some cases, a template nucleotide sequence may comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence of a Spike gene or a Nucleocapsid gene of a reference genome of a virus of family Coronaviridae.

In some instances, a template nucleotide sequence may comprise at least 80% sequence identity to a sequence of a Spike gene or a Nucleocapsid gene of a reference genome of a virus of family Coronaviridae. In some cases, a template nucleotide sequence may comprise at least 85% sequence identity to a sequence of a Spike gene or a Nucleocapsid gene of a reference genome of a virus of family Coronaviridae. In some cases, a template nucleotide sequence may comprise at least 90% sequence identity to a sequence of a Spike gene or a Nucleocapsid gene of a reference genome of a virus of family Coronaviridae. In some cases, a template nucleotide sequence may comprise at least 95% sequence identity to a sequence of a Spike gene or a Nucleocapsid gene of a reference genome of a virus of family Coronaviridae. In some cases, a template nucleotide sequence may comprise at least 96% sequence identity to a sequence of a Spike gene or a Nucleocapsid gene of a reference genome of a virus of family Coronaviridae. In some cases, a template nucleotide sequence may comprise at least 97% sequence identity to a sequence of a Spike gene or a Nucleocapsid gene of a reference genome of a virus of family Coronaviridae. In some cases, a template nucleotide sequence may comprise at least 98% sequence identity to a sequence of a Spike gene or a Nucleocapsid gene of a reference genome of a virus of family Coronaviridae. In some cases, a template nucleotide sequence may comprise at least 99% sequence identity to a sequence of a Spike gene or a Nucleocapsid gene of a reference genome of a virus of family Coronaviridae. In some cases, a template nucleotide sequence may comprise of a reference genome of a virus of family Coronaviridae. In some cases, a template nucleotide sequence may comprise at least 50%, at least 55%, at least 60%, at least 65%, at least 70%, at least 75%, at least 80%, at least 81%, at least 82%, at least 83%, at least 84%, at least 86%, at least 87%, at least 88%, at least 89%, at least 91%, at least 92%, at least 93%, or at least 94% sequence identity to a sequence of a Spike gene or a Nucleocapsid gene of a reference genome of a virus of family Coronaviridae.

In some instances, a template nucleotide sequence may comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence of NC_045512.2 (21563 . . . 29533).

In some instances, a template nucleotide sequence may comprise at least 80% sequence identity to a sequence of NC_045512.2 (21563 . . . 29533). In some cases, a template nucleotide sequence may comprise at least 85% sequence identity to a sequence of NC_045512.2 (21563 . . . 29533). In some cases, a template nucleotide sequence may comprise at least 90% sequence identity to a sequence of NC_045512.2 (21563 . . . 29533. In some cases, a template nucleotide sequence may comprise at least 95% sequence identity to a sequence of NC_045512.2 (21563 . . . 29533). In some cases, a template nucleotide sequence may comprise at least 96% sequence identity to a sequence of NC_045512.2 (21563 . . . 29533). In some cases, a template nucleotide sequence may comprise at least 97% sequence identity to a sequence of NC_045512.2 (21563 . . . 29533). In some cases, a template nucleotide sequence may comprise at least 98% sequence identity to a sequence of NC_045512.2 (21563 . . . 29533). In some cases, a template nucleotide sequence may comprise at least 99% sequence identity to a sequence of NC_045512.2 (21563 . . . 29533). In some cases, a template nucleotide sequence may comprise of a reference genome of a virus of family Coronaviridae. In some cases, a template nucleotide sequence may comprise at least 50%, at least 55%, at least 60%, at least 65%, at least 70%, at least 75%, at least 80%, at least 81%, at least 82%, at least 83%, at least 84%, at least 86%, at least 87%, at least 88%, at least 89%, at least 91%, at least 92%, at least 93%, or at least 94% sequence identity to a sequence of NC_045512.2 (21563 . . . 29533).

In some cases, a plurality of template nucleotide sequences may comprise at least a sequence that comprises at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence of SEQ ID NO. 31. In some cases, a plurality of template nucleotide sequences may comprise at least a sequence that comprises at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence of SEQ ID NO. 32.

In some cases, a plurality of template nucleotide sequences may comprise at least a sequence corresponding to a region spanning across nucleotide positions 331-670, 1027-1306, 1211-1571, 1424-1739, 1435-1972, 1710-1915, 1726-1865, 1997-2250, 2567-2642, 3149-3723, or a combination thereof of SEQ ID NO. 31. In some cases, a plurality of template nucleotide sequences may comprise at least a sequence corresponding to a region spanning across nucleotide positions 331-670 of SEQ ID NO. 31. In some cases, a plurality of template nucleotide sequences may comprise at least a sequence corresponding to a region spanning across nucleotide positions 1027-1306 of SEQ ID NO. 31. In some cases, a plurality of template nucleotide sequences may comprise at least a sequence corresponding to a region spanning across nucleotide positions 1211-1571 of SEQ ID NO. 31. In some cases, a plurality of template nucleotide sequences may comprise at least a sequence corresponding to a region spanning across nucleotide positions 1424-1739 of SEQ ID NO. 31. In some cases, a plurality of template nucleotide sequences may comprise at least a sequence corresponding to a region spanning across nucleotide positions 1435-1972 of SEQ ID NO. 31. In some cases, a plurality of template nucleotide sequences may comprise at least a sequence corresponding to a region spanning across nucleotide positions 1710-1915 of SEQ ID NO. 31. In some cases, a plurality of template nucleotide sequences may comprise at least a sequence corresponding to a region spanning across nucleotide positions 1726-1865 of SEQ ID NO. 31. In some cases, a plurality of template nucleotide sequences may comprise at least a sequence corresponding to a region spanning across nucleotide positions 1997-2250 of SEQ ID NO. 31. In some cases, a plurality of template nucleotide sequences may comprise at least a sequence corresponding to a region spanning across nucleotide positions 2567-2642 of SEQ ID NO. 31. In some cases, a plurality of template nucleotide sequences may comprise at least a sequence corresponding to a region spanning across nucleotide positions 3149-3723 of SEQ ID NO. 31.

In some cases, a plurality of template nucleotide sequences may comprise at least a sequence corresponding to a region spanning across nucleotide positions 28312-28675, 28479-28651, 29044-29427, 29261-29521, or a combination thereof of SEQ ID NO. 32. In some cases, a plurality of template nucleotide sequences may comprise at least a sequence corresponding to a region spanning across nucleotide positions 28312-28675 of SEQ ID NO. 32. In some cases, a plurality of template nucleotide sequences may comprise at least a sequence corresponding to a region spanning across nucleotide positions 28479-28651 of SEQ ID NO. 32. In some cases, a plurality of template nucleotide sequences may comprise at least a sequence corresponding to a region spanning across nucleotide positions 29044-29427 of SEQ ID NO. 32. In some cases, a plurality of template nucleotide sequences may comprise at least a sequence corresponding to a region spanning across nucleotide positions 29261-29521 of SEQ ID NO. 32

In some instances, a template nucleotide sequence may comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence of a Spike gene of a reference genome of a virus of family Coronaviridae. In some instances, a template nucleotide sequence may comprise at least 80% sequence identity to a sequence of a Spike gene of a reference genome of a virus of family Coronaviridae. In some cases, a template nucleotide sequence may comprise at least 85% sequence identity to a sequence of a Spike gene of a reference genome of a virus of family Coronaviridae. In some cases, a template nucleotide sequence may comprise at least 90% sequence identity to a sequence of a Spike gene of a reference genome of a virus of family Coronaviridae. In some cases, a template nucleotide sequence may comprise at least 95% sequence identity to a sequence of a Spike gene of a reference genome of a virus of family Coronaviridae. In some cases, a template nucleotide sequence may comprise at least 96% sequence identity to a sequence of a Spike gene of a reference genome of a virus of family Coronaviridae. In some cases, a template nucleotide sequence may comprise at least 97% sequence identity to a sequence of a Spike gene of a reference genome of a virus of family Coronaviridae. In some cases, a template nucleotide sequence may comprise at least 98% sequence identity to a sequence of a Spike gene of a reference genome of a virus of family Coronaviridae. In some cases, a template nucleotide sequence may comprise at least 99% sequence identity to a sequence of a Spike gene of a reference genome of a virus of family Coronaviridae. In some cases, a template nucleotide sequence may comprise of a reference genome of a virus of family Coronaviridae. In some cases, a template nucleotide sequence may comprise at least 50%, at least 55%, at least 60%, at least 65%, at least 70%, at least 75%, at least 80%, at least 81%, at least 82%, at least 83%, at least 84%, at least 86%, at least 87%, at least 88%, at least 89%, at least 91%, at least 92%, at least 93%, or at least 94% sequence identity to a sequence of a Spike gene of a reference genome of a virus of family Coronaviridae.

In some cases, a template nucleotide sequence may comprise a sequence that encodes at least the K417, L452, E484, or N501 of a Spike protein encoded by a Spike gene of a reference genome of a virus of family Coronaviridae. In some cases, a template nucleotide sequence may comprise a sequence that encodes at least the K417, L452, E484, and N501 of a Spike protein encoded by a Spike gene of a reference genome of a virus of family Coronaviridae. In some cases, a template nucleotide sequence may comprise a sequence that encodes at least the K417, L452, E484, N501, A570, Q613, or D614 of a Spike protein encoded by a Spike gene of a reference genome of a virus of family Coronaviridae. In some cases, a template nucleotide sequence may comprise a sequence that encodes at least the K417, L452, E484, N501, A570, Q613, and D614 of a Spike protein encoded by a Spike gene of a reference genome of a virus of family Coronaviridae.

In some cases, a template nucleotide sequence may comprise a sequence that encodes at least the K417 of a Spike protein encoded by a Spike gene of a reference genome of a virus of family Coronaviridae. In some cases, a template nucleotide sequence may comprise a sequence that encodes at least the L452 of a Spike protein encoded by a Spike gene of a reference genome of a virus of family Coronaviridae. In some cases, a template nucleotide sequence may comprise a sequence that encodes at least the E484 of a Spike protein encoded by a Spike gene of a reference genome of a virus of family Coronaviridae. In some cases, a template nucleotide sequence may comprise a sequence that encodes at least the N501 of a Spike protein encoded by a Spike gene of a reference genome of a virus of family Coronaviridae. In some cases, a template nucleotide sequence may comprise a sequence that encodes at least the A570 of a Spike protein encoded by a Spike gene of a reference genome of a virus of family Coronaviridae. In some cases, a template nucleotide sequence may comprise a sequence that encodes at least the Q613 of a Spike protein encoded by a Spike gene of a reference genome of a virus of family Coronaviridae. In some cases, a template nucleotide sequence may comprise a sequence that encodes at least the D614 of a Spike protein encoded by a Spike gene of a reference genome of a virus of family Coronaviridae. In some cases, a template nucleotide sequence may comprise a sequence that encodes at least the E484 of a Spike protein encoded by a Spike gene of a reference genome of a virus of family Coronaviridae. In some cases, a template nucleotide sequence may comprise a sequence that encodes at least the N501 of a Spike protein encoded by a Spike gene of a reference genome of a virus of family Coronaviridae. In some cases, a template nucleotide sequence may comprise a sequence that encodes at least the A570 of a Spike protein encoded by a Spike gene of a reference genome of a virus of family Coronaviridae. In some cases, a template nucleotide sequence may comprise a sequence that encodes at least the Q613 of a Spike protein encoded by a Spike gene of a reference genome of a virus of family Coronaviridae.

In some cases, a template nucleotide sequence may comprise a sequence that encodes at least the amino acids between the K417 and L452 of a Spike protein encoded by a Spike gene of a reference genome of a virus of family Coronaviridae. In some cases, a template nucleotide sequence may comprise a sequence that encodes at least the amino acids between the K417 and E484 of a Spike protein encoded by a Spike gene of a reference genome of a virus of family Coronaviridae. In some cases, a template nucleotide sequence may comprise a sequence that encodes at least the amino acids between the K417 and N501 of a Spike protein encoded by a Spike gene of a reference genome of a virus of family Coronaviridae. In some cases, a template nucleotide sequence may comprise a sequence that encodes at least the amino acids between the K417 and A570 of a Spike protein encoded by a Spike gene of a reference genome of a virus of family Coronaviridae. In some cases, a template nucleotide sequence may comprise a sequence that encodes at least the amino acids between the K417 and Q613 of a Spike protein encoded by a Spike gene of a reference genome of a virus of family Coronaviridae. In some cases, a template nucleotide sequence may comprise a sequence that encodes at least the amino acids between the K417 and D614 of a Spike protein encoded by a Spike gene of a reference genome of a virus of family Coronaviridae. In some cases, a template nucleotide sequence may comprise a sequence that encodes at least the amino acids between the L452 and D614 of a Spike protein encoded by a Spike gene of a reference genome of a virus of family Coronaviridae. In some cases, a template nucleotide sequence may comprise a sequence that encodes at least the amino acids between the E484 and D614 of a Spike protein encoded by a Spike gene of a reference genome of a virus of family Coronaviridae. In some cases, a template nucleotide sequence may comprise a sequence that encodes at least the amino acids between the N501 and D614 of a Spike protein encoded by a Spike gene of a reference genome of a virus of family Coronaviridae. In some cases, a template nucleotide sequence may comprise a sequence that encodes at least the amino acids between the A570 and D614 of a Spike protein encoded by a Spike gene of a reference genome of a virus of family Coronaviridae. In some cases, a template nucleotide sequence may comprise a sequence that encodes at least the Q613 and D614 of a Spike protein encoded by a Spike gene of a reference genome of a virus of family Coronaviridae.

In some instances, a template nucleotide sequence may comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a sequence of a Nucleocapsid gene of a reference genome of a virus of family Coronaviridae.

In some instances, a template nucleotide sequence may comprise at least 80% sequence identity to a sequence of a Nucleocapsid gene of a reference genome of a virus of family Coronaviridae. In some cases, a template nucleotide sequence may comprise at least 81% sequence identity to a sequence of a Nucleocapsid gene of a reference genome of a virus of family Coronaviridae. In some cases, a template nucleotide sequence may comprise at least 82% sequence identity to a sequence of a Nucleocapsid gene of a reference genome of a virus of family Coronaviridae. In some cases, a template nucleotide sequence may comprise at least 83% sequence identity to a sequence of a Nucleocapsid gene of a reference genome of a virus of family Coronaviridae. In some cases, a template nucleotide sequence may comprise at least 84% sequence identity to a sequence of a Nucleocapsid gene of a reference genome of a virus of family Coronaviridae. In some cases, a template nucleotide sequence may comprise at least 85% sequence identity to a sequence of a Nucleocapsid gene of a reference genome of a virus of family Coronaviridae. In some cases, a template nucleotide sequence may comprise at least 86% sequence identity to a sequence of a Nucleocapsid gene of a reference genome of a virus of family Coronaviridae. In some cases, a template nucleotide sequence may comprise at least 87% sequence identity to a sequence of a Nucleocapsid gene of a reference genome of a virus of family Coronaviridae. In some cases, a template nucleotide sequence may comprise at least 88% sequence identity to a sequence of a Nucleocapsid gene of a reference genome of a virus of family Coronaviridae. In some cases, a template nucleotide sequence may comprise at least 89% sequence identity to a sequence of a Nucleocapsid gene of a reference genome of a virus of family Coronaviridae. In some cases, a template nucleotide sequence may comprise at least 90% sequence identity to a sequence of a Nucleocapsid gene of a reference genome of a virus of family Coronaviridae. In some cases, a template nucleotide sequence may comprise at least 91% sequence identity to a sequence of a Nucleocapsid gene of a reference genome of a virus of family Coronaviridae. In some cases, a template nucleotide sequence may comprise at least 92% sequence identity to a sequence of a Nucleocapsid gene of a reference genome of a virus of family Coronaviridae. In some cases, a template nucleotide sequence may comprise at least 93% sequence identity to a sequence of a Nucleocapsid gene of a reference genome of a virus of family Coronaviridae. In some cases, a template nucleotide sequence may comprise at least 94% sequence identity to a sequence of a Nucleocapsid gene of a reference genome of a virus of family Coronaviridae. In some cases, a template nucleotide sequence may comprise at least 95% sequence identity to a sequence of a Nucleocapsid gene of a reference genome of a virus of family Coronaviridae. In some cases, a template nucleotide sequence may comprise at least 96% sequence identity to a sequence of a Nucleocapsid gene of a reference genome of a virus of family Coronaviridae. In some cases, a template nucleotide sequence may comprise at least 97% sequence identity to a sequence of a Nucleocapsid gene of a reference genome of a virus of family Coronaviridae. In some cases, a template nucleotide sequence may comprise at least 98% sequence identity to a sequence of a Nucleocapsid gene of a reference genome of a virus of family Coronaviridae. In some cases, a template nucleotide sequence may comprise at least 99% sequence identity to a sequence of a Nucleocapsid gene of a reference genome of a virus of family Coronaviridae. In some cases, a template nucleotide sequence may comprise of a reference genome of a virus of family Coronaviridae. In some cases, a template nucleotide sequence may comprise at least 50%, at least 55%, at least 60%, at least 65%, at least 70%, at least 75%, or at least 80% sequence identity to a sequence of a Nucleocapsid gene of a reference genome of a virus of family Coronaviridae.

In some instances, a plurality of template nucleotide sequences may have less than about 1500 nucleotides in length in total. In some cases, a plurality of template nucleotide sequences may have less than about 1450 nucleotides in length in total. In some cases, a plurality of template nucleotide sequences may have less than about 1400 nucleotides in length in total. In some cases, a plurality of template nucleotide sequences may have less than about 1350 nucleotides in length in total. In some cases, a plurality of template nucleotide sequences may have less than about 1300 nucleotides in length in total. In some cases, a plurality of template nucleotide sequences may have less than about 1250 nucleotides in length in total. In some cases, a plurality of template nucleotide sequences may have less than about 1200 nucleotides in length in total. In some cases, a plurality of template nucleotide sequences may have less than about 1150 nucleotides in length in total. In some cases, a plurality of template nucleotide sequences may have less than about 1100 nucleotides in length in total. In some cases, a plurality of template nucleotide sequences may have less than about 1050 nucleotides in length in total. In some cases, a plurality of template nucleotide sequences may have less than about 1000 nucleotides in length in total. In some cases, a plurality of template nucleotide sequences may have less than about 950 nucleotides in length in total. In some cases, a plurality of template nucleotide sequences may have less than about 900 nucleotides in length in total. In some cases, a plurality of template nucleotide sequences may have less than about 850 nucleotides in length in total. In some cases, a plurality of template nucleotide sequences may have less than about 800 nucleotides in length in total. In some cases, a plurality of template nucleotide sequences may have less than about 750 nucleotides in length in total. In some cases, a plurality of template nucleotide sequences may have less than about 700 nucleotides in length in total. In some cases, a plurality of template nucleotide sequences may have less than about 650 nucleotides in length in total. In some cases, a plurality of template nucleotide sequences may have less than about 600 nucleotides in length in total. In some cases, a plurality of template nucleotide sequences may have less than about 550 nucleotides in length in total. In some cases, a plurality of template nucleotide sequences may have less than about 500 nucleotides in length in total. In some cases, a plurality of template nucleotide sequences may have less than about 450 nucleotides in length in total. In some cases, a plurality of template nucleotide sequences may have less than about 400 nucleotides in length in total. In some cases, a plurality of template nucleotide sequences may have less than about 350 nucleotides in length in total. In some cases, a plurality of template nucleotide sequences may have less than about 300 nucleotides in length in total. In some cases, a plurality of template nucleotide sequences may have less than about 250 nucleotides in length in total. In some cases, a plurality of template nucleotide sequences may have less than about 200 nucleotides in length in total. In some cases, a plurality of template nucleotide sequences may have less than about 150 nucleotides in length in total. In some cases, a plurality of template nucleotide sequences may have less than about 100 nucleotides in length in total. In some cases, a plurality of template nucleotide sequences may have less than about 50 nucleotides in length in total. In some cases, a plurality of template nucleotide sequences may have less than about 1550 nucleotides in length in total. In some cases, a plurality of template nucleotide sequences may have less than about 1600 nucleotides in length in total. In some cases, a plurality of template nucleotide sequences may have less than about 1650 nucleotides in length in total. In some cases, a plurality of template nucleotide sequences may have less than about 1700 nucleotides in length in total. In some cases, a plurality of template nucleotide sequences may have less than about 1750 nucleotides in length in total. In some cases, a plurality of template nucleotide sequences may have less than about 1800 nucleotides in length in total. In some cases, a plurality of template nucleotide sequences may have less than about 1850 nucleotides in length in total. In some cases, a plurality of template nucleotide sequences may have less than about 1900 nucleotides in length in total. In some cases, a plurality of template nucleotide sequences may have less than about 1950 nucleotides in length in total. In some cases, a plurality of template nucleotide sequences may have less than about 2000 nucleotides in length in total. In some cases, a plurality of template nucleotide sequences may have less than about 2050 nucleotides in length in total. In some cases, a plurality of template nucleotide sequences may have less than about 2100 nucleotides in length in total. In some cases, a plurality of template nucleotide sequences may have less than about 2150 nucleotides in length in total. In some cases, a plurality of template nucleotide sequences may have less than about 2200 nucleotides in length in total. In some cases, a plurality of template nucleotide sequences may have less than about 2250 nucleotides in length in total. In some cases, a plurality of template nucleotide sequences may have less than about 2300 nucleotides in length in total. In some cases, a plurality of template nucleotide sequences may have less than about 2350 nucleotides in length in total. In some cases, a plurality of template nucleotide sequences may have less than about 2400 nucleotides in length in total. In some cases, a plurality of template nucleotide sequences may have less than about 2450 nucleotides in length in total. In some cases, a plurality of template nucleotide sequences may have less than about 2500 nucleotides in length in total. In some cases, a plurality of template nucleotide sequences may have less than about 2550 nucleotides in length in total. In some cases, a plurality of template nucleotide sequences may have less than about 2600 nucleotides in length in total. In some cases, a plurality of template nucleotide sequences may have less than about 2650 nucleotides in length in total. In some cases, a plurality of template nucleotide sequences may have less than about 2700 nucleotides in length in total. In some cases, a plurality of template nucleotide sequences may have less than about 2750 nucleotides in length in total. In some cases, a plurality of template nucleotide sequences may have less than about 2800 nucleotides in length in total. In some cases, a plurality of template nucleotide sequences may have less than about 2850 nucleotides in length in total. In some cases, a plurality of template nucleotide sequences may have less than about 2900 nucleotides in length in total. In some cases, a plurality of template nucleotide sequences may have less than about 2950 nucleotides in length in total. In some cases, a plurality of template nucleotide sequences may have less than about 3000 nucleotides in length in total. In some cases, a plurality of template nucleotide sequences may have less than at least about 3500 nucleotides in length in total, less than at least about 4000 nucleotides in length in total, less than at least about 4500 nucleotides in length in total, less than at least about 5000 nucleotides in length in total, less than at least about 5500 nucleotides in length in total, less than at least about 6000 nucleotides in length in total, less than at least about 6500 nucleotides in length in total, less than at least about 7000 nucleotides in length in total, less than at least about 7500 nucleotides in length in total, less than at least about 8000 nucleotides in length in total, less than at least about 8500 nucleotides in length in total, less than at least about 9000 nucleotides in length in total, less than at least about 9500 nucleotides in length in total, less than at least about 10000 nucleotides in length in total, less than at least about 15000 nucleotides in length in total, less than at least about 20000 nucleotides in length in total, less than at least about 25000 nucleotides in length in total, less than at least about 30000 nucleotides in length in total, less than at least about 35000 nucleotides in length in total, less than at least about 40000 nucleotides in length in total, less than at least about 45000 nucleotides in length in total, or less than at least about 50000 nucleotides in length in total.

In some instances, a plurality of template nucleotide sequences may have less than about 1450 to about 1550 nucleotides in length in total. In some cases, a plurality of template nucleotide sequences may have less than about 1400 to about 1600 nucleotides in length in total. In some cases, a plurality of template nucleotide sequences may have less than about 1350 to about 1650 nucleotides in length in total. In some cases, a plurality of template nucleotide sequences may have less than about 1300 to about 1700 nucleotides in length in total. In some cases, a plurality of template nucleotide sequences may have less than about 1250 to about 1750 nucleotides in length in total. In some cases, a plurality of template nucleotide sequences may have less than about 1200 to about 1800 nucleotides in length in total. In some cases, a plurality of template nucleotide sequences may have less than about 1150 to about 1850 nucleotides in length in total. In some cases, a plurality of template nucleotide sequences may have less than about 1100 to about 1900 nucleotides in length in total. In some cases, a plurality of template nucleotide sequences may have less than about 1050 to about 1950 nucleotides in length in total. In some cases, a plurality of template nucleotide sequences may have less than about 1000 to about 2000 nucleotides in length in total. In some cases, a plurality of template nucleotide sequences may have less than about 950 to about 2050 nucleotides in length in total. In some cases, a plurality of template nucleotide sequences may have less than about 900 to about 2100 nucleotides in length in total. In some cases, a plurality of template nucleotide sequences may have less than about 850 to about 2150 nucleotides in length in total. In some cases, a plurality of template nucleotide sequences may have less than about 800 to about 2200 nucleotides in length in total. In some cases, a plurality of template nucleotide sequences may have less than about 750 to about 2250 nucleotides in length in total. In some cases, a plurality of template nucleotide sequences may have less than about 700 to about 2300 nucleotides in length in total. In some cases, a plurality of template nucleotide sequences may have less than about 650 to about 2350 nucleotides in length in total. In some cases, a plurality of template nucleotide sequences may have less than about 600 to about 2400 nucleotides in length in total. In some cases, a plurality of template nucleotide sequences may have less than about 550 to about 2450 nucleotides in length in total. In some cases, a plurality of template nucleotide sequences may have less than about 500 to about 2500 nucleotides in length in total. In some cases, a plurality of template nucleotide sequences may have less than about 450 to about 2550 nucleotides in length in total. In some cases, a plurality of template nucleotide sequences may have less than about 400 to about 2600 nucleotides in length in total. In some cases, a plurality of template nucleotide sequences may have less than about 350 to about 2650 nucleotides in length in total. In some cases, a plurality of template nucleotide sequences may have less than about 300 to about 2700 nucleotides in length in total. In some cases, a plurality of template nucleotide sequences may have less than about 250 to about 2750 nucleotides in length in total. In some cases, a plurality of template nucleotide sequences may have less than about 200 to about 2800 nucleotides in length in total. In some cases, a plurality of template nucleotide sequences may have less than about 150 to about 2850 nucleotides in length in total. In some cases, a plurality of template nucleotide sequences may have less than about 100 to about 2900 nucleotides in length in total. In some cases, a plurality of template nucleotide sequences may have less than about 50 to about 2950 nucleotides in length in total. In some cases, a plurality of template nucleotide sequences may have less than about 1 to about 3000 nucleotides in length in total. In some cases, a plurality of template nucleotide sequences may have less than about 700 to about 800 nucleotides in length in total. In some cases, a plurality of template nucleotide sequences may have less than about 650 to about 850 nucleotides in length in total. In some cases, a plurality of template nucleotide sequences may have less than about 600 to about 900 nucleotides in length in total. In some cases, a plurality of template nucleotide sequences may have less than about 550 to about 950 nucleotides in length in total. In some cases, a plurality of template nucleotide sequences may have less than about 500 to about 1000 nucleotides in length in total. In some cases, a plurality of template nucleotide sequences may have less than about 450 to about 1050 nucleotides in length in total. In some cases, a plurality of template nucleotide sequences may have less than about 400 to about 1100 nucleotides in length in total. In some cases, a plurality of template nucleotide sequences may have less than about 350 to about 1150 nucleotides in length in total. In some cases, a plurality of template nucleotide sequences may have less than about 300 to about 1200 nucleotides in length in total. In some cases, a plurality of template nucleotide sequences may have less than about 250 to about 1250 nucleotides in length in total. In some cases, a plurality of template nucleotide sequences may have less than about 200 to about 1300 nucleotides in length in total. In some cases, a plurality of template nucleotide sequences may have less than about 150 to about 1350 nucleotides in length in total. In some cases, a plurality of template nucleotide sequences may have less than about 100 to about 1400 nucleotides in length in total. In some cases, a plurality of template nucleotide sequences may have less than about 50 to about 1450 nucleotides in length in total. In some cases, a plurality of template nucleotide sequences may have less than about 1 to about 1500 nucleotides in length in total.

In some cases, a plurality of template nucleotide sequences may have less than about 100 to about 500 nucleotides in length in total, less than about 100 to about 550 nucleotides in length in total, less than about 100 to about 600 nucleotides in length in total, less than about 100 to about 650 nucleotides in length in total, less than about 100 to about 700 nucleotides in length in total, less than about 100 to about 750 nucleotides in length in total, less than about 100 to about 800 nucleotides in length in total, less than about 100 to about 850 nucleotides in length in total, less than about 100 to about 900 nucleotides in length in total, less than about 100 to about 950 nucleotides in length in total, less than about 100 to about 1000 nucleotides in length in total, less than about 100 to about 1050 nucleotides in length in total, less than about 100 to about 1100 nucleotides in length in total, less than about 100 to about 1150 nucleotides in length in total, less than about 100 to about 1200 nucleotides in length in total, less than about 100 to about 1250 nucleotides in length in total, less than about 100 to about 1300 nucleotides in length in total, less than about 100 to about 1350 nucleotides in length in total, less than about 100 to about 1400 nucleotides in length in total, less than about 100 to about 1450 nucleotides in length in total, less than about 100 to about 1500 nucleotides in length in total, less than about 100 to about 1600 nucleotides in length in total, less than about 100 to about 1700 nucleotides in length in total, less than about 100 to about 1800 nucleotides in length in total, less than about 100 to about 1900 nucleotides in length in total, less than about 100 to about 2000 nucleotides in length in total, less than about 100 to about 3000 nucleotides in length in total, less than about 100 to about 4000 nucleotides in length in total, less than about 100 to about 5000 nucleotides in length in total, less than about 100 to about 6000 nucleotides in length in total, less than about 100 to about 7000 nucleotides in length in total, less than about 100 to about 8000 nucleotides in length in total, less than about 100 to about 9000 nucleotides in length in total, less than about 100 to about 10000 nucleotides in length in total, less than about 100 to about 15000 nucleotides in length in total, less than about 100 to about 20000 nucleotides in length in total, less than about 100 to about 25000 nucleotides in length in total, less than about 100 to about 30000 nucleotides in length in total, less than about 100 to about 35000 nucleotides in length in total, less than about 100 to about 40000 nucleotides in length in total, less than about 100 to about 45000 nucleotides in length in total, or less than about 100 to about 50000 nucleotides in length in total.

In some instances, a plurality of template nucleotide sequences may have at least about 1500 nucleotides in length in total. In some cases, a plurality of template nucleotide sequences may have at least about 1450 nucleotides in length in total. In some cases, a plurality of template nucleotide sequences may have at least about 1400 nucleotides in length in total. In some cases, a plurality of template nucleotide sequences may have at least about 1350 nucleotides in length in total. In some cases, a plurality of template nucleotide sequences may have at least about 1300 nucleotides in length in total. In some cases, a plurality of template nucleotide sequences may have at least about 1250 nucleotides in length in total. In some cases, a plurality of template nucleotide sequences may have at least about 1200 nucleotides in length in total. In some cases, a plurality of template nucleotide sequences may have at least about 1150 nucleotides in length in total. In some cases, a plurality of template nucleotide sequences may have at least about 1100 nucleotides in length in total. In some cases, a plurality of template nucleotide sequences may have at least about 1050 nucleotides in length in total. In some cases, a plurality of template nucleotide sequences may have at least about 1000 nucleotides in length in total. In some cases, a plurality of template nucleotide sequences may have at least about 950 nucleotides in length in total. In some cases, a plurality of template nucleotide sequences may have at least about 900 nucleotides in length in total. In some cases, a plurality of template nucleotide sequences may have at least about 850 nucleotides in length in total. In some cases, a plurality of template nucleotide sequences may have at least about 800 nucleotides in length in total. In some cases, a plurality of template nucleotide sequences may have at least about 750 nucleotides in length in total. In some cases, a plurality of template nucleotide sequences may have at least about 700 nucleotides in length in total. In some cases, a plurality of template nucleotide sequences may have at least about 650 nucleotides in length in total. In some cases, a plurality of template nucleotide sequences may have at least about 600 nucleotides in length in total. In some cases, a plurality of template nucleotide sequences may have at least about 550 nucleotides in length in total. In some cases, a plurality of template nucleotide sequences may have at least about 500 nucleotides in length in total. In some cases, a plurality of template nucleotide sequences may have at least about 450 nucleotides in length in total. In some cases, a plurality of template nucleotide sequences may have at least about 400 nucleotides in length in total. In some cases, a plurality of template nucleotide sequences may have at least about 350 nucleotides in length in total. In some cases, a plurality of template nucleotide sequences may have at least about 300 nucleotides in length in total. In some cases, a plurality of template nucleotide sequences may have at least about 250 nucleotides in length in total. In some cases, a plurality of template nucleotide sequences may have at least about 200 nucleotides in length in total. In some cases, a plurality of template nucleotide sequences may have at least about 150 nucleotides in length in total. In some cases, a plurality of template nucleotide sequences may have at least about 100 nucleotides in length in total. In some cases, a plurality of template nucleotide sequences may have at least about 50 nucleotides in length in total. In some cases, a plurality of template nucleotide sequences may have at least about 1550 nucleotides in length in total. In some cases, a plurality of template nucleotide sequences may have at least about 1600 nucleotides in length in total. In some cases, a plurality of template nucleotide sequences may have at least about 1650 nucleotides in length in total. In some cases, a plurality of template nucleotide sequences may have at least about 1700 nucleotides in length in total. In some cases, a plurality of template nucleotide sequences may have at least about 1750 nucleotides in length in total. In some cases, a plurality of template nucleotide sequences may have at least about 1800 nucleotides in length in total. In some cases, a plurality of template nucleotide sequences may have at least about 1850 nucleotides in length in total. In some cases, a plurality of template nucleotide sequences may have at least about 1900 nucleotides in length in total. In some cases, a plurality of template nucleotide sequences may have at least about 1950 nucleotides in length in total. In some cases, a plurality of template nucleotide sequences may have at least about 2000 nucleotides in length in total. In some cases, a plurality of template nucleotide sequences may have at least about 2050 nucleotides in length in total. In some cases, a plurality of template nucleotide sequences may have at least about 2100 nucleotides in length in total. In some cases, a plurality of template nucleotide sequences may have at least about 2150 nucleotides in length in total. In some cases, a plurality of template nucleotide sequences may have at least about 2200 nucleotides in length in total. In some cases, a plurality of template nucleotide sequences may have at least about 2250 nucleotides in length in total. In some cases, a plurality of template nucleotide sequences may have at least about 2300 nucleotides in length in total. In some cases, a plurality of template nucleotide sequences may have at least about 2350 nucleotides in length in total. In some cases, a plurality of template nucleotide sequences may have at least about 2400 nucleotides in length in total. In some cases, a plurality of template nucleotide sequences may have at least about 2450 nucleotides in length in total. In some cases, a plurality of template nucleotide sequences may have at least about 2500 nucleotides in length in total. In some cases, a plurality of template nucleotide sequences may have at least about 2550 nucleotides in length in total. In some cases, a plurality of template nucleotide sequences may have at least about 2600 nucleotides in length in total. In some cases, a plurality of template nucleotide sequences may have at least about 2650 nucleotides in length in total. In some cases, a plurality of template nucleotide sequences may have at least about 2700 nucleotides in length in total. In some cases, a plurality of template nucleotide sequences may have at least about 2750 nucleotides in length in total. In some cases, a plurality of template nucleotide sequences may have at least about 2800 nucleotides in length in total. In some cases, a plurality of template nucleotide sequences may have at least about 2850 nucleotides in length in total. In some cases, a plurality of template nucleotide sequences may have at least about 2900 nucleotides in length in total. In some cases, a plurality of template nucleotide sequences may have at least about 2950 nucleotides in length in total. In some cases, a plurality of template nucleotide sequences may have at least about 3000 nucleotides in length in total. In some cases, a plurality of template nucleotide sequences may have at least at least about 3500 nucleotides in length in total, at least at least about 4000 nucleotides in length in total, at least at least about 4500 nucleotides in length in total, at least at least about 5000 nucleotides in length in total, at least at least about 5500 nucleotides in length in total, at least at least about 6000 nucleotides in length in total, at least at least about 6500 nucleotides in length in total, at least at least about 7000 nucleotides in length in total, at least at least about 7500 nucleotides in length in total, at least at least about 8000 nucleotides in length in total, at least at least about 8500 nucleotides in length in total, at least at least about 9000 nucleotides in length in total, at least at least about 9500 nucleotides in length in total, at least at least about 10000 nucleotides in length in total, at least at least about 15000 nucleotides in length in total, at least at least about 20000 nucleotides in length in total, at least at least about 25000 nucleotides in length in total, at least at least about 30000 nucleotides in length in total, at least at least about 35000 nucleotides in length in total, at least at least about 40000 nucleotides in length in total, at least at least about 45000 nucleotides in length in total, or at least at least about 50000 nucleotides in length in total.

In some cases, a plurality of template nucleotide sequences may have from about 1 to about 3000 nucleotides in length in total. In some cases, a plurality of template nucleotide sequences may have from about 25 to about 2900 nucleotides in length in total. In some cases, a plurality of template nucleotide sequences may have from about 50 to about 2800 nucleotides in length in total. In some cases, a plurality of template nucleotide sequences may have from about 75 to about 2700 nucleotides in length in total. In some cases, a plurality of template nucleotide sequences may have from about 100 to about 2600 nucleotides in length in total. In some cases, a plurality of template nucleotide sequences may have from about 125 to about 2500 nucleotides in length in total. In some cases, a plurality of template nucleotide sequences may have from about 150 to about 2400 nucleotides in length in total. In some cases, a plurality of template nucleotide sequences may have from about 175 to about 2300 nucleotides in length in total. In some cases, a plurality of template nucleotide sequences may have from about 200 to about 2200 nucleotides in length in total. In some cases, a plurality of template nucleotide sequences may have from about 225 to about 2100 nucleotides in length in total. In some cases, a plurality of template nucleotide sequences may have from about 250 to about 2000 nucleotides in length in total. In some cases, a plurality of template nucleotide sequences may have from about 275 to about 1900 nucleotides in length in total. In some cases, a plurality of template nucleotide sequences may have from about 300 to about 1800 nucleotides in length in total. In some cases, a plurality of template nucleotide sequences may have from about 325 to about 1700 nucleotides in length in total. In some cases, a plurality of template nucleotide sequences may have from about 350 to about 1600 nucleotides in length in total. In some cases, a plurality of template nucleotide sequences may have from about 375 to about 1500 nucleotides in length in total. In some cases, a plurality of template nucleotide sequences may have from about 400 to about 1400 nucleotides in length in total. In some cases, a plurality of template nucleotide sequences may have from about 425 to about 1300 nucleotides in length in total. In some cases, a plurality of template nucleotide sequences may have from about 450 to about 1200 nucleotides in length in total. In some cases, a plurality of template nucleotide sequences may have from about 475 to about 1100 nucleotides in length in total. In some cases, a plurality of template nucleotide sequences may have from about 500 to about 1000 nucleotides in length in total. In some cases, a plurality of template nucleotide sequences may have from about 525 to about 950 nucleotides in length in total. In some cases, a plurality of template nucleotide sequences may have from about 550 to about 900 nucleotides in length in total. In some cases, a plurality of template nucleotide sequences may have from about 575 to about 850 nucleotides in length in total. In some cases, a plurality of template nucleotide sequences may have from about 600 to about 800 nucleotides in length in total. In some cases, a plurality of template nucleotide sequences may have from about 625 to about 750 nucleotides in length in total.

In some instances, a plurality of template nucleotide sequences may have from about 1450 to about 1550 nucleotides in length in total. In some cases, a plurality of template nucleotide sequences may have from about 1400 to about 1600 nucleotides in length in total. In some cases, a plurality of template nucleotide sequences may have from about 1350 to about 1650 nucleotides in length in total. In some cases, a plurality of template nucleotide sequences may have from about 1300 to about 1700 nucleotides in length in total. In some cases, a plurality of template nucleotide sequences may have from about 1250 to about 1750 nucleotides in length in total. In some cases, a plurality of template nucleotide sequences may have from about 1200 to about 1800 nucleotides in length in total. In some cases, a plurality of template nucleotide sequences may have from about 1150 to about 1850 nucleotides in length in total. In some cases, a plurality of template nucleotide sequences may have from about 1100 to about 1900 nucleotides in length in total. In some cases, a plurality of template nucleotide sequences may have from about 1050 to about 1950 nucleotides in length in total. In some cases, a plurality of template nucleotide sequences may have from about 1000 to about 2000 nucleotides in length in total. In some cases, a plurality of template nucleotide sequences may have from about 950 to about 2050 nucleotides in length in total. In some cases, a plurality of template nucleotide sequences may have from about 900 to about 2100 nucleotides in length in total. In some cases, a plurality of template nucleotide sequences may have from about 850 to about 2150 nucleotides in length in total. In some cases, a plurality of template nucleotide sequences may have from about 800 to about 2200 nucleotides in length in total. In some cases, a plurality of template nucleotide sequences may have from about 750 to about 2250 nucleotides in length in total. In some cases, a plurality of template nucleotide sequences may have from about 700 to about 2300 nucleotides in length in total. In some cases, a plurality of template nucleotide sequences may have from about 650 to about 2350 nucleotides in length in total. In some cases, a plurality of template nucleotide sequences may have from about 600 to about 2400 nucleotides in length in total. In some cases, a plurality of template nucleotide sequences may have from about 550 to about 2450 nucleotides in length in total. In some cases, a plurality of template nucleotide sequences may have from about 500 to about 2500 nucleotides in length in total. In some cases, a plurality of template nucleotide sequences may have from about 450 to about 2550 nucleotides in length in total. In some cases, a plurality of template nucleotide sequences may have from about 400 to about 2600 nucleotides in length in total. In some cases, a plurality of template nucleotide sequences may have from about 350 to about 2650 nucleotides in length in total. In some cases, a plurality of template nucleotide sequences may have from about 300 to about 2700 nucleotides in length in total. In some cases, a plurality of template nucleotide sequences may have from about 250 to about 2750 nucleotides in length in total. In some cases, a plurality of template nucleotide sequences may have from about 200 to about 2800 nucleotides in length in total. In some cases, a plurality of template nucleotide sequences may have from about 150 to about 2850 nucleotides in length in total. In some cases, a plurality of template nucleotide sequences may have from about 100 to about 2900 nucleotides in length in total. In some cases, a plurality of template nucleotide sequences may have from about 50 to about 2950 nucleotides in length in total. In some cases, a plurality of template nucleotide sequences may have from about 1 to about 3000 nucleotides in length in total. In some cases, a plurality of template nucleotide sequences may have from about 700 to about 800 nucleotides in length in total. In some cases, a plurality of template nucleotide sequences may have from about 650 to about 850 nucleotides in length in total. In some cases, a plurality of template nucleotide sequences may have from about 600 to about 900 nucleotides in length in total. In some cases, a plurality of template nucleotide sequences may have from about 550 to about 950 nucleotides in length in total. In some cases, a plurality of template nucleotide sequences may have from about 500 to about 1000 nucleotides in length in total. In some cases, a plurality of template nucleotide sequences may have from about 450 to about 1050 nucleotides in length in total. In some cases, a plurality of template nucleotide sequences may have from about 400 to about 1100 nucleotides in length in total. In some cases, a plurality of template nucleotide sequences may have from about 350 to about 1150 nucleotides in length in total. In some cases, a plurality of template nucleotide sequences may have from about 300 to about 1200 nucleotides in length in total. In some cases, a plurality of template nucleotide sequences may have from about 250 to about 1250 nucleotides in length in total. In some cases, a plurality of template nucleotide sequences may have from about 200 to about 1300 nucleotides in length in total. In some cases, a plurality of template nucleotide sequences may have from about 150 to about 1350 nucleotides in length in total. In some cases, a plurality of template nucleotide sequences may have from about 100 to about 1400 nucleotides in length in total. In some cases, a plurality of template nucleotide sequences may have from about 50 to about 1450 nucleotides in length in total. In some cases, a plurality of template nucleotide sequences may have from about 1 to about 1500 nucleotides in length in total.

In some cases, a plurality of template nucleotide sequences may have from about 100 to about 500 nucleotides in length in total, from about 100 to about 550 nucleotides in length in total, from about 100 to about 600 nucleotides in length in total, from about 100 to about 650 nucleotides in length in total, from about 100 to about 700 nucleotides in length in total, from about 100 to about 750 nucleotides in length in total, from about 100 to about 800 nucleotides in length in total, from about 100 to about 850 nucleotides in length in total, from about 100 to about 900 nucleotides in length in total, from about 100 to about 950 nucleotides in length in total, from about 100 to about 1000 nucleotides in length in total, from about 100 to about 1050 nucleotides in length in total, from about 100 to about 1100 nucleotides in length in total, from about 100 to about 1150 nucleotides in length in total, from about 100 to about 1200 nucleotides in length in total, from about 100 to about 1250 nucleotides in length in total, from about 100 to about 1300 nucleotides in length in total, from about 100 to about 1350 nucleotides in length in total, from about 100 to about 1400 nucleotides in length in total, from about 100 to about 1450 nucleotides in length in total, from about 100 to about 1500 nucleotides in length in total, from about 100 to about 1600 nucleotides in length in total, from about 100 to about 1700 nucleotides in length in total, from about 100 to about 1800 nucleotides in length in total, from about 100 to about 1900 nucleotides in length in total, from about 100 to about 2000 nucleotides in length in total, from about 100 to about 3000 nucleotides in length in total, from about 100 to about 4000 nucleotides in length in total, from about 100 to about 5000 nucleotides in length in total, from about 100 to about 6000 nucleotides in length in total, from about 100 to about 7000 nucleotides in length in total, from about 100 to about 8000 nucleotides in length in total, from about 100 to about 9000 nucleotides in length in total, from about 100 to about 10000 nucleotides in length in total, from about 100 to about 15000 nucleotides in length in total, from about 100 to about 20000 nucleotides in length in total, from about 100 to about 25000 nucleotides in length in total, from about 100 to about 30000 nucleotides in length in total, from about 100 to about 35000 nucleotides in length in total, from about 100 to about 40000 nucleotides in length in total, from about 100 to about 45000 nucleotides in length in total, or from about 100 to about 50000 nucleotides in length in total.

In some instances, a plurality of template nucleotide sequences may have from about 1450 to about 1550 nucleotides in length in total. In some cases, a plurality of template nucleotide sequences may have from about 1400 to about 1600 nucleotides in length in total. In some cases, a plurality of template nucleotide sequences may have from about 1350 to about 1650 nucleotides in length in total. In some cases, a plurality of template nucleotide sequences may have from about 1300 to about 1700 nucleotides in length in total. In some cases, a plurality of template nucleotide sequences may have from about 1250 to about 1750 nucleotides in length in total. In some cases, a plurality of template nucleotide sequences may have from about 1200 to about 1800 nucleotides in length in total. In some cases, a plurality of template nucleotide sequences may have from about 1150 to about 1850 nucleotides in length in total. In some cases, a plurality of template nucleotide sequences may have from about 1100 to about 1900 nucleotides in length in total. In some cases, a plurality of template nucleotide sequences may have from about 1050 to about 1950 nucleotides in length in total. In some cases, a plurality of template nucleotide sequences may have from about 1000 to about 2000 nucleotides in length in total. In some cases, a plurality of template nucleotide sequences may have from about 950 to about 2050 nucleotides in length in total. In some cases, a plurality of template nucleotide sequences may have from about 900 to about 2100 nucleotides in length in total. In some cases, a plurality of template nucleotide sequences may have from about 850 to about 2150 nucleotides in length in total. In some cases, a plurality of template nucleotide sequences may have from about 800 to about 2200 nucleotides in length in total. In some cases, a plurality of template nucleotide sequences may have from about 750 to about 2250 nucleotides in length in total. In some cases, a plurality of template nucleotide sequences may have from about 700 to about 2300 nucleotides in length in total. In some cases, a plurality of template nucleotide sequences may have from about 650 to about 2350 nucleotides in length in total. In some cases, a plurality of template nucleotide sequences may have from about 600 to about 2400 nucleotides in length in total. In some cases, a plurality of template nucleotide sequences may have from about 550 to about 2450 nucleotides in length in total. In some cases, a plurality of template nucleotide sequences may have from about 500 to about 2500 nucleotides in length in total. In some cases, a plurality of template nucleotide sequences may have from about 450 to about 2550 nucleotides in length in total. In some cases, a plurality of template nucleotide sequences may have from about 400 to about 2600 nucleotides in length in total. In some cases, a plurality of template nucleotide sequences may have from about 350 to about 2650 nucleotides in length in total. In some cases, a plurality of template nucleotide sequences may have from about 300 to about 2700 nucleotides in length in total. In some cases, a plurality of template nucleotide sequences may have from about 250 to about 2750 nucleotides in length in total. In some cases, a plurality of template nucleotide sequences may have from about 200 to about 2800 nucleotides in length in total. In some cases, a plurality of template nucleotide sequences may have from about 150 to about 2850 nucleotides in length in total. In some cases, a plurality of template nucleotide sequences may have from about 100 to about 2900 nucleotides in length in total. In some cases, a plurality of template nucleotide sequences may have from about 50 to about 2950 nucleotides in length in total. In some cases, a plurality of template nucleotide sequences may have from about 1 to about 3000 nucleotides in length in total. In some cases, a plurality of template nucleotide sequences may have from about 700 to about 800 nucleotides in length in total. In some cases, a plurality of template nucleotide sequences may have from about 650 to about 850 nucleotides in length in total. In some cases, a plurality of template nucleotide sequences may have from about 600 to about 900 nucleotides in length in total. In some cases, a plurality of template nucleotide sequences may have from about 550 to about 950 nucleotides in length in total. In some cases, a plurality of template nucleotide sequences may have from about 500 to about 1000 nucleotides in length in total. In some cases, a plurality of template nucleotide sequences may have from about 450 to about 1050 nucleotides in length in total. In some cases, a plurality of template nucleotide sequences may have from about 400 to about 1100 nucleotides in length in total. In some cases, a plurality of template nucleotide sequences may have from about 350 to about 1150 nucleotides in length in total. In some cases, a plurality of template nucleotide sequences may have from about 300 to about 1200 nucleotides in length in total. In some cases, a plurality of template nucleotide sequences may have from about 250 to about 1250 nucleotides in length in total. In some cases, a plurality of template nucleotide sequences may have from about 200 to about 1300 nucleotides in length in total. In some cases, a plurality of template nucleotide sequences may have from about 150 to about 1350 nucleotides in length in total. In some cases, a plurality of template nucleotide sequences may have from about 100 to about 1400 nucleotides in length in total. In some cases, a plurality of template nucleotide sequences may have from about 50 to about 1450 nucleotides in length in total.

In some cases, a plurality of template nucleotide sequences may have from about 100 to about 30000 nucleotides in length in total, from about 200 to about 30000 nucleotides in length in total, from about 300 to about 30000 nucleotides in length in total, from about 400 to about 30000 nucleotides in length in total, from about 500 to about 30000 nucleotides in length in total, from about 600 to about 30000 nucleotides in length in total, from about 700 to about 30000 nucleotides in length in total, from about 800 to about 30000 nucleotides in length in total, from about 900 to about 30000 nucleotides in length in total, from about 1000 to about 30000 nucleotides in length in total, from about 1100 to about 30000 nucleotides in length in total, from about 1200 to about 30000 nucleotides in length in total, from about 1300 to about 30000 nucleotides in length in total, from about 1400 to about 30000 nucleotides in length in total, from about 1500 to about 30000 nucleotides in length in total, from about 1600 to about 30000 nucleotides in length in total, from about 1700 to about 30000 nucleotides in length in total, from about 1800 to about 30000 nucleotides in length in total, from about 1900 to about 30000 nucleotides in length in total, from about 2000 to about 30000 nucleotides in length in total, from about 2100 to about 30000 nucleotides in length in total, from about 2200 to about 30000 nucleotides in length in total, from about 2300 to about 30000 nucleotides in length in total, from about 2400 to about 30000 nucleotides in length in total, from about 2500 to about 30000 nucleotides in length in total, from about 2600 to about 30000 nucleotides in length in total, from about 2700 to about 30000 nucleotides in length in total, from about 2800 to about 30000 nucleotides in length in total, from about 2900 to about 30000 nucleotides in length in total, from about 3000 to about 30000 nucleotides in length in total, from about 3500 to about 30000 nucleotides in length in total, from about 4000 to about 30000 nucleotides in length in total, from about 4500 to about 30000 nucleotides in length in total, from about 5000 to about 30000 nucleotides in length in total, from about 6000 to about 30000 nucleotides in length in total, from about 7000 to about 30000 nucleotides in length in total, from about 8000 to about 30000 nucleotides in length in total, from about 9000 to about 30000 nucleotides in length in total, from about 10000 to about 30000 nucleotides in length in total, from about 15000 to about 30000 nucleotides in length in total, from about 20000 to about 30000 nucleotides in length in total, or from about 25000 to about 30000 nucleotides in length in total.

In some cases, a template nucleotide sequence may have about 50 nucleotides in length in total. In some cases, a template nucleotide sequence may have about 60 nucleotides in length in total. In some cases, a template nucleotide sequence may have about 70 nucleotides in length in total. In some cases, a template nucleotide sequence may have about 80 nucleotides in length in total. In some cases, a template nucleotide sequence may have about 90 nucleotides in length in total. In some cases, a template nucleotide sequence may have about 100 nucleotides in length in total. In some cases, a template nucleotide sequence may have about 110 nucleotides in length in total. In some cases, a template nucleotide sequence may have about 120 nucleotides in length in total. In some cases, a template nucleotide sequence may have about 130 nucleotides in length in total. In some cases, a template nucleotide sequence may have about 140 nucleotides in length in total. In some cases, a template nucleotide sequence may have about 150 nucleotides in length in total. In some cases, a template nucleotide sequence may have about 160 nucleotides in length in total. In some cases, a template nucleotide sequence may have about 170 nucleotides in length in total. In some cases, a template nucleotide sequence may have about 180 nucleotides in length in total. In some cases, a template nucleotide sequence may have about 190 nucleotides in length in total. In some cases, a template nucleotide sequence may have about 200 nucleotides in length in total. In some cases, a template nucleotide sequence may have about 210 nucleotides in length in total. In some cases, a template nucleotide sequence may have about 220 nucleotides in length in total. In some cases, a template nucleotide sequence may have about 230 nucleotides in length in total. In some cases, a template nucleotide sequence may have about 240 nucleotides in length in total. In some cases, a template nucleotide sequence may have about 250 nucleotides in length in total. In some cases, a template nucleotide sequence may have about 260 nucleotides in length in total. In some cases, a template nucleotide sequence may have about 270 nucleotides in length in total. In some cases, a template nucleotide sequence may have about 280 nucleotides in length in total. In some cases, a template nucleotide sequence may have about 290 nucleotides in length in total. In some cases, a template nucleotide sequence may have about 300 nucleotides in length in total. In some cases, a template nucleotide sequence may have about 310 nucleotides in length in total. In some cases, a template nucleotide sequence may have about 320 nucleotides in length in total. In some cases, a template nucleotide sequence may have about 330 nucleotides in length in total. In some cases, a template nucleotide sequence may have about 340 nucleotides in length in total. In some cases, a template nucleotide sequence may have about 350 nucleotides in length in total. In some cases, a template nucleotide sequence may have about 360 nucleotides in length in total. In some cases, a template nucleotide sequence may have about 370 nucleotides in length in total. In some cases, a template nucleotide sequence may have about 380 nucleotides in length in total. In some cases, a template nucleotide sequence may have about 390 nucleotides in length in total. In some cases, a template nucleotide sequence may have about 400 nucleotides in length in total. In some cases, a template nucleotide sequence may have about 410 nucleotides in length in total. In some cases, a template nucleotide sequence may have about 420 nucleotides in length in total. In some cases, a template nucleotide sequence may have about 430 nucleotides in length in total. In some cases, a template nucleotide sequence may have about 440 nucleotides in length in total. In some cases, a template nucleotide sequence may have about 450 nucleotides in length in total. In some cases, a template nucleotide sequence may have about 460 nucleotides in length in total. In some cases, a template nucleotide sequence may have about 470 nucleotides in length in total. In some cases, a template nucleotide sequence may have about 480 nucleotides in length in total. In some cases, a template nucleotide sequence may have about 490 nucleotides in length in total. In some cases, a template nucleotide sequence may have about 500 nucleotides in length in total. In some cases, a template nucleotide sequence may have about 510 nucleotides in length in total, about 520 nucleotides in length in total, about 530 nucleotides in length in total, about 540 nucleotides in length in total, about 550 nucleotides in length in total, about 560 nucleotides in length in total, about 570 nucleotides in length in total, about 580 nucleotides in length in total, about 590 nucleotides in length in total, about 600 nucleotides in length in total, about 610 nucleotides in length in total, about 620 nucleotides in length in total, about 630 nucleotides in length in total, about 640 nucleotides in length in total, about 650 nucleotides in length in total, about 660 nucleotides in length in total, about 670 nucleotides in length in total, about 680 nucleotides in length in total, about 690 nucleotides in length in total, about 700 nucleotides in length in total, about 710 nucleotides in length in total, about 720 nucleotides in length in total, about 730 nucleotides in length in total, about 740 nucleotides in length in total, about 750 nucleotides in length in total, about 760 nucleotides in length in total, about 770 nucleotides in length in total, about 780 nucleotides in length in total, about 790 nucleotides in length in total, about 800 nucleotides in length in total, about 810 nucleotides in length in total, about 820 nucleotides in length in total, about 830 nucleotides in length in total, about 840 nucleotides in length in total, about 850 nucleotides in length in total, about 860 nucleotides in length in total, about 870 nucleotides in length in total, about 880 nucleotides in length in total, about 890 nucleotides in length in total, about 900 nucleotides in length in total, about 910 nucleotides in length in total, about 920 nucleotides in length in total, about 930 nucleotides in length in total, about 940 nucleotides in length in total, about 950 nucleotides in length in total, about 960 nucleotides in length in total, about 970 nucleotides in length in total, about 980 nucleotides in length in total, about 990 nucleotides in length in total, or about 1000 nucleotides in length in total.

In some cases, a template nucleotide sequence may have from 1 to 100 nucleotides in length in total. In some cases, a template nucleotide sequence may have from 10 to 110 nucleotides in length in total. In some cases, a template nucleotide sequence may have from 20 to 120 nucleotides in length in total. In some cases, a template nucleotide sequence may have from 30 to 130 nucleotides in length in total. In some cases, a template nucleotide sequence may have from 40 to 140 nucleotides in length in total. In some cases, a template nucleotide sequence may have from 50 to 150 nucleotides in length in total. In some cases, a template nucleotide sequence may have from 60 to 160 nucleotides in length in total. In some cases, a template nucleotide sequence may have from 70 to 170 nucleotides in length in total. In some cases, a template nucleotide sequence may have from 80 to 180 nucleotides in length in total. In some cases, a template nucleotide sequence may have from 90 to 190 nucleotides in length in total. In some cases, a template nucleotide sequence may have from 100 to 200 nucleotides in length in total. In some cases, a template nucleotide sequence may have from 110 to 210 nucleotides in length in total. In some cases, a template nucleotide sequence may have from 120 to 220 nucleotides in length in total. In some cases, a template nucleotide sequence may have from 130 to 230 nucleotides in length in total. In some cases, a template nucleotide sequence may have from 140 to 240 nucleotides in length in total. In some cases, a template nucleotide sequence may have from 150 to 250 nucleotides in length in total. In some cases, a template nucleotide sequence may have from 160 to 260 nucleotides in length in total. In some cases, a template nucleotide sequence may have from 170 to 270 nucleotides in length in total. In some cases, a template nucleotide sequence may have from 180 to 280 nucleotides in length in total. In some cases, a template nucleotide sequence may have from 190 to 290 nucleotides in length in total. In some cases, a template nucleotide sequence may have from 200 to 300 nucleotides in length in total. In some cases, a template nucleotide sequence may have from 210 to 310 nucleotides in length in total. In some cases, a template nucleotide sequence may have from 220 to 320 nucleotides in length in total. In some cases, a template nucleotide sequence may have from 230 to 330 nucleotides in length in total. In some cases, a template nucleotide sequence may have from 240 to 340 nucleotides in length in total. In some cases, a template nucleotide sequence may have from 250 to 350 nucleotides in length in total. In some cases, a template nucleotide sequence may have from 260 to 360 nucleotides in length in total. In some cases, a template nucleotide sequence may have from 270 to 370 nucleotides in length in total. In some cases, a template nucleotide sequence may have from 280 to 380 nucleotides in length in total. In some cases, a template nucleotide sequence may have from 290 to 390 nucleotides in length in total. In some cases, a template nucleotide sequence may have from 300 to 400 nucleotides in length in total. In some cases, a template nucleotide sequence may have from 310 to 410 nucleotides in length in total. In some cases, a template nucleotide sequence may have from 320 to 420 nucleotides in length in total. In some cases, a template nucleotide sequence may have from 330 to 430 nucleotides in length in total. In some cases, a template nucleotide sequence may have from 340 to 440 nucleotides in length in total. In some cases, a template nucleotide sequence may have from 350 to 450 nucleotides in length in total. In some cases, a template nucleotide sequence may have from 360 to 460 nucleotides in length in total. In some cases, a template nucleotide sequence may have from 370 to 470 nucleotides in length in total. In some cases, a template nucleotide sequence may have from 380 to 480 nucleotides in length in total. In some cases, a template nucleotide sequence may have from 390 to 490 nucleotides in length in total. In some cases, a template nucleotide sequence may have from 400 to 500 nucleotides in length in total. In some cases, a template nucleotide sequence may have from 410 to 510 nucleotides in length in total. In some cases, a template nucleotide sequence may have from 420 to 520 nucleotides in length in total. In some cases, a template nucleotide sequence may have from 430 to 530 nucleotides in length in total. In some cases, a template nucleotide sequence may have from 440 to 540 nucleotides in length in total. In some cases, a template nucleotide sequence may have from 450 to 550 nucleotides in length in total. In some cases, a template nucleotide sequence may have from 460 to 560 nucleotides in length in total, from 520 to 570 nucleotides in length in total, from 530 to 580 nucleotides in length in total, from 540 to 590 nucleotides in length in total, from 550 to 600 nucleotides in length in total, from 560 to 610 nucleotides in length in total, from 570 to 620 nucleotides in length in total, from 580 to 630 nucleotides in length in total, from 590 to 640 nucleotides in length in total, from 600 to 650 nucleotides in length in total, from 610 to 660 nucleotides in length in total, from 620 to 670 nucleotides in length in total, from 630 to 680 nucleotides in length in total, from 640 to 690 nucleotides in length in total, from 650 to 700 nucleotides in length in total, from 660 to 710 nucleotides in length in total, from 670 to 720 nucleotides in length in total, from 680 to 730 nucleotides in length in total, from 690 to 740 nucleotides in length in total, from 700 to 750 nucleotides in length in total, from 710 to 760 nucleotides in length in total, from 720 to 770 nucleotides in length in total, from 730 to 780 nucleotides in length in total, from 740 to 790 nucleotides in length in total, from 750 to 800 nucleotides in length in total, from 760 to 810 nucleotides in length in total, from 770 to 820 nucleotides in length in total, from 780 to 830 nucleotides in length in total, from 790 to 840 nucleotides in length in total, from 800 to 850 nucleotides in length in total, from 810 to 860 nucleotides in length in total, from 820 to 870 nucleotides in length in total, from 830 to 880 nucleotides in length in total, from 840 to 890 nucleotides in length in total, from 850 to 900 nucleotides in length in total, from 860 to 910 nucleotides in length in total, from 870 to 920 nucleotides in length in total, from 880 to 930 nucleotides in length in total, from 890 to 940 nucleotides in length in total, from 900 to 950 nucleotides in length in total, from 910 to 960 nucleotides in length in total, from 920 to 970 nucleotides in length in total, from 930 to 980 nucleotides in length in total, from 940 to 990 nucleotides in length in total, or from 950 to 1000 nucleotides in length in total.

In some cases, each of a plurality of template nucleotide sequences may have about 50 nucleotides in length in total. In some cases, each of a plurality of template nucleotide sequences may have about 60 nucleotides in length in total. In some cases, each of a plurality of template nucleotide sequences may have about 70 nucleotides in length in total. In some cases, each of a plurality of template nucleotide sequences may have about 80 nucleotides in length in total. In some cases, each of a plurality of template nucleotide sequences may have about 90 nucleotides in length in total. In some cases, each of a plurality of template nucleotide sequences may have about 100 nucleotides in length in total. In some cases, each of a plurality of template nucleotide sequences may have about 110 nucleotides in length in total. In some cases, each of a plurality of template nucleotide sequences may have about 120 nucleotides in length in total. In some cases, each of a plurality of template nucleotide sequences may have about 130 nucleotides in length in total. In some cases, each of a plurality of template nucleotide sequences may have about 140 nucleotides in length in total. In some cases, each of a plurality of template nucleotide sequences may have about 150 nucleotides in length in total. In some cases, each of a plurality of template nucleotide sequences may have about 160 nucleotides in length in total. In some cases, each of a plurality of template nucleotide sequences may have about 170 nucleotides in length in total. In some cases, each of a plurality of template nucleotide sequences may have about 180 nucleotides in length in total. In some cases, each of a plurality of template nucleotide sequences may have about 190 nucleotides in length in total. In some cases, each of a plurality of template nucleotide sequences may have about 200 nucleotides in length in total. In some cases, each of a plurality of template nucleotide sequences may have about 210 nucleotides in length in total. In some cases, each of a plurality of template nucleotide sequences may have about 220 nucleotides in length in total. In some cases, each of a plurality of template nucleotide sequences may have about 230 nucleotides in length in total. In some cases, each of a plurality of template nucleotide sequences may have about 240 nucleotides in length in total. In some cases, each of a plurality of template nucleotide sequences may have about 250 nucleotides in length in total. In some cases, each of a plurality of template nucleotide sequences may have about 260 nucleotides in length in total. In some cases, each of a plurality of template nucleotide sequences may have about 270 nucleotides in length in total. In some cases, each of a plurality of template nucleotide sequences may have about 280 nucleotides in length in total. In some cases, each of a plurality of template nucleotide sequences may have about 290 nucleotides in length in total. In some cases, each of a plurality of template nucleotide sequences may have about 300 nucleotides in length in total. In some cases, each of a plurality of template nucleotide sequences may have about 310 nucleotides in length in total. In some cases, each of a plurality of template nucleotide sequences may have about 320 nucleotides in length in total. In some cases, each of a plurality of template nucleotide sequences may have about 330 nucleotides in length in total. In some cases, each of a plurality of template nucleotide sequences may have about 340 nucleotides in length in total. In some cases, each of a plurality of template nucleotide sequences may have about 350 nucleotides in length in total. In some cases, each of a plurality of template nucleotide sequences may have about 360 nucleotides in length in total. In some cases, each of a plurality of template nucleotide sequences may have about 370 nucleotides in length in total. In some cases, each of a plurality of template nucleotide sequences may have about 380 nucleotides in length in total. In some cases, each of a plurality of template nucleotide sequences may have about 390 nucleotides in length in total. In some cases, each of a plurality of template nucleotide sequences may have about 400 nucleotides in length in total. In some cases, each of a plurality of template nucleotide sequences may have about 410 nucleotides in length in total. In some cases, each of a plurality of template nucleotide sequences may have about 420 nucleotides in length in total. In some cases, each of a plurality of template nucleotide sequences may have about 430 nucleotides in length in total. In some cases, each of a plurality of template nucleotide sequences may have about 440 nucleotides in length in total. In some cases, each of a plurality of template nucleotide sequences may have about 450 nucleotides in length in total. In some cases, each of a plurality of template nucleotide sequences may have about 460 nucleotides in length in total. In some cases, each of a plurality of template nucleotide sequences may have about 470 nucleotides in length in total. In some cases, each of a plurality of template nucleotide sequences may have about 480 nucleotides in length in total. In some cases, each of a plurality of template nucleotide sequences may have about 490 nucleotides in length in total. In some cases, each of a plurality of template nucleotide sequences may have about 500 nucleotides in length in total.

In some cases, each of a plurality of template nucleotide sequences may have from about 100 nucleotides to 350 nucleotides in length in total. In some cases, each of a plurality of template nucleotide sequences may have from about 110 nucleotides to 360 nucleotides in length in total. In some cases, each of a plurality of template nucleotide sequences may have from about 120 nucleotides to 370 nucleotides in length in total. In some cases, each of a plurality of template nucleotide sequences may have from about 130 nucleotides to 380 nucleotides in length in total. In some cases, each of a plurality of template nucleotide sequences may have from about 140 nucleotides to 390 nucleotides in length in total. In some cases, each of a plurality of template nucleotide sequences may have from about 150 nucleotides to 400 nucleotides in length in total. In some cases, each of a plurality of template nucleotide sequences may have from about 160 nucleotides to 410 nucleotides in length in total. In some cases, each of a plurality of template nucleotide sequences may have from about 170 nucleotides to 420 nucleotides in length in total. In some cases, each of a plurality of template nucleotide sequences may have from about 180 nucleotides to 430 nucleotides in length in total. In some cases, each of a plurality of template nucleotide sequences may have from about 190 nucleotides to 440 nucleotides in length in total. In some cases, each of a plurality of template nucleotide sequences may have from about 200 nucleotides to 450 nucleotides in length in total. In some cases, each of a plurality of template nucleotide sequences may have from about 210 nucleotides to 460 nucleotides in length in total. In some cases, each of a plurality of template nucleotide sequences may have from about 220 nucleotides to 470 nucleotides in length in total. In some cases, each of a plurality of template nucleotide sequences may have from about 230 nucleotides to 480 nucleotides in length in total. In some cases, each of a plurality of template nucleotide sequences may have from about 240 nucleotides to 490 nucleotides in length in total. In some cases, each of a plurality of template nucleotide sequences may have from about 250 nucleotides to 500 nucleotides in length in total.

In some cases, a template nucleotide sequence may be a nucleotide sequence that encodes a viral protein involved in the virulence of the virus. For example, a viral protein involved in the virulence of the virus may comprise a viral proteins involved with the virus-host interactions (e.g., the Spike protein of the virus). In other cases, a viral protein involved in the virulence of the virus may comprise a viral protein that is involved in the packaging of the virus (e.g., a viral structural protein such as a Nucleocapsid or Capsid). In some cases, a viral protein involved in the virulence of the virus may also comprise a viral protein that is involved in the replication, transcription, or translation of the virus. In some cases, a mutation in a viral protein involved in the virulence of the virus may increase or decrease the virulence of the virus, the transmissibility of the virus, vaccine resistance, or a combination thereof, relative to a virus without such a mutation.

### Substrate Nucleic Acids

In some instances, substrate nucleic acids may be amplification products of a plurality of template nucleotide sequences. In some cases, substrate nucleic acids may be amplification products of complement sequences of a plurality of template nucleotide sequences. In some cases, substrate nucleic acids may be amplification products of a plurality of template nucleotide sequences or amplification products of complement sequences of the plurality of template nucleotide sequences. In some cases, substrate nucleic acids may be amplification products of template nucleotide sequences and amplification products of complement sequences of the plurality of template nucleotide sequences. In other cases, substrate nucleic acids may also be complement sequences of the amplification products thereof. In other cases, substrate nucleic acids may also be complement sequences of the plurality of template nucleotide sequences thereof. In some instances, substrate nucleic acids may be derived from amplification products of a plurality of template nucleotide sequences, amplification products of complement sequences of a plurality of template nucleotide sequences, complement sequences thereof.

In some instances, substrate nucleic acids may have less than about 1500 nucleotides in length. In some cases, substrate nucleic acids may have less than about 1450 nucleotides in length. In some cases, substrate nucleic acids may have less than about 1400 nucleotides in length. In some cases, substrate nucleic acids may have less than about 1350 nucleotides in length. In some cases, substrate nucleic acids may have less than about 1300 nucleotides in length. In some cases, substrate nucleic acids may have less than about 1250 nucleotides in length. In some cases, substrate nucleic acids may have less than about 1200 nucleotides in length. In some cases, substrate nucleic acids may have less than about 1150 nucleotides in length. In some cases, substrate nucleic acids may have less than about 1100 nucleotides in length. In some cases, substrate nucleic acids may have less than about 1050 nucleotides in length. In some cases, substrate nucleic acids may have less than about 1000 nucleotides in length. In some cases, substrate nucleic acids may have less than about 950 nucleotides in length. In some cases, substrate nucleic acids may have less than about 900 nucleotides in length. In some cases, substrate nucleic acids may have less than about 850 nucleotides in length. In some cases, substrate nucleic acids may have less than about 800 nucleotides in length. In some cases, substrate nucleic acids may have less than about 750 nucleotides in length. In some cases, substrate nucleic acids may have less than about 700 nucleotides in length. In some cases, substrate nucleic acids may have less than about 650 nucleotides in length. In some cases, substrate nucleic acids may have less than about 600 nucleotides in length. In some cases, substrate nucleic acids may have less than about 550 nucleotides in length. In some cases, substrate nucleic acids may have less than about 500 nucleotides in length. In some cases, substrate nucleic acids may have less than about 450 nucleotides in length. In some cases, substrate nucleic acids may have less than about 400 nucleotides in length. In some cases, substrate nucleic acids may have less than about 350 nucleotides in length. In some cases, substrate nucleic acids may have less than about 300 nucleotides in length. In some cases, substrate nucleic acids may have less than about 250 nucleotides in length. In some cases, substrate nucleic acids may have less than about 200 nucleotides in length. In some cases, substrate nucleic acids may have less than about 150 nucleotides in length. In some cases, substrate nucleic acids may have less than about 100 nucleotides in length. In some cases, substrate nucleic acids may have less than about 50 nucleotides in length. In some cases, substrate nucleic acids may have less than about 1550 nucleotides in length. In some cases, substrate nucleic acids may have less than about 1600 nucleotides in length. In some cases, substrate nucleic acids may have less than about 1650 nucleotides in length. In some cases, substrate nucleic acids may have less than about 1700 nucleotides in length. In some cases, substrate nucleic acids may have less than about 1750 nucleotides in length. In some cases, substrate nucleic acids may have less than about 1800 nucleotides in length. In some cases, substrate nucleic acids may have less than about 1850 nucleotides in length. In some cases, substrate nucleic acids may have less than about 1900 nucleotides in length. In some cases, substrate nucleic acids may have less than about 1950 nucleotides in length. In some cases, substrate nucleic acids may have less than about 2000 nucleotides in length. In some cases, substrate nucleic acids may have less than about 2050 nucleotides in length. In some cases, substrate nucleic acids may have less than about 2100 nucleotides in length. In some cases, substrate nucleic acids may have less than about 2150 nucleotides in length. In some cases, substrate nucleic acids may have less than about 2200 nucleotides in length. In some cases, substrate nucleic acids may have less than about 2250 nucleotides in length. In some cases, substrate nucleic acids may have less than about 2300 nucleotides in length. In some cases, substrate nucleic acids may have less than about 2350 nucleotides in length. In some cases, substrate nucleic acids may have less than about 2400 nucleotides in length. In some cases, substrate nucleic acids may have less than about 2450 nucleotides in length. In some cases, substrate nucleic acids may have less than about 2500 nucleotides in length. In some cases, substrate nucleic acids may have less than about 2550 nucleotides in length. In some cases, substrate nucleic acids may have less than about 2600 nucleotides in length. In some cases, substrate nucleic acids may have less than about 2650 nucleotides in length. In some cases, substrate nucleic acids may have less than about 2700 nucleotides in length. In some cases, substrate nucleic acids may have less than about 2750 nucleotides in length. In some cases, substrate nucleic acids may have less than about 2800 nucleotides in length. In some cases, substrate nucleic acids may have less than about 2850 nucleotides in length. In some cases, substrate nucleic acids may have less than about 2900 nucleotides in length. In some cases, substrate nucleic acids may have less than about 2950 nucleotides in length. In some cases, substrate nucleic acids may have less than about 3000 nucleotides in length. In some cases, substrate nucleic acids may have less than at least about 3500 nucleotides in length, less than at least about 4000 nucleotides in length, less than at least about 4500 nucleotides in length, less than at least about 5000 nucleotides in length, less than at least about 5500 nucleotides in length, less than at least about 6000 nucleotides in length, less than at least about 6500 nucleotides in length, less than at least about 7000 nucleotides in length, less than at least about 7500 nucleotides in length, less than at least about 8000 nucleotides in length, less than at least about 8500 nucleotides in length, less than at least about 9000 nucleotides in length, less than at least about 9500 nucleotides in length, less than at least about 10000 nucleotides in length, less than at least about 15000 nucleotides in length, less than at least about 20000 nucleotides in length, less than at least about 25000 nucleotides in length, less than at least about 30000 nucleotides in length, less than at least about 35000 nucleotides in length, less than at least about 40000 nucleotides in length, less than at least about 45000 nucleotides in length, or less than at least about 50000 nucleotides in length.

In some instances, substrate nucleic acids may have less than about 1450 to about 1550 nucleotides in length. In some cases, substrate nucleic acids may have less than about 1400 to about 1600 nucleotides in length. In some cases, substrate nucleic acids may have less than about 1350 to about 1650 nucleotides in length. In some cases, substrate nucleic acids may have less than about 1300 to about 1700 nucleotides in length. In some cases, substrate nucleic acids may have less than about 1250 to about 1750 nucleotides in length. In some cases, substrate nucleic acids may have less than about 1200 to about 1800 nucleotides in length. In some cases, substrate nucleic acids may have less than about 1150 to about 1850 nucleotides in length. In some cases, substrate nucleic acids may have less than about 1100 to about 1900 nucleotides in length. In some cases, substrate nucleic acids may have less than about 1050 to about 1950 nucleotides in length. In some cases, substrate nucleic acids may have less than about 1000 to about 2000 nucleotides in length. In some cases, substrate nucleic acids may have less than about 950 to about 2050 nucleotides in length. In some cases, substrate nucleic acids may have less than about 900 to about 2100 nucleotides in length. In some cases, substrate nucleic acids may have less than about 850 to about 2150 nucleotides in length. In some cases, substrate nucleic acids may have less than about 800 to about 2200 nucleotides in length. In some cases, substrate nucleic acids may have less than about 750 to about 2250 nucleotides in length. In some cases, substrate nucleic acids may have less than about 700 to about 2300 nucleotides in length. In some cases, substrate nucleic acids may have less than about 650 to about 2350 nucleotides in length. In some cases, substrate nucleic acids may have less than about 600 to about 2400 nucleotides in length. In some cases, substrate nucleic acids may have less than about 550 to about 2450 nucleotides in length. In some cases, substrate nucleic acids may have less than about 500 to about 2500 nucleotides in length. In some cases, substrate nucleic acids may have less than about 450 to about 2550 nucleotides in length. In some cases, substrate nucleic acids may have less than about 400 to about 2600 nucleotides in length. In some cases, substrate nucleic acids may have less than about 350 to about 2650 nucleotides in length. In some cases, substrate nucleic acids may have less than about 300 to about 2700 nucleotides in length. In some cases, substrate nucleic acids may have less than about 250 to about 2750 nucleotides in length. In some cases, substrate nucleic acids may have less than about 200 to about 2800 nucleotides in length. In some cases, substrate nucleic acids may have less than about 150 to about 2850 nucleotides in length. In some cases, substrate nucleic acids may have less than about 100 to about 2900 nucleotides in length. In some cases, substrate nucleic acids may have less than about 50 to about 2950 nucleotides in length. In some cases, substrate nucleic acids may have less than about 1 to about 3000 nucleotides in length. In some cases, substrate nucleic acids may have less than about 700 to about 800 nucleotides in length. In some cases, substrate nucleic acids may have less than about 650 to about 850 nucleotides in length. In some cases, substrate nucleic acids may have less than about 600 to about 900 nucleotides in length. In some cases, substrate nucleic acids may have less than about 550 to about 950 nucleotides in length. In some cases, substrate nucleic acids may have less than about 500 to about 1000 nucleotides in length. In some cases, substrate nucleic acids may have less than about 450 to about 1050 nucleotides in length. In some cases, substrate nucleic acids may have less than about 400 to about 1100 nucleotides in length. In some cases, substrate nucleic acids may have less than about 350 to about 1150 nucleotides in length. In some cases, substrate nucleic acids may have less than about 300 to about 1200 nucleotides in length. In some cases, substrate nucleic acids may have less than about 250 to about 1250 nucleotides in length. In some cases, substrate nucleic acids may have less than about 200 to about 1300 nucleotides in length. In some cases, substrate nucleic acids may have less than about 150 to about 1350 nucleotides in length. In some cases, substrate nucleic acids may have less than about 100 to about 1400 nucleotides in length. In some cases, substrate nucleic acids may have less than about 50 to about 1450 nucleotides in length. In some cases, substrate nucleic acids may have less than about 1 to about 1500 nucleotides in length.

In some cases, substrate nucleic acids may have less than about 100 to about 500 nucleotides in length, less than about 100 to about 550 nucleotides in length, less than about 100 to about 600 nucleotides in length, less than about 100 to about 650 nucleotides in length, less than about 100 to about 700 nucleotides in length, less than about 100 to about 750 nucleotides in length, less than about 100 to about 800 nucleotides in length, less than about 100 to about 850 nucleotides in length, less than about 100 to about 900 nucleotides in length, less than about 100 to about 950 nucleotides in length, less than about 100 to about 1000 nucleotides in length, less than about 100 to about 1050 nucleotides in length, less than about 100 to about 1100 nucleotides in length, less than about 100 to about 1150 nucleotides in length, less than about 100 to about 1200 nucleotides in length, less than about 100 to about 1250 nucleotides in length, less than about 100 to about 1300 nucleotides in length, less than about 100 to about 1350 nucleotides in length, less than about 100 to about 1400 nucleotides in length, less than about 100 to about 1450 nucleotides in length, less than about 100 to about 1500 nucleotides in length, less than about 100 to about 1600 nucleotides in length, less than about 100 to about 1700 nucleotides in length, less than about 100 to about 1800 nucleotides in length, less than about 100 to about 1900 nucleotides in length, less than about 100 to about 2000 nucleotides in length, less than about 100 to about 3000 nucleotides in length, less than about 100 to about 4000 nucleotides in length, less than about 100 to about 5000 nucleotides in length, less than about 100 to about 6000 nucleotides in length, less than about 100 to about 7000 nucleotides in length, less than about 100 to about 8000 nucleotides in length, less than about 100 to about 9000 nucleotides in length, less than about 100 to about 10000 nucleotides in length, less than about 100 to about 15000 nucleotides in length, less than about 100 to about 20000 nucleotides in length, less than about 100 to about 25000 nucleotides in length, less than about 100 to about 30000 nucleotides in length, less than about 100 to about 35000 nucleotides in length, less than about 100 to about 40000 nucleotides in length, less than about 100 to about 45000 nucleotides in length, or less than about 100 to about 50000 nucleotides in length.

In some instances, substrate nucleic acids may have at least about 1500 nucleotides in length. In some cases, substrate nucleic acids may have at least about 1450 nucleotides in length. In some cases, substrate nucleic acids may have at least about 1400 nucleotides in length. In some cases, substrate nucleic acids may have at least about 1350 nucleotides in length. In some cases, substrate nucleic acids may have at least about 1300 nucleotides in length. In some cases, substrate nucleic acids may have at least about 1250 nucleotides in length. In some cases, substrate nucleic acids may have at least about 1200 nucleotides in length. In some cases, substrate nucleic acids may have at least about 1150 nucleotides in length. In some cases, substrate nucleic acids may have at least about 1100 nucleotides in length. In some cases, substrate nucleic acids may have at least about 1050 nucleotides in length. In some cases, substrate nucleic acids may have at least about 1000 nucleotides in length. In some cases, substrate nucleic acids may have at least about 950 nucleotides in length. In some cases, substrate nucleic acids may have at least about 900 nucleotides in length. In some cases, substrate nucleic acids may have at least about 850 nucleotides in length. In some cases, substrate nucleic acids may have at least about 800 nucleotides in length. In some cases, substrate nucleic acids may have at least about 750 nucleotides in length. In some cases, substrate nucleic acids may have at least about 700 nucleotides in length. In some cases, substrate nucleic acids may have at least about 650 nucleotides in length. In some cases, substrate nucleic acids may have at least about 600 nucleotides in length. In some cases, substrate nucleic acids may have at least about 550 nucleotides in length. In some cases, substrate nucleic acids may have at least about 500 nucleotides in length. In some cases, substrate nucleic acids may have at least about 450 nucleotides in length. In some cases, substrate nucleic acids may have at least about 400 nucleotides in length. In some cases, substrate nucleic acids may have at least about 350 nucleotides in length. In some cases, substrate nucleic acids may have at least about 300 nucleotides in length. In some cases, substrate nucleic acids may have at least about 250 nucleotides in length. In some cases, substrate nucleic acids may have at least about 200 nucleotides in length. In some cases, substrate nucleic acids may have at least about 150 nucleotides in length. In some cases, substrate nucleic acids may have at least about 100 nucleotides in length. In some cases, substrate nucleic acids may have at least about 50 nucleotides in length. In some cases, substrate nucleic acids may have at least about 1550 nucleotides in length. In some cases, substrate nucleic acids may have at least about 1600 nucleotides in length. In some cases, substrate nucleic acids may have at least about 1650 nucleotides in length. In some cases, substrate nucleic acids may have at least about 1700 nucleotides in length. In some cases, substrate nucleic acids may have at least about 1750 nucleotides in length. In some cases, substrate nucleic acids may have at least about 1800 nucleotides in length. In some cases, substrate nucleic acids may have at least about 1850 nucleotides in length. In some cases, substrate nucleic acids may have at least about 1900 nucleotides in length. In some cases, substrate nucleic acids may have at least about 1950 nucleotides in length. In some cases, substrate nucleic acids may have at least about 2000 nucleotides in length. In some cases, substrate nucleic acids may have at least about 2050 nucleotides in length. In some cases, substrate nucleic acids may have at least about 2100 nucleotides in length. In some cases, substrate nucleic acids may have at least about 2150 nucleotides in length. In some cases, substrate nucleic acids may have at least about 2200 nucleotides in length. In some cases, substrate nucleic acids may have at least about 2250 nucleotides in length. In some cases, substrate nucleic acids may have at least about 2300 nucleotides in length. In some cases, substrate nucleic acids may have at least about 2350 nucleotides in length. In some cases, substrate nucleic acids may have at least about 2400 nucleotides in length. In some cases, substrate nucleic acids may have at least about 2450 nucleotides in length. In some cases, substrate nucleic acids may have at least about 2500 nucleotides in length. In some cases, substrate nucleic acids may have at least about 2550 nucleotides in length. In some cases, substrate nucleic acids may have at least about 2600 nucleotides in length. In some cases, substrate nucleic acids may have at least about 2650 nucleotides in length. In some cases, substrate nucleic acids may have at least about 2700 nucleotides in length. In some cases, substrate nucleic acids may have at least about 2750 nucleotides in length. In some cases, substrate nucleic acids may have at least about 2800 nucleotides in length. In some cases, substrate nucleic acids may have at least about 2850 nucleotides in length. In some cases, substrate nucleic acids may have at least about 2900 nucleotides in length. In some cases, substrate nucleic acids may have at least about 2950 nucleotides in length. In some cases, substrate nucleic acids may have at least about 3000 nucleotides in length. In some cases, substrate nucleic acids may have at least at least about 3500 nucleotides in length, at least at least about 4000 nucleotides in length, at least at least about 4500 nucleotides in length, at least at least about 5000 nucleotides in length, at least at least about 5500 nucleotides in length, at least at least about 6000 nucleotides in length, at least at least about 6500 nucleotides in length, at least at least about 7000 nucleotides in length, at least at least about 7500 nucleotides in length, at least at least about 8000 nucleotides in length, at least at least about 8500 nucleotides in length, at least at least about 9000 nucleotides in length, at least at least about 9500 nucleotides in length, at least at least about 10000 nucleotides in length, at least at least about 15000 nucleotides in length, at least at least about 20000 nucleotides in length, at least at least about 25000 nucleotides in length, at least at least about 30000 nucleotides in length, at least at least about 35000 nucleotides in length, at least at least about 40000 nucleotides in length, at least at least about 45000 nucleotides in length, or at least at least about 50000 nucleotides in length.

In some cases, substrate nucleic acids may have from about 1 to about 3000 nucleotides in length. In some cases, substrate nucleic acids may have from about 25 to about 2900 nucleotides in length. In some cases, substrate nucleic acids may have from about 50 to about 2800 nucleotides in length. In some cases, substrate nucleic acids may have from about 75 to about 2700 nucleotides in length. In some cases, substrate nucleic acids may have from about 100 to about 2600 nucleotides in length. In some cases, substrate nucleic acids may have from about 125 to about 2500 nucleotides in length. In some cases, substrate nucleic acids may have from about 150 to about 2400 nucleotides in length. In some cases, substrate nucleic acids may have from about 175 to about 2300 nucleotides in length. In some cases, substrate nucleic acids may have from about 200 to about 2200 nucleotides in length. In some cases, substrate nucleic acids may have from about 225 to about 2100 nucleotides in length. In some cases, substrate nucleic acids may have from about 250 to about 2000 nucleotides in length. In some cases, substrate nucleic acids may have from about 275 to about 1900 nucleotides in length. In some cases, substrate nucleic acids may have from about 300 to about 1800 nucleotides in length. In some cases, substrate nucleic acids may have from about 325 to about 1700 nucleotides in length. In some cases, substrate nucleic acids may have from about 350 to about 1600 nucleotides in length. In some cases, substrate nucleic acids may have from about 375 to about 1500 nucleotides in length. In some cases, substrate nucleic acids may have from about 400 to about 1400 nucleotides in length. In some cases, substrate nucleic acids may have from about 425 to about 1300 nucleotides in length. In some cases, substrate nucleic acids may have from about 450 to about 1200 nucleotides in length. In some cases, substrate nucleic acids may have from about 475 to about 1100 nucleotides in length. In some cases, substrate nucleic acids may have from about 500 to about 1000 nucleotides in length. In some cases, substrate nucleic acids may have from about 525 to about 950 nucleotides in length. In some cases, substrate nucleic acids may have from about 550 to about 900 nucleotides in length. In some cases, substrate nucleic acids may have from about 575 to about 850 nucleotides in length. In some cases, substrate nucleic acids may have from about 600 to about 800 nucleotides in length. In some cases, substrate nucleic acids may have from about 625 to about 750 nucleotides in length.

In some instances, substrate nucleic acids may have from about 1450 to about 1550 nucleotides in length. In some cases, substrate nucleic acids may have from about 1400 to about 1600 nucleotides in length. In some cases, substrate nucleic acids may have from about 1350 to about 1650 nucleotides in length. In some cases, substrate nucleic acids may have from about 1300 to about 1700 nucleotides in length. In some cases, substrate nucleic acids may have from about 1250 to about 1750 nucleotides in length. In some cases, substrate nucleic acids may have from about 1200 to about 1800 nucleotides in length. In some cases, substrate nucleic acids may have from about 1150 to about 1850 nucleotides in length. In some cases, substrate nucleic acids may have from about 1100 to about 1900 nucleotides in length. In some cases, substrate nucleic acids may have from about 1050 to about 1950 nucleotides in length. In some cases, substrate nucleic acids may have from about 1000 to about 2000 nucleotides in length. In some cases, substrate nucleic acids may have from about 950 to about 2050 nucleotides in length. In some cases, substrate nucleic acids may have from about 900 to about 2100 nucleotides in length. In some cases, substrate nucleic acids may have from about 850 to about 2150 nucleotides in length. In some cases, substrate nucleic acids may have from about 800 to about 2200 nucleotides in length. In some cases, substrate nucleic acids may have from about 750 to about 2250 nucleotides in length. In some cases, substrate nucleic acids may have from about 700 to about 2300 nucleotides in length. In some cases, substrate nucleic acids may have from about 650 to about 2350 nucleotides in length. In some cases, substrate nucleic acids may have from about 600 to about 2400 nucleotides in length. In some cases, substrate nucleic acids may have from about 550 to about 2450 nucleotides in length. In some cases, substrate nucleic acids may have from about 500 to about 2500 nucleotides in length. In some cases, substrate nucleic acids may have from about 450 to about 2550 nucleotides in length. In some cases, substrate nucleic acids may have from about 400 to about 2600 nucleotides in length. In some cases, substrate nucleic acids may have from about 350 to about 2650 nucleotides in length. In some cases, substrate nucleic acids may have from about 300 to about 2700 nucleotides in length. In some cases, substrate nucleic acids may have from about 250 to about 2750 nucleotides in length. In some cases, substrate nucleic acids may have from about 200 to about 2800 nucleotides in length. In some cases, substrate nucleic acids may have from about 150 to about 2850 nucleotides in length. In some cases, substrate nucleic acids may have from about 100 to about 2900 nucleotides in length. In some cases, substrate nucleic acids may have from about 50 to about 2950 nucleotides in length. In some cases, substrate nucleic acids may have from about 1 to about 3000 nucleotides in length. In some cases, substrate nucleic acids may have from about 700 to about 800 nucleotides in length. In some cases, substrate nucleic acids may have from about 650 to about 850 nucleotides in length. In some cases, substrate nucleic acids may have from about 600 to about 900 nucleotides in length. In some cases, substrate nucleic acids may have from about 550 to about 950 nucleotides in length. In some cases, substrate nucleic acids may have from about 500 to about 1000 nucleotides in length. In some cases, substrate nucleic acids may have from about 450 to about 1050 nucleotides in length. In some cases, substrate nucleic acids may have from about 400 to about 1100 nucleotides in length. In some cases, substrate nucleic acids may have from about 350 to about 1150 nucleotides in length. In some cases, substrate nucleic acids may have from about 300 to about 1200 nucleotides in length. In some cases, substrate nucleic acids may have from about 250 to about 1250 nucleotides in length. In some cases, substrate nucleic acids may have from about 200 to about 1300 nucleotides in length. In some cases, substrate nucleic acids may have from about 150 to about 1350 nucleotides in length. In some cases, substrate nucleic acids may have from about 100 to about 1400 nucleotides in length. In some cases, substrate nucleic acids may have from about 50 to about 1450 nucleotides in length. In some cases, substrate nucleic acids may have from about 1 to about 1500 nucleotides in length.

In some cases, substrate nucleic acids may have from about 100 to about 500 nucleotides in length, from about 100 to about 550 nucleotides in length, from about 100 to about 600 nucleotides in length, from about 100 to about 650 nucleotides in length, from about 100 to about 700 nucleotides in length, from about 100 to about 750 nucleotides in length, from about 100 to about 800 nucleotides in length, from about 100 to about 850 nucleotides in length, from about 100 to about 900 nucleotides in length, from about 100 to about 950 nucleotides in length, from about 100 to about 1000 nucleotides in length, from about 100 to about 1050 nucleotides in length, from about 100 to about 1100 nucleotides in length, from about 100 to about 1150 nucleotides in length, from about 100 to about 1200 nucleotides in length, from about 100 to about 1250 nucleotides in length, from about 100 to about 1300 nucleotides in length, from about 100 to about 1350 nucleotides in length, from about 100 to about 1400 nucleotides in length, from about 100 to about 1450 nucleotides in length, from about 100 to about 1500 nucleotides in length, from about 100 to about 1600 nucleotides in length, from about 100 to about 1700 nucleotides in length, from about 100 to about 1800 nucleotides in length, from about 100 to about 1900 nucleotides in length, from about 100 to about 2000 nucleotides in length, from about 100 to about 3000 nucleotides in length, from about 100 to about 4000 nucleotides in length, from about 100 to about 5000 nucleotides in length, from about 100 to about 6000 nucleotides in length, from about 100 to about 7000 nucleotides in length, from about 100 to about 8000 nucleotides in length, from about 100 to about 9000 nucleotides in length, from about 100 to about 10000 nucleotides in length, from about 100 to about 15000 nucleotides in length, from about 100 to about 20000 nucleotides in length, from about 100 to about 25000 nucleotides in length, from about 100 to about 30000 nucleotides in length, from about 100 to about 35000 nucleotides in length, from about 100 to about 40000 nucleotides in length, from about 100 to about 45000 nucleotides in length, or from about 100 to about 50000 nucleotides in length.

In some instances, substrate nucleic acids may have from about 1450 to about 1550 nucleotides in length. In some cases, substrate nucleic acids may have from about 1400 to about 1600 nucleotides in length. In some cases, substrate nucleic acids may have from about 1350 to about 1650 nucleotides in length. In some cases, substrate nucleic acids may have from about 1300 to about 1700 nucleotides in length. In some cases, substrate nucleic acids may have from about 1250 to about 1750 nucleotides in length. In some cases, substrate nucleic acids may have from about 1200 to about 1800 nucleotides in length. In some cases, substrate nucleic acids may have from about 1150 to about 1850 nucleotides in length. In some cases, substrate nucleic acids may have from about 1100 to about 1900 nucleotides in length. In some cases, substrate nucleic acids may have from about 1050 to about 1950 nucleotides in length. In some cases, substrate nucleic acids may have from about 1000 to about 2000 nucleotides in length. In some cases, substrate nucleic acids may have from about 950 to about 2050 nucleotides in length. In some cases, substrate nucleic acids may have from about 900 to about 2100 nucleotides in length. In some cases, substrate nucleic acids may have from about 850 to about 2150 nucleotides in length. In some cases, substrate nucleic acids may have from about 800 to about 2200 nucleotides in length. In some cases, substrate nucleic acids may have from about 750 to about 2250 nucleotides in length. In some cases, substrate nucleic acids may have from about 700 to about 2300 nucleotides in length. In some cases, substrate nucleic acids may have from about 650 to about 2350 nucleotides in length. In some cases, substrate nucleic acids may have from about 600 to about 2400 nucleotides in length. In some cases, substrate nucleic acids may have from about 550 to about 2450 nucleotides in length. In some cases, substrate nucleic acids may have from about 500 to about 2500 nucleotides in length. In some cases, substrate nucleic acids may have from about 450 to about 2550 nucleotides in length. In some cases, substrate nucleic acids may have from about 400 to about 2600 nucleotides in length. In some cases, substrate nucleic acids may have from about 350 to about 2650 nucleotides in length. In some cases, substrate nucleic acids may have from about 300 to about 2700 nucleotides in length. In some cases, substrate nucleic acids may have from about 250 to about 2750 nucleotides in length. In some cases, substrate nucleic acids may have from about 200 to about 2800 nucleotides in length. In some cases, substrate nucleic acids may have from about 150 to about 2850 nucleotides in length. In some cases, substrate nucleic acids may have from about 100 to about 2900 nucleotides in length. In some cases, substrate nucleic acids may have from about 50 to about 2950 nucleotides in length. In some cases, substrate nucleic acids may have from about 1 to about 3000 nucleotides in length. In some cases, substrate nucleic acids may have from about 700 to about 800 nucleotides in length. In some cases, substrate nucleic acids may have from about 650 to about 850 nucleotides in length. In some cases, substrate nucleic acids may have from about 600 to about 900 nucleotides in length. In some cases, substrate nucleic acids may have from about 550 to about 950 nucleotides in length. In some cases, substrate nucleic acids may have from about 500 to about 1000 nucleotides in length. In some cases, substrate nucleic acids may have from about 450 to about 1050 nucleotides in length. In some cases, substrate nucleic acids may have from about 400 to about 1100 nucleotides in length. In some cases, substrate nucleic acids may have from about 350 to about 1150 nucleotides in length. In some cases, substrate nucleic acids may have from about 300 to about 1200 nucleotides in length. In some cases, substrate nucleic acids may have from about 250 to about 1250 nucleotides in length. In some cases, substrate nucleic acids may have from about 200 to about 1300 nucleotides in length. In some cases, substrate nucleic acids may have from about 150 to about 1350 nucleotides in length. In some cases, substrate nucleic acids may have from about 100 to about 1400 nucleotides in length. In some cases, substrate nucleic acids may have from about 50 to about 1450 nucleotides in length.

In some cases, substrate nucleic acids may have from about 100 to about 30000 nucleotides in length, from about 200 to about 30000 nucleotides in length, from about 300 to about 30000 nucleotides in length, from about 400 to about 30000 nucleotides in length, from about 500 to about 30000 nucleotides in length, from about 600 to about 30000 nucleotides in length, from about 700 to about 30000 nucleotides in length, from about 800 to about 30000 nucleotides in length, from about 900 to about 30000 nucleotides in length, from about 1000 to about 30000 nucleotides in length, from about 1100 to about 30000 nucleotides in length, from about 1200 to about 30000 nucleotides in length, from about 1300 to about 30000 nucleotides in length, from about 1400 to about 30000 nucleotides in length, from about 1500 to about 30000 nucleotides in length, from about 1600 to about 30000 nucleotides in length, from about 1700 to about 30000 nucleotides in length, from about 1800 to about 30000 nucleotides in length, from about 1900 to about 30000 nucleotides in length, from about 2000 to about 30000 nucleotides in length, from about 2100 to about 30000 nucleotides in length, from about 2200 to about 30000 nucleotides in length, from about 2300 to about 30000 nucleotides in length, from about 2400 to about 30000 nucleotides in length, from about 2500 to about 30000 nucleotides in length, from about 2600 to about 30000 nucleotides in length, from about 2700 to about 30000 nucleotides in length, from about 2800 to about 30000 nucleotides in length, from about 2900 to about 30000 nucleotides in length, from about 3000 to about 30000 nucleotides in length, from about 3500 to about 30000 nucleotides in length, from about 4000 to about 30000 nucleotides in length, from about 4500 to about 30000 nucleotides in length, from about 5000 to about 30000 nucleotides in length, from about 6000 to about 30000 nucleotides in length, from about 7000 to about 30000 nucleotides in length, from about 8000 to about 30000 nucleotides in length, from about 9000 to about 30000 nucleotides in length, from about 10000 to about 30000 nucleotides in length, from about 15000 to about 30000 nucleotides in length, from about 20000 to about 30000 nucleotides in length, or from about 25000 to about 30000 nucleotides in length.

In some cases, a substrate nucleic acid may have about 50 nucleotides in length. In some cases, a substrate nucleic acid may have about 60 nucleotides in length. In some cases, a substrate nucleic acid may have about 70 nucleotides in length. In some cases, a substrate nucleic acid may have about 80 nucleotides in length. In some cases, a substrate nucleic acid may have about 90 nucleotides in length. In some cases, a substrate nucleic acid may have about 100 nucleotides in length. In some cases, a substrate nucleic acid may have about 110 nucleotides in length. In some cases, a substrate nucleic acid may have about 120 nucleotides in length. In some cases, a substrate nucleic acid may have about 130 nucleotides in length. In some cases, a substrate nucleic acid may have about 140 nucleotides in length. In some cases, a substrate nucleic acid may have about 150 nucleotides in length. In some cases, a substrate nucleic acid may have about 160 nucleotides in length. In some cases, a substrate nucleic acid may have about 170 nucleotides in length. In some cases, a substrate nucleic acid may have about 180 nucleotides in length. In some cases, a substrate nucleic acid may have about 190 nucleotides in length. In some cases, a substrate nucleic acid may have about 200 nucleotides in length. In some cases, a substrate nucleic acid may have about 210 nucleotides in length. In some cases, a substrate nucleic acid may have about 220 nucleotides in length. In some cases, a substrate nucleic acid may have about 230 nucleotides in length. In some cases, a substrate nucleic acid may have about 240 nucleotides in length. In some cases, a substrate nucleic acid may have about 250 nucleotides in length. In some cases, a substrate nucleic acid may have about 260 nucleotides in length. In some cases, a substrate nucleic acid may have about 270 nucleotides in length. In some cases, a substrate nucleic acid may have about 280 nucleotides in length. In some cases, a substrate nucleic acid may have about 290 nucleotides in length. In some cases, a substrate nucleic acid may have about 300 nucleotides in length. In some cases, a substrate nucleic acid may have about 310 nucleotides in length. In some cases, a substrate nucleic acid may have about 320 nucleotides in length. In some cases, a substrate nucleic acid may have about 330 nucleotides in length. In some cases, a substrate nucleic acid may have about 340 nucleotides in length. In some cases, a substrate nucleic acid may have about 350 nucleotides in length. In some cases, a substrate nucleic acid may have about 360 nucleotides in length. In some cases, a substrate nucleic acid may have about 370 nucleotides in length. In some cases, a substrate nucleic acid may have about 380 nucleotides in length. In some cases, a substrate nucleic acid may have about 390 nucleotides in length. In some cases, a substrate nucleic acid may have about 400 nucleotides in length. In some cases, a substrate nucleic acid may have about 410 nucleotides in length. In some cases, a substrate nucleic acid may have about 420 nucleotides in length. In some cases, a substrate nucleic acid may have about 430 nucleotides in length. In some cases, a substrate nucleic acid may have about 440 nucleotides in length. In some cases, a substrate nucleic acid may have about 450 nucleotides in length. In some cases, a substrate nucleic acid may have about 460 nucleotides in length. In some cases, a substrate nucleic acid may have about 470 nucleotides in length. In some cases, a substrate nucleic acid may have about 480 nucleotides in length. In some cases, a substrate nucleic acid may have about 490 nucleotides in length. In some cases, a substrate nucleic acid may have about 500 nucleotides in length. In some cases, a substrate nucleic acid may have about 510 nucleotides in length, about 520 nucleotides in length, about 530 nucleotides in length, about 540 nucleotides in length, about 550 nucleotides in length, about 560 nucleotides in length, about 570 nucleotides in length, about 580 nucleotides in length, about 590 nucleotides in length, about 600 nucleotides in length, about 610 nucleotides in length, about 620 nucleotides in length, about 630 nucleotides in length, about 640 nucleotides in length, about 650 nucleotides in length, about 660 nucleotides in length, about 670 nucleotides in length, about 680 nucleotides in length, about 690 nucleotides in length, about 700 nucleotides in length, about 710 nucleotides in length, about 720 nucleotides in length, about 730 nucleotides in length, about 740 nucleotides in length, about 750 nucleotides in length, about 760 nucleotides in length, about 770 nucleotides in length, about 780 nucleotides in length, about 790 nucleotides in length, about 800 nucleotides in length, about 810 nucleotides in length, about 820 nucleotides in length, about 830 nucleotides in length, about 840 nucleotides in length, about 850 nucleotides in length, about 860 nucleotides in length, about 870 nucleotides in length, about 880 nucleotides in length, about 890 nucleotides in length, about 900 nucleotides in length, about 910 nucleotides in length, about 920 nucleotides in length, about 930 nucleotides in length, about 940 nucleotides in length, about 950 nucleotides in length, about 960 nucleotides in length, about 970 nucleotides in length, about 980 nucleotides in length, about 990 nucleotides in length, or about 1000 nucleotides in length.

In some cases, a substrate nucleic acid may have from 1 to 100 nucleotides in length. In some cases, a substrate nucleic acid may have from 10 to 110 nucleotides in length. In some cases, a substrate nucleic acid may have from 20 to 120 nucleotides in length. In some cases, a substrate nucleic acid may have from 30 to 130 nucleotides in length. In some cases, a substrate nucleic acid may have from 40 to 140 nucleotides in length. In some cases, a substrate nucleic acid may have from 50 to 150 nucleotides in length. In some cases, a substrate nucleic acid may have from 60 to 160 nucleotides in length. In some cases, a substrate nucleic acid may have from 70 to 170 nucleotides in length. In some cases, a substrate nucleic acid may have from 80 to 180 nucleotides in length. In some cases, a substrate nucleic acid may have from 90 to 190 nucleotides in length. In some cases, a substrate nucleic acid may have from 100 to 200 nucleotides in length. In some cases, a substrate nucleic acid may have from 110 to 210 nucleotides in length. In some cases, a substrate nucleic acid may have from 120 to 220 nucleotides in length. In some cases, a substrate nucleic acid may have from 130 to 230 nucleotides in length. In some cases, a substrate nucleic acid may have from 140 to 240 nucleotides in length. In some cases, a substrate nucleic acid may have from 150 to 250 nucleotides in length. In some cases, a substrate nucleic acid may have from 160 to 260 nucleotides in length. In some cases, a substrate nucleic acid may have from 170 to 270 nucleotides in length. In some cases, a substrate nucleic acid may have from 180 to 280 nucleotides in length. In some cases, a substrate nucleic acid may have from 190 to 290 nucleotides in length. In some cases, a substrate nucleic acid may have from 200 to 300 nucleotides in length. In some cases, a substrate nucleic acid may have from 210 to 310 nucleotides in length. In some cases, a substrate nucleic acid may have from 220 to 320 nucleotides in length. In some cases, a substrate nucleic acid may have from 230 to 330 nucleotides in length. In some cases, a substrate nucleic acid may have from 240 to 340 nucleotides in length. In some cases, a substrate nucleic acid may have from 250 to 350 nucleotides in length. In some cases, a substrate nucleic acid may have from 260 to 360 nucleotides in length. In some cases, a substrate nucleic acid may have from 270 to 370 nucleotides in length. In some cases, a substrate nucleic acid may have from 280 to 380 nucleotides in length. In some cases, a substrate nucleic acid may have from 290 to 390 nucleotides in length. In some cases, a substrate nucleic acid may have from 300 to 400 nucleotides in length. In some cases, a substrate nucleic acid may have from 310 to 410 nucleotides in length. In some cases, a substrate nucleic acid may have from 320 to 420 nucleotides in length. In some cases, a substrate nucleic acid may have from 330 to 430 nucleotides in length. In some cases, a substrate nucleic acid may have from 340 to 440 nucleotides in length. In some cases, a substrate nucleic acid may have from 350 to 450 nucleotides in length. In some cases, a substrate nucleic acid may have from 360 to 460 nucleotides in length. In some cases, a substrate nucleic acid may have from 370 to 470 nucleotides in length. In some cases, a substrate nucleic acid may have from 380 to 480 nucleotides in length. In some cases, a substrate nucleic acid may have from 390 to 490 nucleotides in length. In some cases, a substrate nucleic acid may have from 400 to 500 nucleotides in length. In some cases, a substrate nucleic acid may have from 410 to 510 nucleotides in length. In some cases, a substrate nucleic acid may have from 420 to 520 nucleotides in length. In some cases, a substrate nucleic acid may have from 430 to 530 nucleotides in length. In some cases, a substrate nucleic acid may have from 440 to 540 nucleotides in length. In some cases, a substrate nucleic acid may have from 450 to 550 nucleotides in length. In some cases, a substrate nucleic acid may have from 460 to 560 nucleotides in length, from 520 to 570 nucleotides in length, from 530 to 580 nucleotides in length, from 540 to 590 nucleotides in length, from 550 to 600 nucleotides in length, from 560 to 610 nucleotides in length, from 570 to 620 nucleotides in length, from 580 to 630 nucleotides in length, from 590 to 640 nucleotides in length, from 600 to 650 nucleotides in length, from 610 to 660 nucleotides in length, from 620 to 670 nucleotides in length, from 630 to 680 nucleotides in length, from 640 to 690 nucleotides in length, from 650 to 700 nucleotides in length, from 660 to 710 nucleotides in length, from 670 to 720 nucleotides in length, from 680 to 730 nucleotides in length, from 690 to 740 nucleotides in length, from 700 to 750 nucleotides in length, from 710 to 760 nucleotides in length, from 720 to 770 nucleotides in length, from 730 to 780 nucleotides in length, from 740 to 790 nucleotides in length, from 750 to 800 nucleotides in length, from 760 to 810 nucleotides in length, from 770 to 820 nucleotides in length, from 780 to 830 nucleotides in length, from 790 to 840 nucleotides in length, from 800 to 850 nucleotides in length, from 810 to 860 nucleotides in length, from 820 to 870 nucleotides in length, from 830 to 880 nucleotides in length, from 840 to 890 nucleotides in length, from 850 to 900 nucleotides in length, from 860 to 910 nucleotides in length, from 870 to 920 nucleotides in length, from 880 to 930 nucleotides in length, from 890 to 940 nucleotides in length, from 900 to 950 nucleotides in length, from 910 to 960 nucleotides in length, from 920 to 970 nucleotides in length, from 930 to 980 nucleotides in length, from 940 to 990 nucleotides in length, or from 950 to 1000 nucleotides in length.

In some cases, each of substrate nucleic acids may have about 50 nucleotides in length. In some cases, each of substrate nucleic acids may have about 60 nucleotides in length. In some cases, each of substrate nucleic acids may have about 70 nucleotides in length. In some cases, each of substrate nucleic acids may have about 80 nucleotides in length. In some cases, each of substrate nucleic acids may have about 90 nucleotides in length. In some cases, each of substrate nucleic acids may have about 100 nucleotides in length. In some cases, each of substrate nucleic acids may have about 110 nucleotides in length. In some cases, each of substrate nucleic acids may have about 120 nucleotides in length. In some cases, each of substrate nucleic acids may have about 130 nucleotides in length. In some cases, each of substrate nucleic acids may have about 140 nucleotides in length. In some cases, each of substrate nucleic acids may have about 150 nucleotides in length. In some cases, each of substrate nucleic acids may have about 160 nucleotides in length. In some cases, each of substrate nucleic acids may have about 170 nucleotides in length. In some cases, each of substrate nucleic acids may have about 180 nucleotides in length. In some cases, each of substrate nucleic acids may have about 190 nucleotides in length. In some cases, each of substrate nucleic acids may have about 200 nucleotides in length. In some cases, each of substrate nucleic acids may have about 210 nucleotides in length. In some cases, each of substrate nucleic acids may have about 220 nucleotides in length. In some cases, each of substrate nucleic acids may have about 230 nucleotides in length. In some cases, each of substrate nucleic acids may have about 240 nucleotides in length. In some cases, each of substrate nucleic acids may have about 250 nucleotides in length. In some cases, each of substrate nucleic acids may have about 260 nucleotides in length. In some cases, each of substrate nucleic acids may have about 270 nucleotides in length. In some cases, each of substrate nucleic acids may have about 280 nucleotides in length. In some cases, each of substrate nucleic acids may have about 290 nucleotides in length. In some cases, each of substrate nucleic acids may have about 300 nucleotides in length. In some cases, each of substrate nucleic acids may have about 310 nucleotides in length. In some cases, each of substrate nucleic acids may have about 320 nucleotides in length. In some cases, each of substrate nucleic acids may have about 330 nucleotides in length. In some cases, each of substrate nucleic acids may have about 340 nucleotides in length. In some cases, each of substrate nucleic acids may have about 350 nucleotides in length. In some cases, each of substrate nucleic acids may have about 360 nucleotides in length. In some cases, each of substrate nucleic acids may have about 370 nucleotides in length. In some cases, each of substrate nucleic acids may have about 380 nucleotides in length. In some cases, each of substrate nucleic acids may have about 390 nucleotides in length. In some cases, each of substrate nucleic acids may have about 400 nucleotides in length. In some cases, each of substrate nucleic acids may have about 410 nucleotides in length. In some cases, each of substrate nucleic acids may have about 420 nucleotides in length. In some cases, each of substrate nucleic acids may have about 430 nucleotides in length. In some cases, each of substrate nucleic acids may have about 440 nucleotides in length. In some cases, each of substrate nucleic acids may have about 450 nucleotides in length. In some cases, each of substrate nucleic acids may have about 460 nucleotides in length. In some cases, each of substrate nucleic acids may have about 470 nucleotides in length. In some cases, each of substrate nucleic acids may have about 480 nucleotides in length. In some cases, each of substrate nucleic acids may have about 490 nucleotides in length. In some cases, each of substrate nucleic acids may have about 500 nucleotides in length.

In some cases, each of substrate nucleic acids may have from about 100 nucleotides to 350 nucleotides in length. In some cases, each of substrate nucleic acids may have from about 110 nucleotides to 360 nucleotides in length. In some cases, each of substrate nucleic acids may have from about 120 nucleotides to 370 nucleotides in length. In some cases, each of substrate nucleic acids may have from about 130 nucleotides to 380 nucleotides in length. In some cases, each of substrate nucleic acids may have from about 140 nucleotides to 390 nucleotides in length. In some cases, each of substrate nucleic acids may have from about 150 nucleotides to 400 nucleotides in length. In some cases, each of substrate nucleic acids may have from about 160 nucleotides to 410 nucleotides in length. In some cases, each of substrate nucleic acids may have from about 170 nucleotides to 420 nucleotides in length. In some cases, each of substrate nucleic acids may have from about 180 nucleotides to 430 nucleotides in length. In some cases, each of substrate nucleic acids may have from about 190 nucleotides to 440 nucleotides in length. In some cases, each of substrate nucleic acids may have from about 200 nucleotides to 450 nucleotides in length. In some cases, each of substrate nucleic acids may have from about 210 nucleotides to 460 nucleotides in length. In some cases, each of substrate nucleic acids may have from about 220 nucleotides to 470 nucleotides in length. In some cases, each of substrate nucleic acids may have from about 230 nucleotides to 480 nucleotides in length. In some cases, each of substrate nucleic acids may have from about 240 nucleotides to 490 nucleotides in length. In some cases, each of substrate nucleic acids may have from about 250 nucleotides to 500 nucleotides in length.

### Primers

In some instances, generating substrate nucleic acids may comprise a plurality of primers.

In some instances, generating substrate nucleic acids may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 primer pairs. In some instances, generating substrate nucleic acids may comprise 1, 2, 3, 4, 5, 6, 7, 8, 9, 10, 11, 12, 13, 14, 15, 16, 17, 18, 19, 20, 21, 22, 23, 24, or 25 primer pairs. In some instances, generating substrate nucleic acids may comprise 1, 2, 3, 4, 5, 6, 7, 8, 9, 10, 11, 12, 13, 14, 15, 16, 17, 18, 19, or 20 primer pairs. In some instances, generating substrate nucleic acids may comprise 1, 2, 3, 4, 5, 6, 7, 8, 9, 10, 11, 12, 13, 14, or 15 primer pairs. In some instances, generating substrate nucleic acids may comprise 1, 2, 3, 4, 5, 6, 7, 8, 9, or 10 primer pairs. In some instances, generating substrate nucleic acids may comprise 1, 2, 3, 4, 5, 6, 7, 8, or 9 primer pairs. In some instances, generating substrate nucleic acids may comprise 1, 2, 3, 4, 5, 6, 7, or 8 primer pairs. In some instances, generating substrate nucleic acids may comprise 1, 2, 3, 4, 5, 6, or 7 primer pairs. In some instances, generating substrate nucleic acids may comprise 1, 2, 3, 4, 5, or 6 primer pairs. In some instances, generating substrate nucleic acids may comprise 1, 2, 3, 4, or 5 primer pairs. In some instances, generating substrate nucleic acids may comprise 1, 2, 3, or 4 primer pairs. In some instances, generating substrate nucleic acids may comprise 1, 2, or 3 primer pairs. In some instances, generating substrate nucleic acids may comprise 1, or 2 primer pairs.

In some cases, generating substrate nucleic acids may comprise 1 pair of primers. In some cases, generating substrate nucleic acids may comprise 2 primer pairs. In some instances, generating substrate nucleic acids may comprise 3 primer pairs. In some cases, generating substrate nucleic acids may comprise 4 primer pairs. In some cases, generating substrate nucleic acids may comprise 5 primer pairs. In some cases, generating substrate nucleic acids may comprise 6 primer pairs. In some cases, generating substrate nucleic acids may comprise 7 primer pairs. In some cases, generating substrate nucleic acids may comprise 8 primer pairs. In some cases, generating substrate nucleic acids may comprise 9 primer pairs. In some cases, generating substrate nucleic acids may comprise 10 primer pairs. In some cases, generating substrate nucleic acids may comprise 11 primer pairs. In some cases, generating substrate nucleic acids may comprise 12 primer pairs. In some cases, generating substrate nucleic acids may comprise 13 primer pairs. In some cases, generating substrate nucleic acids may comprise 14 primer pairs. In some cases, generating substrate nucleic acids may comprise 15 primer pairs. In some cases, generating substrate nucleic acids may comprise 16 primer pairs. In some cases, generating substrate nucleic acids may comprise 17 primer pairs. In some cases, generating substrate nucleic acids may comprise 18 primer pairs. In some cases, generating substrate nucleic acids may comprise 19 primer pairs. In some cases, generating substrate nucleic acids may comprise 20 primer pairs. In some cases, generating substrate nucleic acids may comprise 21 primer pairs. In some cases, generating substrate nucleic acids may comprise 22 primer pairs. In some cases, generating substrate nucleic acids may comprise 23 primer pairs. In some cases, generating substrate nucleic acids may comprise 24 primer pairs. In some cases, generating substrate nucleic acids may comprise 25 primer pairs. In some cases, generating substrate nucleic acids may comprise 26 primer pairs. In some cases, generating substrate nucleic acids may comprise 27 primer pairs. In some cases, generating substrate nucleic acids may comprise 28 primer pairs. In some cases, generating substrate nucleic acids may comprise 29 primer pairs. In some cases, generating substrate nucleic acids may comprise 30 primer pairs. In some cases, generating substrate nucleic acids may comprise 31 primer pairs. In some cases, generating substrate nucleic acids may comprise 32 primer pairs. In some cases, generating substrate nucleic acids may comprise 33 primer pairs. In some cases, generating substrate nucleic acids may comprise 34 primer pairs. In some cases, generating substrate nucleic acids may comprise 35 primer pairs. In some cases, generating substrate nucleic acids may comprise 36 primer pairs. In some cases, generating substrate nucleic acids may comprise 37 primer pairs. In some cases, generating substrate nucleic acids may comprise 38 primer pairs. In some cases, generating substrate nucleic acids may comprise 39 primer pairs. In some cases, generating substrate nucleic acids may comprise 40 primer pairs. In some cases, generating substrate nucleic acids may comprise 41 primer pairs. In some cases, generating substrate nucleic acids may comprise 42 primer pairs. In some cases, generating substrate nucleic acids may comprise 43 primer pairs. In some cases, generating substrate nucleic acids may comprise 44 primer pairs. In some cases, generating substrate nucleic acids may comprise 45 primer pairs. In some cases, generating substrate nucleic acids may comprise 46 primer pairs. In some cases, generating substrate nucleic acids may comprise 47 primer pairs. In some cases, generating substrate nucleic acids may comprise 48 primer pairs. In some cases, generating substrate nucleic acids may comprise 49 primer pairs. In some cases, generating substrate nucleic acids may comprise 50 primer pairs. In some cases, generating substrate nucleic acids may comprise about 55 primer pairs, about 60 primer pairs, about 65 primer pairs, about 70 primer pairs, about 75 primer pairs, about 80 primer pairs, about 85 primer pairs, about 90 primer pairs, about 95 primer pairs, about 100 primer pairs, about 110 primer pairs, about 120 primer pairs, about 130 primer pairs, about 140 primer pairs, about 150 primer pairs, about 160 primer pairs, about 170 primer pairs, about 180 primer pairs, about 190 primer pairs, about 200 primer pairs, about 210 primer pairs, about 220 primer pairs, about 230 primer pairs, about 240 primer pairs, about 250 primer pairs, about 260 primer pairs, about 270 primer pairs, about 280 primer pairs, about 290 primer pairs, about 300 primer pairs, about 350 primer pairs, about 400 primer pairs, about 450 primer pairs, or about 500 primer pairs.

In some cases, generating substrate nucleic acids may comprise from 1 to 50 primer pairs. In some cases, generating substrate nucleic acids may comprise from 2 to 50 primer pairs. In some cases, generating substrate nucleic acids may comprise from 3 to 50 primer pairs. In some cases, generating substrate nucleic acids may comprise from 4 to 50 primer pairs. In some cases, generating substrate nucleic acids may comprise from 5 to 50 primer pairs. In some cases, generating substrate nucleic acids may comprise from 6 to 50 primer pairs. In some cases, generating substrate nucleic acids may comprise from 7 to 50 primer pairs. In some cases, generating substrate nucleic acids may comprise from 8 to 50 primer pairs. In some cases, generating substrate nucleic acids may comprise from 9 to 50 primer pairs. In some cases, generating substrate nucleic acids may comprise from 10 to 50 primer pairs. In some cases, generating substrate nucleic acids may comprise from 1 to 45 primer pairs. In some cases, generating substrate nucleic acids may comprise from 1 to 40 primer pairs. In some cases, generating substrate nucleic acids may comprise from 1 to 35 primer pairs. In some cases, generating substrate nucleic acids may comprise from 1 to 30 primer pairs. In some cases, generating substrate nucleic acids may comprise from 1 to 25 primer pairs. In some cases, generating substrate nucleic acids may comprise from 1 to 20 primer pairs. In some cases, generating substrate nucleic acids may comprise from 1 to 15 primer pairs. In some cases, generating substrate nucleic acids may comprise from 1 to 10 primer pairs. In some cases, generating substrate nucleic acids may comprise from 1 to 5 primer pairs.

In some instances, generating substrate nucleic acids may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 primers. In some cases, generating substrate nucleic acids may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 primers. In some cases, generating substrate nucleic acids may comprise 1, 2, 3, 4, 5, 6, 7, 8, 9, 10, 11, 12, 13, 14, 15, 16, 17, 18, 19, 20, 21, 22, 23, 24, or 25 primers. In some cases, generating substrate nucleic acids may comprise 1, 2, 3, 4, 5, 6, 7, 8, 9, 10, 11, 12, 13, 14, 15, 16, 17, 18, 19, or 20 primers. In some cases, generating substrate nucleic acids may comprise 1, 2, 3, 4, 5, 6, 7, 8, 9, 10, 11, 12, 13, 14, or 15 primers. In some cases, generating substrate nucleic acids may comprise 1, 2, 3, 4, 5, 6, 7, 8, 9, 10, 11, 12, 13, or 14 primers. In some cases, generating substrate nucleic acids may comprise 1, 2, 3, 4, 5, 6, 7, 8, 9, 10, 11, 12, or 13 primers. In some cases, generating substrate nucleic acids may comprise 1, 2, 3, 4, 5, 6, 7, 8, 9, 10, 11, or 12 primers. In some cases, generating substrate nucleic acids may comprise 1, 2, 3, 4, 5, 6, 7, 8, 9, 10, or 11 primers. In some cases, generating substrate nucleic acids may comprise 1, 2, 3, 4, 5, 6, 7, 8, 9, or 10 primers. In some cases, generating substrate nucleic acids may comprise 1, 2, 3, 4, 5, 6, 7, 8, or 9 primers. In some cases, generating substrate nucleic acids may comprise 1, 2, 3, 4, 5, 6, 7, or 8 primers. In some cases, generating substrate nucleic acids may comprise 1, 2, 3, 4, 5, 6, or 7 primers. In some cases, generating substrate nucleic acids may comprise 1, 2, 3, 4, 5, or 6 primers. In some cases, generating substrate nucleic acids may comprise 1, 2, 3, 4, or 5 primers. In some cases, generating substrate nucleic acids may comprise 1, 2, 3, or 4 primers. In some cases, generating substrate nucleic acids may comprise 1, 2, or 3 primers. In some cases, generating substrate nucleic acids may comprise 1, or 2 primers.

In some cases, generating substrate nucleic acids may comprise 1 primer. In some cases, generating substrate nucleic acids may comprise 2 primers. In some cases, generating substrate nucleic acids may comprise 3 primers. In some cases, generating substrate nucleic acids may comprise 4 primers. In some cases, generating substrate nucleic acids may comprise 5 primers. In some cases, generating substrate nucleic acids may comprise 6 primers. In some cases, generating substrate nucleic acids may comprise 7 primers. In some cases, generating substrate nucleic acids may comprise 8 primers. In some cases, generating substrate nucleic acids may comprise 9 primers. In some cases, generating substrate nucleic acids may comprise 10 primers. In some cases, generating substrate nucleic acids may comprise 11 primers. In some cases, generating substrate nucleic acids may comprise 12 primers. In some cases, generating substrate nucleic acids may comprise 13 primers. In some cases, generating substrate nucleic acids may comprise 14 primers. In some cases, generating substrate nucleic acids may comprise 15 primers. In some cases, generating substrate nucleic acids may comprise 16 primers. In some cases, generating substrate nucleic acids may comprise 17 primers. In some cases, generating substrate nucleic acids may comprise 18 primers. In some cases, generating substrate nucleic acids may comprise 19 primers. In some cases, generating substrate nucleic acids may comprise 20 primers. In some cases, generating substrate nucleic acids may comprise 21 primers. In some cases, generating substrate nucleic acids may comprise 22 primers. In some cases, generating substrate nucleic acids may comprise 23 primers. In some cases, generating substrate nucleic acids may comprise 24 primers. In some cases, generating substrate nucleic acids may comprise 25 primers. In some cases, generating substrate nucleic acids may comprise 26 primers. In some cases, generating substrate nucleic acids may comprise 27 primers. In some cases, generating substrate nucleic acids may comprise 28 primers. In some cases, generating substrate nucleic acids may comprise 29 primers. In some cases, generating substrate nucleic acids may comprise 30 primers. In some cases, generating substrate nucleic acids may comprise 31 primers. In some cases, generating substrate nucleic acids may comprise 32 primers. In some cases, generating substrate nucleic acids may comprise 33 primers. In some cases, generating substrate nucleic acids may comprise 34 primers. In some cases, generating substrate nucleic acids may comprise 35 primers. In some cases, generating substrate nucleic acids may comprise 36 primers. In some cases, generating substrate nucleic acids may comprise 37 primers. In some cases, generating substrate nucleic acids may comprise 38 primers. In some cases, generating substrate nucleic acids may comprise 39 primers. In some cases, generating substrate nucleic acids may comprise 40 primers. In some cases, generating substrate nucleic acids may comprise 41 primers. In some cases, generating substrate nucleic acids may comprise 42 primers. In some cases, generating substrate nucleic acids may comprise 43 primers. In some cases, generating substrate nucleic acids may comprise 44 primers. In some cases, generating substrate nucleic acids may comprise 45 primers. In some cases, generating substrate nucleic acids may comprise 46 primers. In some cases, generating substrate nucleic acids may comprise 47 primers. In some cases, generating substrate nucleic acids may comprise 48 primers. In some cases, generating substrate nucleic acids may comprise 49 primers. In some cases, generating substrate nucleic acids may comprise 50 primers. In some cases, generating substrate nucleic acids may comprise 51 primers, 52 primers, 53 primers, 54 primers, 55 primers, 56 primers, 57 primers, 58 primers, 59 primers, 60 primers, 61 primers, 62 primers, 63 primers, 64 primers, 65 primers, 66 primers, 67 primers, 68 primers, 69 primers, 70 primers, 71 primers, 72 primers, 73 primers, 74 primers, 75 primers, 76 primers, 77 primers, 78 primers, 79 primers, 80 primers, 81 primers, 82 primers, 83 primers, 84 primers, 85 primers, 86 primers, 87 primers, 88 primers, 89 primers, 90 primers, 91 primers, 92 primers, 93 primers, 94 primers, 95 primers, 96 primers, 97 primers, 98 primers, 99 primers, 100 primers, about 110 primers, about 120 primers, about 130 primers, about 140 primers, about 150 primers, about 160 primers, about 170 primers, about 180 primers, about 190 primers, about 200 primers, about 250 primers, about 300 primers, about 350 primers, about 400 primers, about 450 primers, about 500 primers, about 550 primers, about 600 primers, about 650 primers, about 700 primers, about 750 primers, about 800 primers, about 850 primers, about 900 primers, about 950 primers, or about 1000 primers.

In some cases, generating substrate nucleic acids may comprise from 1 to 50 primers. In some cases, generating substrate nucleic acids may comprise from 2 to 50 primers. In some cases, generating substrate nucleic acids may comprise from 3 to 50 primers. In some cases, generating substrate nucleic acids may comprise from 4 to 50 primers. In some cases, generating substrate nucleic acids may comprise from 5 to 50 primers. In some cases, generating substrate nucleic acids may comprise from 6 to 50 primers. In some cases, generating substrate nucleic acids may comprise from 7 to 50 primers. In some cases, generating substrate nucleic acids may comprise from 8 to 50 primers. In some cases, generating substrate nucleic acids may comprise from 9 to 50 primers. In some cases, generating substrate nucleic acids may comprise from 10 to 50 primers. In some cases, generating substrate nucleic acids may comprise from 11 to 50 primers. In some cases, generating substrate nucleic acids may comprise from 12 to 50 primers. In some cases, generating substrate nucleic acids may comprise from 1 to 45 primers. In some cases, generating substrate nucleic acids may comprise from 1 to 40 primers. In some cases, generating substrate nucleic acids may comprise from 1 to 35 primers. In some cases, generating substrate nucleic acids may comprise from 1 to 30 primers. In some cases, generating substrate nucleic acids may comprise from 1 to 25 primers. In some cases, generating substrate nucleic acids may comprise from 1 to 20 primers. In some cases, generating substrate nucleic acids may comprise from 1 to 19 primers. In some cases, generating substrate nucleic acids may comprise from 1 to 18 primers. In some cases, generating substrate nucleic acids may comprise from 1 to 17 primers. In some cases, generating substrate nucleic acids may comprise from 1 to 16 primers. In some cases, generating substrate nucleic acids may comprise from 1 to 15 primers. In some cases, generating substrate nucleic acids may comprise from 1 to 14 primers. In some cases, generating substrate nucleic acids may comprise from 1 to 13 primers. In some cases, generating substrate nucleic acids may comprise from 1 to 12 primers. In some cases, generating substrate nucleic acids may comprise from 1 to 11 primers. In some cases, generating substrate nucleic acids may comprise from 1 to 10 primers.

In some instances, a plurality of primers may comprise at least a nucleotide sequence comprising at least 85%, at least 86%, at least 90%, at least 94%, or 100% identity to a sequence selected from the group consisting of: SEQ ID NOs: SEQ ID NOs: 1-28, as provided in Table 1 below.

TABLE 1
Exemplary primer Sequences for virus detection

| | | | Gene target |
|---|---|---|---|
| | | | (genomic |
| | | Type of | coordinates of |
| SEQ ID NO | Sequence | primers | NC_045512.2) |
| SEQ ID NO: 1 | GTGATGAAGTCAGACAAATCGC | Forward | Spike (22773-23133) |
| SEQ ID NO: 2 | ACAGTTGCTGGTGCATGTAGA | Reverse | Spike (22773-23133) |
| SEQ ID NO: 3 | CCGGTAGCACACCTTGTAATG | Forward | Spike (22986-23301) |
| SEQ ID NO: 4 | CCTTGTAATGGTGTTGAAGGT | Forward | Spike (22997-23534) |
| SEQ ID NO: 5 | AAGCTATAACGCAGCCTGTAA | Reverse | Spike (22589-22868) |
| SEQ ID NO: 6 | TGACACTACTGATGCTGTCC | Forward | Spike (23272-23477) |
| SEQ ID NO: 7 | CTGTAGAATAAACACGCCAAG | Reverse | Spike (23272-23477) |
| SEQ ID NO: 8 | GCTGCATTCAGTTGAATCACC | Reverse | Spike (23559-23812) |
| SEQ ID NO: 9 | GACAAGGCGTTCCAATTAAC | Forward | N (28479-28651) |
| SEQ ID NO: 10 | GTTAGCACCATAGGGAAGTC | Reverse | N (28479-28651) |
| SEQ ID NO: 11 | CCGCATTACGTTTGGTGGAC | Forward | N (28312-28675) |
| SEQ ID NO: 12 | TGCAACCCATATGATGCCGT | Reverse | N (28312-28675) |
| SEQ ID NO: 13 | TGTCCTTCCCTCAGTCAGCA | Forward | Spike (24711-25285) |
| SEQ ID NO: 14 | AACGCCACCAGATTTGCATC | Forward | Spike (22589-22868) |
| SEQ ID NO: 15 | TGTTGACATGTTCAGCCCCT | Reverse | Spike (22997-23534) |
| SEQ ID NO: 16 | GTACCCGCTAACAGTGCAGA | Reverse | Spike (24129-24204) |
| SEQ ID NO: 17 | TTGGTGCAGGTATATGCGCT | Forward | Spike (23559-23812) |
| SEQ ID NO: 18 | ACGGCCTTACTGTTTTGCCA | Forward | Spike (24129-24204) |
| SEQ ID NO: 19 | TGTGGATCACGGACAGCATC | Reverse | Spike (22986-23301) |
| SEQ ID NO: 20 | GTCCGTGATCCACAGACACT | Forward | Spike (23288-23427) |
| SEQ ID NO: 21 | ACAGGGACTTCTGTGCAGTT | Reverse | Spike (23288-23427) |
| SEQ ID NO: 22 | GCCTCGGCAAAAACGTACTG | Forward | N (29044-29427) |
| SEQ ID NO: 23 | CTCTGCGGTAAGGCTTGAGT | Reverse | N (29044-29427) |
| SEQ ID NO: 24 | TGGTTGACCTACACAGGTGC | Forward | N (29261-29521) |
| SEQ ID NO: 25 | TGAGTCAGCACTGCTCATGG | Reverse | N (29261-29521) |
| SEQ ID NO: 26 | ACAGCAACTGGTCATACAGCA | Reverse | Spike (24711-25285) |
| SEQ ID NO: 27 | GATTCGAAGACCCAGTCCCTA | Forward | Spike (21893-22232) |
| SEQ ID NO: 28 | CTAAAGCCGAAAAACCCTGA | Reverse | Spike (21893-22232) |

In some instances, a plurality of primers may comprise at least a nucleotide sequence comprising at least 85% sequence identity to a sequence selected from the group consisting of: SEQ ID NOs: 1-28. In some cases, a plurality of primers may comprise at least a nucleotide sequence comprising at least 86% sequence identity to a sequence selected from the group consisting of: SEQ ID NOs: 1-28. In some cases, a plurality of primers may comprise at least a nucleotide sequence comprising at least 90% sequence identity to a sequence selected from the group consisting of: SEQ ID NOs: 1-28. In some cases, a plurality of primers may comprise at least a nucleotide sequence comprising at least 94% sequence identity to a sequence selected from the group consisting of: SEQ ID NOs: 1-28. In some cases, a plurality of primers may comprise at least a nucleotide sequence comprising a sequence selected from the group consisting of: SEQ ID NOs: 1-28. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 1-28.

In some instances, a primer may comprise at least a nucleotide sequence comprising at least 85% sequence identity to a sequence selected from the group consisting of: SEQ ID NOs: 1-28. In some cases, a primer may comprise at least a nucleotide sequence comprising at least 86% sequence identity to a sequence selected from the group consisting of: SEQ ID NOs: 1-28. In some cases, a primer may comprise at least a nucleotide sequence comprising at least 90% sequence identity to a sequence selected from the group consisting of: SEQ ID NOs: 1-28. In some cases, a primer may comprise at least a nucleotide sequence comprising at least 94% sequence identity to a sequence selected from the group consisting of: SEQ ID NOs: 1-28. In some cases, a primer may comprise at least a nucleotide sequence comprising a sequence selected from the group consisting of: SEQ ID NOs: 1-28. In some cases, a primer may comprise at least a nucleotide sequence comprising at least 50%, at least 55%, at least 60%, at least 65%, at least 70%, at least 75%, at least 80%, at least 81%, at least 82%, at least 83%, or at least 84% sequence identity to a sequence selected from the group consisting of: SEQ ID NOs: 1-28.

In some instances, a plurality of primers may comprise at least two nucleotide sequences comprising at least 85% sequence identity to two sequences selected from the group consisting of: SEQ ID NOs: 1-28. In some cases, a plurality of primers may comprise at least two nucleotide sequences comprising at least 86% sequence identity to two sequences selected from the group consisting of: SEQ ID NOs: 1-28. In some cases, a plurality of primers may comprise at least two nucleotide sequences comprising at least 90% sequence identity to two sequences selected from the group consisting of: SEQ ID NOs: 1-28. In some cases, a plurality of primers may comprise at least two nucleotide sequences comprising at least 94% sequence identity to two sequences selected from the group consisting of: SEQ ID NOs: 1-28. In some cases, a plurality of primers may comprise at least two nucleotide sequences comprising at least two sequences selected from the group consisting of: SEQ ID NOs: 1-28.

In some instances, a plurality of primers may comprise at least three nucleotide sequences comprising at least 85% sequence identity to three sequences selected from the group consisting of: SEQ ID NOs: 1-28. In some cases, a plurality of primers may comprise at least three nucleotide sequences comprising at least 86% sequence identity to three sequences selected from the group consisting of: SEQ ID NOs: 1-28. In some cases, a plurality of primers may comprise at least three nucleotide sequences comprising at least 90% sequence identity to three sequences selected from the group consisting of: SEQ ID NOs: 1-28. In some cases, a plurality of primers may comprise at least three nucleotide sequences comprising at least 94% sequence identity to three sequences selected from the group consisting of: SEQ ID NOs: 1-28. In some cases, a plurality of primers may comprise at least three nucleotide sequences comprising at least three sequences selected from the group consisting of: SEQ ID NOs: 1-28.

In some instances, a plurality of primers may comprise at least four nucleotide sequences comprising at least 85% sequence identity to four sequences selected from the group consisting of: SEQ ID NOs: 1-28. In some cases, a plurality of primers may comprise at least four nucleotide sequences comprising at least 86% sequence identity to four sequences selected from the group consisting of: SEQ ID NOs: 1-28. In some cases, a plurality of primers may comprise at least four nucleotide sequences comprising at least 90% sequence identity to four sequences selected from the group consisting of: SEQ ID NOs: 1-28. In some cases, a plurality of primers may comprise at least four nucleotide sequences comprising at least 94% sequence identity to four sequences selected from the group consisting of: SEQ ID NOs: 1-28. In some cases, a plurality of primers may comprise at least four nucleotide sequences comprising at least four sequences selected from the group consisting of: SEQ ID NOs: 1-28.

In some instances, a plurality of primers may comprise at least five nucleotide sequences comprising at least 85% sequence identity to five sequences selected from the group consisting of: SEQ ID NOs: 1-28. In some cases, a plurality of primers may comprise at least five nucleotide sequences comprising at least 86% sequence identity to five sequences selected from the group consisting of: SEQ ID NOs: 1-28. In some cases, a plurality of primers may comprise at least five nucleotide sequences comprising at least 90% sequence identity to five sequences selected from the group consisting of: SEQ ID NOs: 1-28. In some cases, a plurality of primers may comprise at least five nucleotide sequences comprising at least 94% sequence identity to five sequences selected from the group consisting of: SEQ ID NOs: 1-28. In some cases, a plurality of primers may comprise at least five nucleotide sequences comprising at least five sequences selected from the group consisting of: SEQ ID NOs: 1-28.

In some instances, a plurality of primers may comprise six nucleotide sequences comprising at least 85% sequence identity to six sequences selected from the group consisting of: SEQ ID NOs: 1-28. In some cases, a plurality of primers may comprise six nucleotide sequences comprising at least 86% sequence identity to six sequences selected from the group consisting of: SEQ ID NOs: 1-28. In some cases, a plurality of primers may comprise six nucleotide sequences comprising at least 90% sequence identity to six sequences selected from the group consisting of: SEQ ID NOs: 1-28. In some cases, a plurality of primers may comprise six nucleotide sequences comprising at least 94% sequence identity to six sequences selected from the group consisting of: SEQ ID NOs: 1-28. In some cases, a plurality of primers may comprise six nucleotide sequences comprising at least six sequences selected from the group consisting of: SEQ ID NOs: 1-28.

In some instances, a plurality of primers may comprise at least a nucleotide sequence comprising at least 85% sequence identity to a sequence selected from the group consisting of: SEQ ID NOs: 1-12, 14, 15, 17, and 19. In some cases, a plurality of primers may comprise at least a nucleotide sequence comprising at least 86% sequence identity to a sequence selected from the group consisting of: SEQ ID NOs: 1-12, 14, 15, 17, and 19. In some cases, a plurality of primers may comprise at least a nucleotide sequence comprising at least 90% sequence identity to a sequence selected from the group consisting of: SEQ ID NOs: 1-12, 14, 15, 17, and 19. In some cases, a plurality of primers may comprise at least a nucleotide sequence comprising at least 94% sequence identity to a sequence selected from the group consisting of: SEQ ID NOs: 1-12, 14, 15, 17, and 19. In some cases, a plurality of primers may comprise at least a nucleotide sequence comprising a sequence selected from the group consisting of: SEQ ID NOs: 1-12, 14, 15, 17, and 19. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 1-12, 14, 15, 17, and 19.

In some instances, a primer may comprise at least a nucleotide sequence comprising at least 85% sequence identity to a sequence selected from the group consisting of: SEQ ID NOs: 1-12, 14, 15, 17, and 19. In some cases, a primer may comprise at least a nucleotide sequence comprising at least 86% sequence identity to a sequence selected from the group consisting of: SEQ ID NOs: 1-12, 14, 15, 17, and 19. In some cases, a primer may comprise at least a nucleotide sequence comprising at least 90% sequence identity to a sequence selected from the group consisting of: SEQ ID NOs: 1-12, 14, 15, 17, and 19. In some cases, a primer may comprise at least a nucleotide sequence comprising at least 94% sequence identity to a sequence selected from the group consisting of: SEQ ID NOs: 1-12, 14, 15, 17, and 19. In some cases, a primer may comprise at least a nucleotide sequence comprising a sequence selected from the group consisting of: SEQ ID NOs: 1-12, 14, 15, 17, and 19. In some cases, a primer may comprise at least a nucleotide sequence comprising at least 50%, at least 55%, at least 60%, at least 65%, at least 70%, at least 75%, at least 80%, at least 81%, at least 82%, at least 83%, or at least 84% sequence identity to a sequence selected from the group consisting of: SEQ ID NOs: 1-12, 14, 15, 17, and 19.

In some instances, a plurality of primers may comprise at least two nucleotide sequences comprising at least 85% sequence identity to two sequences selected from the group consisting of: SEQ ID NOs: 1-12, 14, 15, 17, and 19. In some cases, a plurality of primers may comprise at least two nucleotide sequences comprising at least 86% sequence identity to two sequences selected from the group consisting of: SEQ ID NOs: 1-12, 14, 15, 17, and 19. In some cases, a plurality of primers may comprise at least two nucleotide sequences comprising at least 90% sequence identity to two sequences selected from the group consisting of: SEQ ID NOs: 1-12, 14, 15, 17, and 19. In some cases, a plurality of primers may comprise at least two nucleotide sequences comprising at least 94% sequence identity to two sequences selected from the group consisting of: SEQ ID NOs: 1-12, 14, 15, 17, and 19. In some cases, a plurality of primers may comprise at least two nucleotide sequences comprising at least two sequences selected from the group consisting of: SEQ ID NOs: 1-12, 14, 15, 17, and 19.

In some instances, a plurality of primers may comprise at least three nucleotide sequences comprising at least 85% sequence identity to three sequences selected from the group consisting of: SEQ ID NOs: 1-12, 14, 15, 17, and 19. In some cases, a plurality of primers may comprise at least three nucleotide sequences comprising at least 86% sequence identity to three sequences selected from the group consisting of: SEQ ID NOs: 1-12, 14, 15, 17, and 19. In some cases, a plurality of primers may comprise at least three nucleotide sequences comprising at least 90% sequence identity to three sequences selected from the group consisting of: SEQ ID NOs: 1-12, 14, 15, 17, and 19. In some cases, a plurality of primers may comprise at least three nucleotide sequences comprising at least 94% sequence identity to three sequences selected from the group consisting of: SEQ ID NOs: 1-12, 14, 15, 17, and 19. In some cases, a plurality of primers may comprise at least three nucleotide sequences comprising at least three sequences selected from the group consisting of: SEQ ID NOs: 1-12, 14, 15, 17, and 19.

In some instances, a plurality of primers may comprise at least four nucleotide sequences comprising at least 85% sequence identity to four sequences selected from the group consisting of: SEQ ID NOs: 1-12, 14, 15, 17, and 19. In some cases, a plurality of primers may comprise at least four nucleotide sequences comprising at least 86% sequence identity to four sequences selected from the group consisting of: SEQ ID NOs: 1-12, 14, 15, 17, and 19. In some cases, a plurality of primers may comprise at least four nucleotide sequences comprising at least 90% sequence identity to four sequences selected from the group consisting of: SEQ ID NOs: 1-12, 14, 15, 17, and 19. In some cases, a plurality of primers may comprise at least four nucleotide sequences comprising at least 94% sequence identity to four sequences selected from the group consisting of: SEQ ID NOs: 1-12, 14, 15, 17, and 19. In some cases, a plurality of primers may comprise at least four nucleotide sequences comprising at least four sequences selected from the group consisting of: SEQ ID NOs: 1-12, 14, 15, 17, and 19.

In some instances, a plurality of primers may comprise at least five nucleotide sequences comprising at least 85% sequence identity to five sequences selected from the group consisting of: SEQ ID NOs: 1-12, 14, 15, 17, and 19. In some cases, a plurality of primers may comprise at least five nucleotide sequences comprising at least 86% sequence identity to five sequences selected from the group consisting of: SEQ ID NOs: 1-12, 14, 15, 17, and 19. In some cases, a plurality of primers may comprise at least five nucleotide sequences comprising at least 90% sequence identity to five sequences selected from the group consisting of: SEQ ID NOs: 1-12, 14, 15, 17, and 19. In some cases, a plurality of primers may comprise at least five nucleotide sequences comprising at least 94% sequence identity to five sequences selected from the group consisting of: SEQ ID NOs: 1-12, 14, 15, 17, and 19. In some cases, a plurality of primers may comprise at least five nucleotide sequences comprising at least five sequences selected from the group consisting of: SEQ ID NOs: 1-12, 14, 15, 17, and 19.

In some instances, a plurality of primers may comprise six nucleotide sequences comprising at least 85% sequence identity to six sequences selected from the group consisting of: SEQ ID NOs: 1-12, 14, 15, 17, and 19. In some cases, a plurality of primers may comprise six nucleotide sequences comprising at least 86% sequence identity to six sequences selected from the group consisting of: SEQ ID NOs: 1-12, 14, 15, 17, and 19. In some cases, a plurality of primers may comprise six nucleotide sequences comprising at least 90% sequence identity to six sequences selected from the group consisting of: SEQ ID NOs: 1-12, 14, 15, 17, and 19. In some cases, a plurality of primers may comprise six nucleotide sequences comprising at least 94% sequence identity to six sequences selected from the group consisting of: SEQ ID NOs: 1-12, 14, 15, 17, and 19. In some cases, a plurality of primers may comprise six nucleotide sequences comprising at least six sequences selected from the group consisting of: SEQ ID NOs: 1-12, 14, 15, 17, and 19.

In some instances, a plurality of primers may comprise at least a nucleotide sequence comprising at least 85% sequence identity to a sequence selected from the group consisting of: SEQ ID NOs: 1-8 and 13-21, and 26. In some cases, a plurality of primers may comprise at least a nucleotide sequence comprising at least 86% sequence identity to a sequence selected from the group consisting of: SEQ ID NOs: 1-8 and 13-21, and 26. In some cases, a plurality of primers may comprise at least a nucleotide sequence comprising at least 90% sequence identity to a sequence selected from the group consisting of: SEQ ID NOs: 1-8 and 13-21, and 26. In some cases, a plurality of primers may comprise at least a nucleotide sequence comprising at least 94% sequence identity to a sequence selected from the group consisting of: SEQ ID NOs: 1-8 and 13-21, and 26. In some cases, a plurality of primers may comprise at least a nucleotide sequence comprising a sequence selected from the group consisting of: SEQ ID NOs: 1-8 and 13-21, and 26. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 1-8 and 13-21, and 26.

In some instances, a plurality of primers may comprise at least a nucleotide sequence comprising at least 85% sequence identity to a sequence selected from the group consisting of: SEQ ID NOs: 1-8 and 13-21, and 26-28. In some cases, a plurality of primers may comprise at least a nucleotide sequence comprising at least 86% sequence identity to a sequence selected from the group consisting of: SEQ ID NOs: 1-8 and 13-21, and 26-28. In some cases, a plurality of primers may comprise at least a nucleotide sequence comprising at least 90% sequence identity to a sequence selected from the group consisting of: SEQ ID NOs: 1-8 and 13-21, and 26-28. In some cases, a plurality of primers may comprise at least a nucleotide sequence comprising at least 94% sequence identity to a sequence selected from the group consisting of: SEQ ID NOs: 1-8 and 13-21, and 26-28.

In some cases, a plurality of primers may comprise at least a nucleotide sequence comprising a sequence selected from the group consisting of: SEQ ID NOs: 1-8 and 13-21, and 26-28. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 1-8 and 13-21, and 26-28.

In some instances, a plurality of primers may comprise at least a nucleotide sequence comprising at least 85% sequence identity to a sequence selected from the group consisting of: SEQ ID NOs: 1, 3, 4, 6, 13, 14, 17, 18, and 20. In some cases, a plurality of primers may comprise at least a nucleotide sequence comprising at least 86% sequence identity to a sequence selected from the group consisting of: SEQ ID NOs: 1, 3, 4, 6, 13, 14, 17, 18, and 20. In some cases, a plurality of primers may comprise at least a nucleotide sequence comprising at least 90% sequence identity to a sequence selected from the group consisting of: SEQ ID NOs: 1, 3, 4, 6, 13, 14, 17, 18, and 20. In some cases, a plurality of primers may comprise at least a nucleotide sequence comprising at least 94% sequence identity to a sequence selected from the group consisting of: SEQ ID NOs: 1, 3, 4, 6, 13, 14, 17, 18, and 20. In some cases, a plurality of primers may comprise at least a nucleotide sequence comprising a sequence selected from the group consisting of: SEQ ID NOs: 1, 3, 4, 6, 13, 14, 17, 18, and 20. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 1, 3, 4, 6, 13, 14, 17, 18, and 20.

In some instances, a plurality of primers may comprise at least a nucleotide sequence comprising at least 85% sequence identity to a sequence selected from the group consisting of: SEQ ID NOs: 1, 3, 4, 6, 13, 14, 17, 18, 20, and 27. In some cases, a plurality of primers may comprise at least a nucleotide sequence comprising at least 86% sequence identity to a sequence selected from the group consisting of: SEQ ID NOs: 1, 3, 4, 6, 13, 14, 17, 18, 20, and 27. In some cases, a plurality of primers may comprise at least a nucleotide sequence comprising at least 90% sequence identity to a sequence selected from the group consisting of: SEQ ID NOs: 1, 3, 4, 6, 13, 14, 17, 18, 20, and 27. In some cases, a plurality of primers may comprise at least a nucleotide sequence comprising at least 94% sequence identity to a sequence selected from the group consisting of: SEQ ID NOs: 1, 3, 4, 6, 13, 14, 17, 18, 20, and 27. In some cases, a plurality of primers may comprise at least a nucleotide sequence comprising a sequence selected from the group consisting of: SEQ ID NOs: 1, 3, 4, 6, 13, 14, 17, 18, 20, and 27. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 1, 3, 4, 6, 13, 14, 17, 18, 20, and 27.

In some instances, a plurality of primers may comprise at least a nucleotide sequence comprising at least 85% sequence identity to a sequence selected from the group consisting of: SEQ ID NOs: 2, 5, 7, 8, 15, 16, 19, 21, and 26. In some cases, a plurality of primers may comprise at least a nucleotide sequence comprising at least 86% sequence identity to a sequence selected from the group consisting of: SEQ ID NOs: 2, 5, 7, 8, 15, 16, 19, 21, and 26. In some cases, a plurality of primers may comprise at least a nucleotide sequence comprising at least 90% sequence identity to a sequence selected from the group consisting of: SEQ ID NOs: 2, 5, 7, 8, 15, 16, 19, 21, and 26. In some cases, a plurality of primers may comprise at least a nucleotide sequence comprising at least 94% sequence identity to a sequence selected from the group consisting of: SEQ ID NOs: 2, 5, 7, 8, 15, 16, 19, 21, and 26. In some cases, a plurality of primers may comprise at least a nucleotide sequence comprising a sequence selected from the group consisting of: SEQ ID NOs: 2, 5, 7, 8, 15, 16, 19, 21, and 26. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 2, 5, 7, 8, 15, 16, 19, 21, and 26.

In some instances, a plurality of primers may comprise at least a nucleotide sequence comprising at least 85% sequence identity to a sequence selected from the group consisting of: SEQ ID NOs: 2, 5, 7, 8, 15, 16, 19, 21, 26 and 28. In some cases, a plurality of primers may comprise at least a nucleotide sequence comprising at least 86% sequence identity to a sequence selected from the group consisting of: SEQ ID NOs: 2, 5, 7, 8, 15, 16, 19, 21, 26 and 28. In some cases, a plurality of primers may comprise at least a nucleotide sequence comprising at least 90% sequence identity to a sequence selected from the group consisting of: SEQ ID NOs: 2, 5, 7, 8, 15, 16, 19, 21, 26 and 28. In some cases, a plurality of primers may comprise at least a nucleotide sequence comprising at least 94% sequence identity to a sequence selected from the group consisting of: SEQ ID NOs: 2, 5, 7, 8, 15, 16, 19, 21, 26 and 28. In some cases, a plurality of primers may comprise at least a nucleotide sequence comprising a sequence selected from the group consisting of: SEQ ID NOs: 2, 5, 7, 8, 15, 16, 19, 21, 26 and 28. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 2, 5, 7, 8, 15, 16, 19, 21, 26 and 28.

In some instances, a plurality of primers may comprise at least a nucleotide sequence comprising at least 85% sequence identity to a sequence selected from the group consisting of: SEQ ID NOs: 1, 3, 4, 6, 13, 14, 17, 18, and 20; and at least a nucleotide sequence comprising at least 85% sequence identity a sequence selected from the group consisting of SEQ ID NOs: 2, 5, 7, 8, 15, 16, 19, 21, and 26. In some cases, a plurality of primers may comprise at least a nucleotide sequence comprising at least 86% sequence identity to a sequence selected from the group consisting of: SEQ ID NOs: 1, 3, 4, 6, 13, 14, 17, 18, and 20; and at least a nucleotide sequence comprising at least 86% sequence identity a sequence selected from the group consisting of SEQ ID NOs: 2, 5, 7, 8, 15, 16, 19, 21, and 26. In some cases, a plurality of primers may comprise at least a nucleotide sequence comprising at least 90% sequence identity to a sequence selected from the group consisting of: SEQ ID NOs: 1, 3, 4, 6, 13, 14, 17, 18, and 20; and at least a nucleotide sequence comprising at least 90% sequence identity a sequence selected from the group consisting of SEQ ID NOs: 2, 5, 7, 8, 15, 16, 19, 21, and 26. In some cases, a plurality of primers may comprise at least a nucleotide sequence comprising at least 94% sequence identity to a sequence selected from the group consisting of: SEQ ID NOs: 1, 3, 4, 6, 13, 14, 17, 18, and 20; and at least a nucleotide sequence comprising at least 94% sequence identity a sequence selected from the group consisting of SEQ ID NOs: 2, 5, 7, 8, 15, 16, 19, 21, and 26. In some cases, a plurality of primers may comprise at least a nucleotide sequence comprising a sequence selected from the group consisting of: SEQ ID NOs: 1, 3, 4, 6, 13, 14, 17, 18, and 20; and at least a nucleotide sequence comprising a sequence selected from the group consisting of SEQ ID NOs: 2, 5, 7, 8, 15, 16, 19, 21, and 26. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 1, 3, 4, 6, 13, 14, 17, 18, and 20; and at least a nucleotide sequence comprising at least 50%, at least 55%, at least 60%, at least 65%, at least 70%, at least 75%, or at least 80% sequence identity to a sequence selected from the group consisting of SEQ ID NOs: 2, 5, 7, 8, 15, 16, 19, 21, and 26.

In some instances, a plurality of primers may comprise at least a nucleotide sequence comprising at least 85% sequence identity to a sequence selected from the group consisting of: SEQ ID NOs: 1, 3, 4, 6, 13, 14, 17, 18, 20, and 27; and at least a nucleotide sequence comprising at least 85% sequence identity a sequence selected from the group consisting of SEQ ID NOs: 2, 5, 7, 8, 15, 16, 19, 21, 26, and 28. In some cases, a plurality of primers may comprise at least a nucleotide sequence comprising at least 86% sequence identity to a sequence selected from the group consisting of: SEQ ID NOs: 1, 3, 4, 6, 13, 14, 17, 18, 20, and 27; and at least a nucleotide sequence comprising at least 86% sequence identity a sequence selected from the group consisting of SEQ ID NOs: 2, 5, 7, 8, 15, 16, 19, 21, 26, and 28. In some cases, a plurality of primers may comprise at least a nucleotide sequence comprising at least 90% sequence identity to a sequence selected from the group consisting of: SEQ ID NOs: 1, 3, 4, 6, 13, 14, 17, 18, 20, and 27; and at least a nucleotide sequence comprising at least 90% sequence identity a sequence selected from the group consisting of SEQ ID NOs: 2, 5, 7, 8, 15, 16, 19, 21, 26, and 28. In some cases, a plurality of primers may comprise at least a nucleotide sequence comprising at least 94% sequence identity to a sequence selected from the group consisting of: SEQ ID NOs: 1, 3, 4, 6, 13, 14, 17, 18, 20, and 27; and at least a nucleotide sequence comprising at least 94% sequence identity a sequence selected from the group consisting of SEQ ID NOs: 2, 5, 7, 8, 15, 16, 19, 21, 26, and 28. In some cases, a plurality of primers may comprise at least a nucleotide sequence comprising a sequence selected from the group consisting of: SEQ ID NOs: 1, 3, 4, 6, 13, 14, 17, 18, 20, and 27; and at least a nucleotide sequence comprising a sequence selected from the group consisting of SEQ ID NOs: 2, 5, 7, 8, 15, 16, 19, 21, 26, and 28. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 1, 3, 4, 6, 13, 14, 17, 18, 20, and 27; and at least a nucleotide sequence comprising at least 50%, at least 55%, at least 60%, at least 65%, at least 70%, at least 75%, or at least 80% sequence identity to a sequence selected from the group consisting of SEQ ID NOs: 2, 5, 7, 8, 15, 16, 19, 21, 26, and 28.

In some instances, a plurality of primers may comprise at least a nucleotide sequence comprising at least 85% sequence identity to a sequence selected from the group consisting of: SEQ ID NOs: 9-12 and 22-25. In some cases, a plurality of primers may comprise at least a nucleotide sequence comprising at least 86% sequence identity to a sequence selected from the group consisting of: SEQ ID NOs: 9-12 and 22-25. In some cases, a plurality of primers may comprise at least a nucleotide sequence comprising at least 90% sequence identity to a sequence selected from the group consisting of: SEQ ID NOs: 9-12 and 22-25. In some cases, a plurality of primers may comprise at least a nucleotide sequence comprising at least 94% sequence identity to a sequence selected from the group consisting of: SEQ ID NOs: 9-12 and 22-25. In some cases, a plurality of primers may comprise at least a nucleotide sequence comprising a sequence selected from the group consisting of: SEQ ID NOs: 9-12 and 22-25. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 9-12 and 22-25.

In some instances, a plurality of primers may comprise at least a nucleotide sequence comprising at least 85% sequence identity to a sequence selected from the group consisting of: SEQ ID NOs: 9, 11, 22, and 24. In some cases, a plurality of primers may comprise at least a nucleotide sequence comprising at least 86% sequence identity to a sequence selected from the group consisting of: SEQ ID NOs: 9, 11, 22, and 24. In some cases, a plurality of primers may comprise at least a nucleotide sequence comprising at least 90% sequence identity to a sequence selected from the group consisting of: SEQ ID NOs: 9, 11, 22, and 24. In some cases, a plurality of primers may comprise at least a nucleotide sequence comprising at least 94% sequence identity to a sequence selected from the group consisting of: SEQ ID NOs: 9, 11, 22, and 24. In some cases, a plurality of primers may comprise at least a nucleotide sequence comprising a sequence selected from the group consisting of: SEQ ID NOs: 9, 11, 22, and 24. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 9, 11, 22, and 24.

In some instances, a plurality of primers may comprise at least a nucleotide sequence comprising at least 85% sequence identity to a sequence selected from the group consisting of: SEQ ID NOs: 10, 12, 23, and 25. In some cases, a plurality of primers may comprise at least a nucleotide sequence comprising at least 86% sequence identity to a sequence selected from the group consisting of: SEQ ID NOs: 10, 12, 23, and 25. In some cases, a plurality of primers may comprise at least a nucleotide sequence comprising at least 90% sequence identity to a sequence selected from the group consisting of: SEQ ID NOs: 10, 12, 23, and 25. In some cases, a plurality of primers may comprise at least a nucleotide sequence comprising at least 94% sequence identity to a sequence selected from the group consisting of: SEQ ID NOs: 10, 12, 23, and 25. In some cases, a plurality of primers may comprise at least a nucleotide sequence comprising a sequence selected from the group consisting of: SEQ ID NOs: 10, 12, 23, and 25. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 10, 12, 23, and 25.

In some instances, a plurality of primers may comprise at least a nucleotide sequence comprising at least 85% sequence identity to a sequence selected from the group consisting of: SEQ ID NOs: 9, 11, 22, and 24; and at least a nucleotide sequence comprising at least 85% sequence identity to a sequence selected from the group consisting of SEQ ID NOs: 10, 12, 23, and 25. In some cases, a plurality of primers may comprise at least a nucleotide sequence comprising at least 86% sequence identity to a sequence selected from the group consisting of: SEQ ID NOs: 9, 11, 22, and 24; and at least a nucleotide sequence comprising at least 86% sequence identity to a sequence selected from the group consisting of SEQ ID NOs: 10, 12, 23, and 25. In some cases, a plurality of primers may comprise at least a nucleotide sequence comprising at least 90% sequence identity to a sequence selected from the group consisting of: SEQ ID NOs: 9, 11, 22, and 24; and at least a nucleotide sequence comprising at least 90% sequence identity to a sequence selected from the group consisting of SEQ ID NOs: 10, 12, 23, and 25. In some cases, a plurality of primers may comprise at least a nucleotide sequence comprising at least 94% sequence identity to a sequence selected from the group consisting of: SEQ ID NOs: 9, 11, 22, and 24; and at least a nucleotide sequence comprising at least 94% sequence identity to a sequence selected from the group consisting of SEQ ID NOs: 10, 12, 23, and 25. In some cases, a plurality of primers may comprise at least a nucleotide sequence comprising a sequence selected from the group consisting of: SEQ ID NOs: 9, 11, 22, and 24; and at least a nucleotide sequence comprising a sequence selected from the group consisting of SEQ ID NOs: 10, 12, 23, and 25. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: 9, 11, 22, and 24; and at least a nucleotide sequence comprising at least 50%, at least 55%, at least 60%, at least 65%, at least 70%, at least 75%, or at least 80% sequence identity to a sequence selected from the group consisting of SEQ ID NOs: 10, 12, 23, and 25.

In some cases, a plurality of primers may comprise at least a nucleotide sequence comprising at least 85%, 86%, 90%, or 94% sequence identity to a sequence selected from the group consisting of: SEQ ID NOs: 1, 3, 4, 6, 13, 14, 17, 18, and 20; at least a nucleotide sequence comprising at least 85%, 86%, 90%, or 94% sequence identity to a sequence selected from the group consisting of: SEQ ID NOs: 2, 5, 7, 8, 15, 16, 19, 21, and 26; at least a nucleotide sequence comprising at least 85%, 86%, 90%, or 94% sequence identity to a sequence selected from the group consisting of: SEQ ID NOs: 9, 11, 22, and 24; and at least a nucleotide sequence comprising at least 85%, 86%, 90%, or 94% sequence identity to a sequence selected from the group consisting of: SEQ ID NOs: 10, 12, 23, and 25. In some cases, a plurality of primers may comprise at least a nucleotide sequence comprising at least 85%, 86%, 90%, or 94% sequence identity to a sequence selected from the group consisting of: SEQ ID NOs: 1, 3, 4, 6, 13, 14, 17, 18, and 20; at least a nucleotide sequence comprising at least 85%, 86%, 90%, or 94% sequence identity to a sequence selected from the group consisting of: SEQ ID NOs: 2, 5, 7, 8, 15, 16, 19, 21, and 26; at least a nucleotide sequence comprising at least 85%, 86%, 90%, or 94% sequence identity to a sequence selected from the group consisting of: SEQ ID NOs: 9, 11, 22, and 24; and at least a nucleotide sequence comprising at least 85%, 86%, 90%, or 94% sequence identity to a sequence selected from the group consisting of: SEQ ID NOs: 10, 12, 23, and 25. In some cases, a plurality of primers may comprise at least a nucleotide sequence comprising a sequence selected from the group consisting of: SEQ ID NOs: 1, 3, 4, 6, 13, 14, 17, 18, and 20; at least a nucleotide sequence comprising a sequence selected from the group consisting of: SEQ ID NOs: 2, 5, 7, 8, 15, 16, 19, 21, and 26; at least a nucleotide sequence comprising a sequence selected from the group consisting of: SEQ ID NOs: 9, 11, 22, and 24; and at least a nucleotide sequence comprising a sequence selected from the group consisting of: SEQ ID NOs: 10, 12, 23, and 25. In some cases, a plurality of primers may comprise at least a nucleotide sequence comprising a sequence selected from the group consisting of: SEQ ID NOs: 1, 3, 4, 6, 13, 14, 17, 18, and 20; at least a nucleotide sequence comprising a sequence selected from the group consisting of: SEQ ID NOs: 2, 5, 7, 8, 15, 16, 19, 21, and 26; at least a nucleotide sequence comprising a sequence selected from the group consisting of: SEQ ID NOs: 9, 11, 22, and 24; and at least a nucleotide sequence comprising a sequence selected from the group consisting of: SEQ ID NOs: 10, 12, 23, and 25.

In some cases, a plurality of primers may comprise at least a nucleotide sequence comprising at least 85%, 86%, 90%, or 94% sequence identity to a sequence selected from the group consisting of: SEQ ID NOs: 1, 3, 4, 6, 13, 14, 17, 18, 20, and 27; at least a nucleotide sequence comprising at least 85%, 86%, 90%, or 94% sequence identity to a sequence selected from the group consisting of: SEQ ID NOs: 2, 5, 7, 8, 15, 16, 19, 21, 26, and 28; at least a nucleotide sequence comprising at least 85%, 86%, 90%, or 94% sequence identity to a sequence selected from the group consisting of: SEQ ID NOs: 9, 11, 22, and 24; and at least a nucleotide sequence comprising at least 85%, 86%, 90%, or 94% sequence identity to a sequence selected from the group consisting of: SEQ ID NOs: 10, 12, 23, and 25. In some cases, a plurality of primers may comprise at least a nucleotide sequence comprising at least 85%, 86%, 90%, or 94% sequence identity to a sequence selected from the group consisting of: SEQ ID NOs: 1, 3, 4, 6, 13, 14, 17, 18, 20, and 27; at least a nucleotide sequence comprising at least 85%, 86%, 90%, or 94% sequence identity to a sequence selected from the group consisting of: SEQ ID NOs: 2, 5, 7, 8, 15, 16, 19, 21, 26, and 28; at least a nucleotide sequence comprising at least 85%, 86%, 90%, or 94% sequence identity to a sequence selected from the group consisting of: SEQ ID NOs: 9, 11, 22, and 24; and at least a nucleotide sequence comprising at least 85%, 86%, 90%, or 94% sequence identity to a sequence selected from the group consisting of: SEQ ID NOs: 10, 12, 23, and 25. In some cases, a plurality of primers may comprise at least a nucleotide sequence comprising a sequence selected from the group consisting of: SEQ ID NOs: 1, 3, 4, 6, 13, 14, 17, 18, 20, and 27; at least a nucleotide sequence comprising a sequence selected from the group consisting of: SEQ ID NOs: 2, 5, 7, 8, 15, 16, 19, 21, 26, and 28; at least a nucleotide sequence comprising a sequence selected from the group consisting of: SEQ ID NOs: 9, 11, 22, and 24; and at least a nucleotide sequence comprising a sequence selected from the group consisting of: SEQ ID NOs: 10, 12, 23, and 25. In some cases, a plurality of primers may comprise at least a nucleotide sequence comprising a sequence selected from the group consisting of: SEQ ID NOs: 1, 3, 4, 6, 13, 14, 17, 18, 20, and 27; at least a nucleotide sequence comprising a sequence selected from the group consisting of: SEQ ID NOs: 2, 5, 7, 8, 15, 16, 19, 21, 26, and 28; at least a nucleotide sequence comprising a sequence selected from the group consisting of: SEQ ID NOs: 9, 11, 22, and 24; and at least a nucleotide sequence comprising a sequence selected from the group consisting of: SEQ ID NOs: 10, 12, 23, and 25.

In some instances, a plurality of primers may comprise at least a nucleotide sequence comprising at least 85% sequence identity to a sequence selected from the group consisting of: SEQ ID NOs: a) SEQ ID NOs: 1 and 2, b) SEQ ID NOs: 3 and 19, c) SEQ ID NOs: 4 and 15, d) SEQ ID NOs: 5 and 14, e) SEQ ID NOs: 6 and 7, f) SEQ ID NOs: 13 and 26, g) SEQ ID NOs: 16 and 18, h) SEQ ID NOs: 20 and 21, and i) SEQ ID NOs: 27 and 28. In some cases, a plurality of primers may comprise at least a nucleotide sequence comprising at least 86% sequence identity to a sequence selected from the group consisting of: SEQ ID NOs: a) SEQ ID NOs: 1 and 2, b) SEQ ID NOs: 3 and 19, c) SEQ ID NOs: 4 and 15, d) SEQ ID NOs: 5 and 14, e) SEQ ID NOs: 6 and 7, f) SEQ ID NOs: 13 and 26, g) SEQ ID NOs: 16 and 18, h) SEQ ID NOs: 20 and 21, and i) SEQ ID NOs: 27 and 28. In some cases, a plurality of primers may comprise at least a nucleotide sequence comprising at least 90% sequence identity to a sequence selected from the group consisting of: SEQ ID NOs: a) SEQ ID NOs: 1 and 2, b) SEQ ID NOs: 3 and 19, c) SEQ ID NOs: 4 and 15, d) SEQ ID NOs: 5 and 14, e) SEQ ID NOs: 6 and 7, f) SEQ ID NOs: 13 and 26, g) SEQ ID NOs: 16 and 18, h) SEQ ID NOs: 20 and 21, and i) SEQ ID NOs: 27 and 28. In some cases, a plurality of primers may comprise at least a nucleotide sequence comprising at least 94% sequence identity to a sequence selected from the group consisting of: SEQ ID NOs: a) SEQ ID NOs: 1 and 2, b) SEQ ID NOs: 3 and 19, c) SEQ ID NOs: 4 and 15, d) SEQ ID NOs: 5 and 14, e) SEQ ID NOs: 6 and 7, f) SEQ ID NOs: 13 and 26, g) SEQ ID NOs: 16 and 18, h) SEQ ID NOs: 20 and 21, and i) SEQ ID NOs: 27 and 28. In some cases, a plurality of primers may comprise at least a nucleotide sequence comprising a sequence selected from the group consisting of: SEQ ID NOs: a) SEQ ID NOs: 1 and 2, b) SEQ ID NOs: 3 and 19, c) SEQ ID NOs: 4 and 15, d) SEQ ID NOs: 5 and 14, e) SEQ ID NOs: 6 and 7, f) SEQ ID NOs: 13 and 26, g) SEQ ID NOs: 16 and 18, h) SEQ ID NOs: 20 and 21, and i) SEQ ID NOs: 27 and 28. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: a) SEQ ID NOs: 1 and 2, b) SEQ ID NOs: 3 and 19, c) SEQ ID NOs: 4 and 15, d) SEQ ID NOs: 5 and 14, e) SEQ ID NOs: 6 and 7, f) SEQ ID NOs: 13 and 26, g) SEQ ID NOs: 16 and 18, h) SEQ ID NOs: 20 and 21, and i) SEQ ID NOs: 27 and 28.

In some instances, a plurality of primers may comprise at least a nucleotide sequence comprising at least 85% sequence identity to a sequence selected from the group consisting of: SEQ ID NOs: a) SEQ ID NOs: 9 and 10, b) SEQ ID NOs: 11 and 12, c) SEQ ID NOs: 22 and 23, and d) SEQ ID NOs: 24 and 25. In some cases, a plurality of primers may comprise at least a nucleotide sequence comprising at least 86% sequence identity to a sequence selected from the group consisting of: SEQ ID NOs: a) SEQ ID NOs: 9 and 10, b) SEQ ID NOs: 11 and 12, c) SEQ ID NOs: 22 and 23, and d) SEQ ID NOs: 24 and 25. In some cases, a plurality of primers may comprise at least a nucleotide sequence comprising at least 90% sequence identity to a sequence selected from the group consisting of: SEQ ID NOs: a) SEQ ID NOs: 9 and 10, b) SEQ ID NOs: 11 and 12, c) SEQ ID NOs: 22 and 23, and d) SEQ ID NOs: 24 and 25. In some cases, a plurality of primers may comprise at least a nucleotide sequence comprising at least 94% sequence identity to a sequence selected from the group consisting of: SEQ ID NOs: a) SEQ ID NOs: 9 and 10, b) SEQ ID NOs: 11 and 12, c) SEQ ID NOs: 22 and 23, and d) SEQ ID NOs: 24 and 25. In some cases, a plurality of primers may comprise at least a nucleotide sequence comprising a sequence selected from the group consisting of: SEQ ID NOs: a) SEQ ID NOs: 9 and 10, b) SEQ ID NOs: 11 and 12, c) SEQ ID NOs: 22 and 23, and d) SEQ ID NOs: 24 and 25. In some cases, a plurality of primers may comprise at least a nucleotide sequence comprising at least 50%, at least 55%, at least 60%, at least 65%, at least 70%, at least 75%, or at least 80% sequence identity to a sequence selected from the group consisting of: SEQ ID NOs: a) SEQ ID NOs: 9 and 10, b) SEQ ID NOs: 11 and 12, c) SEQ ID NOs: 22 and 23, and d) SEQ ID NOs: 24 and 25.

In some instances, a primer may comprise about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, or about 35 nucleotides in length. In some cases, a primer may comprise about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, or about 30 nucleotides in length. In some cases, a primer may comprise about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, or about 30 nucleotides in length.

In some instances, a primer may comprise about 10 nucleotides in length. In some cases, a primer may comprise about 11 nucleotides in length. In some cases, a primer may comprise about 12 nucleotides in length. In some cases, a primer may comprise about 13 nucleotides in length. In some cases, a primer may comprise about 14 nucleotides in length. In some cases, a primer may comprise about 15 nucleotides in length. In some cases, a primer may comprise about 16 nucleotides in length. In some cases, a primer may comprise about 17 nucleotides in length. In some cases, a primer may comprise about 18 nucleotides in length. In some cases, a primer may comprise about 19 nucleotides in length. In some cases, a primer may comprise about 20 nucleotides in length. In some cases, a primer may comprise about 21 nucleotides in length. In some cases, a primer may comprise about 22 nucleotides in length. In some cases, a primer may comprise about 23 nucleotides in length. In some cases, a primer may comprise about 24 nucleotides in length. In some cases, a primer may comprise about 25 nucleotides in length. In some cases, a primer may comprise about 26 nucleotides in length. In some cases, a primer may comprise about 27 nucleotides in length. In some cases, a primer may comprise about 28 nucleotides in length. In some cases, a primer may comprise about 29 nucleotides in length. In some cases, a primer may comprise about 30 nucleotides in length. In some cases, a primer may comprise about 31 nucleotides in length. In some cases, a primer may comprise about 32 nucleotides in length. In some cases, a primer may comprise about 33 nucleotides in length. In some cases, a primer may comprise about 34 nucleotides in length. In some cases, a primer may comprise about 35 nucleotides in length. In some cases, a primer may comprise about 36 nucleotides in length. In some cases, a primer may comprise about 37 nucleotides in length. In some cases, a primer may comprise about 38 nucleotides in length. In some cases, a primer may comprise about 39 nucleotides in length. In some cases, a primer may comprise about 40 nucleotides in length, about 41 nucleotides in length, about 42 nucleotides in length, about 43 nucleotides in length, about 44 nucleotides in length, about 45 nucleotides in length, about 46 nucleotides in length, about 47 nucleotides in length, about 48 nucleotides in length, or about 49 nucleotides in length. In some cases, a primer may comprise about 50 nucleotides in length. In some cases, a primer may comprise about 55 nucleotides in length, about 60 nucleotides in length, about 65 nucleotides in length, about 70 nucleotides in length, about 75 nucleotides in length, about 80 nucleotides in length, about 85 nucleotides in length, about 90 nucleotides in length, about 95 nucleotides in length, or about 100 nucleotides in length. In some cases, a primer may comprise from about 10 nucleotides in length to about 15 nucleotides in length. In some cases, a primer may comprise from about 10 nucleotides in length to about 20 nucleotides in length. In some cases, a primer may comprise from about 10 nucleotides in length to about 25 nucleotides in length. In some cases, a primer may comprise from about 10 nucleotides in length to about 30 nucleotides in length. In some cases, a primer may comprise from about 10 nucleotides in length to about 35 nucleotides in length. In some cases, a primer may comprise from about 10 nucleotides in length to about 40 nucleotides in length. In some cases, a primer may comprise from about 10 nucleotides in length to about 45 nucleotides in length. In some cases, a primer may comprise from about 10 nucleotides in length to about 50 nucleotides in length, from about 10 nucleotides in length to about 55 nucleotides in length, from about 10 nucleotides in length to about 60 nucleotides in length, from about 10 nucleotides in length to about 65 nucleotides in length, from about 10 nucleotides in length to about 70 nucleotides in length, from about 10 nucleotides in length to about 75 nucleotides in length, from about 10 nucleotides in length to about 80 nucleotides in length, from about 10 nucleotides in length to about 85 nucleotides in length, from about 10 nucleotides in length to about 90 nucleotides in length, from about 10 nucleotides in length to about 95 nucleotides in length, or from about 10 nucleotides in length to about 100 nucleotides in length.

In some cases, a primer may have a guanine-cytosine content (GC content). The GC content may comprise the percentage of nitrogenous bases in a nucleic acid molecule (e.g., the primer) that are either guanine or cytosine. The GC content of a primer may be at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% or more. The GC content of a primer may be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%. The GC content of a primer may be about 20-40%. The GC content of a primer may be about 21-41%. The GC content of a primer may be about 22-42%. The GC content of a primer may be about 23-43%. The GC content of a primer may be about 24-44%. The GC content of a primer may be about 25-45%. The GC content of a primer may be about 26-46%. The GC content of a primer may be about 27-47%. The GC content of a primer may be about 28-48%. The GC content of a primer may be about 29-49%. The GC content of a primer may be about 30-50%. The GC content of a primer may be about 31-51%. The GC content of a primer may be about 32-52%. The GC content of a primer may be about 33-53%. The GC content of a primer may be about 34-54%. The GC content of a primer may be about 35-55%. The GC content of a primer may be about 36-56%. The GC content of a primer may be about 37-57%. The GC content of a primer may be about 38-58%. The GC content of a primer may be about 39-59%. The GC content of a primer may be about 40-60%. The GC content of a primer may be about 41-61%. The GC content of a primer may be about 42-62%. The GC content of a primer may be about 43-63%. The GC content of a primer may be about 44-64%. The GC content of a primer may be about 45-65%. The GC content of a primer may be about 46-66%. The GC content of a primer may be about 47-67%. The GC content of a primer may be about 48-68%. The GC content of a primer may be about 49-69%. The GC content of a primer may be about 50-70%. The GC content of a primer may be about 51-71%. The GC content of a primer may be about 52-72%. The GC content of a primer may be about 53-73%. The GC content of a primer may be about 54-74%. The GC content of a primer may be about 55-75%. The GC content of a primer may be about 56-76%. The GC content of a primer may be about 57-77%. The GC content of a primer may be about 58-78%. The GC content of a primer may be about 59-79%. The GC content of a primer may be about 60-80%. The GC content of a primer may be about 61-81%. The GC content of a primer may be about 62-82%. The GC content of a primer may be about 63-83%. The GC content of a primer may be about 64-84%. The GC content of a primer may be about 65-85%. The GC content of a primer may be about 66-86%. The GC content of a primer may be about 67-87%. The GC content of a primer may be about 68-88%. The GC content of a primer may be about 69-89%. The GC content of a primer may be about 70-90%.

In some instances, a primer may have a melting temperature (Tₘ). The Tₘ of a primer may be the temperature at which 50% of the primer molecules become single-stranded. The Tₘ of a primer may be the temperature at which 50% of the primer molecules not forming nucleic acid duplex (e.g., a DNA duplex of the primer and its annealed DNA target molecule comprising the target sequences). The Tₘ of a primer may be at least about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C. or more. The Tₘ of a primer may be at most about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C. The Tₘ of a primer may be about 20-40° C. The Tₘ of a primer may be about 21-46° C. The Tₘ of a primer may be about 22-47° C. The Tₘ of a primer may be about 23-48° C. The Tₘ of a primer may be about 24-49° C. The Tₘ of a primer may be about 25-50° C. The Tₘ of a primer may be about 26-51° C. The Tₘ of a primer may be about 27-52° C. The Tₘ of a primer may be about 28-53° C. The Tₘ of a primer may be about 29-54° C. The Tₘ of a primer may be about 30-55° C. The Tₘ of a primer may be about 31-56° C. The Tₘ of a primer may be about 32-57° C. The Tₘ of a primer may be about 33-58° C. The Tₘ of a primer may be about 34-59° C. The Tₘ of a primer may be about 35-60° C. The Tₘ of a primer may be about 36-61° C. The Tₘ of a primer may be about 37-62° C. The Tₘ of a primer may be about 38-63° C. The Tₘ of a primer may be about 39-64° C. The Tₘ of a primer may be about 40-65° C. The Tₘ of a primer may be about 41-66° C. The Tₘ of a primer may be about 42-67° C. The Tₘ of a primer may be about 43-68° C. The Tₘ of a primer may be about 44-69° C. The Tₘ of a primer may be about 45-70° C. The Tₘ of a primer may be about 46-71° C. The Tₘ of a primer may be about 47-72° C. The Tₘ of a primer may be about 48-73° C. The Tₘ of a primer may be about 49-74° C. The Tₘ of a primer may be about 50-75° C. The Tₘ of a primer may be about 51-76° C. The Tₘ of a primer may be about 52-77° C. The Tₘ of a primer may be about 53-78° C. The Tₘ of a primer may be about 54-79° C. The Tₘ of a primer may be about 55-80° C. The Tₘ of a primer may be about 56-81° C. The Tₘ of a primer may be about 57-82° C. The Tₘ of a primer may be about 58-83° C. The Tₘ of a primer may be about 59-84° C. The Tₘ of a primer may be about 60-85° C. The Tₘ of a primer may be about 61-86° C. The Tₘ of a primer may be about 62-87° C. The Tₘ of a primer may be about 63-88° C. The Tₘ of a primer may be about 64-89° C. The Tₘ of a primer may be about 65-90° C. The Tₘ of a primer may be about 66-91° C. The Tₘ of a primer may be about 67-92° C. The Tₘ of a primer may be about 68-93° C. The Tₘ of a primer may be about 69-94° C. The Tₘ of a primer may be about 70-95° C.

In some instances, a pair of primer may comprise a first primer comprising a nucleotide sequence complementary to a first strand of a double-stranded target nucleotide sequence and a second primer comprising a nucleotide sequence complementary to a second strand of the double-stranded target nucleotide sequence, wherein the first strand of the double-stranded target nucleotide sequence and the second strand of the double-stranded target nucleotide sequence are complementary to each other. In some cases, the first strand of the double-stranded target nucleotide sequence and the second strand of the double-stranded target nucleotide sequence may form a duplex.

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of a reference genome of a virus. A reference genome of a virus may comprise a wild type strain or variant strain of a virus. A variant strain of a virus may be novel (e.g., not reported, identified, determined, or known before). Such a variant strain of a virus may not be known to circulate/present worldwide, in a certain geographic region, and/or at a certain temporal period. Such a variant strain of a virus may not be predicted or suspected to circulate/present worldwide, in a certain geographic region, and/or at a certain temporal period.

In some instances, a plurality of primers may comprise at least a nucleotide sequence comprising at least 80% identical to a sequence of a reference genome of a virus. In some cases, a plurality of primers may comprise at least a nucleotide sequence comprising at least 81% identical to a sequence of a reference genome of a virus. In some cases, a plurality of primers may comprise at least a nucleotide sequence comprising at least 82% identical to a sequence of a reference genome of a virus. In some cases, a plurality of primers may comprise at least a nucleotide sequence comprising at least 83% identical to a sequence of a reference genome of a virus. In some cases, a plurality of primers may comprise at least a nucleotide sequence comprising at least 84% identical to a sequence of a reference genome of a virus. In some cases, a plurality of primers may comprise at least a nucleotide sequence comprising at least 85% identical to a sequence of a reference genome of a virus. In some cases, a plurality of primers may comprise at least a nucleotide sequence comprising at least 86% identical to a sequence of a reference genome of a virus. In some cases, a plurality of primers may comprise at least a nucleotide sequence comprising at least 87% identical to a sequence of a reference genome of a virus. In some cases, a plurality of primers may comprise at least a nucleotide sequence comprising at least 88% identical to a sequence of a reference genome of a virus. In some cases, a plurality of primers may comprise at least a nucleotide sequence comprising at least 89% identical to a sequence of a reference genome of a virus. In some cases, a plurality of primers may comprise at least a nucleotide sequence comprising at least 90% identical to a sequence of a reference genome of a virus. In some cases, a plurality of primers may comprise at least a nucleotide sequence comprising at least 91% identical to a sequence of a reference genome of a virus. In some cases, a plurality of primers may comprise at least a nucleotide sequence comprising at least 92% identical to a sequence of a reference genome of a virus. In some cases, a plurality of primers may comprise at least a nucleotide sequence comprising at least 93% identical to a sequence of a reference genome of a virus. In some cases, a plurality of primers may comprise at least a nucleotide sequence comprising at least 94% identical to a sequence of a reference genome of a virus. In some cases, a plurality of primers may comprise at least a nucleotide sequence comprising at least 95% identical to a sequence of a reference genome of a virus. In some cases, a plurality of primers may comprise at least a nucleotide sequence comprising at least 96% identical to a sequence of a reference genome of a virus. In some cases, a plurality of primers may comprise at least a nucleotide sequence comprising at least 97% identical to a sequence of a reference genome of a virus. In some cases, a plurality of primers may comprise at least a nucleotide sequence comprising at least 98% identical to a sequence of a reference genome of a virus. In some cases, a plurality of primers may comprise at least a nucleotide sequence comprising at least 99% identical to a sequence of a reference genome of a virus. In some cases, a plurality of primers may comprise at least a nucleotide sequence comprising a sequence of a reference genome of a virus. In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of a reference genome of a virus.

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of a reference genome of a virus of family Coronaviridae.

In some instances, a plurality of primers may comprise at least a nucleotide sequence comprising at least 80% identical to a sequence of a reference genome of a virus of family Coronaviridae. In some cases, a plurality of primers may comprise at least a nucleotide sequence comprising at least 81% identical to a sequence of a reference genome of a virus of family Coronaviridae. In some cases, a plurality of primers may comprise at least a nucleotide sequence comprising at least 82% identical to a sequence of a reference genome of a virus of family Coronaviridae. In some cases, a plurality of primers may comprise at least a nucleotide sequence comprising at least 83% identical to a sequence of a reference genome of a virus of family Coronaviridae. In some cases, a plurality of primers may comprise at least a nucleotide sequence comprising at least 84% identical to a sequence of a reference genome of a virus of family Coronaviridae. In some cases, a plurality of primers may comprise at least a nucleotide sequence comprising at least 85% identical to a sequence of a reference genome of a virus of family Coronaviridae. In some cases, a plurality of primers may comprise at least a nucleotide sequence comprising at least 86% identical to a sequence of a reference genome of a virus of family Coronaviridae. In some cases, a plurality of primers may comprise at least a nucleotide sequence comprising at least 87% identical to a sequence of a reference genome of a virus of family Coronaviridae. In some cases, a plurality of primers may comprise at least a nucleotide sequence comprising at least 88% identical to a sequence of a reference genome of a virus of family Coronaviridae. In some cases, a plurality of primers may comprise at least a nucleotide sequence comprising at least 89% identical to a sequence of a reference genome of a virus of family Coronaviridae. In some cases, a plurality of primers may comprise at least a nucleotide sequence comprising at least 90% identical to a sequence of a reference genome of a virus of family Coronaviridae. In some cases, a plurality of primers may comprise at least a nucleotide sequence comprising at least 91% identical to a sequence of a reference genome of a virus of family Coronaviridae. In some cases, a plurality of primers may comprise at least a nucleotide sequence comprising at least 92% identical to a sequence of a reference genome of a virus of family Coronaviridae. In some cases, a plurality of primers may comprise at least a nucleotide sequence comprising at least 93% identical to a sequence of a reference genome of a virus of family Coronaviridae. In some cases, a plurality of primers may comprise at least a nucleotide sequence comprising at least 94% identical to a sequence of a reference genome of a virus of family Coronaviridae. In some cases, a plurality of primers may comprise at least a nucleotide sequence comprising at least 95% identical to a sequence of a reference genome of a virus of family Coronaviridae. In some cases, a plurality of primers may comprise at least a nucleotide sequence comprising at least 96% identical to a sequence of a reference genome of a virus of family Coronaviridae. In some cases, a plurality of primers may comprise at least a nucleotide sequence comprising at least 97% identical to a sequence of a reference genome of a virus of family Coronaviridae. In some cases, a plurality of primers may comprise at least a nucleotide sequence comprising at least 98% identical to a sequence of a reference genome of a virus of family Coronaviridae. In some cases, a plurality of primers may comprise at least a nucleotide sequence comprising at least 99% identical to a sequence of a reference genome of a virus of family Coronaviridae. In some cases, a plurality of primers may comprise at least a nucleotide sequence comprising a sequence of a reference genome of a virus of family Coronaviridae. In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of a reference genome of a virus of family Coronaviridae.

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of a reference genome of a virus of subfamily Orthocoronavirinae.

In some instances, a plurality of primers may comprise at least a nucleotide sequence comprising at least 80% identical to a sequence of a reference genome of a virus of subfamily Orthocoronavirinae. In some cases, a plurality of primers may comprise at least a nucleotide sequence comprising at least 81% identical to a sequence of a reference genome of a virus of subfamily Orthocoronavirinae. In some cases, a plurality of primers may comprise at least a nucleotide sequence comprising at least 82% identical to a sequence of a reference genome of a virus of subfamily Orthocoronavirinae. In some cases, a plurality of primers may comprise at least a nucleotide sequence comprising at least 83% identical to a sequence of a reference genome of a virus of subfamily Orthocoronavirinae. In some cases, a plurality of primers may comprise at least a nucleotide sequence comprising at least 84% identical to a sequence of a reference genome of a virus of subfamily Orthocoronavirinae. In some cases, a plurality of primers may comprise at least a nucleotide sequence comprising at least 85% identical to a sequence of a reference genome of a virus of subfamily Orthocoronavirinae. In some cases, a plurality of primers may comprise at least a nucleotide sequence comprising at least 86% identical to a sequence of a reference genome of a virus of subfamily Orthocoronavirinae. In some cases, a plurality of primers may comprise at least a nucleotide sequence comprising at least 87% identical to a sequence of a reference genome of a virus of subfamily Orthocoronavirinae. In some cases, a plurality of primers may comprise at least a nucleotide sequence comprising at least 88% identical to a sequence of a reference genome of a virus of subfamily Orthocoronavirinae. In some cases, a plurality of primers may comprise at least a nucleotide sequence comprising at least 89% identical to a sequence of a reference genome of a virus of subfamily Orthocoronavirinae. In some cases, a plurality of primers may comprise at least a nucleotide sequence comprising at least 90% identical to a sequence of a reference genome of a virus of subfamily Orthocoronavirinae. In some cases, a plurality of primers may comprise at least a nucleotide sequence comprising at least 91% identical to a sequence of a reference genome of a virus of subfamily Orthocoronavirinae. In some cases, a plurality of primers may comprise at least a nucleotide sequence comprising at least 92% identical to a sequence of a reference genome of a virus of subfamily Orthocoronavirinae. In some cases, a plurality of primers may comprise at least a nucleotide sequence comprising at least 93% identical to a sequence of a reference genome of a virus of subfamily Orthocoronavirinae. In some cases, a plurality of primers may comprise at least a nucleotide sequence comprising at least 94% identical to a sequence of a reference genome of a virus of subfamily Orthocoronavirinae. In some cases, a plurality of primers may comprise at least a nucleotide sequence comprising at least 95% identical to a sequence of a reference genome of a virus of subfamily Orthocoronavirinae. In some cases, a plurality of primers may comprise at least a nucleotide sequence comprising at least 96% identical to a sequence of a reference genome of a virus of subfamily Orthocoronavirinae. In some cases, a plurality of primers may comprise at least a nucleotide sequence comprising at least 97% identical to a sequence of a reference genome of a virus of subfamily Orthocoronavirinae. In some cases, a plurality of primers may comprise at least a nucleotide sequence comprising at least 98% identical to a sequence of a reference genome of a virus of subfamily Orthocoronavirinae. In some cases, a plurality of primers may comprise at least a nucleotide sequence comprising at least 99% identical to a sequence of a reference genome of a virus of subfamily Orthocoronavirinae. In some cases, a plurality of primers may comprise at least a nucleotide sequence comprising a sequence of a reference genome of a virus of subfamily Orthocoronavirinae. In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of a reference genome of a virus of subfamily Orthocoronavirinae.

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of a reference genome of a virus of species coronavirus.

In some instances, a plurality of primers may comprise at least a nucleotide sequence comprising at least 80% identical to a sequence of a reference genome of a virus of species coronavirus. In some cases, a plurality of primers may comprise at least a nucleotide sequence comprising at least 81% identical to a sequence of a reference genome of a virus of species coronavirus. In some cases, a plurality of primers may comprise at least a nucleotide sequence comprising at least 82% identical to a sequence of a reference genome of a virus of species coronavirus. In some cases, a plurality of primers may comprise at least a nucleotide sequence comprising at least 83% identical to a sequence of a reference genome of a virus of species coronavirus. In some cases, a plurality of primers may comprise at least a nucleotide sequence comprising at least 84% identical to a sequence of a reference genome of a virus of species coronavirus. In some cases, a plurality of primers may comprise at least a nucleotide sequence comprising at least 85% identical to a sequence of a reference genome of a virus of species coronavirus. In some cases, a plurality of primers may comprise at least a nucleotide sequence comprising at least 86% identical to a sequence of a reference genome of a virus of species coronavirus. In some cases, a plurality of primers may comprise at least a nucleotide sequence comprising at least 87% identical to a sequence of a reference genome of a virus of species coronavirus. In some cases, a plurality of primers may comprise at least a nucleotide sequence comprising at least 88% identical to a sequence of a reference genome of a virus of species coronavirus. In some cases, a plurality of primers may comprise at least a nucleotide sequence comprising at least 89% identical to a sequence of a reference genome of a virus of species coronavirus. In some cases, a plurality of primers may comprise at least a nucleotide sequence comprising at least 90% identical to a sequence of a reference genome of a virus of species coronavirus. In some cases, a plurality of primers may comprise at least a nucleotide sequence comprising at least 91% identical to a sequence of a reference genome of a virus of species coronavirus. In some cases, a plurality of primers may comprise at least a nucleotide sequence comprising at least 92% identical to a sequence of a reference genome of a virus of species coronavirus. In some cases, a plurality of primers may comprise at least a nucleotide sequence comprising at least 93% identical to a sequence of a reference genome of a virus of species coronavirus. In some cases, a plurality of primers may comprise at least a nucleotide sequence comprising at least 94% identical to a sequence of a reference genome of a virus of species coronavirus. In some cases, a plurality of primers may comprise at least a nucleotide sequence comprising at least 95% identical to a sequence of a reference genome of a virus of species coronavirus. In some cases, a plurality of primers may comprise at least a nucleotide sequence comprising at least 96% identical to a sequence of a reference genome of a virus of species coronavirus. In some cases, a plurality of primers may comprise at least a nucleotide sequence comprising at least 97% identical to a sequence of a reference genome of a virus of species coronavirus. In some cases, a plurality of primers may comprise at least a nucleotide sequence comprising at least 98% identical to a sequence of a reference genome of a virus of species coronavirus. In some cases, a plurality of primers may comprise at least a nucleotide sequence comprising at least 99% identical to a sequence of a reference genome of a virus of species coronavirus. In some cases, a plurality of primers may comprise at least a nucleotide sequence comprising a sequence of a reference genome of a virus of species coronavirus. In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of a reference genome of a virus of species coronavirus.

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of a reference genome of a Severe acute respiratory syndrome-related coronavirus.

In some instances, a plurality of primers may comprise at least a nucleotide sequence comprising at least 80% identical to a sequence of a reference genome of a Severe acute respiratory syndrome-related coronavirus. In some cases, a plurality of primers may comprise at least a nucleotide sequence comprising at least 81% identical to a sequence of a reference genome of a Severe acute respiratory syndrome-related coronavirus. In some cases, a plurality of primers may comprise at least a nucleotide sequence comprising at least 82% identical to a sequence of a reference genome of a Severe acute respiratory syndrome-related coronavirus. In some cases, a plurality of primers may comprise at least a nucleotide sequence comprising at least 83% identical to a sequence of a reference genome of a Severe acute respiratory syndrome-related coronavirus. In some cases, a plurality of primers may comprise at least a nucleotide sequence comprising at least 84% identical to a sequence of a reference genome of a Severe acute respiratory syndrome-related coronavirus. In some cases, a plurality of primers may comprise at least a nucleotide sequence comprising at least 85% identical to a sequence of a reference genome of a Severe acute respiratory syndrome-related coronavirus. In some cases, a plurality of primers may comprise at least a nucleotide sequence comprising at least 86% identical to a sequence of a reference genome of a Severe acute respiratory syndrome-related coronavirus. In some cases, a plurality of primers may comprise at least a nucleotide sequence comprising at least 87% identical to a sequence of a reference genome of a Severe acute respiratory syndrome-related coronavirus. In some cases, a plurality of primers may comprise at least a nucleotide sequence comprising at least 88% identical to a sequence of a reference genome of a Severe acute respiratory syndrome-related coronavirus. In some cases, a plurality of primers may comprise at least a nucleotide sequence comprising at least 89% identical to a sequence of a reference genome of a Severe acute respiratory syndrome-related coronavirus. In some cases, a plurality of primers may comprise at least a nucleotide sequence comprising at least 90% identical to a sequence of a reference genome of a Severe acute respiratory syndrome-related coronavirus. In some cases, a plurality of primers may comprise at least a nucleotide sequence comprising at least 91% identical to a sequence of a reference genome of a Severe acute respiratory syndrome-related coronavirus. In some cases, a plurality of primers may comprise at least a nucleotide sequence comprising at least 92% identical to a sequence of a reference genome of a Severe acute respiratory syndrome-related coronavirus. In some cases, a plurality of primers may comprise at least a nucleotide sequence comprising at least 93% identical to a sequence of a reference genome of a Severe acute respiratory syndrome-related coronavirus. In some cases, a plurality of primers may comprise at least a nucleotide sequence comprising at least 94% identical to a sequence of a reference genome of a Severe acute respiratory syndrome-related coronavirus. In some cases, a plurality of primers may comprise at least a nucleotide sequence comprising at least 95% identical to a sequence of a reference genome of a Severe acute respiratory syndrome-related coronavirus. In some cases, a plurality of primers may comprise at least a nucleotide sequence comprising at least 96% identical to a sequence of a reference genome of a Severe acute respiratory syndrome-related coronavirus. In some cases, a plurality of primers may comprise at least a nucleotide sequence comprising at least 97% identical to a sequence of a reference genome of a Severe acute respiratory syndrome-related coronavirus. In some cases, a plurality of primers may comprise at least a nucleotide sequence comprising at least 98% identical to a sequence of a reference genome of a Severe acute respiratory syndrome-related coronavirus. In some cases, a plurality of primers may comprise at least a nucleotide sequence comprising at least 99% identical to a sequence of a reference genome of a Severe acute respiratory syndrome-related coronavirus. In some cases, a plurality of primers may comprise at least a nucleotide sequence comprising a sequence of a reference genome of a Severe acute respiratory syndrome-related coronavirus. In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of a reference genome of a Severe acute respiratory syndrome-related coronavirus.

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of a reference genome of Severe acute respiratory syndrome coronavirus 2 (SARS-CoV-2).

In some instances, a plurality of primers may comprise at least a nucleotide sequence comprising at least 80% identical to a sequence of a reference genome of SARS-CoV-2. In some cases, a plurality of primers may comprise at least a nucleotide sequence comprising at least 81% identical to a sequence of a reference genome of SARS-CoV-2. In some cases, a plurality of primers may comprise at least a nucleotide sequence comprising at least 82% identical to a sequence of a reference genome of SARS-CoV-2. In some cases, a plurality of primers may comprise at least a nucleotide sequence comprising at least 83% identical to a sequence of a reference genome of SARS-CoV-2. In some cases, a plurality of primers may comprise at least a nucleotide sequence comprising at least 84% identical to a sequence of a reference genome of SARS-CoV-2. In some cases, a plurality of primers may comprise at least a nucleotide sequence comprising at least 85% identical to a sequence of a reference genome of SARS-CoV-2. In some cases, a plurality of primers may comprise at least a nucleotide sequence comprising at least 86% identical to a sequence of a reference genome of SARS-CoV-2. In some cases, a plurality of primers may comprise at least a nucleotide sequence comprising at least 87% identical to a sequence of a reference genome of SARS-CoV-2. In some cases, a plurality of primers may comprise at least a nucleotide sequence comprising at least 88% identical to a sequence of a reference genome of SARS-CoV-2. In some cases, a plurality of primers may comprise at least a nucleotide sequence comprising at least 89% identical to a sequence of a reference genome of SARS-CoV-2. In some cases, a plurality of primers may comprise at least a nucleotide sequence comprising at least 90% identical to a sequence of a reference genome of SARS-CoV-2. In some cases, a plurality of primers may comprise at least a nucleotide sequence comprising at least 91% identical to a sequence of a reference genome of SARS-CoV-2. In some cases, a plurality of primers may comprise at least a nucleotide sequence comprising at least 92% identical to a sequence of a reference genome of SARS-CoV-2. In some cases, a plurality of primers may comprise at least a nucleotide sequence comprising at least 93% identical to a sequence of a reference genome of SARS-CoV-2. In some cases, a plurality of primers may comprise at least a nucleotide sequence comprising at least 94% identical to a sequence of a reference genome of SARS-CoV-2. In some cases, a plurality of primers may comprise at least a nucleotide sequence comprising at least 95% identical to a sequence of a reference genome of SARS-CoV-2. In some cases, a plurality of primers may comprise at least a nucleotide sequence comprising at least 96% identical to a sequence of a reference genome of SARS-CoV-2. In some cases, a plurality of primers may comprise at least a nucleotide sequence comprising at least 97% identical to a sequence of a reference genome of SARS-CoV-2. In some cases, a plurality of primers may comprise at least a nucleotide sequence comprising at least 98% identical to a sequence of a reference genome of SARS-CoV-2. In some cases, a plurality of primers may comprise at least a nucleotide sequence comprising at least 99% identical to a sequence of a reference genome of SARS-CoV-2. In some cases, a plurality of primers may comprise at least a nucleotide sequence comprising a sequence of a reference genome of SARS-CoV-2. In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of a reference genome of SARS-CoV-2.

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of a Spike gene or a Nucleocapsid gene of a reference genome of a virus of family Coronaviridae.

In some instances, a plurality of primers may comprise at least a nucleotide sequence comprising at least 80% identical to a sequence of a Spike gene or a Nucleocapsid gene of a reference genome of a virus of family Coronaviridae. In some cases, a plurality of primers may comprise at least a nucleotide sequence comprising at least 81% identical to a sequence of a Spike gene or a Nucleocapsid gene of a reference genome of a virus of family Coronaviridae. In some cases, a plurality of primers may comprise at least a nucleotide sequence comprising at least 82% identical to a sequence of a Spike gene or a Nucleocapsid gene of a reference genome of a virus of family Coronaviridae. In some cases, a plurality of primers may comprise at least a nucleotide sequence comprising at least 83% identical to a sequence of a Spike gene or a Nucleocapsid gene of a reference genome of a virus of family Coronaviridae. In some cases, a plurality of primers may comprise at least a nucleotide sequence comprising at least 84% identical to a sequence of a Spike gene or a Nucleocapsid gene of a reference genome of a virus of family Coronaviridae. In some cases, a plurality of primers may comprise at least a nucleotide sequence comprising at least 85% identical to a sequence of a Spike gene or a Nucleocapsid gene of a reference genome of a virus of family Coronaviridae. In some cases, a plurality of primers may comprise at least a nucleotide sequence comprising at least 86% identical to a sequence of a Spike gene or a Nucleocapsid gene of a reference genome of a virus of family Coronaviridae. In some cases, a plurality of primers may comprise at least a nucleotide sequence comprising at least 87% identical to a sequence of a Spike gene or a Nucleocapsid gene of a reference genome of a virus of family Coronaviridae. In some cases, a plurality of primers may comprise at least a nucleotide sequence comprising at least 88% identical to a sequence of a Spike gene or a Nucleocapsid gene of a reference genome of a virus of family Coronaviridae. In some cases, a plurality of primers may comprise at least a nucleotide sequence comprising at least 89% identical to a sequence of a Spike gene or a Nucleocapsid gene of a reference genome of a virus of family Coronaviridae. In some cases, a plurality of primers may comprise at least a nucleotide sequence comprising at least 90% identical to a sequence of a Spike gene or a Nucleocapsid gene of a reference genome of a virus of family Coronaviridae. In some cases, a plurality of primers may comprise at least a nucleotide sequence comprising at least 91% identical to a sequence of a Spike gene or a Nucleocapsid gene of a reference genome of a virus of family Coronaviridae. In some cases, a plurality of primers may comprise at least a nucleotide sequence comprising at least 92% identical to a sequence of a Spike gene or a Nucleocapsid gene of a reference genome of a virus of family Coronaviridae. In some cases, a plurality of primers may comprise at least a nucleotide sequence comprising at least 93% identical to a sequence of a Spike gene or a Nucleocapsid gene of a reference genome of a virus of family Coronaviridae. In some cases, a plurality of primers may comprise at least a nucleotide sequence comprising at least 94% identical to a sequence of a Spike gene or a Nucleocapsid gene of a reference genome of a virus of family Coronaviridae. In some cases, a plurality of primers may comprise at least a nucleotide sequence comprising at least 95% identical to a sequence of a Spike gene or a Nucleocapsid gene of a reference genome of a virus of family Coronaviridae. In some cases, a plurality of primers may comprise at least a nucleotide sequence comprising at least 96% identical to a sequence of a Spike gene or a Nucleocapsid gene of a reference genome of a virus of family Coronaviridae. In some cases, a plurality of primers may comprise at least a nucleotide sequence comprising at least 97% identical to a sequence of a Spike gene or a Nucleocapsid gene of a reference genome of a virus of family Coronaviridae. In some cases, a plurality of primers may comprise at least a nucleotide sequence comprising at least 98% identical to a sequence of a Spike gene or a Nucleocapsid gene of a reference genome of a virus of family Coronaviridae. In some cases, a plurality of primers may comprise at least a nucleotide sequence comprising at least 99% identical to a sequence of a Spike gene or a Nucleocapsid gene of a reference genome of a virus of family Coronaviridae. In some cases, a plurality of primers may comprise at least a nucleotide sequence comprising a sequence of a reference genome of a virus of family Coronaviridae. In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of a Spike gene or a Nucleocapsid gene of a reference genome of a virus of family Coronaviridae.

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of NC_045512.2 (21563 . . . 29533).

In some instances, a plurality of primers may comprise at least a nucleotide sequence comprising at least 80% identical to a sequence of NC_045512.2 (21563 . . . 29533). In some cases, a plurality of primers may comprise at least a nucleotide sequence comprising at least 81% identical to a sequence of NC_045512.2 (21563 . . . 29533). In some cases, a plurality of primers may comprise at least a nucleotide sequence comprising at least 82% identical to a sequence of NC_045512.2 (21563 . . . 29533). In some cases, a plurality of primers may comprise at least a nucleotide sequence comprising at least 83% identical to a sequence of NC_045512.2 (21563 . . . 29533). In some cases, a plurality of primers may comprise at least a nucleotide sequence comprising at least 84% identical to a sequence of NC_045512.2 (21563 . . . 29533). In some cases, a plurality of primers may comprise at least a nucleotide sequence comprising at least 85% identical to a sequence of NC_045512.2 (21563 . . . 29533). In some cases, a plurality of primers may comprise at least a nucleotide sequence comprising at least 86% identical to a sequence of NC_045512.2 (21563 . . . 29533). In some cases, a plurality of primers may comprise at least a nucleotide sequence comprising at least 87% identical to a sequence of NC_045512.2 (21563 . . . 29533). In some cases, a plurality of primers may comprise at least a nucleotide sequence comprising at least 88% identical to a sequence of NC_045512.2 (21563 . . . 29533). In some cases, a plurality of primers may comprise at least a nucleotide sequence comprising at least 89% identical to a sequence of NC_045512.2 (21563 . . . 29533). In some cases, a plurality of primers may comprise at least a nucleotide sequence comprising at least 90% identical to a sequence of NC_045512.2 (21563 . . . 29533). In some cases, a plurality of primers may comprise at least a nucleotide sequence comprising at least 91% identical to a sequence of NC_045512.2 (21563 . . . 29533). In some cases, a plurality of primers may comprise at least a nucleotide sequence comprising at least 92% identical to a sequence of NC_045512.2 (21563 . . . 29533). In some cases, a plurality of primers may comprise at least a nucleotide sequence comprising at least 93% identical to a sequence of NC_045512.2 (21563 . . . 29533). In some cases, a plurality of primers may comprise at least a nucleotide sequence comprising at least 94% identical to a sequence of NC_045512.2 (21563 . . . 29533). In some cases, a plurality of primers may comprise at least a nucleotide sequence comprising at least 95% identical to a sequence of NC_045512.2 (21563 . . . 29533). In some cases, a plurality of primers may comprise at least a nucleotide sequence comprising at least 96% identical to a sequence of NC_045512.2 (21563 . . . 29533). In some cases, a plurality of primers may comprise at least a nucleotide sequence comprising at least 97% identical to a sequence of NC_045512.2 (21563 . . . 29533). In some cases, a plurality of primers may comprise at least a nucleotide sequence comprising at least 98% identical to a sequence of NC_045512.2 (21563 . . . 29533). In some cases, a plurality of primers may comprise at least a nucleotide sequence comprising at least 99% identical to a sequence of NC_045512.2 (21563 . . . 29533). In some cases, a plurality of primers may comprise at least a nucleotide sequence comprising a sequence of a sequence of NC_045512.2 (21563 . . . 29533). In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of NC_045512.2 (21563 . . . 29533).

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of a Spike gene of a reference genome of a virus of family Coronaviridae.

In some instances, a plurality of primers may comprise at least a nucleotide sequence comprising at least 80% identical to a sequence of a Spike gene of a reference genome of a virus of family Coronaviridae. In some cases, a plurality of primers may comprise at least a nucleotide sequence comprising at least 81% identical to a sequence of a Spike gene of a reference genome of a virus of family Coronaviridae. In some cases, a plurality of primers may comprise at least a nucleotide sequence comprising at least 82% identical to a sequence of a Spike gene of a reference genome of a virus of family Coronaviridae. In some cases, a plurality of primers may comprise at least a nucleotide sequence comprising at least 83% identical to a sequence of a Spike gene of a reference genome of a virus of family Coronaviridae. In some cases, a plurality of primers may comprise at least a nucleotide sequence comprising at least 84% identical to a sequence of a Spike gene of a reference genome of a virus of family Coronaviridae. In some cases, a plurality of primers may comprise at least a nucleotide sequence comprising at least 85% identical to a sequence of a Spike gene of a reference genome of a virus of family Coronaviridae. In some cases, a plurality of primers may comprise at least a nucleotide sequence comprising at least 86% identical to a sequence of a Spike gene of a reference genome of a virus of family Coronaviridae. In some cases, a plurality of primers may comprise at least a nucleotide sequence comprising at least 87% identical to a sequence of a Spike gene of a reference genome of a virus of family Coronaviridae. In some cases, a plurality of primers may comprise at least a nucleotide sequence comprising at least 88% identical to a sequence of a Spike gene of a reference genome of a virus of family Coronaviridae. In some cases, a plurality of primers may comprise at least a nucleotide sequence comprising at least 89% identical to a sequence of a Spike gene of a reference genome of a virus of family Coronaviridae. In some cases, a plurality of primers may comprise at least a nucleotide sequence comprising at least 90% identical to a sequence of a Spike gene of a reference genome of a virus of family Coronaviridae. In some cases, a plurality of primers may comprise at least a nucleotide sequence comprising at least 91% identical to a sequence of a Spike gene of a reference genome of a virus of family Coronaviridae. In some cases, a plurality of primers may comprise at least a nucleotide sequence comprising at least 92% identical to a sequence of a Spike gene of a reference genome of a virus of family Coronaviridae. In some cases, a plurality of primers may comprise at least a nucleotide sequence comprising at least 93% identical to a sequence of a Spike gene of a reference genome of a virus of family Coronaviridae. In some cases, a plurality of primers may comprise at least a nucleotide sequence comprising at least 94% identical to a sequence of a Spike gene of a reference genome of a virus of family Coronaviridae. In some cases, a plurality of primers may comprise at least a nucleotide sequence comprising at least 95% identical to a sequence of a Spike gene of a reference genome of a virus of family Coronaviridae. In some cases, a plurality of primers may comprise at least a nucleotide sequence comprising at least 96% identical to a sequence of a Spike gene of a reference genome of a virus of family Coronaviridae. In some cases, a plurality of primers may comprise at least a nucleotide sequence comprising at least 97% identical to a sequence of a Spike gene of a reference genome of a virus of family Coronaviridae. In some cases, a plurality of primers may comprise at least a nucleotide sequence comprising at least 98% identical to a sequence of a Spike gene of a reference genome of a virus of family Coronaviridae. In some cases, a plurality of primers may comprise at least a nucleotide sequence comprising at least 99% identical to a sequence of a Spike gene of a reference genome of a virus of family Coronaviridae. In some cases, a plurality of primers may comprise at least a nucleotide sequence comprising a sequence a Spike gene of a reference genome of a virus of family Coronaviridae. In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of a Spike gene of a reference genome of a virus of family Coronaviridae.

In some instances, a plurality of primers may comprise at least a nucleotide sequence comprising at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence of a Nucleocapsid gene of a reference genome of a virus of family Coronaviridae.

In some instances, a plurality of primers may comprise at least a nucleotide sequence comprising at least 80% identical to a sequence of a Nucleocapsid gene of a reference genome of a virus of family Coronaviridae. In some cases, a plurality of primers may comprise at least a nucleotide sequence comprising at least 81% identical to a sequence of a Nucleocapsid gene of a reference genome of a virus of family Coronaviridae. In some cases, a plurality of primers may comprise at least a nucleotide sequence comprising at least 82% identical to a sequence of a Nucleocapsid gene of a reference genome of a virus of family Coronaviridae. In some cases, a plurality of primers may comprise at least a nucleotide sequence comprising at least 83% identical to a sequence of a Nucleocapsid gene of a reference genome of a virus of family Coronaviridae. In some cases, a plurality of primers may comprise at least a nucleotide sequence comprising at least 84% identical to a sequence of a Nucleocapsid gene of a reference genome of a virus of family Coronaviridae. In some cases, a plurality of primers may comprise at least a nucleotide sequence comprising at least 85% identical to a sequence of a Nucleocapsid gene of a reference genome of a virus of family Coronaviridae. In some cases, a plurality of primers may comprise at least a nucleotide sequence comprising at least 86% identical to a sequence of a Nucleocapsid gene of a reference genome of a virus of family Coronaviridae. In some cases, a plurality of primers may comprise at least a nucleotide sequence comprising at least 87% identical to a sequence of a Nucleocapsid gene of a reference genome of a virus of family Coronaviridae. In some cases, a plurality of primers may comprise at least a nucleotide sequence comprising at least 88% identical to a sequence of a Nucleocapsid gene of a reference genome of a virus of family Coronaviridae. In some cases, a plurality of primers may comprise at least a nucleotide sequence comprising at least 89% identical to a sequence of a Nucleocapsid gene of a reference genome of a virus of family Coronaviridae. In some cases, a plurality of primers may comprise at least a nucleotide sequence comprising at least 90% identical to a sequence of a Nucleocapsid gene of a reference genome of a virus of family Coronaviridae. In some cases, a plurality of primers may comprise at least a nucleotide sequence comprising at least 91% identical to a sequence of a Nucleocapsid gene of a reference genome of a virus of family Coronaviridae. In some cases, a plurality of primers may comprise at least a nucleotide sequence comprising at least 92% identical to a sequence of a Nucleocapsid gene of a reference genome of a virus of family Coronaviridae. In some cases, a plurality of primers may comprise at least a nucleotide sequence comprising at least 93% identical to a sequence of a Nucleocapsid gene of a reference genome of a virus of family Coronaviridae. In some cases, a plurality of primers may comprise at least a nucleotide sequence comprising at least 94% identical to a sequence of a Nucleocapsid gene of a reference genome of a virus of family Coronaviridae. In some cases, a plurality of primers may comprise at least a nucleotide sequence comprising at least 95% identical to a sequence of a Nucleocapsid gene of a reference genome of a virus of family Coronaviridae. In some cases, a plurality of primers may comprise at least a nucleotide sequence comprising at least 96% identical to a sequence of a Nucleocapsid gene of a reference genome of a virus of family Coronaviridae. In some cases, a plurality of primers may comprise at least a nucleotide sequence comprising at least 97% identical to a sequence of a Nucleocapsid gene of a reference genome of a virus of family Coronaviridae. In some cases, a plurality of primers may comprise at least a nucleotide sequence comprising at least 98% identical to a sequence of a Nucleocapsid gene of a reference genome of a virus of family Coronaviridae. In some cases, a plurality of primers may comprise at least a nucleotide sequence comprising at least 99% identical to a sequence of a Nucleocapsid gene of a reference genome of a virus of family Coronaviridae. In some cases, a plurality of primers may comprise at least a nucleotide sequence comprising a sequence a Nucleocapsid gene of a reference genome of a virus of family Coronaviridae. In some cases, a plurality of primers may comprise at least a nucleotide sequence comprising at least 50% identical, at least 55% identical, at least 60% identical, at least 65% identical, at least 70% identical, at least 75% identical, or at least 80% identical to a sequence of a Nucleocapsid gene of a reference genome of a virus of family Coronaviridae.

In some instances, a primer may not comprise a nucleotide sequence comprising at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical or complementary to a sequence that is not part of a genome of a virus of family Coronaviridae. A primer may not comprise a nucleotide sequence comprising at least about 50%, 51%, 52%, 53%, 54%, 55%, 56%, 57%, 58%, 59%, 60%, 61%, 62%, 63%, 64%, 65%, 660%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical or complementary to a sequence that is not part of a genome of Severe acute respiratory syndrome-related coronavirus. A primer may not comprise a nucleotide sequence comprising at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical or complementary to a sequence of that is not part of a genome of species coronavirus. A primer may not comprise a nucleotide sequence comprising at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical or complementary to a sequence that is not part of a genome of a virus of subfamily Orthocoronavirinae. A primer may not comprise a nucleotide sequence comprising at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical or complementary to a sequence that is not part of a genome of a virus. A primer may not comprise a nucleotide sequence comprising at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical or complementary to a sequence of a subject or a microorganism of the subject.

A primer may comprise a nucleotide sequence comprising at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% identical or complementary to a sequence that is not part of a genome of a virus of family Coronaviridae. A primer may comprise a nucleotide sequence comprising at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% identical or complementary to a sequence of that is not part of a genome of Severe acute respiratory syndrome-related coronavirus. A primer may comprise a nucleotide sequence comprising at most about 1%, 2%, 30%, 40%, 50%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 290%, 30%, 31%, 32%, 33%, 34%, 35%, 36%, 37%, 38%, 39%, 40%, 41%, 42%, 43%, 44%, 45%, 46%, 47%, 48%, 49%, or 50% identical or complementary to a sequence of that is not part of a genome of species coronavirus. A primer may comprise a nucleotide sequence comprising at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% identical or complementary to a sequence of that is not part of a genome of virus of subfamily Orthocoronavirinae. A primer may comprise a nucleotide sequence comprising at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% identical or complementary to a sequence of that is not part of a genome of a virus. A primer may comprise a nucleotide sequence comprising at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% identical or complementary to a sequence of a subject or a microorganism of the subject.

In some instances, a pair of primers for generating an amplicon may be selected such that it will generate at most 1 amplicon from a template nucleotide sequence. In some cases, a pair of primers for generating an amplicon may be selected such that it will generate at most 1 amplicon from a template nucleotide sequence by using Primer-BLAST. In some cases, a pair of primers for generating an amplicon may be selected such that it will prevent amplification of additional non-specific targets using Primer-BLAST. In some instances, a pair of primers for generating an amplicon may generate at most 1 amplicon from a template nucleotide sequence. In some cases, a pair of primers for generating an amplicon may generate at most 1 amplicon from a template nucleotide sequence based on the determination or prediction of Primer-BLAST. In some cases, a pair of primers for generating an amplicon may prevent amplification of additional non-specific targets based on the determination or prediction of Primer-BLAST.

In some instances, a primer may hybridize or anneal to a region adjacent to a conserved region of a virus. In some cases, a primer may hybridize or anneal to a region at least about 1 nt, 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, 55 nt, 60 nt, 65 nt, 70 nt, 75 nt, 80 nt, 85 nt, 90 nt, 95 nt, 100 nt, 150 nt, 200 nt or more 5′ or 3′ to a conserved region of a virus. In some cases, a primer may hybridize or anneal to a region at most about 1 nt, 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, 55 nt, 60 nt, 65 nt, 70 nt, 75 nt, 80 nt, 85 nt, 90 nt, 95 nt, 100 nt, 150 nt, or 200 nt 5′ or 3′ to a conserved region of a virus.

In some cases, a conserved region of a virus may comprises a region with at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to sequences of the same region among the reference genomes of a virus family Coronaviridae. In some cases, a conserved region of a virus may comprises a region with at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to sequences of the same region among the reference genomes of a virus of subfamily Orthocoronavirinae. In some cases, a conserved region of a virus may comprises a region with at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to sequences of the same region among the reference genomes of a virus of species coronavirus. In some cases, a conserved region of a virus may comprises a region with at least about 80%, 81%, 82%, 83%, 84%, 85%, 86%, 870%, 88%, 89%, 90%, 91%, 92%, 93%, 94%, 95%, 96%, 97%, 98%, 99%, or 100% identical to sequences of the same region among the reference genomes of a Severe acute respiratory syndrome-related coronavirus. In some cases, a conserved region of a virus may comprises a region with at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to sequences of the same region among the reference genomes Severe acute respiratory syndrome coronavirus 2.

In some cases, a primer may not form a primer dimer. In some case, a primer may not form a primer dimer with another primer comprising the same sequences (i.e., a self-dimer). For example, a self-dimer of a primer may form when a sequence or portion of a primer is complimentary to another sequence or portion of the primer. In this cases, two primer molecules comprising the sequences of the complimentary regions may allow the two primer molecules having the same sequence to anneal and/or hybridize to each other and form the self-dimer. In some cases, the complimentary sequences or portions of a primer may be at most about 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. In some case, a primer may not form a primer dimer with another primer comprising a different sequence but with at least a portion of complementary region (i.e., a cross-dimer). For example, a cross-dimer of two primers may form when a sequence or portion of a primer is complimentary to a sequence or portion of another primer. In this cases, two primer molecules comprising the sequences of the complimentary regions may allow the two primer molecules having different sequences to anneal and/or hybridize to each other and form the cross-dimer. In some cases, the complimentary sequences or portions of the two primers may be at most about 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. In some instances, a formation of primer dimer can be predicted and/or determined by an algorithm. Such an algorithm may comprise AutoDimer, Lasergene, Multiple Primer Analyzer, NetPrimer, OligoAnalyzer, OligoEvaluator, PanelPlex, Primer3, PrimerPlex, PrimerSuite, or a combination thereof. In some instances, a primer may not form a primer dimer based on the prediction or determination of an algorithm. In some instances, a primer may not form a primer dimer based on the prediction or determination of AutoDimer, Lasergene, Multiple Primer Analyzer, NetPrimer, OligoAnalyzer, OligoEvaluator, PanelPlex, Primer3, PrimerPlex, PrimerSuite, or a combination thereof

### Amplicon Generation

In some instances, a substrate nucleic acid (e.g., an amplicon) may be generated by an amplification reaction. In some cases, an amplification may comprise a reverse transcription, an asymmetric amplification, a helicase-dependent amplification (HDA), a ligase chain reaction (LCR), a loop mediated isothermal amplification (LAMP), a multiple displacement amplification (MDA), a nucleic acid sequence based amplification (NASBA), a polymerase chain reaction (PCR), a primer extension, a recombinase polymerase amplification (RPA), a rolling circle amplification (RCA), a self-sustained sequence replication (3SR), a strand displacement amplification (SDA), any derivatives thereof, or any combinations thereof. In some cases, an amplification may comprise a reverse transcription or derivatives thereof. In some cases, an amplification may comprise an asymmetric amplification or derivatives thereof. In some cases, an amplification may comprise a HAD or derivatives thereof. In some cases, an amplification may comprise an LCR or derivatives thereof. In some cases, an amplification may comprise a LAMP or derivatives thereof. In some cases, an amplification may comprise an MDA or derivatives thereof. In some cases, an amplification may comprise a NASBA or derivatives thereof. In some cases, an amplification may comprise a PCR or derivatives thereof. In some cases, an amplification may comprise a primer extension or derivatives thereof. In some cases, an amplification may comprise an RPA or derivatives thereof. In some cases, an amplification may comprise an RCA or derivatives thereof. In some cases, an amplification may comprise a 3SR or derivatives thereof. In some cases, an amplification may comprise an SDA or derivatives thereof. Other chemical reactions may also be used to generate a derivative of a molecule in a biological sample.

In some instances, a PCR may comprise an allele-specific PCR, an assembly PCR, an asymmetric PCR, a COLD-PCR (co-amplification at lower denaturation temperature-PCR), a dial-out PCR, a digital PCR, a digital PCR, an emulsion PCR, a helicase-dependent PCR, a hot start PCR, an inverse PCR, a Klenow-based PCR, a ligation-mediated PCR, a methylation-specific PCR, a miniprimer PCR, a multiplex PCR, a nested PCR, a nested PCR, an overlap-extension PCR, a quantitative PCR, a real-time PCR, a thermal asymmetric interlaced PCR and touchdown PCR, a touchdown PCR, or a two-tailed PCR. In some cases, a PCR may comprise an allele-specific PCR. In some cases, a PCR may comprise an assembly PCR. In some cases, a PCR may comprise an asymmetric PCR. In some cases, a PCR may comprise a COLD-PCR (co-amplification at lower denaturation temperature-PCR). In some cases, a PCR may comprise a dial-out PCR. In some cases, a PCR may comprise a digital PCR. In some cases, a PCR may comprise a digital PCR. In some cases, a PCR may comprise an emulsion PCR. In some cases, a PCR may comprise a helicase-dependent PCR. In some cases, a PCR may comprise a hot start PCR. In some cases, a PCR may comprise an inverse PCR. In some cases, a PCR may comprise a Klenow-based PCR. In some cases, a PCR may comprise a ligation-mediated PCR. In some cases, a PCR may comprise a methylation-specific PCR. In some cases, a PCR may comprise a miniprimer PCR. In some cases, a PCR may comprise a multiplex PCR. In some cases, a PCR may comprise a nested PCR. In some cases, a PCR may comprise a nested PCR. In some cases, a PCR may comprise an overlap-extension PCR. In some cases, a PCR may comprise a quantitative PCR. In some cases, a PCR may comprise a real-time PCR. In some cases, a PCR may comprise a thermal asymmetric interlaced PCR and touchdown PCR. In some cases, a PCR may comprise a touchdown PCR. In some cases, a PCR may comprise a two-tailed PCR.

In some cases, a PCR may comprise 21 thermal cycles. In some cases, a PCR may comprise 22 thermal cycles. In some cases, a PCR may comprise 23 thermal cycles. In some cases, a PCR may comprise 24 thermal cycles. In some cases, a PCR may comprise 25 thermal cycles. In some cases, a PCR may comprise 26 thermal cycles. In some cases, a PCR may comprise 27 thermal cycles. In some cases, a PCR may comprise 28 thermal cycles. In some cases, a PCR may comprise 29 thermal cycles. In some cases, a PCR may comprise 30 thermal cycles. In some cases, a PCR may comprise 31 thermal cycles. In some cases, a PCR may comprise 32 thermal cycles. In some cases, a PCR may comprise 33 thermal cycles. In some cases, a PCR may comprise 34 thermal cycles. In some cases, a PCR may comprise 35 thermal cycles. In some cases, a PCR may comprise 36 thermal cycles. In some cases, a PCR may comprise 37 thermal cycles. In some cases, a PCR may comprise 38 thermal cycles. In some cases, a PCR may comprise 39 thermal cycles. In some cases, a PCR may comprise 40 thermal cycles. In some cases, a PCR may comprise 41 thermal cycles. In some cases, a PCR may comprise 42 thermal cycles. In some cases, a PCR may comprise 43 thermal cycles. In some cases, a PCR may comprise 44 thermal cycles. In some cases, a PCR may comprise 45 thermal cycles. In some cases, a PCR may comprise 46 thermal cycles. In some cases, a PCR may comprise 47 thermal cycles. In some cases, a PCR may comprise 48 thermal cycles. In some cases, a PCR may comprise 49 thermal cycles. In some cases, a PCR may comprise 50 thermal cycles. In some cases, a PCR may comprise 51 thermal cycles. In some cases, a PCR may comprise 52 thermal cycles. In some cases, a PCR may comprise 53 thermal cycles. In some cases, a PCR may comprise 54 thermal cycles. In some cases, a PCR may comprise 55 thermal cycles. In some cases, a PCR may comprise 56 thermal cycles. In some cases, a PCR may comprise 57 thermal cycles. In some cases, a PCR may comprise 58 thermal cycles. In some cases, a PCR may comprise 59 thermal cycles. In some cases, a PCR may comprise 60 thermal cycles. In some cases, a PCR may comprise 10 thermal cycles, 11 thermal cycles, 12 thermal cycles, 13 thermal cycles, 14 thermal cycles, 15 thermal cycles, 16 thermal cycles, 17 thermal cycles, 18 thermal cycles, 19 thermal cycles, or 20 thermal cycles. In some cases, a PCR may comprise 61 thermal cycles, 62 thermal cycles, 63 thermal cycles, 64 thermal cycles, 65 thermal cycles, 66 thermal cycles, 67 thermal cycles, 68 thermal cycles, 69 thermal cycles, 70 thermal cycles, 71 thermal cycles, 72 thermal cycles, 73 thermal cycles, 74 thermal cycles, 75 thermal cycles, 76 thermal cycles, 77 thermal cycles, 78 thermal cycles, 79 thermal cycles, 80 thermal cycles, 81 thermal cycles, 82 thermal cycles, 83 thermal cycles, 84 thermal cycles, 85 thermal cycles, 86 thermal cycles, 87 thermal cycles, 88 thermal cycles, 89 thermal cycles, 90 thermal cycles, 91 thermal cycles, 92 thermal cycles, 93 thermal cycles, 94 thermal cycles, 95 thermal cycles, 96 thermal cycles, 97 thermal cycles, 98 thermal cycles, 99 thermal cycles, 100 or more thermal cycles.

In some cases, a PCR may comprise 10 to 75 thermal cycles. In some cases, a PCR may comprise 10 to 70 thermal cycles. In some cases, a PCR may comprise 10 to 65 thermal cycles. In some cases, a PCR may comprise 10 to 60 thermal cycles. In some cases, a PCR may comprise 10 to 55 thermal cycles. In some cases, a PCR may comprise 10 to 50 thermal cycles. In some cases, a PCR may comprise 10 to 45 thermal cycles. In some cases, a PCR may comprise 10 to 40 thermal cycles. In some cases, a PCR may comprise 10 to 35 thermal cycles. In some cases, a PCR may comprise 10 to 30 thermal cycles. In some cases, a PCR may comprise 10 to 25 thermal cycles. In some cases, a PCR may comprise 10 to 20 thermal cycles. In some cases, a PCR may comprise 10 to 15 thermal cycles. In some cases, a PCR may comprise 15 to 75 thermal cycles. In some cases, a PCR may comprise 20 to 75 thermal cycles. In some cases, a PCR may comprise 25 to 75 thermal cycles. In some cases, a PCR may comprise 30 to 75 thermal cycles. In some cases, a PCR may comprise 35 to 75 thermal cycles. In some cases, a PCR may comprise 40 to 75 thermal cycles. In some cases, a PCR may comprise 10 to 50 thermal cycles. In some cases, a PCR may comprise 12 to 49 thermal cycles. In some cases, a PCR may comprise 14 to 48 thermal cycles. In some cases, a PCR may comprise 16 to 47 thermal cycles. In some cases, a PCR may comprise 18 to 46 thermal cycles. In some cases, a PCR may comprise 20 to 45 thermal cycles. In some cases, a PCR may comprise 22 to 44 thermal cycles. In some cases, a PCR may comprise 24 to 43 thermal cycles. In some cases, a PCR may comprise 26 to 42 thermal cycles. In some cases, a PCR may comprise 28 to 41 thermal cycles. In some cases, a PCR may comprise 30 to 40 thermal cycles.

In some instances, generating substrate nucleic acids may comprise a chemical reaction to generate a derivative of a molecule in a biological sample. In some cases, the derivative may comprise the template nucleotide sequences described thereof. In some cases, a chemical reaction for generating substrate nucleic acids may comprise an oligonucleotide described herein. In some cases, a chemical reaction for generating substrate nucleic acids may comprise a reverse transcription. For example, a viral genome may comprise RNA molecules. The viral RNA molecule, in some cases, may comprise mRNA molecules. The viral mRNA molecules may be converted to complementary DNA (cDNA) molecules using a reverse transcription. A reverse transcription (RT), in some instance, may comprise priming an mRNA template nucleotide sequence with the oligonucleotide. One example of an oligonucleotide may comprise an RT primer. In some cases, an RT primer may comprise a DNA nucleotide sequence. In some cases, an RT primer may comprise an oligo-dT primer (e.g., a single-stranded sequence of deoxythymine), a primer with a random nucleotide sequence (e.g., a random hexamer), or a gene-specific primer with a gene-specific nucleotide sequence. In one case, an RT primer may be single-stranded.

In some cases, generating substrate nucleic acids may comprise an oligonucleotide. In some cases, an oligonucleotide may bind nucleic acid. In some cases, an oligonucleotide may bind DNAs. In some cases, an oligonucleotide may bind RNAs. In some cases, an oligonucleotide that can bind RNAs may comprise an oligonucleotide that can bind mRNAs.

In some instances, an oligonucleotide may comprise about 5 nucleotides in length. In some cases, an oligonucleotide may comprise about 6 nucleotides in length. In some cases, an oligonucleotide may comprise about 7 nucleotides in length. In some cases, an oligonucleotide may comprise about 8 nucleotides in length. In some cases, an oligonucleotide may comprise about 9 nucleotides in length. In some cases, an oligonucleotide may comprise about 10 nucleotides in length. In some cases, an oligonucleotide may comprise about 11 nucleotides in length. In some cases, an oligonucleotide may comprise about 12 nucleotides in length. In some cases, an oligonucleotide may comprise about 13 nucleotides in length. In some cases, an oligonucleotide may comprise about 14 nucleotides in length. In some cases, an oligonucleotide may comprise about 15 nucleotides in length. In some cases, an oligonucleotide may comprise about 16 nucleotides in length. In some cases, an oligonucleotide may comprise about 17 nucleotides in length. In some cases, an oligonucleotide may comprise about 18 nucleotides in length. In some cases, an oligonucleotide may comprise about 19 nucleotides in length. In some cases, an oligonucleotide may comprise about 20 nucleotides in length. In some cases, an oligonucleotide may comprise about 21 nucleotides in length. In some cases, an oligonucleotide may comprise about 22 nucleotides in length. In some cases, an oligonucleotide may comprise about 23 nucleotides in length. In some cases, an oligonucleotide may comprise about 24 nucleotides in length. In some cases, an oligonucleotide may comprise about 25 nucleotides in length. In some cases, an oligonucleotide may comprise about 26 nucleotides in length. In some cases, an oligonucleotide may comprise about 27 nucleotides in length. In some cases, an oligonucleotide may comprise about 28 nucleotides in length. In some cases, an oligonucleotide may comprise about 29 nucleotides in length. In some cases, an oligonucleotide may comprise about 30 nucleotides in length. In some cases, an oligonucleotide may comprise from about 10 nucleotides in length to about 15 nucleotides in length. In some cases, an oligonucleotide may comprise from about 10 nucleotides in length to about 20 nucleotides in length. In some cases, an oligonucleotide may comprise from about 10 nucleotides in length to about 25 nucleotides in length. In some cases, an oligonucleotide may comprise from about 10 nucleotides in length to about 30 nucleotides in length.

In some cases, an oligonucleotide may comprise from 1 to 25 nucleotides in length. In some cases, an oligonucleotide may comprise from 2 to 25 nucleotides in length. In some cases, an oligonucleotide may comprise from 3 to 25 nucleotides in length. In some cases, an oligonucleotide may comprise from 4 to 25 nucleotides in length. In some cases, an oligonucleotide may comprise from 5 to 25 nucleotides in length. In some cases, an oligonucleotide may comprise from 6 to 25 nucleotides in length. In some cases, an oligonucleotide may comprise from 7 to 25 nucleotides in length. In some cases, an oligonucleotide may comprise from 8 to 25 nucleotides in length. In some cases, an oligonucleotide may comprise from 9 to 25 nucleotides in length. In some cases, an oligonucleotide may comprise from 10 to 25 nucleotides in length. In some cases, an oligonucleotide may comprise from 1 to 25 nucleotides in length. In some cases, an oligonucleotide may comprise from 1 to 24 nucleotides in length. In some cases, an oligonucleotide may comprise from 1 to 23 nucleotides in length. In some cases, an oligonucleotide may comprise from 1 to 22 nucleotides in length. In some cases, an oligonucleotide may comprise from 1 to 21 nucleotides in length. In some cases, an oligonucleotide may comprise from 1 to 20 nucleotides in length. In some cases, an oligonucleotide may comprise from 1 to 19 nucleotides in length. In some cases, an oligonucleotide may comprise from 1 to 18 nucleotides in length. In some cases, an oligonucleotide may comprise from 1 to 17 nucleotides in length. In some cases, an oligonucleotide may comprise from 1 to 16 nucleotides in length. In some cases, an oligonucleotide may comprise from 1 to 15 nucleotides in length. In some cases, an oligonucleotide may comprise from 1 to 14 nucleotides in length. In some cases, an oligonucleotide may comprise from 1 to 13 nucleotides in length. In some cases, an oligonucleotide may comprise from 1 to 12 nucleotides in length. In some cases, an oligonucleotide may comprise from 1 to 11 nucleotides in length. In some cases, an oligonucleotide may comprise from 1 to 10 nucleotides in length.

In some instances, generating substrate nucleic acids may comprise barcoding the substrate nucleic acids with unique nucleotide sequences (e.g., barcode nucleotide sequence). A barcode nucleotide sequence, in some cases, may comprise a pre-determined nucleotide sequence for identifying the barcode nucleotide sequence, or any molecules attached to the barcode nucleotide sequence. For example, a first amplicon generated from a template nucleotide sequence from a first biological sample may be attached to a first barcode nucleotide sequence, and a second amplicon generated from a template nucleotide sequence generated from a second biological sample may be attached to a second barcode nucleotide sequence. By determining the barcode nucleotide sequence of a substrate nucleic acid, the identity of the biological sample containing the template nucleotide sequence may be identified. In some cases, a barcoding reaction may comprise a barcoding probe comprising a barcode nucleotide sequence. In some cases, a barcoding reaction may comprise attaching or linking a barcoding probe to a substrate nucleic acid. In other cases, a barcoding reaction may comprise attaching or linking a barcoding probe to a template nucleotide sequence. In some cases, a barcoding probe may be attached or linked to a substrate nucleic acid or a template nucleotide sequence by ligation. In some cases, a barcoding probe may be attached or linked to a substrate nucleic acid or a template nucleotide sequence by any amplification reactions thereof. For example, a barcoding reaction may comprise a PCR reaction using a pair of primers comprising at least a primer with a barcode nucleotide sequence.

In some instances, a barcoding probe may comprise about 5 nucleotides in length. In some cases, a barcoding probe may comprise about 6 nucleotides in length. In some cases, a barcoding probe may comprise about 7 nucleotides in length. In some cases, a barcoding probe may comprise about 8 nucleotides in length. In some cases, a barcoding probe may comprise about 9 nucleotides in length. In some cases, a barcoding probe may comprise about 10 nucleotides in length. In some cases, a barcoding probe may comprise about 11 nucleotides in length. In some cases, a barcoding probe may comprise about 12 nucleotides in length. In some cases, a barcoding probe may comprise about 13 nucleotides in length. In some cases, a barcoding probe may comprise about 14 nucleotides in length. In some cases, a barcoding probe may comprise about 15 nucleotides in length. In some cases, a barcoding probe may comprise about 16 nucleotides in length. In some cases, a barcoding probe may comprise about 17 nucleotides in length. In some cases, a barcoding probe may comprise about 18 nucleotides in length. In some cases, a barcoding probe may comprise about 19 nucleotides in length. In some cases, a barcoding probe may comprise about 20 nucleotides in length. In some cases, a barcoding probe may comprise about 21 nucleotides in length. In some cases, a barcoding probe may comprise about 22 nucleotides in length. In some cases, a barcoding probe may comprise about 23 nucleotides in length. In some cases, a barcoding probe may comprise about 24 nucleotides in length. In some cases, a barcoding probe may comprise about 25 nucleotides in length. In some cases, a barcoding probe may comprise about 26 nucleotides in length. In some cases, a barcoding probe may comprise about 27 nucleotides in length. In some cases, a barcoding probe may comprise about 28 nucleotides in length. In some cases, a barcoding probe may comprise about 29 nucleotides in length. In some cases, a barcoding probe may comprise about 30 nucleotides in length. In some cases, a barcoding probe may comprise from about 10 nucleotides in length to about 15 nucleotides in length. In some cases, a barcoding probe may comprise from about 10 nucleotides in length to about 20 nucleotides in length. In some cases, a barcoding probe may comprise from about 10 nucleotides in length to about 25 nucleotides in length. In some cases, a barcoding probe may comprise from about 10 nucleotides in length to about 30 nucleotides in length.

In some instances, a barcoding reaction may comprise 3 unique barcode sequences. In some cases, a barcoding reaction may comprise 1 unique barcode sequence. In some cases, a barcoding reaction may comprise 2 unique barcode sequences. In some cases, a barcoding reaction may comprise 4 unique barcode sequences. In some cases, a barcoding reaction may comprise 5 unique barcode sequences. In some cases, a barcoding reaction may comprise 6 unique barcode sequences. In some cases, a barcoding reaction may comprise 7 unique barcode sequences. In some cases, a barcoding reaction may comprise 8 unique barcode sequences. In some cases, a barcoding reaction may comprise 9 unique barcode sequences. In some cases, a barcoding reaction may comprise 10 unique barcode sequences. In some cases, a barcoding reaction may comprise 11 unique barcode sequences. In some cases, a barcoding reaction may comprise 12 unique barcode sequences. In some cases, a barcoding reaction may comprise 13 unique barcode sequences. In some cases, a barcoding reaction may comprise 14 unique barcode sequences. In some cases, a barcoding reaction may comprise 15 unique barcode sequences. In some cases, a barcoding reaction may comprise 16 unique barcode sequences. In some cases, a barcoding reaction may comprise 17 unique barcode sequences. In some cases, a barcoding reaction may comprise 18 unique barcode sequences. In some cases, a barcoding reaction may comprise 19 unique barcode sequences. In some cases, a barcoding reaction may comprise 20 unique barcode sequences. In some cases, a barcoding reaction may comprise 21 unique barcode sequences. In some cases, a barcoding reaction may comprise 22 unique barcode sequences. In some cases, a barcoding reaction may comprise 23 unique barcode sequences. In some cases, a barcoding reaction may comprise 24 unique barcode sequences. In some cases, a barcoding reaction may comprise 25 unique barcode sequences. In some cases, a barcoding reaction may comprise 26 unique barcode sequences. In some cases, a barcoding reaction may comprise 27 unique barcode sequences. In some cases, a barcoding reaction may comprise 28 unique barcode sequences. In some cases, a barcoding reaction may comprise 29 unique barcode sequences. In some cases, a barcoding reaction may comprise 30 unique barcode sequences. In some cases, a barcoding reaction may comprise 31 unique barcode sequences. In some cases, a barcoding reaction may comprise 32 unique barcode sequences. In some cases, a barcoding reaction may comprise 33 unique barcode sequences. In some cases, a barcoding reaction may comprise 34 unique barcode sequences. In some cases, a barcoding reaction may comprise 35 unique barcode sequences. In some cases, a barcoding reaction may comprise 36 unique barcode sequences. In some cases, a barcoding reaction may comprise 37 unique barcode sequences. In some cases, a barcoding reaction may comprise 38 unique barcode sequences. In some cases, a barcoding reaction may comprise 39 unique barcode sequences. In some cases, a barcoding reaction may comprise 40 unique barcode sequences. In some cases, a barcoding reaction may comprise 41 unique barcode sequences. In some cases, a barcoding reaction may comprise 42 unique barcode sequences. In some cases, a barcoding reaction may comprise 43 unique barcode sequences. In some cases, a barcoding reaction may comprise 44 unique barcode sequences. In some cases, a barcoding reaction may comprise 45 unique barcode sequences. In some cases, a barcoding reaction may comprise 46 unique barcode sequences. In some cases, a barcoding reaction may comprise 47 unique barcode sequences. In some cases, a barcoding reaction may comprise 48 unique barcode sequences. In some cases, a barcoding reaction may comprise 49 unique barcode sequences. In some cases, a barcoding reaction may comprise 50 unique barcode sequences. In some cases, a barcoding reaction may comprise about 55 unique barcode sequences, about 60 unique barcode sequences, about 65 unique barcode sequences, about 70 unique barcode sequences, about 75 unique barcode sequences, about 80 unique barcode sequences, about 85 unique barcode sequences, about 90 unique barcode sequences, about 95 unique barcode sequences, about 100 unique barcode sequences, about 110 unique barcode sequences, about 120 unique barcode sequences, about 130 unique barcode sequences, about 140 unique barcode sequences, about 150 unique barcode sequences, about 160 unique barcode sequences, about 170 unique barcode sequences, about 180 unique barcode sequences, about 190 unique barcode sequences, about 200 unique barcode sequences, about 210 unique barcode sequences, about 220 unique barcode sequences, about 230 unique barcode sequences, about 240 unique barcode sequences, about 250 unique barcode sequences, about 260 unique barcode sequences, about 270 unique barcode sequences, about 280 unique barcode sequences, about 290 unique barcode sequences, about 300 unique barcode sequences, about 350 unique barcode sequences, about 400 unique barcode sequences, about 450 unique barcode sequences, about 500 unique barcode sequences, about 1000 unique barcode sequences, about 1500 unique barcode sequences, about 2000 unique barcode sequences, about 2500 unique barcode sequences, about 3000 unique barcode sequences, about 3500 unique barcode sequences, about 4000 unique barcode sequences, about 4500 unique barcode sequences, or about 5000 unique barcode sequences.

In some cases, a barcoding reaction may comprise from 1 to 50 unique barcode sequences. In some cases, a barcoding reaction may comprise from 2 to 50 unique barcode sequences. In some cases, a barcoding reaction may comprise from 3 to 50 unique barcode sequences. In some cases, a barcoding reaction may comprise from 4 to 50 unique barcode sequences. In some cases, a barcoding reaction may comprise from 5 to 50 unique barcode sequences. In some cases, a barcoding reaction may comprise from 6 to 50 unique barcode sequences. In some cases, a barcoding reaction may comprise from 7 to 50 unique barcode sequences. In some cases, a barcoding reaction may comprise from 8 to 50 unique barcode sequences. In some cases, a barcoding reaction may comprise from 9 to 50 unique barcode sequences. In some cases, a barcoding reaction may comprise from 10 to 50 unique barcode sequences. In some cases, a barcoding reaction may comprise from 1 to 45 unique barcode sequences. In some cases, a barcoding reaction may comprise from 1 to 40 unique barcode sequences. In some cases, a barcoding reaction may comprise from 1 to 35 unique barcode sequences. In some cases, a barcoding reaction may comprise from 1 to 30 unique barcode sequences. In some cases, a barcoding reaction may comprise from 1 to 25 unique barcode sequences. In some cases, a barcoding reaction may comprise from 1 to 20 unique barcode sequences. In some cases, a barcoding reaction may comprise from 1 to 15 unique barcode sequences. In some cases, a barcoding reaction may comprise from 1 to 10 unique barcode sequences. In some cases, a barcoding reaction may comprise from 1 to 5 unique barcode sequences.

### Sequencing

In some instances, the sequence of a substrate nucleic acid may be determined. In some cases, the complement sequence of a substrate nucleic acid may be determined. In some cases, determining the sequence of a substrate nucleic acid or the complement sequence of a substrate nucleic acid may comprise a sequencing reaction. In some cases, determining the sequence of an amplification product of a template nucleotide sequence or a complement of a template nucleotide sequence may comprise a sequencing reaction.

In some instances, a sequencing reaction may comprise a chain termination sequencing, a high-throughput sequencing, a mass spectrophotometry sequencing, a massively parallel signature sequencing, a Maxam-Gilbert sequencing, a nanopore sequencing, a primer walking, a pyrosequencing, a Sanger sequencing, a semiconductor sequencing, a sequencing-by-hybridization, a sequencing-by-ligation, a sequencing-by-synthesis, a single-molecule sequencing, or a shotgun sequencing. In some cases, a sequencing reaction may comprise a chain termination sequencing. In some cases, a sequencing reaction may comprise a high-throughput sequencing. In some cases, a sequencing reaction may comprise a mass spectrophotometry sequencing. In some cases, a sequencing reaction may comprise a massively parallel signature sequencing. In some cases, a sequencing reaction may comprise a Maxam-Gilbert sequencing. In some cases, a sequencing reaction may comprise a nanopore sequencing. In some cases, a sequencing reaction may comprise a primer walking. In some cases, a sequencing reaction may comprise a pyrosequencing. In some cases, a sequencing reaction may comprise a Sanger sequencing. In some cases, a sequencing reaction may comprise a semiconductor sequencing. In some cases, a sequencing reaction may comprise a sequencing-by-hybridization. In some cases, a sequencing reaction may comprise a sequencing-by-ligation. In some cases, a sequencing reaction may comprise a sequencing-by-synthesis. In some cases, a sequencing reaction may comprise a single-molecule sequencing. In some cases, a sequencing reaction may comprise a shotgun sequencing.

In some cases, a sequencing reaction may be conducted in a sequencing platform. A sequencing platform, in some cases, may comprise an Illumina sequencing platform, an ABI SOliD sequencing platform, a Pacific Biosciences sequencing platform, a 454 Life Sciences sequencing platform, an Ion Torrent sequencing platform, a Helicos sequencing platform, or a nanopore sequencing platform. In some cases, a sequencing platform may comprise a 454 Life Sciences sequencing platform. In some cases, a sequencing platform may comprise an ABI SOliD sequencing platform. In some cases, a sequencing platform may comprise a Helicos sequencing platform. In some cases, a sequencing platform may comprise an Illumina sequencing platform. In some cases, a sequencing platform may comprise an Ion Torrent sequencing platform. In some cases, a sequencing platform may comprise a nanopore sequencing platform. In some cases, a sequencing platform may comprise a Pacific Biosciences sequencing platform. In some cases, a sequencing reaction may comprise a first-generation sequencing reaction, a second-generation sequencing reaction, or a third-generation sequencing reaction. In some cases, a sequencing reaction may comprise a first-generation sequencing reaction. In some cases, a sequencing reaction may comprise a second-generation sequencing reaction. In some cases, a sequencing reaction may comprise a third-generation sequencing reaction.

In some instances, a sequencing probe may be configured to be used to sequence a substrate nucleic acid, a complement sequence of a substrate nucleic acid, or derivatives thereof. In some instances, a sequencing probe may comprise about 10 nucleotides in length. In some cases, a sequencing probe may comprise about 11 nucleotides in length. In some cases, a sequencing probe may comprise about 12 nucleotides in length. In some cases, a sequencing probe may comprise about 13 nucleotides in length. In some cases, a sequencing probe may comprise about 14 nucleotides in length. In some cases, a sequencing probe may comprise about 15 nucleotides in length. In some cases, a sequencing probe may comprise about 16 nucleotides in length. In some cases, a sequencing probe may comprise about 17 nucleotides in length. In some cases, a sequencing probe may comprise about 18 nucleotides in length. In some cases, a sequencing probe may comprise about 19 nucleotides in length. In some cases, a sequencing probe may comprise about 20 nucleotides in length. In some cases, a sequencing probe may comprise about 21 nucleotides in length. In some cases, a sequencing probe may comprise about 22 nucleotides in length. In some cases, a sequencing probe may comprise about 23 nucleotides in length. In some cases, a sequencing probe may comprise about 24 nucleotides in length. In some cases, a sequencing probe may comprise about 25 nucleotides in length. In some cases, a sequencing probe may comprise about 26 nucleotides in length. In some cases, a sequencing probe may comprise about 27 nucleotides in length. In some cases, a sequencing probe may comprise about 28 nucleotides in length. In some cases, a sequencing probe may comprise about 29 nucleotides in length. In some cases, a sequencing probe may comprise about 30 nucleotides in length. In some cases, a sequencing probe may comprise from about 10 nucleotides in length to about 15 nucleotides in length. In some cases, a sequencing probe may comprise from about 10 nucleotides in length to about 20 nucleotides in length. In some cases, a sequencing probe may comprise from about 10 nucleotides in length to about 25 nucleotides in length. In some cases, a sequencing probe may comprise from about 10 nucleotides in length to about 30 nucleotides in length.

In some instances, a sequence of a virus may be compared to a reference genome to identify a mutation or a difference in nucleotide. For example, a sequence of a virus may be aligned to a reference genome. The difference between the sequence of the virus and the reference genome may be identified.

In some instances, a substrate nucleic acid may be detected without determining the sequence of the amplicon. In some cases, a substrate nucleic acid may be detected by a quantitative PCR or a real time PCR. In some cases, a substrate nucleic acid may be detected by a Southern blot. In other cases, a substrate nucleic acid may also be detected by a Northern blot. In some cases, a substrate nucleic acid may be detected by gel electrophoresis. In one case, gel electrophoresis may comprise agarose electrophoresis. In another case, capillary gel electrophoresis may be used to detect a substrate nucleic acid. In one case, a substrate nucleic acid may be detected by UV absorbance. In another cases, a substrate nucleic acid may be detected by a fluorescent dye. One such dye, in some cases, may comprise PicoGreen or SYBRGreen. In some cases, a substrate nucleic acid may be detected by diphenylamine.

### Virus Reference Genome

In some instances, a reference genome of a virus may comprise a reference genome of a virus from family Coronaviridae. In some cases, a reference genome of a virus may comprise a reference genome of a virus from subfamily Orthocoronavirinae. In some cases, a reference genome of a virus may comprise a reference genome of a virus from species coronavirus. In some cases, a reference genome of a virus may comprise a reference genome of a Severe acute respiratory syndrome-related coronavirus. In some cases, a reference genome of a virus may comprise a reference genome of Severe acute respiratory syndrome coronavirus 2. In some cases, a reference genome of a virus may comprise a reference genome of a Severe acute respiratory syndrome coronavirus. In some cases, a reference genome of a virus may comprise a reference genome of Middle East respiratory syndrome-related coronavirus. In some cases, a reference genome of a virus may comprise a reference genome of Human coronavirus OC43. In some cases, a reference genome of a virus may comprise a reference genome of Human coronavirus HKU1. In some cases, a reference genome of a virus may comprise a reference genome of Human coronavirus 229E. In some cases, a reference genome of a virus may comprise a reference genome of Human coronavirus NL63. In some cases, a reference genome of a virus may comprise a reference genome of Severe acute respiratory syndrome coronavirus 2, Severe acute respiratory syndrome coronavirus, Middle East respiratory syndrome-related coronavirus, Human coronavirus OC43, Human coronavirus HKU1, Human coronavirus 229E, or Human coronavirus NL63.

In some instances, a reference genome of a virus may comprise the sequence of NC_045512.2.

In some instances, a reference genome of a virus may comprise a reference genome of a Severe acute respiratory syndrome coronavirus lineage. In some cases, a lineage of a virus may comprise a substantial sequence identity of viral structural proteins encoded by viral structural genes. In some cases, a substantial sequence identity may comprise at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or more than at least 99% sequence identity. In some cases, a viral structural gene may comprise a Spike gene, a Nucleocapsid gene, a Membrane glycoprotein M gene, or an Envelope gene of a virus. In some cases, a viral structural gene may also comprise hemagglutinin-esterase.

In some cases, a reference genome of a virus may comprise a reference genome of a Severe acute respiratory syndrome coronavirus A.1, A.2, A.3, A.4, A.5, A.6, B.1, B.2, B.3, B.4, B.5, B.6, B.7, B.8, B.9, B.10, B.11, B.12, B.13, B.14, B.15, or B.16. In some cases, a reference genome of a virus may comprise a reference genome of a Severe acute respiratory syndrome coronavirus A.1. In some cases, a reference genome of a virus may comprise a reference genome of a Severe acute respiratory syndrome coronavirus A.2. In some cases, a reference genome of a virus may comprise a reference genome of a Severe acute respiratory syndrome coronavirus A.3. In some cases, a reference genome of a virus may comprise a reference genome of a Severe acute respiratory syndrome coronavirus A.4. In some cases, a reference genome of a virus may comprise a reference genome of a Severe acute respiratory syndrome coronavirus A.5. In some cases, a reference genome of a virus may comprise a reference genome of a Severe acute respiratory syndrome coronavirus A.6. In some cases, a reference genome of a virus may comprise a reference genome of a Severe acute respiratory syndrome coronavirus B.1. In some cases, a reference genome of a virus may comprise a reference genome of a Severe acute respiratory syndrome coronavirus B.2. In some cases, a reference genome of a virus may comprise a reference genome of a Severe acute respiratory syndrome coronavirus B.3. In some cases, a reference genome of a virus may comprise a reference genome of a Severe acute respiratory syndrome coronavirus B.4. In some cases, a reference genome of a virus may comprise a reference genome of a Severe acute respiratory syndrome coronavirus B.5. In some cases, a reference genome of a virus may comprise a reference genome of a Severe acute respiratory syndrome coronavirus B.6. In some cases, a reference genome of a virus may comprise a reference genome of a Severe acute respiratory syndrome coronavirus B.7. In some cases, a reference genome of a virus may comprise a reference genome of a Severe acute respiratory syndrome coronavirus B.8. In some cases, a reference genome of a virus may comprise a reference genome of a Severe acute respiratory syndrome coronavirus B.9. In some cases, a reference genome of a virus may comprise a reference genome of a Severe acute respiratory syndrome coronavirus B.10. In some cases, a reference genome of a virus may comprise a reference genome of a Severe acute respiratory syndrome coronavirus B.11. In some cases, a reference genome of a virus may comprise a reference genome of a Severe acute respiratory syndrome coronavirus B.12. In some cases, a reference genome of a virus may comprise a reference genome of a Severe acute respiratory syndrome coronavirus B.13. In some cases, a reference genome of a virus may comprise a reference genome of a Severe acute respiratory syndrome coronavirus B.14. In some cases, a reference genome of a virus may comprise a reference genome of a Severe acute respiratory syndrome coronavirus B.15. In some cases, a reference genome of a virus may comprise a reference genome of a Severe acute respiratory syndrome coronavirus B.16. In other cases, a reference genome of a virus may comprise a reference genome of a Severe acute respiratory syndrome coronavirus lineage different from the lineages thereof.

### Detecting Virus Variants

In some instances, a virus may comprise a virus variant. In some cases, a virus variant may be determined by the methods and compositions described in this disclosure. In some cases, a virus variant may be identified by comparing or aligning the sequences generated from the genome of the virus variant with a reference genome of a virus.

In some cases, a virus variant may be a version of virus with a substantial difference in the nucleotide sequence in the genome, a particular set of genes, or a gene. The genome may comprise any genomes described elsewhere in this disclosure. The particular set of genes or the gene may comprise any genes described elsewhere in this disclosure. The substantial difference in the nucleotide sequence may comprise a difference of about 1 nucleotide, about 2 nucleotides, about 3 nucleotides, about 4 nucleotides, about 5 nucleotides, about 6 nucleotides, about 7 nucleotides, about 8 nucleotides, about 9 nucleotides, about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1000 nucleotides, about 2000 nucleotides, about 3000 nucleotides, about 4000 nucleotides, about 5000 nucleotides, about 6000 nucleotides, about 7000 nucleotides, about 8000 nucleotides, about 9000 nucleotides, or about 10000 nucleotides.

In some instances, a Severe acute respiratory syndrome coronavirus variant may comprise B.1.1.7, B.1.1.207, Cluster 5, B.1.351, P.1, B.1525, NS3, WIV04/2019, or CAL.20C. In some cases, a Severe acute respiratory syndrome coronavirus variant may comprise B.1.1.7. In some cases, a Severe acute respiratory syndrome coronavirus variant may comprise B.1.1.207. In some cases, a Severe acute respiratory syndrome coronavirus variant may comprise Cluster 5. In some cases, a Severe acute respiratory syndrome coronavirus variant may comprise B.1.351. In some cases, a Severe acute respiratory syndrome coronavirus variant may comprise P.1. In some cases, a Severe acute respiratory syndrome coronavirus variant may comprise B.1525. In some cases, a Severe acute respiratory syndrome coronavirus variant may comprise NS3. In some cases, a Severe acute respiratory syndrome coronavirus variant may comprise WIV04/2019. In some cases, a Severe acute respiratory syndrome coronavirus variant may comprise CAL.20C. In other cases, a Severe acute respiratory syndrome coronavirus variant may comprise a variant different from the variants thereof.

In some instances, a Severe acute respiratory syndrome coronavirus variant may comprise a K417N mutation in a Spike protein encoded by a Spike gene. In some cases, a Severe acute respiratory syndrome coronavirus variant may comprise a K417T mutation in a Spike protein encoded by a Spike gene. In some cases, a Severe acute respiratory syndrome coronavirus variant may comprise a E484K mutation in a Spike protein encoded by a Spike gene. In some cases, a Severe acute respiratory syndrome coronavirus variant may comprise a N501Y mutation in a Spike protein encoded by a Spike gene. In some cases, a Severe acute respiratory syndrome coronavirus variant may comprise a D641G mutation in a Spike protein encoded by a Spike gene. In some cases, a Severe acute respiratory syndrome coronavirus variant may comprise a K417N mutation, a K417T mutation, a E484K mutation, a N501Y mutation, or a D641G mutation in a Spike protein encoded by a Spike gene of the virus. In some cases, a Severe acute respiratory syndrome coronavirus variant may also comprise a K417N mutation, a K417T mutation, a E484K mutation, a E484Q mutation, a N501Y mutation, a L452R mutation, a N440K mutation, a S447G mutation, a S447N mutation, a P681H mutation, a P681R mutation, a A701V mutation, a A570D mutation, a deletion of the 69^{th} or 70^{th} amino acid, a D641G mutation, or a Q613H mutation in a Spike protein encoded by a Spike gene of the virus. In some instances, a Severe acute respiratory syndrome coronavirus variant may comprise a K417N mutation in a Spike protein encoded by a Spike gene of the virus. In some cases, a Severe acute respiratory syndrome coronavirus variant may comprise a K417T mutation in a Spike protein encoded by a Spike gene of the virus. In some cases, a Severe acute respiratory syndrome coronavirus variant may comprise a E484K mutation in a Spike protein encoded by a Spike gene of the virus. In some cases, a Severe acute respiratory syndrome coronavirus variant may comprise a N501Y mutation in a Spike protein encoded by a Spike gene of the virus. In some cases, a Severe acute respiratory syndrome coronavirus variant may comprise a D641G mutation in a Spike protein encoded by a Spike gene of the virus. In some cases, a Severe acute respiratory syndrome coronavirus variant may comprise a E484Q mutation in a Spike protein encoded by a Spike gene of the virus. In some cases, a Severe acute respiratory syndrome coronavirus variant may comprise a L452R mutation in a Spike protein encoded by a Spike gene of the virus. In some cases, a Severe acute respiratory syndrome coronavirus variant may comprise a N440K mutation in a Spike protein encoded by a Spike gene of the virus. In some cases, a Severe acute respiratory syndrome coronavirus variant may comprise a S447G mutation in a Spike protein encoded by a Spike gene of the virus. In some cases, a Severe acute respiratory syndrome coronavirus variant may comprise a S447N mutation in a Spike protein encoded by a Spike gene of the virus. In some cases, a Severe acute respiratory syndrome coronavirus variant may comprise a P681H mutation in a Spike protein encoded by a Spike gene of the virus. In some cases, a Severe acute respiratory syndrome coronavirus variant may comprise a P681R mutation in a Spike protein encoded by a Spike gene of the virus. In some cases, a Severe acute respiratory syndrome coronavirus variant may comprise a A701V mutation in a Spike protein encoded by a Spike gene of the virus. In some cases, a Severe acute respiratory syndrome coronavirus variant may comprise a A570D mutation in a Spike protein encoded by a Spike gene of the virus. In some cases, a Severe acute respiratory syndrome coronavirus variant may comprise a Q613H mutation in a Spike protein encoded by a Spike gene of the virus. In some cases, a Severe acute respiratory syndrome coronavirus variant may comprise a deletion of the 69^{th} or 70^{th} amino acid in a Spike protein encoded by a Spike gene of the virus.

In some instances, a method for detecting virus sequences may comprise providing a report. In some cases, a report may comprise a determination of a presence or an absence of sequences of a virus in a sample or a presence or an absence of sequences of a virus variant of a virus in a sample. In some cases, a report may comprise a determination of a presence or an absence of sequences of a virus in a sample. In some cases, a report may comprise a determination of a presence or an absence of sequences of a virus variant of a virus in a sample. In some cases, a report may comprise a determination of a presence or an absence of sequences of a virus in a sample and a presence or an absence of sequences of a virus variant of a virus in a sample. In some cases, a determination of a presence or an absence of sequences of a virus in a sample or a presence or an absence of sequences of a virus variant of a virus in a sample may comprise a probability of the confidence level of the determination. The probability may depend on the accuracy of the sequencing of sequences of a virus or a virus variant. In some cases, the probability may be calculated using method described in Example 2.

In some instances, a genome of a wildtype strain of SARS-CoV-2 may comprise the sequence of any one of SEQ ID NOs. 29-32. In some instances, a genome of a wildtype strain of SARS-CoV-2 may comprise the sequence of SED ID NO: 29. In some instances, a genome of a wildtype strain of SARS-CoV-2 may comprise the sequence of SED ID NO: 30. In some instances, a genome of a wildtype strain of SARS-CoV-2 may comprise the sequence of SED ID NO: 31. In some instances, a genome of a wildtype strain of SARS-CoV-2 may comprise the sequence of SED ID NO: 32. In some instances, a genome of a wildtype strain of SARS-CoV-2 may not comprise a variation or mutation in the sequence of SED ID NOs: 29-32. In some instances, a genome of a wildtype strain of SARS-CoV-2 may not comprise a variation or mutation in the sequence of SED ID NO: 29. In some instances, a genome of a wildtype strain of SARS-CoV-2 may not comprise a variation or mutation in the sequence of SED ID NO: 30. In some instances, a genome of a wildtype strain of SARS-CoV-2 may not comprise a variation or mutation in the sequence of SED ID NO: 31. In some instances, a genome of a wildtype strain of SARS-CoV-2 may not comprise a variation or mutation in the sequence of SED ID NO: 32.

In some instance, a genome of a variant of SARS-CoV-2 may comprise a variation or mutation in the sequence of any one of SED ID NOs: 29-32. In some instances, a genome of a variant of SARS-CoV-2 may comprise a variation or mutation in the sequence of any one of SED ID NO: 29. In some instances, a genome of a variant of SARS-CoV-2 may comprise a variation or mutation in the sequence of any one of SED ID NO: 30. In some instances, a genome of a variant of SARS-CoV-2 may comprise a variation or mutation in the sequence of any one of SED ID NO: 31. In some instances, a genome of a variant of SARS-CoV-2 may comprise a variation or mutation in the sequence of any one of SED ID NO: 32.

In some instances, a genome of a G-clade variant of SARS-CoV-2 may comprise a variation or mutation in the sequence of SED ID NO: 29 or 31. In some instances, a genome of a G-clade variant of SARS-CoV-2 may comprise a variation or mutation in the sequence of SED ID NO: 29. In some instances, a genome of a G-clade variant of SARS-CoV-2 may comprise a variation or mutation in the sequence of SED ID NO: 31. In some cases, the variation or mutation of a G-clade variant of SARS-CoV-2 may comprise nucleotides that encode a Glycine (G) at a position corresponding to position 614 of a sequence of SEQ ID NO. 29.

In some cases, when a sequence of a substrate nucleic acid of a plurality of template nucleotide sequences of a virus of a sample comprises the variation or mutation that comprise nucleotides that encode a Glycine (G) at a position corresponding to position 614 of SEQ ID NO. 29, the virus is a G-clade variant of SARS-CoV-2.

In some instances, a genome of an Alpha variant of SARS-CoV-2 may comprise a variation or mutation in the sequence of SED ID NO: 29 or 31. In some instances, a genome of an Alpha variant of SARS-CoV-2 may comprise a variation or mutation in the sequence of SED ID NO: 29. In some instances, a genome of an Alpha variant of SARS-CoV-2 may comprise a variation or mutation in the sequence of SED ID NO: 31. In some cases, the variation or mutation of an Alpha variant of SARS-CoV-2 may comprise nucleotides that encode a Glycine (G) at a position corresponding to position 614 of a sequence of SEQ ID NO. 29, a deletion of nucleotides that encode an amino acid at a position corresponding to position 144 of a sequence of SEQ ID NO: 29, or nucleotides that encode a Tyrosine (Y) at a position corresponding to position 501 of a sequence of SEQ ID NO: 29. In some cases, the variation or mutation of an Alpha variant of SARS-CoV-2 may comprise nucleotides that encode a Glycine (G) at a position corresponding to position 614 of a sequence of SEQ ID NO. 29. In some cases, the variation or mutation of an Alpha variant of SARS-CoV-2 may comprise a deletion of nucleotides that encode an amino acid at a position corresponding to position 144 of a sequence of SEQ ID NO: 29. In some cases, the variation or mutation of an Alpha variant of SARS-CoV-2 may comprise nucleotides that encode a Tyrosine (Y) at a position corresponding to position 501 of a sequence of SEQ ID NO: 29. In some cases, the variation or mutation of an Alpha variant of SARS-CoV-2 may comprise nucleotides that encode a Glycine (G) at a position corresponding to position 614 of a sequence of SEQ ID NO. 29, a deletion of nucleotides that encode an amino acid at a position corresponding to position 144 of a sequence of SEQ ID NO: 29, and nucleotides that encode a Tyrosine (Y) at a position corresponding to position 501 of a sequence of SEQ ID NO: 29. In some cases, the variation or mutation of an Alpha variant of SARS-CoV-2 may consist of nucleotides that encode a Glycine (G) at a position corresponding to position 614 of a sequence of SEQ ID NO. 29, a deletion of nucleotides that encode an amino acid at a position corresponding to position 144 of a sequence of SEQ ID NO: 29, and nucleotides that encode a Tyrosine (Y) at a position corresponding to position 501 of a sequence of SEQ ID NO: 29.

In some cases, when a sequence of a substrate nucleic acid of a plurality of template nucleotide sequences of a virus of a sample comprises the variation or mutation that comprise nucleotides that encode a Glycine (G) at a position corresponding to position 614 of a sequence of SEQ ID NO. 29, a deletion of nucleotides that encode an amino acid at a position corresponding to position 144 of a sequence of SEQ ID NO: 29, or nucleotides that encode a Tyrosine (Y) at a position corresponding to position 501 of a sequence of SEQ ID NO: 29, the virus is an Alpha variant of SARS-CoV-2. In some cases, when a sequence of a substrate nucleic acid of a plurality of template nucleotide sequences of a virus of a sample comprises the variation or mutation that comprise nucleotides that encode a Glycine (G) at a position corresponding to position 614 of a sequence of SEQ ID NO. 29, the virus is an Alpha variant of SARS-CoV-2. In some cases, when a sequence of a substrate nucleic acid of a plurality of template nucleotide sequences of a virus of a sample comprises the variation or mutation that comprise a deletion of nucleotides that encode an amino acid at a position corresponding to position 144 of a sequence of SEQ ID NO: 29, the virus is an Alpha variant of SARS-CoV-2. In some cases, when a sequence of a substrate nucleic acid of a plurality of template nucleotide sequences of a virus of a sample comprises the variation or mutation that comprise nucleotides that encode a Tyrosine (Y) at a position corresponding to position 501 of a sequence of SEQ ID NO: 29, the virus is an Alpha variant of SARS-CoV-2. In some cases, when a sequence of a substrate nucleic acid of a plurality of template nucleotide sequences of a virus of a sample comprises the variation or mutation that comprise nucleotides that encode a Glycine (G) at a position corresponding to position 614 of a sequence of SEQ ID NO. 29, a deletion of nucleotides that encode an amino acid at a position corresponding to position 144 of a sequence of SEQ ID NO: 29, and nucleotides that encode a Tyrosine (Y) at a position corresponding to position 501 of a sequence of SEQ ID NO: 29, the virus is an Alpha variant of SARS-CoV-2. In some cases, when a sequence of a substrate nucleic acid of a plurality of template nucleotide sequences of a virus of a sample comprises the variation or mutation that consists of nucleotides that encode a Glycine (G) at a position corresponding to position 614 of a sequence of SEQ ID NO. 29, a deletion of nucleotides that encode an amino acid at a position corresponding to position 144 of a sequence of SEQ ID NO: 29, and nucleotides that encode a Tyrosine (Y) at a position corresponding to position 501 of a sequence of SEQ ID NO: 29, the virus is an Alpha variant of SARS-CoV-2.

In some instances, a genome of a Delta variant of SARS-CoV-2 may comprise a variation or mutation in the sequence of SED ID NO: 29 or 31. In some instances, a genome of a Delta variant of SARS-CoV-2 may comprise a variation or mutation in the sequence of SED ID NO: 29. In some instances, a genome of a Delta variant of SARS-CoV-2 may comprise a variation or mutation in the sequence of SED ID NO: 31.

In some cases, the variation or mutation of a Delta variant of SARS-CoV-2 may comprise nucleotides that encode a Aspartic acid (D) at a position corresponding to position 142 of a sequence of SEQ ID NO: 29, a deletion of nucleotides that encode an amino acid at a position corresponding to position 157 of a sequence of SEQ ID NO: 29, a deletion of nucleotides that encode an amino acid at a position corresponding to position 158 of a sequence of SEQ ID NO: 29, nucleotides that encode an Arginine (R) at a position corresponding to position 452 of SEQ ID NO: 29, nucleotides that encode an Lysine (K) at a position corresponding to position 478 of a sequence of SEQ ID NO: 29, or nucleotides that encode a G at a position corresponding to position 614 of a sequence of SEQ ID NO: 29. In some cases, the variation or mutation of a Delta variant of SARS-CoV-2 may comprise nucleotides that encode a Aspartic acid (D) at a position corresponding to position 142 of a sequence of SEQ ID NO: 29, a deletion of nucleotides that encode an amino acid at a position corresponding to position 157 of a sequence of SEQ ID NO: 29, a deletion of nucleotides that encode an amino acid at a position corresponding to position 158 of a sequence of SEQ ID NO: 29, nucleotides that encode an Arginine (R) at a position corresponding to position 452 of SEQ ID NO: 29, nucleotides that encode an Lysine (K) at a position corresponding to position 478 of a sequence of SEQ ID NO: 29, and nucleotides that encode a G at a position corresponding to position 614 of a sequence of SEQ ID NO: 29. In some cases, the variation or mutation of a Delta variant of SARS-CoV-2 may consist of nucleotides that encode a Aspartic acid (D) at a position corresponding to position 142 of a sequence of SEQ ID NO: 29, a deletion of nucleotides that encode an amino acid at a position corresponding to position 157 of a sequence of SEQ ID NO: 29, a deletion of nucleotides that encode an amino acid at a position corresponding to position 158 of a sequence of SEQ ID NO: 29, nucleotides that encode an Arginine (R) at a position corresponding to position 452 of SEQ ID NO: 29, nucleotides that encode an Lysine (K) at a position corresponding to position 478 of a sequence of SEQ ID NO: 29, or nucleotides that encode a G at a position corresponding to position 614 of a sequence of SEQ ID NO: 29. In some cases, the variation or mutation of a Delta variant of SARS-CoV-2 may comprise nucleotides that encode a Aspartic acid (D) at a position corresponding to position 142 of a sequence of SEQ ID NO: 29. In some cases, the variation or mutation of a Delta variant of SARS-CoV-2 may comprise a deletion of nucleotides that encode an amino acid at a position corresponding to position 157 of a sequence of SEQ ID NO: 29. In some cases, the variation or mutation of a Delta variant of SARS-CoV-2 may comprise a deletion of nucleotides that encode an amino acid at a position corresponding to position 158 of a sequence of SEQ ID NO: 29. In some cases, the variation or mutation of a Delta variant of SARS-CoV-2 may comprise nucleotides that encode an Arginine (R) at a position corresponding to position 452 of SEQ ID NO: 29. In some cases, the variation or mutation of a Delta variant of SARS-CoV-2 may comprise nucleotides that encode an Lysine (K) at a position corresponding to position 478 of a sequence of SEQ ID NO: 29. In some cases, the variation or mutation of a Delta variant of SARS-CoV-2 may comprise nucleotides that encode a G at a position corresponding to position 614 of a sequence of SEQ ID NO: 29.

In some cases, when a sequence of a substrate nucleic acid of a plurality of template nucleotide sequences of a virus of a sample comprises the variation or mutation that comprises nucleotides that encode a Aspartic acid (D) at a position corresponding to position 142 of a sequence of SEQ ID NO: 29, a deletion of nucleotides that encode an amino acid at a position corresponding to position 157 of a sequence of SEQ ID NO: 29, a deletion of nucleotides that encode an amino acid at a position corresponding to position 158 of a sequence of SEQ ID NO: 29, nucleotides that encode an Arginine (R) at a position corresponding to position 452 of SEQ ID NO: 29, nucleotides that encode an Lysine (K) at a position corresponding to position 478 of a sequence of SEQ ID NO: 29, or nucleotides that encode a G at a position corresponding to position 614 of a sequence of SEQ ID NO: 29, the virus is a Delta variant of SARS-CoV-2. In some cases, when a sequence of a substrate nucleic acid of a plurality of template nucleotide sequences of a virus of a sample comprises the variation or mutation that comprises nucleotides that encode a Aspartic acid (D) at a position corresponding to position 142 of a sequence of SEQ ID NO: 29, a deletion of nucleotides that encode an amino acid at a position corresponding to position 157 of a sequence of SEQ ID NO: 29, a deletion of nucleotides that encode an amino acid at a position corresponding to position 158 of a sequence of SEQ ID NO: 29, nucleotides that encode an Arginine (R) at a position corresponding to position 452 of SEQ ID NO: 29, nucleotides that encode an Lysine (K) at a position corresponding to position 478 of a sequence of SEQ ID NO: 29, and nucleotides that encode a G at a position corresponding to position 614 of a sequence of SEQ ID NO: 29, the virus is a Delta variant of SARS-CoV-2. In some cases, when a sequence of a substrate nucleic acid of a plurality of template nucleotide sequences of a virus of a sample comprises the variation or mutation that consists of nucleotides that encode a Aspartic acid (D) at a position corresponding to position 142 of a sequence of SEQ ID NO: 29, a deletion of nucleotides that encode an amino acid at a position corresponding to position 157 of a sequence of SEQ ID NO: 29, a deletion of nucleotides that encode an amino acid at a position corresponding to position 158 of a sequence of SEQ ID NO: 29, nucleotides that encode an Arginine (R) at a position corresponding to position 452 of SEQ ID NO: 29, nucleotides that encode an Lysine (K) at a position corresponding to position 478 of a sequence of SEQ ID NO: 29, and nucleotides that encode a G at a position corresponding to position 614 of a sequence of SEQ ID NO: 29, the virus is a Delta variant of SARS-CoV-2. In some cases, when a sequence of a substrate nucleic acid of a plurality of template nucleotide sequences of a virus of a sample comprises the variation or mutation that comprises nucleotides that encode a Aspartic acid (D) at a position corresponding to position 142 of a sequence of SEQ ID NO: 29, the virus is a Delta variant of SARS-CoV-2. In some cases, when a sequence of a substrate nucleic acid of a plurality of template nucleotide sequences of a virus of a sample comprises the variation or mutation that comprises a deletion of nucleotides that encode an amino acid at a position corresponding to position 157 of a sequence of SEQ ID NO: 29, the virus is a Delta variant of SARS-CoV-2. In some cases, when a sequence of a substrate nucleic acid of a plurality of template nucleotide sequences of a virus of a sample comprises the variation or mutation that comprises a deletion of nucleotides that encode an amino acid at a position corresponding to position 158 of a sequence of SEQ ID NO: 29, the virus is a Delta variant of SARS-CoV-2. In some cases, when a sequence of a substrate nucleic acid of a plurality of template nucleotide sequences of a virus of a sample comprises the variation or mutation that comprises nucleotides that encode an Arginine (R) at a position corresponding to position 452 of SEQ ID NO: 29, the virus is a Delta variant of SARS-CoV-2. In some cases, when a sequence of a substrate nucleic acid of a plurality of template nucleotide sequences of a virus of a sample comprises the variation or mutation that comprises nucleotides that encode an Lysine (K) at a position corresponding to position 478 of a sequence of SEQ ID NO: 29, the virus is a Delta variant of SARS-CoV-2. In some cases, when a sequence of a substrate nucleic acid of a plurality of template nucleotide sequences of a virus of a sample comprises the variation or mutation that comprises and nucleotides that encode a G at a position corresponding to position 614 of a sequence of SEQ ID NO: 29, the virus is a Delta variant of SARS-CoV-2.

In some instances, a genome of an Omicron variant of SARS-CoV-2 may comprise a variation or mutation in the sequence of SED ID NO: 29 or 31. In some instances, a genome of an Omicron variant of SARS-CoV-2 may comprise a variation or mutation in the sequence of SED ID NO: 29. In some instances, a genome of an Omicron variant of SARS-CoV-2 may comprise a variation or mutation in the sequence of SED ID NO: 31.

In some cases, the variation or mutation of an Omicron variant of SARS-CoV-2 may comprise a deletion of nucleotides that encode an amino acid at a position corresponding to position 42 of a sequence of SEQ ID NO: 29, a deletion of nucleotides that encode an amino acid at a position corresponding to position 43 of a sequence of SEQ ID NO: 29, a deletion of nucleotides that encode an amino acid at a position corresponding to position 44 of a sequence of SEQ ID NO: 29, nucleotides that encode a D at a position corresponding to position 145 of a sequence of SEQ ID NO: 29, a deletion of nucleotides that encode an amino acid at a position corresponding to position 211 of a sequence of SEQ ID NO: 29, nucleotides that encode an Isoleucine (I) at a position corresponding to position 212 of a sequence of SEQ ID NO: 29, nucleotides that encode an Asparagine (N) at a position corresponding to position 417 of a sequence of SEQ ID NO: 29, nucleotides that encode a K at a position corresponding to position 440 of a sequence of SEQ ID NO: 29, nucleotides that encode a Serine (S) at a position corresponding to position 446 of a sequence of SEQ ID NO: 29, nucleotides that encode an N at a position corresponding to position 477 of a sequence of SEQ ID NO: 29, nucleotides that encode a K at a position corresponding to position 478 of a sequence of SEQ ID NO: 29, nucleotides that encode an Alanine (A) at a position corresponding to position 484 of a sequence of SEQ ID NO: 29, nucleotides that encode an R at a position corresponding to position 493 of a sequence of SEQ ID NO: 29, nucleotides that encode a S at a position corresponding to position 496 of a sequence of SEQ ID NO: 29, nucleotides that encode an R at a position corresponding to position 498 of a sequence of SEQ ID NO: 29, nucleotides that encode a Y at a position corresponding to position 501 of a sequence of SEQ ID NO: 29, nucleotides that encode a Histidine (H) at a position corresponding to position 505 of a sequence of SEQ ID NO: 29, or nucleotides that encode a G at a position corresponding to position 614 of a sequence of SEQ ID NO: 29. In some cases, the variation or mutation of an Omicron variant of SARS-CoV-2 may comprise a deletion of nucleotides that encode an amino acid at a position corresponding to position 42 of a sequence of SEQ ID NO: 29, a deletion of nucleotides that encode an amino acid at a position corresponding to position 43 of a sequence of SEQ ID NO: 29, a deletion of nucleotides that encode an amino acid at a position corresponding to position 44 of a sequence of SEQ ID NO: 29, nucleotides that encode a D at a position corresponding to position 145 of a sequence of SEQ ID NO: 29, a deletion of nucleotides that encode an amino acid at a position corresponding to position 211 of a sequence of SEQ ID NO: 29, nucleotides that encode an Isoleucine (I) at a position corresponding to position 212 of a sequence of SEQ ID NO: 29, nucleotides that encode an Asparagine (N) at a position corresponding to position 417 of a sequence of SEQ ID NO: 29, nucleotides that encode a K at a position corresponding to position 440 of a sequence of SEQ ID NO: 29, nucleotides that encode a Serine (S) at a position corresponding to position 446 of a sequence of SEQ ID NO: 29, nucleotides that encode an N at a position corresponding to position 477 of a sequence of SEQ ID NO: 29, nucleotides that encode a K at a position corresponding to position 478 of a sequence of SEQ ID NO: 29, nucleotides that encode an Alanine (A) at a position corresponding to position 484 of a sequence of SEQ ID NO: 29, nucleotides that encode an R at a position corresponding to position 493 of a sequence of SEQ ID NO: 29, nucleotides that encode a S at a position corresponding to position 496 of a sequence of SEQ ID NO: 29, nucleotides that encode an R at a position corresponding to position 498 of a sequence of SEQ ID NO: 29, nucleotides that encode a Y at a position corresponding to position 501 of a sequence of SEQ ID NO: 29, nucleotides that encode a Histidine (H) at a position corresponding to position 505 of a sequence of SEQ ID NO: 29, and nucleotides that encode a G at a position corresponding to position 614 of a sequence of SEQ ID NO: 29. In some cases, the variation or mutation of an Omicron variant of SARS-CoV-2 may consist of a deletion of nucleotides that encode an amino acid at a position corresponding to position 42 of a sequence of SEQ ID NO: 29, a deletion of nucleotides that encode an amino acid at a position corresponding to position 43 of a sequence of SEQ ID NO: 29, a deletion of nucleotides that encode an amino acid at a position corresponding to position 44 of a sequence of SEQ ID NO: 29, nucleotides that encode a D at a position corresponding to position 145 of a sequence of SEQ ID NO: 29, a deletion of nucleotides that encode an amino acid at a position corresponding to position 211 of a sequence of SEQ ID NO: 29, nucleotides that encode an Isoleucine (I) at a position corresponding to position 212 of a sequence of SEQ ID NO: 29, nucleotides that encode an Asparagine (N) at a position corresponding to position 417 of a sequence of SEQ ID NO: 29, nucleotides that encode a K at a position corresponding to position 440 of a sequence of SEQ ID NO: 29, nucleotides that encode a Serine (S) at a position corresponding to position 446 of a sequence of SEQ ID NO: 29, nucleotides that encode an N at a position corresponding to position 477 of a sequence of SEQ ID NO: 29, nucleotides that encode a K at a position corresponding to position 478 of a sequence of SEQ ID NO: 29, nucleotides that encode an Alanine (A) at a position corresponding to position 484 of a sequence of SEQ ID NO: 29, nucleotides that encode an R at a position corresponding to position 493 of a sequence of SEQ ID NO: 29, nucleotides that encode a S at a position corresponding to position 496 of a sequence of SEQ ID NO: 29, nucleotides that encode an R at a position corresponding to position 498 of a sequence of SEQ ID NO: 29, nucleotides that encode a Y at a position corresponding to position 501 of a sequence of SEQ ID NO: 29, nucleotides that encode a Histidine (H) at a position corresponding to position 505 of a sequence of SEQ ID NO: 29, and nucleotides that encode a G at a position corresponding to position 614 of a sequence of SEQ ID NO: 29. In some cases, the variation or mutation of an Omicron variant of SARS-CoV-2 may comprise a deletion of nucleotides that encode an amino acid at a position corresponding to position 42 of a sequence of SEQ ID NO: 29. In some cases, the variation or mutation of an Omicron variant of SARS-CoV-2 may comprise a deletion of nucleotides that encode an amino acid at a position corresponding to position 43 of a sequence of SEQ ID NO: 29. In some cases, the variation or mutation of an Omicron variant of SARS-CoV-2 may comprise a deletion of nucleotides that encode an amino acid at a position corresponding to position 44 of a sequence of SEQ ID NO: 29. In some cases, the variation or mutation of an Omicron variant of SARS-CoV-2 may comprise nucleotides that encode a D at a position corresponding to position 145 of a sequence of SEQ ID NO: 29. In some cases, the variation or mutation of an Omicron variant of SARS-CoV-2 may comprise a deletion of nucleotides that encode an amino acid at a position corresponding to position 211 of a sequence of SEQ ID NO: 29. In some cases, the variation or mutation of an Omicron variant of SARS-CoV-2 may comprise nucleotides that encode an Isoleucine (I) at a position corresponding to position 212 of a sequence of SEQ ID NO: 29. In some cases, the variation or mutation of an Omicron variant of SARS-CoV-2 may comprise nucleotides that encode an Asparagine (N) at a position corresponding to position 417 of a sequence of SEQ ID NO: 29. In some cases, the variation or mutation of an Omicron variant of SARS-CoV-2 may comprise nucleotides that encode a K at a position corresponding to position 440 of a sequence of SEQ ID NO: 29. In some cases, the variation or mutation of an Omicron variant of SARS-CoV-2 may comprise nucleotides that encode a Serine (S) at a position corresponding to position 446 of a sequence of SEQ ID NO: 29. In some cases, the variation or mutation of an Omicron variant of SARS-CoV-2 may comprise nucleotides that encode an N at a position corresponding to position 477 of a sequence of SEQ ID NO: 29. In some cases, the variation or mutation of an Omicron variant of SARS-CoV-2 may comprise nucleotides that encode a K at a position corresponding to position 478 of a sequence of SEQ ID NO: 29. In some cases, the variation or mutation of an Omicron variant of SARS-CoV-2 may comprise nucleotides that encode an Alanine (A) at a position corresponding to position 484 of a sequence of SEQ ID NO: 29. In some cases, the variation or mutation of an Omicron variant of SARS-CoV-2 may comprise nucleotides that encode an R at a position corresponding to position 493 of a sequence of SEQ ID NO: 29. In some cases, the variation or mutation of an Omicron variant of SARS-CoV-2 may comprise nucleotides that encode a S at a position corresponding to position 496 of a sequence of SEQ ID NO: 29. In some cases, the variation or mutation of an Omicron variant of SARS-CoV-2 may comprise nucleotides that encode an R at a position corresponding to position 498 of a sequence of SEQ ID NO: 29. In some cases, the variation or mutation of an Omicron variant of SARS-CoV-2 may comprise nucleotides that encode a Y at a position corresponding to position 501 of a sequence of SEQ ID NO: 29. In some cases, the variation or mutation of an Omicron variant of SARS-CoV-2 may comprise nucleotides that encode a Histidine (H) at a position corresponding to position 505 of a sequence of SEQ ID NO: 29. In some cases, the variation or mutation of an Omicron variant of SARS-CoV-2 may comprise nucleotides that encode a G at a position corresponding to position 614 of a sequence of SEQ ID NO: 29.

In some cases, when a sequence of a substrate nucleic acid of a plurality of template nucleotide sequences of a virus of a sample comprises the variation or mutation that comprises a deletion of nucleotides that encode an amino acid at a position corresponding to position 42 of a sequence of SEQ ID NO: 29, a deletion of nucleotides that encode an amino acid at a position corresponding to position 43 of a sequence of SEQ ID NO: 29, a deletion of nucleotides that encode an amino acid at a position corresponding to position 44 of a sequence of SEQ ID NO: 29, nucleotides that encode a D at a position corresponding to position 145 of a sequence of SEQ ID NO: 29, a deletion of nucleotides that encode an amino acid at a position corresponding to position 211 of a sequence of SEQ ID NO: 29, nucleotides that encode an Isoleucine (I) at a position corresponding to position 212 of a sequence of SEQ ID NO: 29, nucleotides that encode an Asparagine (N) at a position corresponding to position 417 of a sequence of SEQ ID NO: 29, nucleotides that encode a K at a position corresponding to position 440 of a sequence of SEQ ID NO: 29, nucleotides that encode a Serine (S) at a position corresponding to position 446 of a sequence of SEQ ID NO: 29, nucleotides that encode an N at a position corresponding to position 477 of a sequence of SEQ ID NO: 29, nucleotides that encode a K at a position corresponding to position 478 of a sequence of SEQ ID NO: 29, nucleotides that encode an Alanine (A) at a position corresponding to position 484 of a sequence of SEQ ID NO: 29, nucleotides that encode an R at a position corresponding to position 493 of a sequence of SEQ ID NO: 29, nucleotides that encode a S at a position corresponding to position 496 of a sequence of SEQ ID NO: 29, nucleotides that encode an R at a position corresponding to position 498 of a sequence of SEQ ID NO: 29, nucleotides that encode a Y at a position corresponding to position 501 of a sequence of SEQ ID NO: 29, nucleotides that encode a Histidine (H) at a position corresponding to position 505 of a sequence of SEQ ID NO: 29, or nucleotides that encode a G at a position corresponding to position 614 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises a deletion of nucleotides that encode an amino acid at a position corresponding to position 42 of a sequence of SEQ ID NO: 29, a deletion of nucleotides that encode an amino acid at a position corresponding to position 43 of a sequence of SEQ ID NO: 29, a deletion of nucleotides that encode an amino acid at a position corresponding to position 44 of a sequence of SEQ ID NO: 29, nucleotides that encode a D at a position corresponding to position 145 of a sequence of SEQ ID NO: 29, a deletion of nucleotides that encode an amino acid at a position corresponding to position 211 of a sequence of SEQ ID NO: 29, nucleotides that encode an Isoleucine (I) at a position corresponding to position 212 of a sequence of SEQ ID NO: 29, nucleotides that encode an Asparagine (N) at a position corresponding to position 417 of a sequence of SEQ ID NO: 29, nucleotides that encode a K at a position corresponding to position 440 of a sequence of SEQ ID NO: 29, nucleotides that encode a Serine (S) at a position corresponding to position 446 of a sequence of SEQ ID NO: 29, nucleotides that encode an N at a position corresponding to position 477 of a sequence of SEQ ID NO: 29, nucleotides that encode a K at a position corresponding to position 478 of a sequence of SEQ ID NO: 29, nucleotides that encode an Alanine (A) at a position corresponding to position 484 of a sequence of SEQ ID NO: 29, nucleotides that encode an R at a position corresponding to position 493 of a sequence of SEQ ID NO: 29, nucleotides that encode a S at a position corresponding to position 496 of a sequence of SEQ ID NO: 29, nucleotides that encode an R at a position corresponding to position 498 of a sequence of SEQ ID NO: 29, nucleotides that encode a Y at a position corresponding to position 501 of a sequence of SEQ ID NO: 29, nucleotides that encode a Histidine (H) at a position corresponding to position 505 of a sequence of SEQ ID NO: 29, and nucleotides that encode a G at a position corresponding to position 614 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that consists of a deletion of nucleotides that encode an amino acid at a position corresponding to position 42 of a sequence of SEQ ID NO: 29, a deletion of nucleotides that encode an amino acid at a position corresponding to position 43 of a sequence of SEQ ID NO: 29, a deletion of nucleotides that encode an amino acid at a position corresponding to position 44 of a sequence of SEQ ID NO: 29, nucleotides that encode a D at a position corresponding to position 145 of a sequence of SEQ ID NO: 29, a deletion of nucleotides that encode an amino acid at a position corresponding to position 211 of a sequence of SEQ ID NO: 29, nucleotides that encode an Isoleucine (I) at a position corresponding to position 212 of a sequence of SEQ ID NO: 29, nucleotides that encode an Asparagine (N) at a position corresponding to position 417 of a sequence of SEQ ID NO: 29, nucleotides that encode a K at a position corresponding to position 440 of a sequence of SEQ ID NO: 29, nucleotides that encode a Serine (S) at a position corresponding to position 446 of a sequence of SEQ ID NO: 29, nucleotides that encode an N at a position corresponding to position 477 of a sequence of SEQ ID NO: 29, nucleotides that encode a K at a position corresponding to position 478 of a sequence of SEQ ID NO: 29, nucleotides that encode an Alanine (A) at a position corresponding to position 484 of a sequence of SEQ ID NO: 29, nucleotides that encode an R at a position corresponding to position 493 of a sequence of SEQ ID NO: 29, nucleotides that encode a S at a position corresponding to position 496 of a sequence of SEQ ID NO: 29, nucleotides that encode an R at a position corresponding to position 498 of a sequence of SEQ ID NO: 29, nucleotides that encode a Y at a position corresponding to position 501 of a sequence of SEQ ID NO: 29, nucleotides that encode a Histidine (H) at a position corresponding to position 505 of a sequence of SEQ ID NO: 29, and nucleotides that encode a G at a position corresponding to position 614 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises a deletion of nucleotides that encode an amino acid at a position corresponding to position 42 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises a deletion of nucleotides that encode an amino acid at a position corresponding to position 43 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises a deletion of nucleotides that encode an amino acid at a position corresponding to position 44 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode a D at a position corresponding to position 145 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises a deletion of nucleotides that encode an amino acid at a position corresponding to position 211 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode an Isoleucine (I) at a position corresponding to position 212 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode an Asparagine (N) at a position corresponding to position 417 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode a K at a position corresponding to position 440 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode a Serine (S) at a position corresponding to position 446 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode an N at a position corresponding to position 477 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode a K at a position corresponding to position 478 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode an Alanine (A) at a position corresponding to position 484 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode an R at a position corresponding to position 493 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode a S at a position corresponding to position 496 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode an R at a position corresponding to position 498 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode a Y at a position corresponding to position 501 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode a Histidine (H) at a position corresponding to position 505 of a sequence of SEQ ID NO: 29, the virus is an Omicron variant of SARS-CoV-2. In some cases, when a sequence of a substrate nucleic acid of a plurality of template nucleotide sequences of a virus of a sample comprises the variation or mutation that comprises nucleotides that encode a G at a position corresponding to position 614 of a sequence of SEQ ID NO: 29, the virus is an Omicron variant of SARS-CoV-2.

### Samples

In some instances, sequences of a virus in a sample may be identified. In some cases, a sample may be isolated from a human subject or an animal, water, a surface, droplets, or a food. A sample may be a biological sample from a subject (human or animal). In other cases, a sample is an environmental sample obtained from an inanimate source, such as water, sewage, or surfaces. In some cases, a sample may be isolated from a human subject or an animal. In some cases, a sample may be isolated from water. In some cases, a sample may be isolated from a surface. In some cases, a sample may be isolated from droplets. In some cases, a sample may be isolated from a food. The sample, in some cases, may also be obtained, extracted, purified from the animals, water, surfaces, droplets, or foods thereof.

In some cases, a sample may be isolated from drinking water or sewage water. In some cases, a sample may be isolated from drinking water. In some cases, a sample may be isolated from sewage water. In some cases, a sample may be isolated from toilet water (e.g., the water to flush a toilet).

In some instances, a sample may be isolated from a surface of an indoor compartment, a surface of a food packaging material, a surface of a mask, a surface of a medical equipment, or a surface of a furniture. In some cases, a sample may be isolated from a surface of an indoor compartment. In some cases, a sample may be isolated from a surface of a food packaging material. In some cases, a sample may be isolated from a surface of a mask. In some cases, a sample may be isolated from a surface of a medical equipment. In some cases, a sample may be isolated from a surface of a furniture. In some cases, a sample may be isolated from a surface of an elevator. In some cases, a sample may be isolated from a button of an elevator.

In some cases, a sample may be isolated from a surface of a metal, a surface of a wood, a surface of a plastic, a surface of a paper, a surface of a glass, a surface of a ceramic, a surface of a fabric, a surface of a leather, or a surface of a shoe. In some cases, a sample may be isolated from a surface of a metal. In some cases, a sample may be isolated from a surface of a wood. In some cases, a sample may be isolated from a surface of a plastic. In some cases, a sample may be isolated from a surface of a paper. In some cases, a sample may be isolated from a surface of a glass. In some cases, a sample may be isolated from a surface of a ceramic In some cases, a sample may be isolated from a surface of a fabric In some cases, a sample may be isolated from a surface of a leather In some cases, a sample may be isolated from a surface of a shoe. In some cases, a sample may be isolated from a surface of a door handle or a doorknob. In some cases, a sample may also be isolated from the surface of a container, a cutlery, a sink, a cookware, a jewelry, a coin, a bill, a chair, a table, a handle, a water bottle, a mirror, a cup, drinking glass, a window, a syringe, a glove, a coat, or any combinations thereof.

In some cases, a biological sample may comprise a blood sample, a tissue sample, a nasal swab sample, or an anal swab sample. In some cases, a biological sample may comprise a blood sample. In some cases, a biological sample may comprise a tissue sample. In some cases, a biological sample may comprise a nasal swab sample. In some cases, a biological sample may comprise an anal swab sample.

In some cases, a tissue sample may comprise a body fluid sample. In some cases, a tissue sample may comprise a muscle sample. In some cases, a tissue sample may comprise a skin sample. In some cases, a tissue sample may comprise a lung sample. In some cases, a tissue sample may comprise a brain sample. In some cases, a tissue sample may comprise a nervous system sample. In some cases, a tissue sample may comprise a gastrointestinal sample. In some cases, a tissue sample may comprise a skin sample. In some cases, a tissue sample may comprise an extraction of tissues or a group cells from a subject. In some cases, an extraction may comprise harvesting a tissue or a group of cells from a biopsy of tissues from an individual. In some cases, a biological sample may be obtained by a biopsy. In some cases, a biopsy may comprise a blood biopsy. In some cases, a biopsy may comprise a body fluid biopsy. In some cases, a biopsy may comprise a muscle biopsy. In some cases, a biopsy may comprise a skin biopsy. In some cases, a biopsy may comprise a hair biopsy. In some cases, a biopsy may comprise a lung biopsy. In some cases, a biopsy may comprise a brain biopsy. In some cases, a biopsy may comprise a nervous system biopsy. In some cases, a biopsy may comprise a gastrointestinal biopsy. In some cases, a biopsy may comprise a skin biopsy. In some cases, a biopsy may comprise a blood biopsy, a body fluid biopsy, a muscle biopsy, a lung biopsy, a brain biopsy, a nervous system biopsy, a gastrointestinal biopsy, a hair sample, or a skin biopsy. In other cases, a biopsy may also comprise a bone biopsy, a bone marrow biopsy, a breast biopsy, a liver biopsy, a prostate biopsy, a urogenital biopsy, a lymph node biopsy, a cardiac biopsy, an endometrial biopsy, an open biopsy, a sentinel lymph node biopsy, any derivatives thereof, or any combinations thereof. In some cases, a biopsy may comprise a fine needle aspiration biopsy, a core needle biopsy, a vacuum-assisted biopsy, an excisional biopsy, a shave biopsy, a punch biopsy, an endoscopic biopsy, a laparoscopic biopsy, a bone marrow aspiration biopsy, a liquid biopsy, any derivatives herein and thereof, or any combinations herein and thereof. In other cases, a biopsy may comprise an incisional biopsy or an excisional biopsy.

In some instances, a biological sample may be collected at a hospital, a clinic, a home, a virus screening site, a laboratory, or a nursing home. In some cases, a biological sample may be collected at a hospital. In some cases, a biological sample may be collected at a clinic. In some cases, a biological sample may be collected at a home. In some cases, a biological sample may be collected at a virus screening site. In some cases, a biological sample may be collected at a laboratory. In some cases, a biological sample may be collected at a nursing home.

In some cases, a subject may comprise a human subject. In some cases, a subject may comprise a mammal. In some cases, a subject may also comprise a monkey, a chimpanzee, a rat, a rabbit, a pig, a guinea pig, a horse, a dog, a cat, a cow, a goat, or a bat. A human subject may be an infant of an adult. In some cases, a subject may be healthy. In some cases, a subject may not be healthy. In some cases, a subject may have a disease. In some cases, a disease may comprise an infection of a virus described herein and thereof. In other cases, a subject may have a disease other than an infection of a virus described herein and thereof. In some cases, a subject may be hospitalized. In some cases, a subject may be treated at an intensive care unit. In some cases, a subject may be in a nursing home.

In some instances, a subject may have an infection of a virus from family Coronaviridae. In some cases, a subject may have an infection of a virus from subfamily Orthocoronavirinae. In some cases, a subject may have an infection of a virus from species coronavirus. In some cases, a subject may have an infection of a Severe acute respiratory syndrome-related coronavirus. In some cases, a subject may have an infection of SARS-CoV-2. In some cases, a subject may have an infection of a Severe acute respiratory syndrome coronavirus. In some cases, a subject may have an infection of Middle East respiratory syndrome-related coronavirus. In some cases, a subject may have an infection of Human coronavirus OC43. In some cases, a subject may have an infection of Human coronavirus HKU1. In some cases, a subject may have an infection of Human coronavirus 229E. In some cases, a subject may have an infection of Human coronavirus NL63. In some cases, a subject may have an infection of SARS-CoV-2, Severe acute respiratory syndrome coronavirus, Middle East respiratory syndrome-related coronavirus, Human coronavirus OC43, Human coronavirus HKU1, Human coronavirus 229E, or Human coronavirus NL63.

In some instances, a subject may be suspected of having an infection of a virus from family Coronaviridae. In some cases, a subject may be suspected of having an infection of a virus from subfamily Orthocoronavirinae. In some cases, a subject may be suspected of having an infection of a virus from species coronavirus. In some cases, a subject may be suspected of having an infection of a Severe acute respiratory syndrome-related coronavirus. In some cases, a subject may be suspected of having an infection of SARS-CoV-2. In some cases, a subject may be suspected of having an infection of a Severe acute respiratory syndrome coronavirus. In some cases, a subject may be suspected of having an infection of Middle East respiratory syndrome-related coronavirus. In some cases, a subject may be suspected of having an infection of Human coronavirus OC43. In some cases, a subject may be suspected of having an infection of Human coronavirus HKU1. In some cases, a subject may be suspected of having an infection of Human coronavirus 229E. In some cases, a subject may be suspected of having an infection of Human coronavirus NL63. In some cases, a subject may be suspected of having an infection of SARS-CoV-2, Severe acute respiratory syndrome coronavirus, Middle East respiratory syndrome-related coronavirus, Human coronavirus OC43, Human coronavirus HKU1, Human coronavirus 229E, or Human coronavirus NL63.

In some instances, a subject may be at risk of having an infection of a virus from family Coronaviridae. In some cases, a subject may be at risk of having an infection of a virus from subfamily Orthocoronavirinae. In some cases, a subject may be at risk of having an infection of a virus from species coronavirus. In some cases, a subject may be at risk of having an infection of a Severe acute respiratory syndrome-related coronavirus. In some cases, a subject may be at risk of having an infection of SARS-CoV-2. In some cases, a subject may be at risk of having an infection of a Severe acute respiratory syndrome coronavirus. In some cases, a subject may be at risk of having an infection of Middle East respiratory syndrome-related coronavirus. In some cases, a subject may be at risk of having an infection of Human coronavirus OC43. In some cases, a subject may be at risk of having an infection of Human coronavirus HKU1. In some cases, a subject may be at risk of having an infection of Human coronavirus 229E. In some cases, a subject may be at risk of having an infection of Human coronavirus NL63. In some cases, a subject may be at risk of having an infection of SARS-CoV-2, Severe acute respiratory syndrome coronavirus, Middle East respiratory syndrome-related coronavirus, Human coronavirus OC43, Human coronavirus HKU1, Human coronavirus 229E, or Human coronavirus NL63. In some instances, a subject may be at risk of having an infection of a virus if the subject has been in contact with a second subject having an infection of the virus, being suspected of having the infection of the virus, or at risk of having the infection of the virus.

In some cases, a subject may be in contact with a second subject if the subject and the second subject are located within less than about 50 meters, less than about 45 meters, less than about 40 meters, less than about 35 meters, less than about 30 meters, less than about 25 meters, less than about 20 meters, less than about 19 meters, less than about 18 meters, less than about 17 meters, less than about 16 meters, less than about 15 meters, less than about 14 meters, less than about 13 meters, less than about 12 meters, less than about 11 meters, less than about 10 meters, less than about 9 meters, less than about 8 meters, less than about 7 meters, less than about 6 meters, less than about 5 meters, less than about 4 meters, less than about 3 meters, less than about 2 meters, or less than about 1 meter without any barriers between the first subject and the second subject. In some cases, a subject may be in contact with a second subject if the subject and the second subject are located within less than about 50 meters, less than about 45 meters, less than about 40 meters, less than about 35 meters, less than about 30 meters, less than about 25 meters, less than about 20 meters, less than about 19 meters, less than about 18 meters, less than about 17 meters, less than about 16 meters, less than about 15 meters, less than about 14 meters, less than about 13 meters, less than about 12 meters, less than about 11 meters, less than about 10 meters, less than about 9 meters, less than about 8 meters, less than about 7 meters, less than about 6 meters, less than about 5 meters, less than about 4 meters, less than about 3 meters, less than about 2 meters, or less than about 1 meter in an indoor space.

In some instances, a subject may have a symptom associated with an infection of a virus described thereof. In some cases, a subject may have a fever, a chill, a cough, a shortness of breath or difficulty breathing, fatigue, a muscle or body ache, a headache, a new loss of taste or smell, a sore throat, a congestion or runny nose, a nausea or vomiting, a diarrhea, trouble breathing, a persistent pain or pressure in the chest, a new confusion, an inability to wake or stay awake, pale, gray, or blue-colored skin, lips, or nail beds depending on skin tone. In some cases, a subject may have a fever. In some cases, a subject may have a chill. In some cases, a subject may have a cough. In some cases, a subject may have a shortness of breath or difficulty breathing. In some cases, a subject may have fatigue. In some cases, a subject may have a muscle or body ache. In some cases, a subject may have a headache. In some cases, a subject may have a new loss of taste or smell. In some cases, a subject may have a sore throat. In some cases, a subject may have a congestion or runny nose. In some cases, a subject may have a nausea or vomiting. In some cases, a subject may have a diarrhea. In some cases, a subject may have trouble breathing. In some cases, a subject may have a persistent pain or pressure in the chest. In some cases, a subject may have a new confusion. In some cases, a subject may have an inability to wake or stay awake. In some cases, a subject may have pale, gray, or blue-colored skin, lips, or nail beds depending on skin tone.

In some cases, a subject may have at least two of: a fever, a chill, a cough, a shortness of breath or difficulty breathing, fatigue, a muscle or body ache, a headache, a new loss of taste or smell, a sore throat, a congestion or runny nose, a nausea or vomiting, a diarrhea, trouble breathing, a persistent pain or pressure in the chest, a new confusion, an inability to wake or stay awake, pale, gray, or blue-colored skin, lips, or nail beds depending on skin tone. In some cases, a subject may have at least three of: a fever, a chill, a cough, a shortness of breath or difficulty breathing, fatigue, a muscle or body ache, a headache, a new loss of taste or smell, a sore throat, a congestion or runny nose, a nausea or vomiting, a diarrhea, trouble breathing, a persistent pain or pressure in the chest, a new confusion, an inability to wake or stay awake, pale, gray, or blue-colored skin, lips, or nail beds depending on skin tone. In some cases, a subject may have at least four of: a fever, a chill, a cough, a shortness of breath or difficulty breathing, fatigue, a muscle or body ache, a headache, a new loss of taste or smell, a sore throat, a congestion or runny nose, a nausea or vomiting, a diarrhea, trouble breathing, a persistent pain or pressure in the chest, a new confusion, an inability to wake or stay awake, pale, gray, or blue-colored skin, lips, or nail beds depending on skin tone. In some cases, a subject may have at least five of: a fever, a chill, a cough, a shortness of breath or difficulty breathing, fatigue, a muscle or body ache, a headache, a new loss of taste or smell, a sore throat, a congestion or runny nose, a nausea or vomiting, a diarrhea, trouble breathing, a persistent pain or pressure in the chest, a new confusion, an inability to wake or stay awake, pale, gray, or blue-colored skin, lips, or nail beds depending on skin tone.

A subject, in some cases, may have an age of at least about 1 day old, at least about 2 days old, at least about 3 days old, at least about 4 days old, at least about 5 days old, at least about 6 days old, at least about 1 week old, at least about 2 weeks old, at least about 3 weeks old, at least about 4 weeks old, at least about 1 month old, at least about 2 months old, at least about 3 months old, at least about 4 months old, at least about 5 months old, at least about 6 months old, at least about 7 months old, at least about 8 months old, at least about 9 months old, at least about 10 months old, at least about 11 months old, at least about 1 year old, at least about 2 years old, at least about 3 years old, at least about 4 years old, at least about 5 years old, at least about 6 years old, at least about 7 years old, at least about 8 years old, at least about 9 years old, at least about 10 years old, at least about 11 years old, at least about 12 years old, at least about 13 years old, at least about 14 years old, at least about 15 years old, at least about 16 years old, at least about 17 years old, at least about 18 years old, at least about 19 years old, at least about 20 years old, at least about 21 years old, at least about 22 years old, at least about 23 years old, at least about 24 years old, at least about 25 years old, at least about 26 years old, at least about 27 years old, at least about 28 years old, at least about 29 years old, at least about 30 years old, at least about 31 years old, at least about 32 years old, at least about 33 years old, at least about 34 years old, at least about 35 years old, at least about 36 years old, at least about 37 years old, at least about 38 years old, at least about 39 years old, at least about 40 years old, at least about 41 years old, at least about 42 years old, at least about 43 years old, at least about 44 years old, at least about 45 years old, at least about 46 years old, at least about 47 years old, at least about 48 years old, at least about 49 years old, at least about 50 years old, at least about 51 years old, at least about 52 years old, at least about 53 years old, at least about 54 years old, at least about 55 years old, at least about 56 years old, at least about 57 years old, at least about 58 years old, at least about 59 years old, at least about 60 years old, at least about 61 years old, at least about 62 years old, at least about 63 years old, at least about 64 years old, at least about 65 years old, at least about 66 years old, at least about 67 years old, at least about 68 years old, at least about 69 years old, at least about 70 years old, at least about 71 years old, at least about 72 years old, at least about 73 years old, at least about 74 years old, at least about 75 years old, at least about 76 years old, at least about 77 years old, at least about 78 years old, at least about 79 years old, at least about 80 years old, at least about 81 years old, at least about 82 years old, at least about 83 years old, at least about 84 years old, at least about 85 years old, at least about 86 years old, at least about 87 years old, at least about 88 years old, at least about 89 years old, at least about 90 years old, at least about 91 years old, at least about 92 years old, at least about 93 years old, at least about 94 years old, at least about 95 years old, at least about 96 years old, at least about 97 years old, at least about 98 years old, at least about 99 years old, or at least about 100 years old.

### Compositions and Kits

In some instances, a composition may comprise any primers described elsewhere in this disclosure. In some cases, a composition may comprise any primer pairs described elsewhere in this disclosure. In some cases, a composition may comprise a mixture of a plurality of primers described elsewhere in this disclosure. In some cases, the plurality of primers may be configured to generate substrate nucleic acids. In some cases, the substrate nucleic acids may be amplification products of a plurality of template nucleotide sequences in a sample, or amplification products of complement sequences of the plurality of template nucleotide sequences, and each of the plurality of template nucleotide sequences has at least a sequence aligning to a separate portion in a reference genome of a virus. In some cases, the substrate nucleic acids are amplification products of a plurality of template nucleotide sequences in a sample, or amplification products of complement sequences of the plurality of template nucleotide sequences, and the substrate nucleic acids have less than about 1500 nucleotides in length. In some cases, the plurality of primers may also comprise at least two nucleotide sequences comprising at least about 80% sequence identity to two sequences selected from the group consisting of SEQ ID NOs: 1-28.

In some instances, a composition may comprise any probes described elsewhere in this disclosure. A probe, in some cases, may comprise a barcoding probe. In other cases, a probe may also comprise a sequencing probe. In some cases, a composition may comprise any oligonucleotides described elsewhere in this disclosure. In some instances, any of the compositions may be used to identify sequences of a virus. In some cases, any of the compositions may be used to generate substrate nucleic acids.

In some instances, a kit may comprise any of the compositions thereof. In some cases, a kit may comprise any primers described elsewhere in this disclosure. In some cases, a kit may comprise any primer pairs described elsewhere in this disclosure. In some cases, a kit may comprise a mixture of a plurality of primers described elsewhere in this disclosure. In some cases, the plurality of primers may be configured to generate substrate nucleic acids. In some cases, the substrate nucleic acids may be amplification products of a plurality of template nucleotide sequences in a sample, or amplification products of complement sequences of the plurality of template nucleotide sequences, and each of the plurality of template nucleotide sequences has at least a sequence aligning to a separate portion in a reference genome of a virus. In some cases, the substrate nucleic acids are amplification products of a plurality of template nucleotide sequences in a sample, or amplification products of complement sequences of the plurality of template nucleotide sequences, and the substrate nucleic acids have less than about 1500 nucleotides in length. In some cases, the plurality of primers may also comprise at least two nucleotide sequences comprising at least about 80% sequence identity to two sequences selected from the group consisting of SEQ ID NOs: 1-28. A probe, in some cases, may comprise a barcoding probe. In other cases, a probe may also comprise a sequencing probe. In some cases, a kit may comprise any oligonucleotides described elsewhere in this disclosure. In some instances, a kit may comprise any probes described elsewhere in this disclosure. In some instances, any of the kits may be used to identify sequences of a virus. In some cases, any of the kits may be used to generate substrate nucleic acids.

### Solid Support Beads

In some instances, a kit may comprise a solid support. In some cases, a solid support may bind to a nucleic acid molecule with a substantial affinity. In some cases, a solid support may bind to DNA or RNA with a substantial affinity. In some cases, a solid support may bind to DNA with a substantial affinity. In some cases, a solid support may bind to RNA with a substantial affinity. In some cases, a solid support may bind to DNA and RNA with a substantial affinity. A substantial affinity may comprise a dissociation constant (K_{d}) less than 1×10\hat{ }−6 M, less than 1×10\hat{ }−7 M, less than 1×10\hat{ }−8 M, less than 1×10\hat{ }−9 M, less than 1×10\hat{ }−10 M, less than 1×10\hat{ }−11 M, less than 1×10\hat{ }−12 M, less than 1×10\hat{ }−13 M, less than 1×10\hat{ }−14 M, less than 1×10\hat{ }−15 M, less than 1×10\hat{ }−16 M, less than 1×10\hat{ }−17 M, less than 1×10\hat{ }−18 M, less than 1×10\hat{ }−19 M, or less than 1×10\hat{ }−20 M. In some cases, a solid support may not bind to molecules other than a nucleic acid molecule with a substantial affinity. In some cases, a solid support may be hollow, non-porous, porous, semi-solid, solid, or any combinations thereof.

In some instances, the solid support may be a bead. In some cases, a bead may comprise a magnetic bead. In some cases, a magnetic bead may comprise superparamagnetic properties. A magnetic bead, in some cases, may comprise iron. In some cases, a magnetic bead may comprise iron oxide. In other cases, a bead may also comprise a gel bead or a hydrogel bead. In some cases, a bead may be fluidic, semi-fluidic, semi-solid, solid, or any combinations thereof.

While various embodiments of the present disclosure have been shown and described herein, it will be obvious to those skilled in the art that such embodiments are provided by way of example only. Numerous variations, changes, and substitutions may occur to those skilled in the art without departing from the present disclosure. It should be understood that various alternatives to the embodiments of the present disclosure described herein may be employed.

### EXAMPLES

These examples are provided for illustrative purposes only and not to limit the scope of the claims provided herein.

### EXAMPLE 1—Identifying Sequences of Severe Acute Respiratory Syndrome Coronavirus 2

This example illustrates exemplary methods and compositions for identifying Severe acute respiratory syndrome coronavirus 2 sequences.

### General Workflow

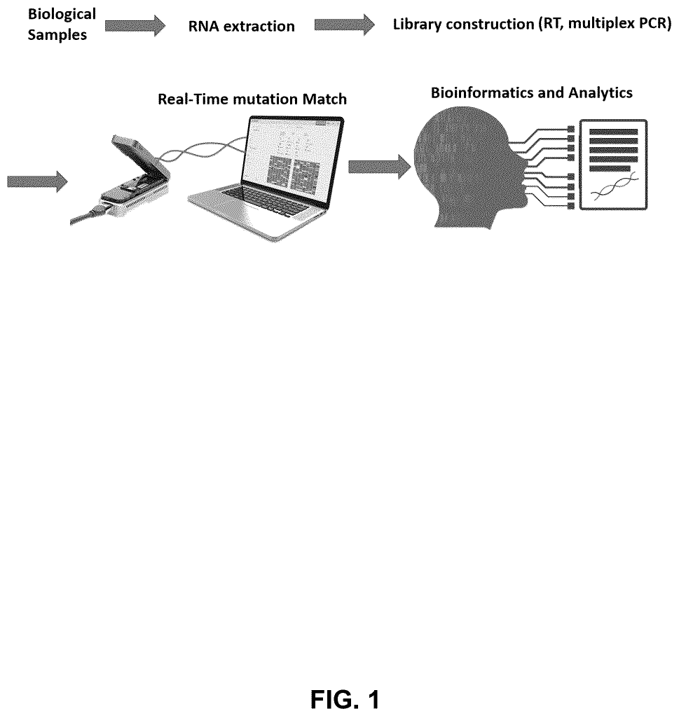

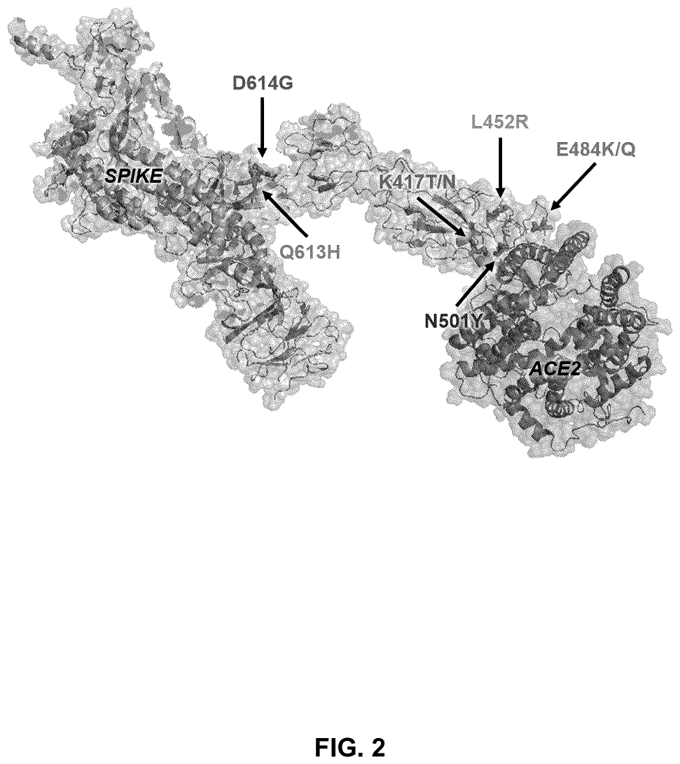

The general workflow of the for identifying SARS-CoV-2 is shown in FIG. 1: RNA is first extracted from a sample (e.g., from a patient or an environment). The RNA is converted into DNA by a reverse transcription. The converted DNA, which serves as template nucleotide sequences, is used in a multiplex PCR amplification reaction to generate substrate nucleic acids (e.g., amplicons). The substrate nucleic acids are converted into sequencing library for the subsequent sequencing reaction. The sequencing reads are compared to reference sequence for real-time mutation match and/or downstream bioinformatics and analytics. The sequences or variants of SARS-CoV-2 are identified. Some of the exemplary variants of the Spike protein are shown in FIG. 2, including, but not limited to, K417T/N, L452R, E484K/Q, N501Y, A570D, Q613H, and D614G. The K417, L452, E484, and N501 amino acids locate close to the binding interface with ACE2.

Reverse Transcription of RNA Extracted from Samples

Total RNA is extracted and purified from a nasal swab sample from patients suspected of or at risk of Severe acute respiratory syndrome coronavirus 2 infection. A pre-mix reaction for reverse transcription (RT) is assembled at 4° C. according to Table 2.

TABLE 2
A recipe for a pre-mix for reverse transcription

| Material | Volume (μl) |
|---|---|
| RNA | 11 |
| dNTP (10 mM) | 1 |
| Random hexamers (50 mM) | 1 |

The pre-mix reaction is incubated at 65° C. for 5 min and then 4° C. for at least for 1 min in a PCR machine. A secondary mix for RT according to Table 3 is added to the pre-mix to form an RT mixture.

TABLE 3
A recipe for a secondary mix for reverse transcription

| Material | Volume (μl) |
|---|---|
| SSIV buffer (5x; | 4 |
| ThermoFisher Scientific, | |
| Catalog# 18090050) | |
| DTT (100 mM) | 1 |
| RNase OUT (40 U/ml; | 1 |
| ThermoFisher Scientific, | |
| Catalog# 10777019) | |
| SuperScript IV Reverse | 1 |
| Transcriptase (200 U/ml; | |
| ThermoFisher Scientific, | |
| Catalog# 18090050) | |

The RT mixture is incubated at 23° C. for 10 minutes (min), 50° C. for 10 min, and the 80° C. for 10 min. 1 ml RNase H is added to the mixture. The RT mixture is mixed by pipetting and incubated at 37° C. for 20 min. The RT mixture might be stored at 4° C. overnight or continued with a multiplex PCR reaction.

### Multiplex PCR Reaction

Primers of SEQ ID NOs: 1-12, 14, 15, 17, and 19 are diluted in TE buffer or nuclease-free water to a concentration of 10 μM. A primer mix with equal ratio of each of the primers is prepared. A 50 μl multiplex PCR reaction mixture is set up according to Table 4.

TABLE 4
A recipe for a multiplex PCR reaction mixture

| Material | Volume (μl) |
|---|---|
| RT mixture | 10 |
| High-Fidelity 2x Master Mix | 25 |
| (New England BioLabs, | |
| Catalog# M0492L) | |
| Primer mix (final concentration: | 5 |
| 0.167 μM each primer) | |
| Nuclease-free water | 20 |

The multiplex PCR reaction mixture is incubated in the thermal cycles described in Table 5.

TABLE 5
Thermal cycles for a multiplex PCR reaction

| Cycle # | Temperature (° C.) | Time (seconds) |
|---|---|---|
| 1 | 98 | 30 |
| 2* | 98 | 10 |
| 3* | 55 | 30 |
| 4* | 72 | 15 |
| 5 | 72 | 15 |
| 6 | 4 | ∞ |

*repeated 40-45 times

### PCR Product Cleanup

The multiplex PCR reaction product after the thermal cycles is purified using the RNAClean XP beads (Beckman Coulter, Catalog #A63987). The beads are first incubated at room temperature for 10 min. 50 μl RNAClean XP beads is added to the PCR reaction product and mixed by pipetting. The mixture is transferred to a clean 1.5 ml microcentrifuge tube and incubated at room temperature for 5 min. The tube is placed on a magnet for 5 min. The supernatant is removed without disturbing the magnetic beads. The tube is kept on the magnet and washed twice with 200 μl 80% ethanol. The remaining ethanol is aspirated. The magnetic beads are air-dried for 10 min or until the remaining liquid had completely evaporated. The tube is removed from the magnet. 9 μl nuclease-free water is added to the beads. The entire content is mixed by pipetting and incubated at room temperature for 5 min. The tube is then placed on the magnet for 2-3 min. About 7 ml nuclease-free water containing DNA is aspirated without disturbing magnetic beads and transferred to a clean tube. The DNA concentration is measured using a Nanodrop.

### Nanopore Library Preparation

The barcodes of a Rapid barcoding kit (Oxford Nanopore Technologies, catalog #SQK-RBK004) are placed on ice. The other reagents of the Rapid barcoding kit and a Flow cell priming kit are thawed and stored on ice. A flongle/MinION flow cell check is performed using the MinKNOW software. The amount of DNA among all samples is maximized and normalized to a total volume of 4 μl (flongle; Oxford Nanopore Technologies, catalog #FLO-FLG001) or 8 μl (MinION; Oxford Nanopore Technologies, catalog #FLO-MIN106D) and transferred to a clean PCR tube (nuclease-free water is used to top up the volume if necessary). 1 μl (flongle) or 2 μl (MinION) barcode reagent is added to the DNA and placed on ice. The reaction mixture is incubated at 30° C. for 3 min and 80° C. for 1 min; and cooled down to 4° C. in the PCR machine. The sample is collected on ice. All barcoded samples are pooled and mixed. An equal volume of RNAXP Clean beads (the beads are incubated at room temperature for 10 min before use) is added to the pooled sample. The sample mixture is mixed by pipetting and incubated at room temperature for 5 min. The PCR tube is placed on the magnet for 5 min. The supernatant is removed without disturbing the magnet beads. The tube is kept on the magnet washed twice with 200 μl 80% ethanol and the remaining ethanol is aspirated. The tube is air-dried for 10 min or until any remaining liquid had completely evaporated. The tube is then removed from the magnet. 7 μl (flongle) or 12 μl (MinION) nuclease-free water is added to the dried reaction mixture. The entire content in the tube is mixed by pipetting and incubated at room temperature for 5 min. About 5 μl (flongle) or 10 μl (MinION) nuclease-free water containing the DNA is aspirated without disturbing the magnetic beads and transferred to a clean PCR tube. 0.5 ml (MinION) or 1 ml (MinION) RAP is added. The entire content in the tube is mixed by pipetting and incubated at room temperature for 5 min.

### Nanopore Sequencing

### Option #1: Nanopore Sequencing Using Flongle Flow Cell

117 μl FB and 3 ml FLT are mixed by pipetting to prime and load the flow cell. A DNA sequencing library is prepared in a clean tube according to the recipe of Table 6.

TABLE 6
A recipe for preparing a DNA sequencing
library using flongle flow cell

| Material | Volume (μl) |
|---|---|
| SQB | 13.5 |
| LB (Oxford Nanopore Technologies, | 11 |
| Catalog# SQK-RBK-004; mixed | |
| and vortexed immediately | |
| before use) | |
| DNA prepared with flongle reagents | 5.5 |

The prepared library is loaded to the flow cell and sequenced on the MinKNOW software using the following parameters: 1. Fast-basecalling; 2. Enable barcoding with SQK-RBK-004 if applicable. The sequencing is continued for 3-6 hours (can be continued for at most about 2 hours) or until sufficient number of reads are obtained.

### Option #2: Nanopore Sequencing Using MinION Flow Cell

30 μl FLT is added to a tube of FB and mixed by pipetting to prime and load the flow cell. The flow cell priming is performed using the manufacturer protocol. A DNA sequencing library is prepared in a clean tube according to the recipe of Table 7.

TABLE 7
A recipe for preparing a DNA sequencing
library using MinION flow cell

| Material | Volume (μl) |
|---|---|
| SQB | 34 |
| LB (Oxford Nanopore Technologies, | 25.5 |
| Catalog# SQK-RBK-004; mixed | |
| and vortexed immediately | |
| before use) | |
| DNA prepared with MinION reagents | 11 |
| Nuclease-free water | 4.5 |

The prepared library is loaded to the flow cell and sequenced on the MinKNOW software using the following parameters: 1. Fast-basecalling; 2. Enable barcoding with SQK-RBK-004 if applicable. The sequencing is continued for 1-3 hours (can be continued for at most about 30 minutes) or until sufficient number of reads are obtained.

### Data Analysis

Raw fastq files are converted and combined into a fasta format file. Cloud-server BLAST/minimap2 alignment to a reference SARS-CoV-2 genome is performed using output format “5.” For the alignment, the alignment length needed to be greater than 100 nucleotides, and the sequence homology needed to be greater than 80%. An in-house computer script is run using the alignment results of the input file to obtain COVID-19 mutant and strain information per barcode ID. A report form is generated automatically to summarize the result.

The method can also be applied to detecting sequences of other viruses described in this disclosure. Any reference genomes or primers described in this disclosure may also be used.

### EXAMPLE 2—Multiplex Polymerase Chain Reaction of Severe Acute Respiratory Syndrome Coronavirus 2 Variants

This example illustrates methods and compositions for generating multiple PCR amplification products using SARS-CoV-2 nucleic acid sequences as templates.

Six primers were chosen from SEQ ID NOs: 1-12, 14, 15, 17, and 19. Each primer was diluted in TE buffer or nuclease-free water to a concentration of 10 μM. Four primer mixes, each with equal ratio of primers, were prepared according to Table 8. Table 8 also lists the template sequence and the size of the amplification product for each pair of primer using multiplex PCR reactions.

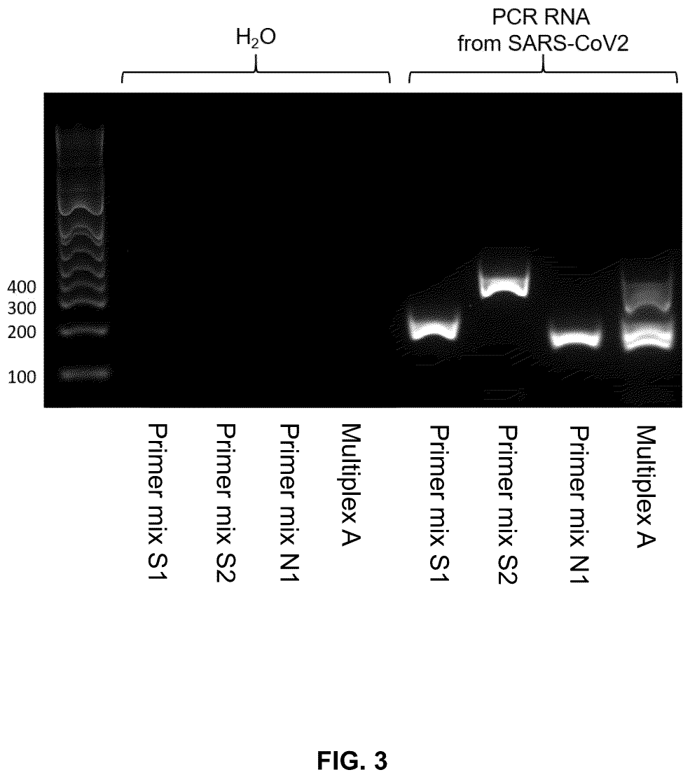

TABLE 8
Primer Mixes for Multiplex PCR Reaction in FIG. 3

| | Template Sequence Amplified | Amplification |
|---|---|---|
| Name of Primer Mixes | (Coordinates of NCBI | Product Size |
| (Primer Pair) | NC_045512.2) | (Base Pairs) |
| S1 | 23272-23477 | 206 |
| S2 | 22773-23133 | 361 |
| N1 | 28479-28651 | 173 |
| Multiplex A | 23272-23477; 22773- | 206; 361; |
| (S1 + S2 + N1) | 23133; 28479-28651 | 173 |

The multiplex primer mixes could amplify the two regions of the S genes (S1 and S2) and one region of the N gene (N1). The individual primer mixes were used as a control. A 50 μl multiplex PCR reaction mixture (multiplex A) was set up for each primer mix according to Table 4. RNA from SARS-CoV-2 infected patients were used as the template for the PCR reaction. The PCR reaction mixtures were incubated in the thermal cycles described in Table 5. As shown in FIG. 3, the amplification products of three separate portions of the genome template were amplified.

To further test different primer mixes for multiplex PCR, twelve primers were chosen from SEQ ID NOs: 1-28. Each primer was diluted in TE buffer or nuclease-free water to a concentration of 10 mM. Four primer mixes, each with equal ratio of primers, were prepared according to Table 9. Table 9 also lists the template sequence and the size of the amplification product for each pair of primer.

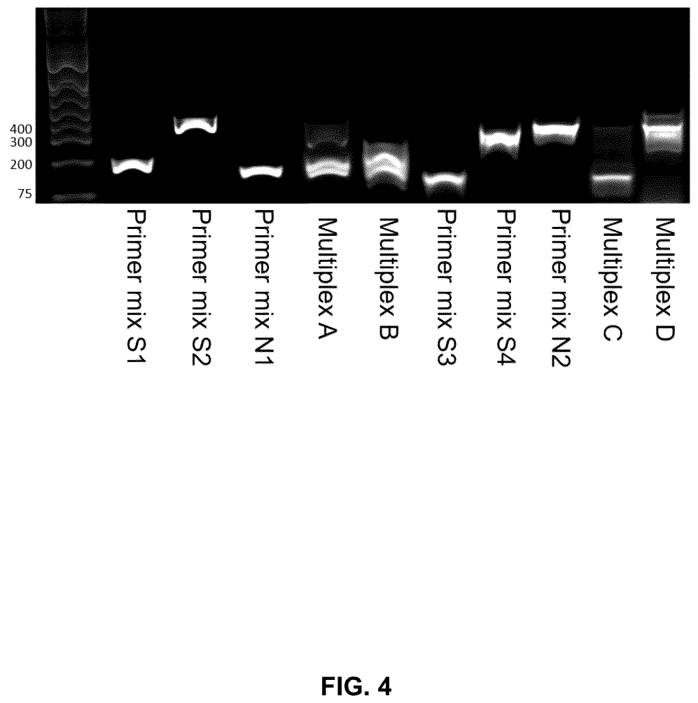

TABLE 9
Primer Mixes for Multiplex PCR Reaction in FIG. 4

| | Template Sequence Amplified | Amplification |
|---|---|---|
| Name of Primer Mixes | (Coordinates of NCBI | Product Size |
| (Primer Pair) | NC_045512.2) | (Base Pairs) |
| S1 | 23272-23477 | 206 |
| S2 | 22773-23133 | 361 |
| N1 | 28479-28651 | 173 |
| Multiplex A | 23272-23477; 22773- | 361; 206; |
| (S1 + S2 + N1) | 23133; 28479-28651 | 173 |
| Multiplex B | 23272-23477; | 206; 173 |
| (S1 + N1) | 28479-28651 | |
| S3 | 23288-23427 | 140 |
| S4 | 22589-22868 | 280 |
| N2 | 29044-29427 | 384 |
| Multiplex C | 23288-23427; 22589- | 140; 280; |
| (S3 + S4 + N2) | 22868; 29044-29427 | 384 |
| Multiplex D | 22589-22868; | 280; 384 |
| (S4 + N2) | 29044-29427 | |

The multiplex primer mixes could amplify the various regions of the S genes (S1, S2, S3, and S4) and one region of the N gene (N1 and N2). The individual primer mixes were used as a control. 50 μl multiplex PCR reaction mixtures (multiplex A, multiplex B, multiplex C, and multiplex D) were set up according to Table 4. The PCR reaction mixtures were incubated in the thermal cycles described in Table 5. As shown in FIG. 4, the amplification products of multiple separate portions of the genome template were amplified using multiplex PCR reactions.

Primer mixes with various pairs of primers or targeting/amplifying different portion of SARS-CoV-2 or different virus can also be used in the multiplex PCR reaction described thereof.

### EXAMPLE 3—Identifying Sequences of Severe Acute Respiratory Syndrome Coronavirus 2 Variants

This example illustrates an exemplary method for identifying SARS-CoV-2 infection and variants according to some embodiments of the present disclosure, using multiplex polymerase chain reaction coupled with high throughput sequencing of the amplification products. The experimental results demonstrate the feasibility and accuracy of the method provided herein. To determine the accuracy of the method of Example 1 in detecting SARS-CoV-2 variants, 500 positive reads were generated for each of a positive control (genomic RNA from COVID19, BEI, catalog #NR-55244), a negative control (saliva from healthy individuals) and 2 samples. No positive read was detected using negative control. Experiments of positive control and each sample were repeated 6 times respectively. Accuracy rate of each mutation's detection was calculated as number of hits of the base pair of each mutant divided by the number of hits of the SARS-CoV-2. The result was summarized in Table 10.

TABLE 10
Accuracy of SARS-CoV-2 Variant Detection

| Detection (SARS- | | Std | No. of |
|---|---|---|---|
| CoV-2 strains) | Accuracy | Error | Test |
| D614G | 96.59% | 0.58% | 6 |
| N501Y | 91.97% | 0.84% | 6 |
| E484A | 91.53% | 0.81% | 6 |
| K417N | 96.73% | 1.46% | 6 |
| L452R | 93.55% | 0.81% | 6 |

Each of the D164, N501, E484 and K417 variants was detected with more than 91% accuracy. These results show that the methods and compositions of Example 1 can detect mutations at D164, N501, E484 and K417 with high rate of effectiveness and accuracy. The method can also be applied to detecting sequences of other viruses described in this disclosure. Any reference genomes or primers described in this disclosure may also be used.

Therefore, the methods and compositions described in this disclosure can identify SARS-CoV-2 strains or variants with high accuracy.

### EXAMPLE 4—Identifying Sequences of Severe Acute Respiratory Syndrome Coronavirus 2 Variants from Sewage Water

This example illustrates methods and compositions for identifying SARS-CoV-2 sequences or variants present in the environment using detection of SARS-CoV-2 variants using multiplex polymerase chain reaction coupled with high throughput sequencing of the amplification products described in this disclosure.

To identify SARS-CoV-2 sequences or variants present in sewage water, RNA from sewage water samples of resident buildings were extracted, reverse transcribed, amplified in multiplexed PCR, sequenced, and analyzed as described in Example 1. Genomic RNA from COVID19 (BEI, catalog #NR-55244) was used as a positive control, and nucleic acid free water was used as a negative control. Table 11 shows a summary of the result.

TABLE 11
Identification of N501Y and D614G in sewage
water samples from residential buildings

| | | SARS-CoV-2 | SARS-CoV-2 | CT value of |
|---|---|---|---|---|
| | SARS-CoV- | variant N501Y | variant D614G | the QPCR |
| Sample | 2 present? | present? | present? | confirmation |
| Negative control | No | No | No | nd |
| Sample 0407-17 | Yes | Yes | Yes | 36 |
| Sample 0405-17 | Yes | No | Yes | 39 |
| Sample 0402-16 | No | No | No | 40 |
| Positive control | Yes | Yes | Yes | nd |

nd: not determined

SARS-CoV-2 variant D614G was detected in two of the sewage water samples, while SARS CoV-2 variant N501Y was detected in one of the sewage water samples. Consistent with the data, when the RNAs of the sewage water sample were then tested with the primers specific for the two variants in a quantitative PCR (QPCR) experiment, the samples with the variants had CT (cycle threshold) values fewer than background (CT value=40).

Therefore, the methods and compositions described in this disclosure can identify SARS-CoV-2 strains or variants with high accuracy in a wide range of sample including environmental samples.

### EXAMPLE 5—Identifying Severe Acute Respiratory Syndrome Coronavirus 2 Variants in Clinical Samples

This example illustrates methods and compositions for identifying SARS-CoV-2 infection and variants in clinical samples using detection of SARS-CoV-2 variants using multiplex polymerase chain reaction coupled with high throughput sequencing of the amplification products described in this disclosure.

### Variant Samples

Heat-inactivated samples containing human lung epithelial cells infected with SARS-CoV-2 strains were obtained from the American Type Culture Collection (ATCC) and included the original wild type SARS-CoV-2 strain, G-clade variant, Alpha variant, Delta variant and Omicron variant. The G-clade variant can be characterized by D614G mutation. The Alpha variant can be characterized by Deletion 144 (Del144), N51Y, D614G mutation, or a combination thereof. The Delta variant can be characterized by G142D, Del157, Del158, L452R, T478K, D614G, or a combination thereof. The Omicron variant can be characterized by Del142, Del143, Del144, Y145D, Del211, L212I, K417N, N440K, G446S, S477N, T478K, E484A, Q493R, G496S, Q498R, N501Y, Y505H, D614G, or a combination thereof.

For library generation, purified RNA was reverse transcribed and amplified with primers of SEQ ID NOs. 6, 7, 11, 12, 14, 15, 27, and 28. to enrich target regions for variant determination. RT-PCR products were barcoded and pooled together to initiate the sequencing run on the Nanopore flow cell. A bioinformatic analysis pipeline was utilized to process sequencing data in real-time, which automatically can provide qualitative results for SARS-CoV-2 diagnosis and variant determination. A summary of the analysis results is given in Table 12 below.

TABLE 12
Identification of SARS-CoV-2 strains

| | SARS-CoV-2 | | | | SARS-CoV-2 |
|---|---|---|---|---|---|
| | hits (numbers | | | | variant |
| SARS- | of SARS-CoV- | | | | determined by |
| CoV-2 | 2 sequences | Alignment | | Sequencing | alignment and |
| variant | detected)* | length | Mutations | depth* | bioinformatics |
| Wild type | 9688 | 208 | — | — | Wild type |
| G clade | 9770 | 200 | D614G | 489x | G-clade (B.1) |
| Alpha | 9641 | 202 | Del144 | 2779x | Alpha (B.1.1.7) |
| | | | N501Y | 1572x | |
| | | | D614G | 531x | |
| Delta | 9674 | 205 | G142D | 2206x | Delta (B.1.617.2) |
| | | | Del157 | 2394x | |
| | | | Del158 | 2262x | |
| | | | L452R | 2821x | |
| | | | T478K | 2984x | |
| | | | D614G | 474x | |
| Omicron | 8233 | 185 | Del142 | 2367x | Omicron |
| | | | Del143 | 2384x | (B.1.1.529.1) |
| | | | Del144 | 2386x | |
| | | | Y145D | 671x | |
| | | | Del211 | 313x | |
| | | | L212I | 303x | |
| | | | K417N | 1459x | |
| | | | N440K | 1656x | |
| | | | G446S | 1611x | |
| | | | S477N | 1802x | |
| | | | T478K | 1736x | |
| | | | E484A | 1694x | |
| | | | Q493R | 1672x | |
| | | | G496S | 1552x | |
| | | | Q498R | 1511x | |
| | | | N501Y | 1344x | |
| | | | Y505H | 1515x | |
| | | | D614G | 479x | |

*number of SARS-CoV-2 hits and sequencing depth is calculated per 10,000 reads. Additional sequencing depth can be achieved by increasing sequencing time.

For the original wild type SARS-CoV-2 strain, 9688 sequencing reads were aligned to the SARS-CoV-2 genome with an average alignment length of 208 bp. Mutations were not detected for this sample. The analysis pipeline determined that the sample contains the wild type variant of the SARS-CoV-2 strain.

For the G-clade variant, 9770 reads were aligned to the SARS-CoV-2 genome with an average alignment length of 200 bp. The D614G mutation was detected with a sequencing depth of 489X. The analysis pipeline determined that the sample contains the G-clade (B.1) variant of SARS-CoV-2 strain.

For the Alpha variant, 9641 reads were aligned to the SARS-CoV-2 genome with an average alignment length of 202 bp. The Del144 deletion was detected with a sequencing depth of 2779X. The N501Y mutation was detected with a sequencing depth of 1572X. The D614G mutation was detected with a sequencing depth of 531X. The analysis pipeline determined that the sample contains the Alpha variant (B.1.1.7) variant of SARS-CoV-2 strain.

For the Delta variant, 9674 reads were aligned to the SARS-CoV-2 genome with an average alignment length of 205 bp. The G142D mutation was detected with a sequencing depth of 2206X. The Del157 deletion was detected with a sequencing depth of 2394x. The Del158 deletion was detected with a sequencing depth of 2262X. The L452R mutation was detected with a sequencing depth of 2821X. The T478K mutation was detected with a sequencing depth of 2984x. The D614G mutation was detected with a sequencing depth of 474X. The analysis pipeline determined that the sample contains the Delta variant (B.1.617.2) of SARS-CoV-2 strain.

For the Omicron variant, 8233 reads were aligned to the SARS-CoV-2 genome with an average alignment length of 185 bp. The Del142 deletion was detected with a sequencing depth of 2367X. The Del143 deletion was detected with a sequencing depth of 2384X. The Del144 mutation was detected with a sequencing depth of 2386X. The Y145D mutation was detected with a sequencing depth of 671X. The Del211 deletion was detected with a sequencing depth of 313X. The L212I mutation was detected with a sequencing depth of 303X. The K417N mutation was detected with a sequencing depth of 1459X. The N440K mutation was detected with a sequencing depth of 1656X. The G446S mutation was detected with a sequencing depth of 1611X. The S477N mutation was detected with a sequencing depth of 1802X. The T478K mutation was detected with a sequencing depth of 1736X. The E484A mutation was detected with a sequencing depth of 1694x. The Q493R mutation was detected with a sequencing depth of 1672x. The G496S mutation was detected with a sequencing depth of 1552X. The Q498R mutation was detected with a sequencing depth of 1511X. The N501Y mutation was detected with a sequencing depth of 1344X. The Y505H mutation was detected with a sequencing depth of 1515X. The D614G mutations was detected with a sequencing depth of 479X. The analysis pipeline determined that the sample contains the Omicron variant (B.1.1.529.1) of SARS-CoV-2.

### Clinical Evaluation

The clinical performance of the primers and methods described in this disclosure was evaluated in a clinical study using four remnant positive upper respiratory clinical samples (Positive 1-4) and 7 negative upper respiratory clinical samples (Negative 1-7) in viral transport medium. Of the 4 samples positive for SARS-CoV-2, 4/4 (100%) patient samples were detected to contain the Omicron BA.2 subvariant (B.1.1.529.2) of SARS-CoV-2. Of the 7 samples negative for SARS-CoV-2, an insufficient number of reads aligning to the SARS-CoV-2 genome (below 0.1% per 10,000 reads) was detected in 7/7 (100%) samples and they were determined to be negative for SARS-CoV-2. Results are summarized in Table 13 below.

TABLE 13
Identification of SARS-CoV-2 strains in clinical samples

| | SARS-CoV-2 | | | | |
|---|---|---|---|---|---|
| | sequence hits | | | | |
| | (numbers of | | | | |
| | SARS-CoV-2 | Alignment | | | |
| Clinical | sequences | length | | Sequencing | SARS-CoV-2 |
| sample | detected)* | (bp) | Mutations | depth* | Strain |
| Positive 1 | 9140 | 215 | G142D | 2207x | Omicron BA.2 |
| | | | V213G | 1286x | (B.1.1.529.2) |
| | | | K417N | 2379x | |
| | | | N440K | 2731x | |
| | | | S477N | 3007x | |
| | | | T478K | 3053x | |
| | | | E484A | 2887x | |
| | | | Q493R | 2666x | |
| | | | Q498R | 2410x | |
| | | | N501Y | 2174x | |
| | | | Y505H | 2318x | |
| | | | D614G | 482x | |
| Positive 2 | 5289 | 217 | G142D | 1399x | Omicron BA.2 |
| | | | V213G | 826x | (B.1.1.529.2) |
| | | | K417N | 1250x | |
| | | | N440K | 1460x | |
| | | | S477N | 1715x | |
| | | | T478K | 1734x | |
| | | | E484A | 1636x | |
| | | | Q493R | 1539x | |
| | | | Q498R | 1391x | |
| | | | N501Y | 1242x | |
| | | | Y505H | 1310x | |
| | | | D614G | 274x | |
| Positive 3 | 5614 | 207 | G142D | 1402x | Omicron BA.2 |
| | | | V213G | 851x | (B.1.1.529.2) |
| | | | K417N | 1239x | |
| | | | N440K | 1394x | |
| | | | S477N | 1549x | |
| | | | T478K | 1532x | |
| | | | E484A | 1487x | |
| | | | Q493R | 1395x | |
| | | | Q498R | 1242x | |
| | | | N501Y | 1123x | |
| | | | Y505H | 1186x | |
| | | | D614G | 372x | |
| Positive 4 | 6442 | 193 | G142D | 2319x | Omicron BA.2 |
| | | | V213G | 738x | (B.1.1.529.2) |
| | | | K417N | 876x | |
| | | | N440K | 921x | |
| | | | S477N | 1213x | |
| | | | T478K | 1165x | |
| | | | E484A | 1068x | |
| | | | Q493R | 1078x | |
| | | | Q498R | 964x | |
| | | | N501Y | 787x | |
| | | | Y505H | 950x | |
| | | | D614G | 311x | |
| Negative 1 | 4 | — | — | — | Negative |
| Negative 2 | 3 | — | — | — | Negative |
| Negative 3 | 4 | — | — | — | Negative |
| Negative 4 | 5 | — | — | — | Negative |
| Negative 5 | 6 | — | — | — | Negative |
| Negative 6 | 6 | — | — | — | Negative |
| Negative 7 | 4 | — | — | — | Negative |

*number of SARS-CoV-2 hits and sequencing depth is calculated per 10,000 reads. Additional sequencing depth can be achieved by increasing sequencing time.

Conclusively, all five variants including the original wild type coronavirus strain and the other prominent variants in nucleic acid samples or those present in the clinical samples were successfully identified by using the primers and methods described in this disclosure. The strain-defining mutations at enriched genome locations displayed sufficient sequencing depth and relative abundance over wild type for variant calling as determined by this analysis.

### EMBODIMENTS

Embodiment 1. A method for identifying sequences of a virus of family Coronaviridae in a sample, comprising:
a) providing substrate nucleic acids, wherein said substrate nucleic acids are amplification products of a plurality of template nucleotide sequences in said sample from said subject, or amplification products of complement sequences of said plurality of template nucleotide sequences; and
b) determining sequences of said substrate nucleic acids, wherein each of said plurality of template nucleotide sequences has a sequence aligning to a separate portion in a reference genome of said virus.

Embodiment 2. A method for identifying sequences of a virus of family Coronaviridae in a sample, comprising:
a) providing substrate nucleic acids, wherein said substrate nucleic acids are amplification products of a plurality of template nucleotide sequences in said sample from said subject, or amplification products of complement sequences of said plurality of template nucleotide sequences, wherein said plurality of template nucleotide sequences has less than about 1500 nucleotides in length in total; and
b) determining sequences of said substrate nucleic acids, wherein said plurality of template nucleotide sequences has sequences aligning to a Spike gene or a Nucleocapsid gene in a reference genome of said virus.

Embodiment 3. The method of embodiment 1 or 2, wherein said plurality of template nucleotide sequences has at least about 500 nucleotides in length in total.

Embodiment 4. The method of any one of embodiments 1-3, wherein said plurality of template nucleotide sequences has at least a sequence aligning to a Spike gene and at least a sequence aligning to a Nucleocapsid gene in a reference genome of said virus.

Embodiment 5. The method of any one of embodiments 1-4, wherein said providing comprises amplification reactions involving a plurality of primers.

Embodiment 6. The method of embodiment 5, wherein said plurality of primers comprises fewer than 50 primer pairs.

Embodiment 7. The method of embodiment 6, wherein said plurality of primers comprises fewer than 9 primer pairs.

Embodiment 8. The method of embodiment 7, wherein said plurality of primers comprises fewer than 5 primer pairs.

Embodiment 9. The method of embodiment 8, wherein said plurality of primers comprises 3 primer pairs.

Embodiment 10. The method of any one of embodiments 1-9, wherein said plurality of template nucleotide sequences comprises at most 50 template nucleotide sequences.

Embodiment 11. The method of embodiment 10, wherein said plurality of template nucleotide sequences comprises fewer than 9 template nucleotide sequences.

Embodiment 12. The method of embodiment 11, wherein said plurality of template nucleotide sequences comprises fewer than 5 template nucleotide sequences.

Embodiment 13. The method of embodiment 12, wherein said plurality of template nucleotide sequences comprises 3 template nucleotide sequences.

Embodiment 14. A method for analyzing a sample, comprising:
a) providing substrate nucleic acids by performing amplification reactions involving primers that comprise at least a nucleotide sequence having at least about 85% identity to a sequence selected from the group consisting of SEQ ID NOs: 1-28.

Embodiment 15. The method of any one of embodiments 5-14, wherein said primers comprise at least a nucleotide sequence comprising at least about 86% sequence identity to a sequence selected from the group consisting of SEQ ID NOs: 1-28.

Embodiment 16. The method of embodiment 15, wherein said primers comprise at least a nucleotide sequence comprising at least about 90% sequence identity to a sequence selected from the group consisting of SEQ ID NOs: 1-28.

Embodiment 17. The method of embodiment 16, wherein said primers comprise at least a nucleotide sequence comprising at least about 94% sequence identity to a sequence selected from the group consisting of SEQ ID NOs: 1-28.

Embodiment 18. The method of embodiment 17, wherein said primers comprise at least a nucleotide sequence comprising a sequence selected from the group consisting of SEQ ID NOs: 1-28.

Embodiment 19. The method of embodiment 14, wherein said plurality of primers comprises at least a nucleotide sequence comprising a sequence selected from the group consisting of SEQ ID NOs: 1-8 and 13-21, and 26-28.

Embodiment 20. The method of embodiment 19, wherein said plurality of primers comprises at least a nucleotide sequence comprising a sequence selected from the group consisting of SEQ ID NOs: 1, 3, 4, 6, 13, 14, 17, 18, 20, and 27.

Embodiment 21. The method of embodiment 19, wherein said plurality of primers comprises at least a nucleotide sequence comprising a sequence selected from the group consisting of SEQ ID NOs: 2, 5, 7, 8, 15, 16, 19, 21, 26, and 28.

Embodiment 22. The method of embodiment 19, wherein said plurality of primers comprises at least a nucleotide sequence comprising a sequence selected from the group consisting of SEQ ID NOs: 1, 3, 4, 6, 13, 14, 17, 18, 20, and 27; and at least a nucleotide sequence comprising a sequence selected from the group consisting of SEQ ID NOs: 2, 5, 7, 8, 15, 16, 19, 21, 26, and 28.

Embodiment 23. The method of embodiment 14, wherein said plurality of primers comprises at least a nucleotide sequence comprising a sequence selected from the group consisting of SEQ ID NOs: 9-12 and 22-25.

Embodiment 24. The method of embodiment 23, wherein said plurality of primers comprises at least a nucleotide sequence comprising a sequence selected from the group consisting of SEQ ID NOs: 9, 11, 22, and 24. The method of embodiment 23, wherein said plurality of primers comprises at least a nucleotide sequence comprising a sequence selected from the group consisting of SEQ ID NOs: 10, 12, 23, and 25.

Embodiment 25. The method of embodiment 23, wherein said plurality of primers comprises at least a nucleotide sequence comprising a sequence selected from the group consisting of SEQ ID NOs: 9, 11, 22, and 24; and at least a nucleotide sequence comprising a sequence selected from the group consisting of SEQ ID NOs: 10, 12, 23, and 25.

Embodiment 26. The method of any one of embodiments 19-25, wherein said plurality of primers comprises at least a nucleotide sequence comprising a sequence selected from the group consisting of SEQ ID NOs: 1, 3, 4, 6, 13, 14, 17, 18, 20, and 27; at least a nucleotide sequence comprising a sequence selected from the group consisting of SEQ ID NOs: 2, 5, 7, 8, 15, 16, 19, 21, 26, and 28; at least a nucleotide sequence comprising a sequence selected from the group consisting of SEQ ID NOs: 9, 11, 22, and 24; and at least a nucleotide sequence comprising a sequence selected from the group consisting of SEQ ID NOs: 10, 12, 23, and 25.

Embodiment 27. The method of embodiment 14, wherein said plurality of primers comprises a first primer pair selected from the group consisting of:
a) SEQ ID NOs: 1 and 2,
b) SEQ ID NOs: 3 and 19,
c) SEQ ID NOs: 4 and 15,
d) SEQ ID NOs: 5 and 14,
e) SEQ ID NOs: 6 and 7,
f) SEQ ID NOs 13 and 26,
g) SEQ ID NOs: 16 and 18,
h) SEQ ID NOs: 20 and 21, and
i) SEQ ID NOs: 27 and 28.

Embodiment 28. The method of embodiment 27, wherein said plurality of primers further comprises a second primer pair selected from the group consisting of:
a) SEQ ID NOs: 9 and 10,
b) SEQ ID NOs: 11 and 12,
c) SEQ ID NOs: 22 and 23, and
d) SEQ ID NOs: 24 and 25.

Embodiment 29. The method of any one of embodiments 14-28, further comprising b) determining sequences of said substrate nucleic acids.

Embodiment 30. The method of any one of embodiments 1-29, further comprising c) providing a report, wherein said report comprises a determination of a presence or an absence of sequences of said virus or a variant of said virus in said sample.

Embodiment 31. The method of any one of embodiments 1-30, wherein said providing comprises an amplification reaction.

Embodiment 32. The method of embodiment 31, wherein said amplification reaction comprises a reverse transcription, an asymmetric amplification, a helicase-dependent amplification (HDA), a ligase chain reaction (LCR), a loop mediated isothermal amplification (LAMP), a multiple displacement amplification (MDA), a nucleic acid sequence based amplification (NASBA), a polymerase chain reaction (PCR), a primer extension, a recombinase polymerase amplification (RPA), a rolling circle amplification (RCA), a self-sustained sequence replication (3SR), or a strand displacement amplification (SDA).

Embodiment 33. The method of embodiment 32, wherein said amplification reaction comprises said PCR.

Embodiment 34. The method of embodiment 33, wherein said PCR comprises a multiplex PCR.

Embodiment 35. The method of any one of embodiments 1-34, wherein said providing further comprises a reverse transcription.

Embodiment 36. The method of any one of embodiments 1-35, wherein said providing comprises a barcoding reaction.

Embodiment 37. The method of any one of embodiments 1-13 or 29-36, wherein said determining comprises sequencing said substrate nucleic acids, or amplification products thereof.

Embodiment 38. The method of embodiment 37, wherein said sequencing comprises a chain termination sequencing, a high-throughput sequencing, a mass spectrophotometry sequencing, a massively parallel signature sequencing, a Maxam-Gilbert sequencing, a nanopore sequencing, a primer walking, a pyrosequencing, a Sanger sequencing, a semiconductor sequencing, a sequencing-by-hybridization, a sequencing-by-ligation, a sequencing-by-synthesis, a single-molecule sequencing, or a shotgun sequencing.

Embodiment 39. The method of embodiment 38, wherein said sequencing comprises said nanopore sequencing.

Embodiment 40. The method of any one of embodiments 1-39, wherein said virus from family Coronaviridae comprises a virus of subfamily Orthocoronavirinae.

Embodiment 41. The method of any one of embodiments 1-40, wherein said virus from family Coronaviridae comprises a virus of species coronavirus.

Embodiment 42. The method of any one of embodiments 1-41, wherein said virus from family Coronaviridae comprises a Severe acute respiratory syndrome-related coronavirus.

Embodiment 43. The method of any one of embodiments 1-42, wherein said virus from family Coronaviridae comprises a Severe acute respiratory syndrome coronavirus 2.

Embodiment 44. The method of any one of embodiments 1-43, wherein said sample is isolated from an animal, water, a surface, droplets or a food.

Embodiment 45. The method of embodiment 44, wherein said water comprises sewage water or drinking water.

Embodiment 46. The method of embodiment 45, wherein said water comprises said sewage water.

Embodiment 47. The method of embodiment 44, wherein said surface comprises a surface of an indoor compartment, a surface of a food packaging material, a surface of a mask, a surface of a medical equipment, or a surface of a furniture.

Embodiment 48. The method of embodiment 47, wherein said surface comprises a surface of said indoor compartment.

Embodiment 49. The method of embodiment 48, wherein said surface of said indoor compartment comprises a surface of an elevator.

Embodiment 50. The method of embodiment 49, wherein said surface of said elevator comprises a button of said elevator.

Embodiment 51. The method of embodiment 44, where said surface comprises a surface of a metal, a surface of a wood, a surface of a plastic, a surface of a paper, a surface of a glass, a surface of a ceramic, a surface of a fabric, or a surface of a shoe.

Embodiment 52. The method of embodiment 51, wherein said surface comprises a surface of said metal.

Embodiment 53. The method of embodiment 52, wherein said surface of said metal comprises a surface of a door handle or a doorknob.

Embodiment 54. The method of any one of embodiments 1-53, wherein said sample comprises a biological sample.

Embodiment 55. The method of embodiment 54, wherein said biological sample is from said subject.

Embodiment 56. The method of embodiment 55, wherein said subject is a human.

Embodiment 57. The method of any one of embodiments 54-56, wherein said biological sample comprises a blood sample, a tissue sample, a nasal swab sample, or an anal swab sample.

Embodiment 58. The method of embodiment 57, wherein said tissue sample may comprise a body fluid sample, a muscle sample, a skin sample, a lung sample, a brain sample, a nervous system sample, a gastrointestinal sample, a hair sample, or a skin sample.

Embodiment 59. The method of any one of embodiments 54-58, wherein said biological sample may be obtained by a biopsy.

Embodiment 60. The method of embodiment 59, wherein said biopsy may comprise a blood biopsy, a body fluid biopsy, a muscle biopsy, a skin biopsy, a lung biopsy, a brain biopsy, a nervous system biopsy, a gastrointestinal biopsy, or a skin biopsy.

Embodiment 61. The method of embodiment 59, wherein said biopsy may comprise a fine needle aspiration biopsy, a core needle biopsy, a vacuum-assisted biopsy, an excisional biopsy, a shave biopsy, a punch biopsy, an endoscopic biopsy, a laparoscopic biopsy, a bone marrow aspiration biopsy, a liquid biopsy.

Embodiment 62. The method of embodiment 61, wherein said biopsy may comprise an incisional biopsy or an excisional biopsy.

Embodiment 63. The method of any one of embodiments 1-62, wherein said plurality of template nucleotide sequences comprises at least a sequence corresponding to a region spanning across nucleotide positions 331 to 670, 1027 to 1306, 1211 to 1571, 1424 to 1739, 1435 to 1972, 1710 to 1915, 1726 to 1865, 1997 to 2250, 2567 to 2642, or 3149 to 3723 of SEQ ID NO. 31.

Embodiment 64. The method of any one of embodiments 1-63, wherein said plurality of template nucleotide sequences comprises at least a sequence that encodes a peptide having at least about 70% identity to a sequence of SEQ ID NO. 29.

Embodiment 65. The method of embodiment 64, further comprising determining nucleotides at a region of at least one sequence of said sequences of said substrate nucleic acids that corresponds to a region of SEQ ID NO: 31 that encodes an amino acid at any one of positions 142, 143, 144, 145, 157, 158, 211, 212, 417, 440, 446, 452, 477, 478, 484, 493, 496, 498, 501, 505, 614, or any combination thereof, of SEQ ID NO. 29.

Embodiment 66. The method of embodiment 65, wherein the method further comprises: when said at least one sequence comprises a variation relative to SEQ ID NO: 31,
a) determining said virus of family Coronaviridae in said sample is a G-clade variant of Severe acute respiratory syndrome coronavirus 2 (SARS-CoV-2), when said variation comprises nucleotides that encode a Glycine (G) at a position corresponding to position 614 of said sequence of SEQ ID NO. 29;
b) determining said virus of family Coronaviridae in said sample is an Alpha variant of SARS-CoV-2, when said variation consists of a deletion of nucleotides that encode an amino acid at a position corresponding to position 144 of said sequence of SEQ ID NO: 29, nucleotides that encode a Tyrosine (Y) at a position corresponding to position 501 of said sequence of SEQ ID NO: 29, and nucleotides that encode a G at a position corresponding to position 614 of said sequence of SEQ ID NO: 29;
c) determining said virus of family Coronaviridae in said sample is a Delta variant of SARS-CoV-2, when said variation consists of nucleotides that encode a Aspartic acid (D) at a position corresponding to position 142 of said sequence of SEQ ID NO: 29, a deletion of nucleotides that encode an amino acid at a position corresponding to position 157 of said sequence of SEQ ID NO: 29, a deletion of nucleotides that encode an amino acid at a position corresponding to position 158 of said sequence of SEQ ID NO: 29, nucleotides that encode an Arginine (R) at a position corresponding to position 452 of SEQ ID NO: 29, nucleotides that encode an Lysine (K) at a position corresponding to position 478 of said sequence of SEQ ID NO: 29, and nucleotides that encode a G at a position corresponding to position 614 of said sequence of SEQ ID NO: 29; or
d) determining said virus of family Coronaviridae in said sample is an Omicron variant of SARS-CoV-2, when said variation consists of a deletion of nucleotides that encode an amino acid at a position corresponding to position 42 of said sequence of SEQ ID NO: 29, a deletion of nucleotides that encode an amino acid at a position corresponding to position 43 of said sequence of SEQ ID NO: 29, a deletion of nucleotides that encode an amino acid at a position corresponding to position 44 of said sequence of SEQ ID NO: 29, nucleotides that encode a D at a position corresponding to position 145 of said sequence of SEQ ID NO: 29, a deletion of nucleotides that encode an amino acid at a position corresponding to position 211 of said sequence of SEQ ID NO: 29, nucleotides that encode an Isoleucine (I) at a position corresponding to position 212 of said sequence of SEQ ID NO: 29, nucleotides that encode an Asparagine (N) at a position corresponding to position 417 of said sequence of SEQ ID NO: 29, nucleotides that encode a K at a position corresponding to position 440 of said sequence of SEQ ID NO: 29, nucleotides that encode a Serine (S) at a position corresponding to position 446 of said sequence of SEQ ID NO: 29, nucleotides that encode an N at a position corresponding to position 477 of said sequence of SEQ ID NO: 29, nucleotides that encode a K at a position corresponding to position 478 of said sequence of SEQ ID NO: 29, nucleotides that encode an Alanine (A) at a position corresponding to position 484 of said sequence of SEQ ID NO: 29, nucleotides that encode an R at a position corresponding to position 493 of said sequence of SEQ ID NO: 29, nucleotides that encode a S at a position corresponding to position 496 of said sequence of SEQ ID NO: 29, nucleotides that encode an R at a position corresponding to position 498 of said sequence of SEQ ID NO: 29, nucleotides that encode a Y at a position corresponding to position 501 of said sequence of SEQ ID NO: 29, nucleotides that encode a Histidine (H) at a position corresponding to position 505 of said sequence of SEQ ID NO: 29, and nucleotides that encode a G at a position corresponding to said position 614 of said sequence of SEQ ID NO: 29.

Embodiment 67. A composition comprising a mixture of a plurality of primers, wherein said plurality of primers is configured to generate substrate nucleic acids, wherein said substrate nucleic acids are amplification products of a plurality of template nucleotide sequences, or amplification products of complement sequences of said plurality of template nucleotide sequences, and wherein each of said plurality of template nucleotide sequences has a sequence aligning to a separate portion in a reference genome of a virus of family Coronaviridae.

Embodiment 68. A composition comprising a mixture of a plurality of primers, wherein said plurality of primers is configured to generate substrate nucleic acids, wherein said substrate nucleic acids are amplification products of a plurality of template nucleotide sequences, or amplification products of complement sequences of said plurality of template nucleotide sequences, wherein said plurality of template nucleotide sequences has less than about 1500 nucleotides in length in total, and wherein each of said plurality of template nucleotide sequences has a sequence aligning to a separate portion in a reference genome of a virus of family Coronaviridae.

Embodiment 69. The composition of embodiment 67 or 68, wherein said plurality of primers comprises fewer than 50 primer pairs.

Embodiment 70. The composition of embodiment 69, wherein said plurality of primers comprises fewer than 9 primer pairs.

Embodiment 71. The composition of embodiment 70, wherein said plurality of primers comprises fewer than 5 primer pairs.

Embodiment 72. The composition of embodiment 71, wherein said plurality of primers comprises 3 primer pairs.

Embodiment 73. The composition of any one of embodiments 67-72, wherein said plurality of primers comprises a primer comprising at least a nucleotide sequence comprising at least about 85% sequence identity to a sequence of a virus of subfamily Orthocoronavirinae.

Embodiment 74. The composition of any one of embodiments 67-73, wherein said plurality of primers comprises a primer comprising at least a nucleotide sequence comprising at least about 85% sequence identity to a sequence of a virus of species coronavirus.

Embodiment 75. The composition of any one of embodiments 67-74, wherein said plurality of primers comprises a primer comprising at least a nucleotide sequence comprising at least about 85% sequence identity to a sequence of Severe acute respiratory syndrome-related coronavirus.

Embodiment 76. The composition of any one of embodiments 67-75, wherein said plurality of primers comprises a primer comprising at least a nucleotide sequence comprising at least about 85% sequence identity to a sequence of a Severe acute respiratory syndrome coronavirus 2.

Embodiment 77. A composition comprising: a mixture of a plurality of primers, wherein said plurality of primers comprises at least two nucleotide sequences comprising at least about 85% sequence identity to two sequences selected from the group consisting of SEQ ID NOs: 1-28.

Embodiment 78. The composition of any one of embodiments 67-77, wherein said plurality of primers comprises at least two nucleotide sequences comprising at least about 86% sequence identity to two sequences selected from the group consisting of SEQ ID NOs: 1-28.

Embodiment 79. The composition of any one of embodiments 67-78, wherein said plurality of primers comprises at least two nucleotide sequences comprising at least about 90% sequence identity to two sequences selected from the group consisting of SEQ ID NOs: 1-28.

Embodiment 80. The composition of any one of embodiments 67-79, wherein said plurality of primers comprises at least two nucleotide sequences comprising at least about 94% sequence identity to two sequences selected from the group consisting of SEQ ID NOs: 1-28.

Embodiment 81. The composition of any one of embodiments 67-80, wherein said plurality of primers comprises at least two nucleotide sequences selected from the group consisting of SEQ ID NOs: 1-28.

Embodiment 82. The composition of any one of embodiments 67-81, wherein said plurality of primers comprises at least four nucleotide sequences comprising at least about 85% sequence identity to four sequences selected from the group consisting of SEQ ID NOs: 1-28.

Embodiment 83. The composition of any one of embodiments 67-82, wherein said plurality of primers comprises at least four nucleotide sequences comprising at least about 86% sequence identity to four sequences selected from the group consisting of SEQ ID NOs: 1-28.

Embodiment 84. The composition of any one of embodiments 67-83, wherein said plurality of primers comprises at least four nucleotide sequences comprising at least about 90% sequence identity to four sequences selected from the group consisting of SEQ ID NOs: 1-28.

Embodiment 85. The composition of any one of embodiments 67-84, wherein said plurality of primers comprises at least four nucleotide sequences comprising at least about 94% sequence identity to four sequences selected from the group consisting of SEQ ID NOs: 1-28.

Embodiment 86. The composition of any one of embodiments 67-85, wherein said plurality of primers comprises at least four nucleotide sequences selected from the group consisting of SEQ ID NOs: 1-28.

Embodiment 87. The composition of any one of embodiments 67-86, wherein said plurality of primers comprises at least six nucleotide sequences comprising at least about 85% sequence identity to the group consisting of SEQ ID NOs: 1-28.

Embodiment 88. The composition of any one of embodiments 67-87, wherein said plurality of primers comprises at least six nucleotide sequences comprising at least about 86% sequence identity to the group consisting of SEQ ID NOs: 1-28.

Embodiment 89. The composition of any one of embodiments 67-88, wherein said plurality of primers comprises at least six nucleotide sequences comprising at least about 90% sequence identity to the group consisting of SEQ ID NOs: 1-28.

Embodiment 90. The composition of any one of embodiments 67-89, wherein said plurality of primers comprises at least six nucleotide sequences comprising at least about 94% sequence identity to the group consisting of SEQ ID NOs: 1-28.

Embodiment 91. The composition of any one of embodiments 67-90, wherein said plurality of primers comprises at least six nucleotide sequences selected from the group consisting of SEQ ID NOs: 1-28.

Embodiment 92. The composition of any one of embodiments 67-91, wherein said plurality of primers comprises at least a nucleotide sequence comprising a sequence selected from the group consisting of SEQ ID NOs: 1, 3, 4, 6, 13, 14, 17, 18, 20, and 27.

Embodiment 93. The composition of any one of embodiments 67-92, wherein said plurality of primers comprises at least a nucleotide sequence comprising a sequence selected from the group consisting of SEQ ID NOs: 2, 5, 7, 8, 15, 16, 19, 21, 26, and 28.

Embodiment 94. The composition of any one of embodiments 67-93, wherein said plurality of primers comprises at least a nucleotide sequence comprising a sequence selected from the group consisting of SEQ ID NOs: 1, 3, 4, 6, 13, 14, 17, 18, 20, and 27; and at least a nucleotide sequence comprising a sequence selected from the group consisting of SEQ ID NOs: 2, 5, 7, 8, 15, 16, 19, 21, 26, and 28.

Embodiment 95. The composition of any one of embodiments 67-94, wherein said plurality of primers comprises at least a nucleotide sequence comprising a sequence selected from the group consisting of SEQ ID NOs: 9, 11, 22, and 24.

Embodiment 96. The composition of any one of embodiments 67-95, wherein said plurality of primers comprises at least a nucleotide sequence comprising a sequence selected from the group consisting of SEQ ID NOs: 10, 12, 23, and 25.

Embodiment 97. The composition of any one of embodiments 67-96, wherein said plurality of primers comprises at least a nucleotide sequence comprising a sequence selected from the group consisting of SEQ ID NOs: 9, 11, 22, and 24; and at least a nucleotide sequence comprising a sequence selected from the group consisting of SEQ ID NOs: 10, 12, 23, and 25.

Embodiment 98. The composition of any one of embodiments 67-97, wherein said plurality of primers comprises at least a nucleotide sequence comprising a sequence selected from the group consisting of SEQ ID NOs: 1, 3, 4, 6, 13, 14, 17, 18, 20, and 27; at least a nucleotide sequence comprising a sequence selected from the group consisting of SEQ ID NOs: 2, 5, 7, 8, 15, 16, 19, 21, 26, and 28; at least a nucleotide sequence comprising a sequence selected from the group consisting of SEQ ID NOs: 9, 11, 22, and 24; and at least a nucleotide sequence comprising a sequence selected from the group consisting of SEQ ID NOs: 10, 12, 23, and 25.

Embodiment 99. The combination of any one of embodiments 67-98, wherein said plurality of primers comprises a first primer pair selected from the group consisting of:
a) SEQ ID NOs: 1 and 2,
b) SEQ ID NOs: 3 and 19,
c) SEQ ID NOs: 4 and 15,
d) SEQ ID NOs: 5 and 14,
e) SEQ ID NOs: 6 and 7,
f) SEQ ID NOs: 13 and 26,
g) SEQ ID NOs: 16 and 18,
h) SEQ ID NOs: 20 and 21, and
i) SEQ ID NOs: 27 and 28.

Embodiment 100. The combination of any one of embodiments 67-99, wherein said plurality of primers further comprises a second primer pair selected from the group consisting of:
a) SEQ ID NOs: 9 and 10,
b) SEQ ID NOs: 11 and 12,
c) SEQ ID NOs: 22 and 23, and
d) SEQ ID NOs: 24 and 25.

Embodiment 101. The composition of any one of embodiments 67-100, wherein said plurality of primers is configured to generate substrate nucleic acids.

Embodiment 102. The composition of any one of embodiments 67-101, wherein said plurality of primers is configured to generate said substrate nucleic acids via an amplification reaction.

Embodiment 103. The composition of embodiment 102, wherein said amplification reaction comprises an asymmetric amplification, a helicase-dependent amplification (HDA), a ligase chain reaction (LCR), a loop mediated isothermal amplification (LAMP), a multiple displacement amplification (MDA), a nucleic acid sequence based amplification (NASBA), a polymerase chain reaction (PCR), a primer extension, a recombinase polymerase amplification (RPA), a rolling circle amplification (RCA), a self-sustained sequence replication (3SR), or a strand displacement amplification (SDA).

Embodiment 104. The composition of embodiment 103, wherein said amplification reaction comprises said PCR.

Embodiment 105. The composition of embodiment 104, wherein said PCR comprises a multiplex PCR.

Embodiment 106. A kit, comprising said composition of any one of embodiments 67-105.

Embodiment 107. The kit of any one of embodiments 67-106, further comprising an oligonucleotide that is configured to bind RNAs.

Embodiment 108. The kit of embodiment 107, wherein said oligonucleotide is configured to bind messenger RNAs (mRNAs).

Embodiment 109. The kit of embodiment 107 or 108, wherein said oligonucleotide is attached to a solid support.

Embodiment 110. The kit of embodiment 109, wherein said solid support is a magnetic bead.

Embodiment 111. The kit of any one of embodiments 106-110, further comprising a barcoding probe.

Embodiment 112. The kit of any one of embodiments 106-111, further comprising a sequencing probe.

Embodiment 113. The kit of embodiment 112, wherein said sequencing probe is configured to facilitate sequencing said substrate nucleic acids in a sequencing reaction.

Embodiment 114. The kit of embodiment 113, wherein said sequencing reaction comprises a chain termination sequencing, a high-throughput sequencing, a mass spectrophotometry sequencing, a massively parallel signature sequencing, a Maxam-Gilbert sequencing, a nanopore sequencing, a primer walking, a pyrosequencing, a Sanger sequencing, a semiconductor sequencing, a sequencing-by-hybridization, a sequencing-by-ligation, a sequencing-by-synthesis, a single-molecule sequencing, or a shotgun sequencing.

Embodiment 115. The kit of embodiment 114, wherein said sequencing reaction comprises said nanopore sequencing.

While preferred embodiments of the present disclosure have been shown and described herein, it will be obvious to those skilled in the art that such embodiments are provided by way of example only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein may be employed in practicing the present disclosure. It is therefore contemplated that the present disclosure shall also cover any such alternatives, modifications, variations or equivalents. It is intended that the following claims define the scope of the present disclosure and that methods and structures within the scope of these claims and their equivalents be covered thereby.

1.-115. (canceled)

116. A method for identifying sequences of a virus of family Coronaviridae in a sample, comprising:
a) providing substrate nucleic acids, wherein said substrate nucleic acids are amplification products of a plurality of template nucleotide sequences in said sample, or amplification products of complement sequences of said plurality of template nucleotide sequences; and
b) determining sequences of said substrate nucleic acids,
wherein each of said plurality of template nucleotide sequences has a sequence aligning to a separate portion in a reference genome of said virus.

117. The method of claim 116, wherein said plurality of template nucleotide sequences has less than about 1500 nucleotides in length in total.

118. The method of claim 116, wherein said plurality of template nucleotide sequences has sequences aligning to a Spike gene or a Nucleocapsid gene in a reference genome of said virus.

119. The method of claim 116, wherein said providing comprises a amplification reaction of said plurality of template nucleotide sequences.

120. The method of claim 119, wherein said amplification comprises a plurality of primers, and wherein said plurality of primers comprises fewer than 50 primer pairs.

121. The method of claim 119, wherein said amplification comprises a plurality of primers, and wherein said plurality of primers comprises at least a nucleotide sequence having at least about 85% identity to a sequence selected from the group consisting of SEQ ID NOs: 1-28.

122. The method of claim 119, wherein said amplification reaction comprises a reverse transcription, an asymmetric amplification, a helicase-dependent amplification (HDA), a ligase chain reaction (LCR), a loop mediated isothermal amplification (LAMP), a multiple displacement amplification (MDA), a nucleic acid sequence based amplification (NASBA), a polymerase chain reaction (PCR), a primer extension, a recombinase polymerase amplification (RPA), a rolling circle amplification (RCA), a self-sustained sequence replication (3SR), or a strand displacement amplification (SDA).

123. The method of claim 119, wherein said amplification reaction comprises PCR, and wherein said PCR comprises a multiplex PCR.

124. The method of claim 116, wherein said providing further comprises a reverse transcription or a barcoding reaction.

125. The method of claim 116, wherein said determining comprises sequencing said substrate nucleic acids, or amplification products thereof.

126. The method of claim 125, wherein said sequencing comprises a chain termination sequencing, a high-throughput sequencing, a mass spectrophotometry sequencing, a massively parallel signature sequencing, a Maxam-Gilbert sequencing, a nanopore sequencing, a primer walking, a pyrosequencing, a Sanger sequencing, a semiconductor sequencing, a sequencing-by-hybridization, a sequencing-by-ligation, a sequencing-by-synthesis, a single-molecule sequencing, or a shotgun sequencing.

127. The method of claim 126, wherein said sequencing comprises said nanopore sequencing.

128. The method of claim 116, wherein said sample is isolated from an animal, water, a surface, droplets or a food.

129. The method of claim 116, wherein said sample is isolated from sewage water or drinking water.

130. The method of claim 116, wherein said sample is isolated from a surface of an indoor compartment, a surface of a food packaging material, a surface of a mask, a surface of a medical equipment, or a surface of a furniture.

131. The method of claim 116, where said sample is isolated from a surface of a metal, a surface of a wood, a surface of a plastic, a surface of a paper, a surface of a glass, a surface of a ceramic, a surface of a fabric, or a surface of a shoe.

132. The method of claim 116, wherein said sample comprises a biological sample of a human.

133. The method of claim 132, wherein said biological sample comprises a blood sample, a tissue sample, a nasal swab sample, an anal swab sample, or a biopsy sample.

134. A composition comprising a mixture of a plurality of primers,
wherein said plurality of primers is configured to generate substrate nucleic acids,
wherein said substrate nucleic acids are amplification products of a plurality of template nucleotide sequences, or amplification products of complement sequences of said plurality of template nucleotide sequences, and
wherein each of said plurality of template nucleotide sequences has a sequence aligning to a separate portion in a reference genome of a virus of family Coronaviridae.

135. A kit comprising a mixture of a plurality of primers,
wherein said plurality of primers is configured to generate substrate nucleic acids,
wherein said substrate nucleic acids are amplification products of a plurality of template nucleotide sequences, or amplification products of complement sequences of said plurality of template nucleotide sequences, and
wherein each of said plurality of template nucleotide sequences has a sequence aligning to a separate portion in a reference genome of a virus of family Coronaviridae.
